SEQUENCE LISTING (616 sequences)

SEQ ID NO: 1 - protein, 30 aa, Llama glama
QVQLVESGGGLVQPGGSLRLSCAASGFTFS

SEQ ID NO: 2 - protein, 30 aa, Llama glama
EVQVQESGGGLVQPGGSLRLSCAASGFTFS

SEQ ID NO: 3 - protein, 30 aa, Llama glama
EVQLQESGGGLVQPGGSLRLSCAASGFTFD

SEQ ID NO: 4 - protein, 30 aa, Llama glama
QLQLVESGGGLVQPGGSLRLSCAASGFTFD

SEQ ID NO: 5 - protein, 30 aa, Llama glama
QVQLQESGPGLVKPSQTLSLTCTVSGGSIT

SEQ ID NO: 6 - protein, 30 aa, Llama glama
EVQLQESGPGLVKPSQTLSLTCTVSGGSIT

SEQ ID NO: 7 - protein, 30 aa, Llama glama
EVQLVESGGGLVQPGGTLRLSCAASGFTFS

SEQ ID NO: 8 - protein, 5 aa, Llama glama
SYWMY

SEQ ID NO: 9 - protein, 5 aa, Llama glama
DYAMS

SEQ ID NO: 10 - protein, 7 aa, Llama glama
TSFYAWS

SEQ ID NO: 11 - protein, 5 aa, Llama glama
NYGWS

SEQ ID NO: 12 - protein, 7 aa, Llama glama
NSYYASS

SEQ ID NO: 13 - protein, 5 aa, Llama glama
NYWMH

SEQ ID NO: 14 - protein, 14 aa, Llama glama
WVRQAPGKGLEWVS

SEQ ID NO: 15 - protein, 14 aa, Llama glama
WIRQPPGKGLEWMG

SEQ ID NO: 16 - protein, 17 aa, Llama glama
AINTGGGSTYYADSVKG

SEQ ID NO: 17 - protein, 17 aa, Llama glama
AISWNGGSTYYAESMKG

SEQ ID NO: 18 - protein, 16 aa, Llama glama
VIAYDGRTFYSPSLKS

SEQ ID NO: 19 - protein, 16 aa, Llama glama
VIAYDGSTYYSPSLKS

SEQ ID NO: 20 - protein, 16 aa, Llama glama
VIIYDGSTYYSPSLKS

SEQ ID NO: 21 - protein, 17 aa, Llama glama
VIDRGGGGTRYADSVKG

SEQ ID NO: 22 - protein, 32 aa, Llama glama
RFTISRDNAKNTLYLQMNSLKPEDTALYYCAR

SEQ ID NO: 23 - protein, 32 aa, Llama glama
RFTISRDNAKNTLYLQMNSLKSKDTAVYYCAR

SEQ ID NO: 24 - protein, 32 aa, Llama glama
RTHISRDTSKNQFSLQLSSVTPEDTAVYYCAR

SEQ ID NO: 25 - protein, 32 aa, Llama glama
RTSISRDTSKNQFSLQLSSVTPEDTAVYYCAS

SEQ ID NO: 26 - protein, 32 aa, Llama glama
RTSISRDTSKNQFSLQLSSVTPEDTAVYYCAR

SEQ ID NO: 27 - protein, 32 aa, Llama glama
RFTISRDNAKNTLYLRMNSLKPEDTALYYCTR

SEQ ID NO: 28 - protein, 14 aa, Llama glama
GPGYSGSYIEGWAY

SEQ ID NO: 29 - protein, 14 aa, Llama glama
GPGYSGKYIEGWAY

SEQ ID NO: 30 - protein, 11 aa, Llama glama
RRYGLGSEYDY

SEQ ID NO: 31 - protein, 10 aa, Llama glama
GGVVAWSYDY

SEQ ID NO: 32 - protein, 15 aa, Llama glama
AMVTTSSPWVKTFGA

SEQ ID NO: 33 - protein, 14 aa, Llama glama
GLRVAGSYFTDFGT

SEQ ID NO: 34 - protein, 8 aa, Llama glama
DRNGEYDY

SEQ ID NO: 35 - protein, 11 aa, Llama glama
WGQGTQVTVSS

SEQ ID NO: 36 - protein, 11 aa, Llama glama
WGRGTQVTVSS

SEQ ID NO: 37 - protein, 23 aa, Llama glama
DIVMTQSPTSVTASVGEKVTINC

SEQ ID NO: 38 - protein, 23 aa, Llama glama
DIVMTQSPRSVTASVGEKVFINC

SEQ ID NO: 39 - protein, 23 aa, Llama glama
DIVMTQSPSSVTASVGEKVTINC

SEQ ID NO: 40 - protein, 22 aa, Llama glama
NFMLTQPSAVSVSLGQTARITC

SEQ ID NO: 41 - protein, 22 aa, Llama glama
HSAVTQPSAVSVSLGQTARITC

SEQ ID NO: 42 - protein, 22 aa, Llama glama
SYELTQSPSVSVALRQTAKITC

SEQ ID NO: 43 - protein, 22 aa, Llama glama
QAGLTQPPSVSGSPGKTVTISC

SEQ ID NO: 44 - protein, 22 aa, Llama glama
SYELTQSPSVSVALRQAAKITC

SEQ ID NO: 45 - protein, 23 aa, Llama glama
DIQMTQSPSSVTASAGEKVTINC

SEQ ID NO: 46 - protein, 17 aa, Llama glama
KSSQSVVSGSNQKSYLN

SEQ ID NO: 47 - protein, 17 aa, Llama glama
KSSQSVVSESNQRSYLN

SEQ ID NO: 48 - protein, 17 aa, Llama glama
KSSQSVVSGSKQKSYLN

SEQ ID NO: 49 - protein, 11 aa, Llama glama
QGSSFGSSYAH

SEQ ID NO: 50 - protein, 11 aa, Llama glama
QGTNLRSSYVH

SEQ ID NO: 51 - protein, 11 aa, Llama glama
GGNDIGSKSTQ

SEQ ID NO: 52 - protein, 14 aa, Llama glama
AGTSSDVGYGNYVS

SEQ ID NO: 53 - protein, 11 aa, Llama glama
GGDNIASKHAH

SEQ ID NO: 54 - protein, 17 aa, Llama glama
KSSQSVLYSSNQKNYLA

SEQ ID NO: 55 - protein, 15 aa, Llama glama
WYQQRPGQSPRLLIY

SEQ ID NO: 56 - protein, 15 aa, Llama glama
WYQQMPGQAPILVIY

SEQ ID NO: 57 - protein, 15 aa, Llama glama
WYQQMPGQAPVLVIY

SEQ ID NO: 58 - protein, 15 aa, Llama glama
WYQQKAGQAPVLVIY

SEQ ID NO: 59 - protein, 15 aa, Llama glama
WYQQLPGMAPKLLLY

SEQ ID NO: 60 - protein, 15 aa, Llama glama
WYQQKPGQSPVLVIY

SEQ ID NO: 61 - protein, 15 aa, Llama glama
WYQQRLGQSPRLLIY

SEQ ID NO: 62 - protein, 7 aa, Llama glama
HASTQES

SEQ ID NO: 63 - protein, 7 aa, Llama glama
YASTQES

SEQ ID NO: 64 - protein, 7 aa, Llama glama
RDSERPS

SEQ ID NO: 65 - protein, 7 aa, Llama glama
ADSRRPS

SEQ ID NO: 66 - protein, 7 aa, Llama glama
SVNKRAS

SEQ ID NO: 67 - protein, 7 aa, Llama glama
KDSNRPS

SEQ ID NO: 68 - protein, 7 aa, Llama glama
WASTRES

SEQ ID NO: 69 - protein, 32 aa, Llama glama
GIPDRFSGSGSTTDFTLTISSVQPEDAAVYYC

SEQ ID NO: 70 - protein, 32 aa, Llama glama
GLPDRFSGSGSTTDFTLTISSVQPEDAAVYYC

SEQ ID NO: 71 - protein, 32 aa, Llama glama
GIPDRFSGSNSGGTATLTISGAQAEDEADYYC

SEQ ID NO: 72 - protein, 32 aa, Llama glama
GIPERFSGSSSGGTATLAISGAQAEDEADYYC

SEQ ID NO: 73 - protein, 32 aa, Llama glama
GIPERFSGSNSGNTATLTISGAQAEDEADYYC

SEQ ID NO: 74 - protein, 32 aa, Llama glama
GIADRFSGSKSGNTASLTISGLQSEDEADYYC

SEQ ID NO: 75 - protein, 32 aa, Llama glama
GIPERFSGSNSGNTATLTVSGAQAEDEADYYC

SEQ ID NO: 76 - protein, 32 aa, Llama glama
GVPDRFSGSGSTTDFTLTINNFQPEDAAVYYC

SEQ ID NO: 77 - protein, 9 aa, Llama glama
QQAYNNPYS

SEQ ID NO: 78 - protein, 9 aa, Llama glama
QQAYSAPYS

SEQ ID NO: 79 - protein, 12 aa, Llama glama
QSGSSSGNAHAV

SEQ ID NO: 80 - protein, 10 aa, Llama glama
QSAHRSESAV

SEQ ID NO: 81 - protein, 11 aa, Llama glama
QVWDSRADAAV

SEQ ID NO: 82 - protein, 11 aa, Llama glama
GCYDSSLSTPV

SEQ ID NO: 83 - protein, 11 aa, Llama glama
QVWDSSANAAV

SEQ ID NO: 84 - protein, 9 aa, Llama glama
QQGYSAPLT

SEQ ID NO: 85 - protein, 10 aa, Llama glama
FGSGTRLEIK

SEQ ID NO: 86 - protein, 11 aa, Llama glama
FGGGTHLTVLG

SEQ ID NO: 87 - protein, 11 aa, Llama glama
FGGGTKLTVLG

SEQ ID NO: 88 - protein, 10 aa, Llama glama
FGQGTKVELK

SEQ ID NO: 89 - protein, 30 aa, Llama glama
EVQVQESGPGLVTPSQTLSLTCTVSGGSIK

SEQ ID NO: 90 - protein, 30 aa, Llama glama
EVQLQESGPGLVKPSQTLSLTCTVSGGSIS

SEQ ID NO: 91 - protein, 30 aa, Llama glama
EVQLVQPGAELRNPGASVKVSCKASGDTFT

SEQ ID NO: 92 - protein, 30 aa, Llama glama
EVQLVQPGAELRNPGASVKVSCKASGYTYT

SEQ ID NO: 93 - protein, 30 aa, Llama glama
EVQLVQPGAEVRRPGAAVKVSCKASGFALI

SEQ ID NO: 94 - protein, 30 aa, Llama glama
EVQLVQPGAELRNPGTSVKVSCKTSGYYFT

SEQ ID NO: 95 - protein, 30 aa, Llama glama; identical to SEQ ID NO: 93

SEQ ID NO: 96 - protein, 30 aa, Llama glama; identical to SEQ ID NO: 93

SEQ ID NO: 97 - protein, 30 aa, Llama glama
EVQLVQPGAELTKPGASVKVSCNTSGYTFT

SEQ ID NO: 98 - protein, 30 aa, Llama glama
EVQLVQPGAELRNPGASVKVSCKASGYIFT

SEQ ID NO: 99 - protein, 30 aa, Llama glama
QLQLVESGGGLVQPGGSLRLSCAASGFTFS

SEQ ID NO: 100 - protein, 7 aa, Llama glama
NNYYYWN

SEQ ID NO: 101 - protein, 7 aa, Llama glama
TSGTGWS

SEQ ID NO: 102 - protein, 5 aa, Llama glama
DYYIH

SEQ ID NO: 103 - protein, 5 aa, Llama glama
SAYIV

SEQ ID NO: 104 - protein, 5 aa, Llama glama
SSYID

SEQ ID NO: 105 - protein, 5 aa, Llama glama
SSDIQ

SEQ ID NO: 106 - protein, 5 aa, Llama glama
SSYID

SEQ ID NO: 107 - protein, 5 aa, Llama glama
GYYID

SEQ ID NO: 108 - protein, 5 aa, Llama glama
SAYID

SEQ ID NO: 109 - protein, 7 aa, Llama glama
FSEYVMS

SEQ ID NO: 110 - protein, 14 aa, Llama glama
WIRQPPGKGLEWMG

SEQ ID NO: 111 - protein, 14 aa, Llama glama
WVRQAPGQGLEWMG

SEQ ID NO: 112 - protein, 14 aa, Llama glama
WVRQAPGEGLEWMG

SEQ ID NO: 113 - protein, 14 aa, Llama glama
WVRQAPAQGLEWMG

SEQ ID NO: 114 - protein, 14 aa, Llama glama
WVRQAPGQGLEWMG

SEQ ID NO: 115 - protein, 14 aa, Llama glama
WVRQVPGEGLEWMG

SEQ ID NO: 116 - protein, 14 aa, Llama glama
WVRQAPGQGLEWMG

SEQ ID NO: 117 - protein, 14 aa, Llama glama
WVRQAPGKGLEWVS

SEQ ID NO: 118 - protein, 14 aa, Llama glama
WVRQAPGKGLEWVS

SEQ ID NO: 119 - protein, 16 aa, Llama glama
AIAYSGSTYYSPSLQS

SEQ ID NO: 120 - protein, 16 aa, Llama glama
IIGYDGGTYYNPSLKS

SEQ ID NO: 121 - protein, 17 aa, Llama glama
RIDPEDGGTKYTQKFQG

SEQ ID NO: 122 - protein, 17 aa, Llama glama
RIDPEDGGTKYAQKFQG

SEQ ID NO: 123 - protein, 17 aa, Llama glama
RIDPEDGGTSYAQKFQG

SEQ ID NO: 124 - protein, 17 aa, Llama glama
RIDPEDGATKYAPKFQG

SEQ ID NO: 125 - protein, 17 aa, Llama glama
RIDPEDGGTSYAQKFQG

SEQ ID NO: 126 - protein, 17 aa, Llama glama
RIDPEDGDTKYAQKFQG

SEQ ID NO: 127 - protein, 17 aa, Llama glama
RIDPEDGETRYAHQFRD

SEQ ID NO: 128 - protein, 17 aa, Llama glama
DINSGGEITAYADSVKG

SEQ ID NO: 129 - protein, 17 aa, Llama glama
DINSGGEITAYADSVKG

SEQ ID NO: 130 - protein, 32 aa, Llama glama
RTSISRDTSKNQFTLQLSSVTPEDTAVYYCAR

SEQ ID NO: 131 - protein, 32 aa, Llama glama
RTSISRDTSKNQFSLQLTSVTPEDTAVYYCAR

SEQ ID NO: 132 - protein, 32 aa, Llama glama
RVTFTADTSTSTVYLELDSLRSEDTAVYYCVG

SEQ ID NO: 133 - protein, 32 aa, Llama glama
RVTFTADTSTNTAYVELSSLRSDDTAVYYCTA

SEQ ID NO: 134 - protein, 32 aa, Llama glama
RVTVTVDTATSTAYVELSSLRSDDTGVYFCMV

SEQ ID NO: 135 - protein, 32 aa, Llama glama
RVTFTADTFTRTAYVELSNLRSDDTAVYYCMT

SEQ ID NO: 136 - protein, 32 aa, Llama glama
RVTVTVDTATSTVYVELSSLRSDDTGVYFCMV

SEQ ID NO: 137 - protein, 32 aa, Llama glama
RVTITADTSTTTAYMELSSLRSEDTAVYYCSL

SEQ ID NO: 138 - protein, 32 aa, Llama glama
RVTFTADTSTSTVYAELNSLRSEDTAVYYCAI

SEQ ID NO: 139 - protein, 32 aa, Llama glama
RFTVSRDNTKNTVYLQMNSLKPEDTAVYYCAQ

SEQ ID NO: 140 - protein, 32 aa, Llama glama; identical to SEQ ID NO: 139

SEQ ID NO: 141 - protein, 14 aa, Llama glama
VPSVFGLPYGGMNY

SEQ ID NO: 142 - protein, 4 aa, Llama glama
GLGP

SEQ ID NO: 143 - protein, 4 aa, Llama glama
DMDY

SEQ ID NO: 144 - protein, 4 aa, Llama glama
GLDY

SEQ ID NO: 145 - protein, 5 aa, Llama glama
GDSGY

SEQ ID NO: 146 - protein, 5 aa, Llama glama
AGGEY

SEQ ID NO: 147 - protein, 4 aa, Llama glama
GVDY

SEQ ID NO: 148 - protein, 5 aa, Llama glama
TIEGF

SEQ ID NO: 149 - protein, 5 aa, Llama glama
GSFGA

SEQ ID NO: 150 - protein, 11 aa, Llama glama
WGKGTLVTVSS

SEQ ID NO: 151 - protein, 11 aa, Llama glama
WGQGIQVTVSS

SEQ ID NO: 152 - protein, 11 aa, Llama glama
WGQGTQVTVSS

SEQ ID NO: 153 - protein, 22 aa, Llama glama
SYELTQSPSVTVALRQTAKITC

SEQ ID NO: 154 - protein, 23 aa, Llama glama
DIQMTQSPSSLSASLGDRVTITC

SEQ ID NO: 155 - protein, 22 aa, Llama glama
QTVVTQEPSLSVSPGGTVTLTC

SEQ ID NO: 156 - protein, 23 aa, Llama glama
ATMLTQSPGSLSVVPGESASISC

SEQ ID NO: 157 - protein, 22 aa, Llama glama
QPVLNQPSALSVSLGQTARITC

SEQ ID NO: 158 - protein, 23 aa, Llama glama
ATMLTQSPASLSVVPGESASISC

SEQ ID NO: 159 - protein, 23 aa, Llama glama
ATMLTQSPGSLSVGPGESASISC

SEQ ID NO: 160 - protein, 23 aa, Llama glama
ATMLTQSPGSLSVVPGESATIAC

SEQ ID NO: 161 - protein, 11 aa, Llama glama
GGSRIGSKSVQ

SEQ ID NO: 162 - protein, 11 aa, Llama glama
QASQGISKYLA

SEQ ID NO: 163 - protein, 14 aa, Llama glama
GLSSGSVTFGNYPS

SEQ ID NO: 164 - protein, 16 aa, Llama glama
KASQSLVHSDGMTYLY

SEQ ID NO: 165 - protein, 16 aa, Llama glama
KASQSLVHTDGKTYLS

SEQ ID NO: 166 - protein, 11 aa, Llama glama
QGGDFRNYYNN

SEQ ID NO: 167 - protein, 16 aa, Llama glama
KASQSLVHSDGKTYLY

SEQ ID NO: 168 - protein, 16 aa, Llama glama
KAGQSLTHPNGKTYLS

SEQ ID NO: 169 - protein, 16 aa, Llama glama
KTSQSLVHSDGKTYLY

SEQ ID NO: 170 - protein, 16 aa, Llama glama
KTSRSLVHSDGKTYLS

SEQ ID NO: 171 - protein, 16 aa, Llama glama
KASQSLVHSDGKTYLY

SEQ ID NO: 172 - protein, 16 aa, Llama glama
KANESIVHPGGKTYLY

SEQ ID NO: 173 - protein, 15 aa, Llama glama
WYQQKPGQVPVLVIY

SEQ ID NO: 174 - protein, 15 aa, Llama glama
WYQQKPGQAPKLLIY

SEQ ID NO: 175 - protein, 15 aa, Llama glama
WYQQTPGQAPRTLIY

SEQ ID NO: 176 - protein, 15 aa, Llama glama
WLQQKPGQSPQRLIY

SEQ ID NO: 177 - protein, 15 aa, Llama glama
WLLQKPGQSPQRLIY

SEQ ID NO: 178 - protein, 15 aa, Llama glama
WYQQKPGQAPVLVIY

SEQ ID NO: 179 - protein, 15 aa, Llama glama
WLLQKPGQSPRRLIY

SEQ ID NO: 180 - protein, 15 aa, Llama glama
WLRQKPGQSPQRLIY

SEQ ID NO: 181 - protein, 7 aa, Llama glama
ADNRRPS

SEQ ID NO: 182 - protein, 7 aa, Llama glama
SASRLET

SEQ ID NO: 183 - protein, 7 aa, Llama glama
TTNSRHS

SEQ ID NO: 184 - protein, 7 aa, Llama glama
RVSRRGS

SEQ ID NO: 185 - protein, 7 aa, Llama glama
QVSTRGS

SEQ ID NO: 186 - protein, 7 aa, Llama glama
EDSERPS

SEQ ID NO: 187 - protein, 7 aa, Llama glama
QVSNRGS

SEQ ID NO: 188 - protein, 7 aa, Llama glama
ETSNRDP

SEQ ID NO: 189 - protein, 7 aa, Llama glama
QVSNRDS

SEQ ID NO: 190 - protein, 7 aa, Llama glama
QVSNRGS

SEQ ID NO: 191 - protein, 7 aa, Llama glama
QVSRRDS

SEQ ID NO: 192 - protein, 7 aa, Llama glama
QVSNRAS

SEQ ID NO: 193 - protein, 32 aa, Llama glama
RIPERFSGSNSGNTATLIISGAQAEDEADYYC

SEQ ID NO: 194 - protein, 32 aa, Llama glama
GVPSRFSGSGSGTTFTLVISGLEAEDAGSYYC

SEQ ID NO: 195 - protein, 32 aa, Llama glama
GVPSRFSGSISGNKATLTISGAQPEDEADFYC

SEQ ID NO: 196 - protein, 32 aa, Llama glama
GVPDRFTGSGSGTDFTLNISDMKAEDAGVYYC

SEQ ID NO: 197 - protein, 32 aa, Llama glama
GVPDRVTGSGSGTDFTLKISGVKAEDAGVYYC

SEQ ID NO: 198 - protein, 32 aa, Llama glama
GIPERFSGSSSGDTVILTVSRVQADDEADYYC

SEQ ID NO: 199 - protein, 32 aa, Llama glama
GVPDRFTGSGSGTDFTLKISGVKAEDAGVYYC

SEQ ID NO: 200 - protein, 32 aa, Llama glama
GVPDRFTGSGSGTDFTLRIVGVTVEDAGVYYC

SEQ ID NO: 201 - protein, 33 aa, Llama glama
GVPDRFTGSGSGTDFTLKISGVKAEDAGVYYCA

SEQ ID NO: 202 - protein, 32 aa, Llama glama
GVPDRFTGSGSGTDFTLKISGVKAEDAGLYYC

SEQ ID NO: 203 - protein, 32 aa, Llama glama
GVPDRFTGSGSGTDFTLKISGLKAEDAGVYYC

SEQ ID NO: 204 - protein, 9 aa, Llama glama
QVWDSSAAV

SEQ ID NO: 205 - protein, 9 aa, Llama glama
QQYYRAPAT

SEQ ID NO: 206 - protein, 12 aa, Llama glama
ALSRVSGTYGTV

SEQ ID NO: 207 - protein, 9 aa, Llama glama
AQDTYYPYS

SEQ ID NO: 208 - protein, 9 aa, Llama glama
AQTTYYPPT

SEQ ID NO: 209 - protein, 10 aa, Llama glama
QSSDSTDNAV

SEQ ID NO: 210 - protein, 9 aa, Llama glama
AQHTYYPPS

SEQ ID NO: 211 - protein, 9 aa, Llama glama
AQTTYFPIA

SEQ ID NO: 212 - protein, 9 aa, Llama glama
AQTTYDPVT

SEQ ID NO: 213 - protein, 8 aa, Llama glama
QATYYPLS

SEQ ID NO: 214 - protein, 9 aa, Llama glama
AQATYVPLG

SEQ ID NO: 215 - protein, 9 aa, Llama glama
AQATFKKIT

SEQ ID NO: 216 - protein, 11 aa, Llama glama
FGGGTHLTVLG

SEQ ID NO: 217 - protein, 10 aa, Llama glama
FGQGTKVELK

SEQ ID NO: 218 - protein, 123 aa, Llama glama
QVQLVESGGGLVQPGGSLRLSCAASGFTFSSYWMYWVRQAPGKGLEWVSAINTGGGSTYYADSVKGRFTISRDNAKNTLYLQMNSLKPEDTALYYCARGPGYSGSYIEGWAYWGQGTQVTVSS

SEQ ID NO: 219 - protein, 123 aa, Llama glama
QVQLVESGGGLVQPGGSLRLSCAASGFTFSSYWMYWVRQAPGKGLEWVSAINTGGGSTYYADSVKGRFTISRDNAKNTLYLQMNSLKPEDTALYYCARGPGYSGKYIEGWAYWGQGTQVTVSS

SEQ ID NO: 220 - protein, 123 aa, Llama glama
EVQVQESGGGLVQPGGSLRLSCAASGFTFSSYWMYWVRQAPGKGLEWVSAINTGGGSTYYADSVKGRFTISRDNAKNTLYLQMNSLKPEDTALYYCARGPGYSGKYIEGWAYWGQGTQVTVSS

SEQ ID NO: 221 - protein, 120 aa, Llama glama
EVQLQESGGGLVQPGGSLRLSCAASGFTFDDYAMSWVRQAPGKGLEWVSAISWNGGSTYYAESMKGRFTISRDNAKNTLYLQMNSLKSKDTAVYYCARRRYGLGSEYDYWGQGTQVTVSS

SEQ ID NO: 222 - protein, 120 aa, Llama glama
QLQLVESGGGLVQPGGSLRLSCAASGFTFDDYAMSWVRQAPGKGLEWVSAISWNGGSTYYAESMKGRFTISRDNAKNTLYLQMNSLKSKDTAVYYCARRRYGLGSEYDYWGQGTQVTVSS

SEQ ID NO: 223 - protein, 120 aa, Llama glama
QVQLQESGPGLVKPSQTLSLTCTVSGGSITTSFYAWSWIRQPPGKGLEWMGVIAYDGRTFYSPSLKSRTHISRDTSKNQFSLQLSSVTPEDTAVYYCARGGVVAWSYDYWGQGTQVTVSS

SEQ ID NO: 224 - protein, 123 aa, Llama glama
QVQLQESGPGLVKPSQTLSLTCTVSGGSITNYGWSWIRQPPGKGLEWMGVIAYDGSTYYSPSLKSRTSISRDTSKNQFSLQLSSVTPEDTAVYYCASAMVTTSSPWVKTFGAWGQGTQVTVSS

SEQ ID NO: 225 - protein, 124 aa, Llama glama
EVQLQESGPGLVKPSQTLSLTCTVSGGSITNSYYASSWIRQPPGKGLEWMGVIIYDGSTYYSPSLKSRTSISRDTSKNQFSLQLSSVTPEDTAVYYCARGLRVAGSYFTDFGTWGQGTQVTVSS

SEQ ID NO: 226 - protein, 117 aa, Llama glama
EVQLVESGGGLVQPGGTLRLSCAASGFTFSNYWMHWVRQAPGKGLEWVSVIDRGGGGTRYADSVKGRFTISRDNAKNTLYLRMNSLKPEDTALYYCTRDRNGEYDYWGRGTQVTVSS

SEQ ID NO: 227 - protein, 113 aa, Llama glama
DIVMTQSPTSVTASVGEKVTINCKSSQSVVSGSNQKSYLNWYQQRPGQSPRLLIYHASTQESGIPDRFSGSGSTTDFTLTISSVQPEDAAVYYCQQAYNNPYSFGSGTRLEIK

SEQ ID NO: 228 - protein, 113 aa, Llama glama
DIVMTQSPRSVTASVGEKVFINCKSSQSVVSESNQRSYLNWYQQRPGQSPRLLIYYASTQESGLPDRFSGSGSTTDFTLTISSVQPEDAAVYYCQQAYSAPYSFGSGTRLEIK

SEQ ID NO: 229 - protein, 113 aa, Llama glama
DIVMTQSPSSVTASVGEKVTINCKSSQSVVSGSKQKSYLNWYQQRPGQSPRLLIYYASTQESGIPDRFSGSGSTTDFTLTISSVQPEDAAVYYCQQAYSAPYSFGSGTRLEIK

SEQ ID NO: 230 - protein, 110 aa, Llama glama
NFMLTQPSAVSVSLGQTARITCQGSSFGSSYAHWYQQMPGQAPILVIYRDSERPSGIPDRFSGSNSGGTATLTISGAQAEDEADYYCQSGSSSGNAHAVFGGGTHLTVLG

SEQ ID NO: 231 - protein, 108 aa, Llama glama
HSAVTQPSAVSVSLGQTARITCQGTNLRSSYVHWYQQMPGQAPVLVIYRDSERPSGIPERFSGSSSGGTATLAISGAQAEDEADYYCQSAHRSESAVFGGGTHLTVLG

SEQ ID NO: 232 - protein, 109 aa, Llama glama
SYELTQSPSVSVALRQTAKITCGGNDIGSKSTQWYQQKAGQAPVLVIYADSRRPSGIPERFSGSNSGNTATLTISGAQAEDEADYYCQVWDSRADAAVFGGGTHLTVLG

SEQ ID NO: 233 - protein, 112 aa, Llama glama
QAGLTQPPSVSGSPGKTVTISCAGTSSDVGYGNYVSWYQQLPGMAPKLLLYSVNKRASGIADRFSGSKSGNTASLTISGLQSEDEADYYCGCYDSSLSTPVFGGGTKLTVLG

SEQ ID NO: 234 - protein, 109 aa, Llama glama
SYELTQSPSVSVALRQAAKITCGGDNIASKHAHWYQQKPGQSPVLVIYKDSNRPSGIPERFSGSNSGNTATLTVSGAQAEDEADYYCQVWDSSANAAVFGGGTHLTVLG

SEQ ID NO: 235 - protein, 113 aa, Llama glama
DIQMTQSPSSVTASAGEKVTINCKSSQSVLYSSNQKNYLAWYQQRLGQSPRLLIYWASTRESGVPDRFSGSGSTTDFTLTINNFQPEDAAVYYCQQGYSAPLTFGQGTKVELK

SEQ ID NO: 236 - protein, 124 aa, Llama glama
EVQVQESGPGLVTPSQTLSLTCTVSGGSIKNNYYYWNWIRQPPGKGLEWMGAIAYSGSTYYSPSLQSRTSISRDTSKNQFTLQLSSVTPEDTAVYYCARVPSVFGLPYGGMNYWGKGTLVTVSS

SEQ ID NO: 237 - protein, 114 aa, Llama glama
EVQLQESGPGLVKPSQTLSLTCTVSGGSISTSGTGWSWIRQPPGKGLEWMGIIGYDGGTYYNPSLKSRTSISRDTSKNQFSLQLTSVTPEDTAVYYCARGLGPWGQGIQVTVSS

SEQ ID NO: 238 - protein, 113 aa, Llama glama
EVQLVQPGAELRNPGASVKVSCKASGDTFTDYYIHWVRQAPGQGLEWMGRIDPEDGGTKYTQKFQGRVTFTADTSTSTVYLELDSLRSEDTAVYYCVGDMDYWGKGTLVTVSS

SEQ ID NO: 239 - protein, 113 aa, Llama glama
EVQLVQPGAELRNPGASVKVSCKASGYTYTSAYIVWVRQAPGEGLEWMGRIDPEDGGTKYAQKFQGRVTFTADTSTNTAYVELSSLRSDDTAVYYCTAGLDYWGKGTLVTVSS

SEQ ID NO: 240 - protein, 114 aa, Llama glama
EVQLVQPGAEVRRPGAAVKVSCKASGFALISSYIDWVRQAPGQGLEWMGRIDPEDGGTSYAQKFQGRVTVTVDTATSTAYVELSSLRSDDTGVYFCMVGDSGYWGQGTQVTVSS

SEQ ID NO: 241 - protein, 114 aa, Llama glama
EVQLVQPGAELRNPGTSVKVSCKTSGYYFTSSDIQWVRQAPAQGLEWMGRIDPEDGATKYAPKFQGRVTFTADTFTRTAYVELSNLRSDDTAVYYCMTAGGEYWGKGTLVTVSS

SEQ ID NO: 242 - protein, 114 aa, Llama glama
EVQLVQPGAEVRRPGAAVKVSCKASGFALISSYIDWVRQAPGQGLEWMGRIDPEDGGTSYAQKFQGRVTVTVDTATSTVYVELSSLRSDDTGVYFCMVGDSGYWGQGTQVTVSS

SEQ ID NO: 243 - protein, 114 aa, Llama glama; identical to SEQ ID NO: 242

SEQ ID NO: 244 - protein, 113 aa, Llama glama
EVQLVQPGAELTKPGASVKVSCNTSGYTFTGYYIDWVRQVPGEGLEWMGRIDPEDGDTKYAQKFQGRVTITADTSTTTAYMELSSLRSEDTAVYYCSLGVDYWGQGTQVTVSS

SEQ ID NO: 245 - protein, 114 aa, Llama glama
EVQLVQPGAELRNPGASVKVSCKASGYIFTSAYIDWVRQAPGQGLEWMGRIDPEDGETRYAHQFRDRVTFTADTSTSTVYAELNSLRSEDTAVYYCAITIEGFWGQGTQVTVSS

SEQ ID NO: 246 - protein, 116 aa, Llama glama
EVQLVESGGGLVQPGGSLRLSCAASGFTFSFSEYVMSWVRQAPGKGLEWVSDINSGGEITAYADSVKGRFTVSRDNTKNTVYLQMNSLKPEDTAVYYCAQGSFGAWGQGTQVTVSS

SEQ ID NO: 247 - protein, 116 aa, Llama glama
QLQLVESGGGLVQPGGSLRLSCAASGFTFSFSEYVMSWVRQAPGKGLEWVSDINSGGEITAYADSVKGRFTVSRDNTKNTVYLQMNSLKPEDTAVYYCAQGSFGAWGQGTQVTVSS

SEQ ID NO: 248 - protein, 114 aa, Llama glama
EVQVQESGPGLVKPSQTLSLTCTVSGASIADKSSAWSWIRQPPGKGLEWMGVIRYDGNTRYSPTLKSRTTISRDTSKNQISLQLTSVTPDDTAVYYCSREITLWGQGTQVTVSS

SEQ ID NO: 249 - protein, 107 aa, Llama glama
SYELTQSPSVTVALRQTAKITCGGSRIGSKSVQWYQQKPGQVPVLVIYADNRRPSRIPERFSGSNSGNTATLIISGAQAEDEADYYCQVWDSSAAVFGGGTHLTVLG

SEQ ID NO: 250 - protein, 107 aa, Llama glama
DIQMTQSPSSLSASLGDRVTITCQASQGISKYLAWYQQKPGQAPKLLIYSASRLETGVPSRFSGSGSGTTFTLVISGLEAEDAGSYYCQQYYRAPATFGQGTKVELK

SEQ ID NO: 251 - protein, 113 aa, Llama glama
QTVVTQEPSLSVSPGGTVTLTCGLSSGSVTFGNYPSWYQQTPGQAPRTLIYTTNSRHSGVPSRFSGSISGNKATLTISGAQPEDEADFYCALSRVSGTYGTVFGGGTHLTVLG

SEQ ID NO: 252 - protein, 112 aa, Llama glama
ATMLTQSPGSLSVVPGESASISCKASQSLVHSDGMTYLYWLQQKPGQSPQRLIYRVSRRGSGVPDRFTGSGSGTDFTLNISDMKAEDAGVYYCAQDTYYPYSFGSGTSVEIK

SEQ ID NO: 253 - protein, 112 aa, Llama glama
ATMLTQSPGSLSVVPGESASISCKASQSLVHTDGKTYLSWLLQKPGQSPQRLIYQVSTRGSGVPDRVTGSGSGTDFTLKISGVKAEDAGVYYCAQTTYYPPTFGQGTKVELK

SEQ ID NO: 254 - protein, 108 aa, Llama glama
QPVLNQPSALSVSLGQTARITCQGGDFRNYYNNWYQQKPGQAPVLVIYEDSERPSGIPERFSGSSSGDTVILTVSRVQADDEADYYCQSSDSTDNAVFGGGTHLTVLG

SEQ ID NO: 255 - protein, 112 aa, Llama glama
ATMLTQSPASLSVVPGESASISCKASQSLVHSDGKTYLYWLLQKPGQSPQRLIYQVSNRGSGVPDRFTGSGSGTDFTLKISGVKAEDAGVYYCAQHTYYPPSFGSGTRLEIK

SEQ ID NO: 256 - protein, 112 aa, Llama glama
ATMLTQSPGSLSVGPGESASISCKAGQSLTHPNGKTYLSWLLQKPGQSPRRLIYETSNRDPGVPDRFTGSGSGTDFTLRIVGVTVEDAGVYYCAQTTYFPIAFGQGTKVELK

SEQ ID NO: 257 - protein, 112 aa, Llama glama
ATMLTQSPGSLSVVPGESASISCKTSQSLVHSDGKTYLYWLLQKPGQSPQRLIYQVSNRDSGVPDRFTGSGSGTDFTLKISGVKAEDAGVYYCAQTTYDPVTFGQGTKVELK

SEQ ID NO: 258 - protein, 112 aa, Llama glama
ATMLTQSPGSLSVVPGESASISCKTSRSLVHSDGKTYLSWLLQKPGQSPQRLIYQVSNRGSGVPDRFTGSGSGTDFTLKISGVKAEDAGVYYCAQATYYPLSFGSGTRVEIK

SEQ ID NO: 259 - protein, 112 aa, Llama glama
ATMLTQSPGSLSVVPGESATIACKANESIVHPGGKTYLYWLLQKPGQSPQRLIYQVSNRASGVPDRFTGSGSGTDFTLKISGLKAEDAGVYYCAQATFKKITFGQGTKVELK

SEQ ID NO: 260 - protein, 112 aa, Llama glama
ATMLTQSPGSLSVVPGESASISCKASQSLVHSDGKTYLYWLRQKPGQSPQRLIYQVSRRDSGVPDRFTGSGSGTDFTLKISGVKAEDAGLYYCAQATYVPLGFGSGTRLEIK

SEQ ID NO: 261 - protein, 111 aa, Llama glama
ATMLTQSPGSLSVVPGESASISCKASQSLVHSDGKTYLYWLLQKPGQSPQRLIYQVSNRGSGVPDRFTGSGSGTDFTLKISGVKAEDAGVYYCAQATYYPTFGQGTKVELK

SEQ ID NO: 262 - protein, 51 aa, Homo sapiens
GNLKHKCFRNSLENNETLESIMNTLESEEDFRKYFYYLEGSKDALLCGFST

SEQ ID NO: 263 - protein, 13 aa, Homo sapiens
EHHPMTEEFKNVL

SEQ ID NO: 264 - protein, 14 aa, Homo sapiens
EDIYIERKKTIKII

SEQ ID NO: 265 - protein, 14 aa, Homo sapiens
VEKEGQSQHMTEVL

SEQ ID NO: 266 - protein, 87 aa, Homo sapiens
GKFYECINTTDGSRFPASQVPNRSECFALMNVSQNVRWKNLKVNFDNVGLGYLSLLQVATFKGWTIIMYAAVDSVNVDKQPKYEYSL

SEQ ID NO: 267 - protein, 288 aa, Artificial
  chimeric polypeptide
GNLKHKCFRNSLENNETLESIMNTLESEEDFRKYFYYLEGSKDALLCGFSTIEGRDMDPHGGCTCPQCPAPELPGGPSVFVFPPKPKDVLSISGRPEVTCVVVDVGKEDPEVNFNWYIDGVEVRTANTKPKEEQFNSTYRVVSVLPIQHQDWLTGKEFKCKVNNKALPAPIERTISKAKGQTREPQVYTLAPHREELAKDTVSVTCLVKGFYPADINVEWQRNGQPESEGTYANTPPQLDNDGTYFLYSKLSVGKNTWQRGETLTCVVMHEALHNHYTQKSISQSPGK

SEQ ID NO: 268 - protein, 302 aa, Artificial
  chimeric polypeptide
GGSCGSEHHPMTEEFKNVLGGSGGSEDIYIERKKTIKIIGGSGGSVEKEGQSQHMTEVLGGSCGSIEGRDMDPHGGCTCPQCPAPELPGGPSVFVFPPKPKDVLSISGRPEVTCVVVDVGKEDPEVNFNWYIDGVEVRTANTKPKEEQFNSTYRVVSVLPIQHQDWLTGKEFKCKVNNKALPAPIERTISKAKGQTREPQVYTLAPHREELAKDTVSVTCLVKGFYPADINVEWQRNGQPESEGTYANTPPQLDNDGTYFLYSKLSVGKNTWQRGETLTCVVMHEALHNHYTQKSISQSPGK

SEQ ID NO: 269 - protein, 324 aa, Artificial
  chimeric polypeptide
GKFYECINTTDGSRFPASQVPNRSECFALMNVSQNVRWKNLKVNFDNVGLGYLSLLQVATFKGWTIIMYAAVDSVNVDKQPKYEYSLIEGRDMDPHGGCTCPQCPAPELPGGPSVFVFPPKPKDVLSISGRPEVTCVVVDVGKEDPEVNFNWYIDGVEVRTANTKPKEEQFNSTYRVVSVLPIQHQDWLTGKEFKCKVNNKALPAPIERTISKAKGQTREPQVYTLAPHREELAKDTVSVTCLVKGFYPADINVEWQRNGQPESEGTYANTPPQLDNDGTYFLYSKLSVGKNTWQRGETLTCVVMHEALHNHYTQKSISQSPGK

SEQ ID NO: 270 - protein, 294 aa, Artificial
  chimeric polypeptide
GNLRHKCVRNFTALNGTNGSVEADGLVWESLDLYLSDPENYLLKNGTSDVLLCGNSSIEGRDMDPHGGCTCPQCPAPELPGGPSVFVFPPKPKDVLSISGRPEVTCVVVDVGKEDPEVNFNWYIDGVEVRTANTKPKEEQFNSTYRVVSVLPIQHQDWLTGKEFKCKVNNKALPAPIERTISKAKGQTREPQVYTLAPHREELAKDTVSVTCLVKGFYPADINVEWQRNGQPESEGTYANTPPQLDNDGTYFLYSKLSVGKNTWQRGETLTCVVMHEALHNHYTQKSISQSPGK

SEQ ID NO: 271 - protein, 90 aa, Artificial
  chimeric polypeptide
NLRNKCLQWPPDNSSFEINITSFFNNSLDGNGTTFNRTVSIFNWDEYIEDKSHFYFLEGQNDALLCGNSSDAGQCPEGYICVKAGRNPNY

SEQ ID NO: 272 - protein, 71 aa, Artificial
  chimeric polypeptide
GNLKHKCFRNSLENNETLESIMNTLESEEDFRKYFYYLEGSKDALLCGFSTDSGQCPEGYTCVKIGRNPDY

SEQ ID NO: 273 - protein, 10 aa, Homo sapiens
EPKSCDKTHT

SEQ ID NO: 274 - protein, 5 aa, Homo sapiens
CPPCP

SEQ ID NO: 275 - protein, 8 aa, Homo sapiens
APELLGGP

SEQ ID NO: 276 - protein, 12 aa, Homo sapiens
ELKTPLGDTTHT

SEQ ID NO: 277 - protein, 50 aa, Homo sapiens
CPRCPEPKSCDTPPPCPRCPEPKSCDTPPPCPRCPEPKSCDTPPPCPRCP

SEQ ID NO: 278 - protein, 8 aa, Homo sapiens
APELLGGP

SEQ ID NO: 279 - protein, 7 aa, Homo sapiens
ESKYGPP

SEQ ID NO: 280 - protein, 5 aa, Homo sapiens
CPSCP

SEQ ID NO: 281 - protein, 8 aa, Homo sapiens
APEFLGGP

SEQ ID NO: 282 - protein, 3 aa, Homo sapiens
ERK

SEQ ID NO: 283 - protein, 10 aa, Homo sapiens
CCVECPPPCP

SEQ ID NO: 284 - protein, 7 aa, Homo sapiens
APPVAGP

SEQ ID NO: 285 - protein, 302 aa, Artificial
  chimeric polypeptide
GGSCGSEHHPMTEEFKNVLGGSGGSEDIYIEKKKTIKIIGGSGGSVEKEGQTEYMDYVLGGSCGSIEGRDMDPHGGCTCPQCPAPELPGGPSVFVFPPKPKDVLSISGRPEVTCVVVDVGKEDPEVNFNWYIDGVEVRTANTKPKEEQFNSTYRVVSVLPIQHQDWLTGKEFKCKVNNKALPAPIERTISKAKGQTREPQVYTLAPHREELAKDTVSVTCLVKGFYPADINVEWQRNGQPESEGTYANTPPQLDNDGTYFLYSKLSVGKNTWQRGETLTCVVMHEALHNHYTQKSISQSPGK

SEQ ID NO: 286 - protein, 288 aa, Artificial
  chimeric polypeptide
GNLKHKCFRKELEENETLESIMNTAESEEELKKYFYYLEGSKDALLCGFSTIEGRDMDPHGGCTCPQCPAPELPGGPSVFVFPPKPKDVLSISGRPEVTCVVVDVGKEDPEVNFNWYIDGVEVRTANTKPKEEQFNSTYRVVSVLPIQHQDWLTGKEFKCKVNNKALPAPIERTISKAKGQTREPQVYTLAPHREELAKDTVSVTCLVKGFYPADINVEWQRNGQPESEGTYANTPPQLDNDGTYFLYSKLSVGKNTWQRGETLTCVVMHEALHNHYTQKSISQSPGK

SEQ ID NO: 287 - protein, 302 aa, Artificial
  chimeric polypeptide
GGSCGSEHYNMTSEFEEMLGGSGGSEDIYLEERKTIKVLGGSGGSVETDDQSPEKINILGGSCGSIEGRDMDPHGGCTCPQCPAPELPGGPSVFVFPPKPKDVLSISGRPEVTCVVVDVGKEDPEVNFNWYIDGVEVRTANTKPKEEQFNSTYRVVSVLPIQHQDWLTGKEFKCKVNNKALPAPIERTISKAKGQTREPQVYTLAPHREELAKDTVSVTCLVKGFYPADINVEWQRNGQPESEGTYANTPPQLDNDGTYFLYSKLSVGKNTWQRGETLTCVVMHEALHNHYTQKSISQSPGK

SEQ ID NO: 288 - protein, 30 aa, Llama glama
EVQVQESGPGLVKPSQTLSLTCTVSGASIA

SEQ ID NO: 289 - protein, 7 aa, Llama glama
DKSSAWS

SEQ ID NO: 290 - protein, 14 aa, Llama glama
WIRQPPGKGLEWMG

SEQ ID NO: 291 - protein, 16 aa, Llama glama
VIRYDGNTRYSPTLKS

SEQ ID NO: 292 - protein, 32 aa, Llama glama
RTTISRDTSKNQISLQLTSVTPDDTAVYYCSR

SEQ ID NO: 293 - protein, 4 aa, Llama glama
EITL

SEQ ID NO: 294 - protein, 11 aa, Llama glama
WGQGTQVTVSS

SEQ ID NO: 295 - protein, 23 aa, Llama glama
ATMLTQSPGSLSVVPGESASISC

SEQ ID NO: 296 - protein, 16 aa, Llama glama
KASQSLVHSDGKTYLY

SEQ ID NO: 297 - protein, 15 aa, Llama glama
WLLQKPGQSPQRLIY

SEQ ID NO: 298 - protein, 7 aa, Llama glama
QVSNRGS

SEQ ID NO: 299 - protein, 32 aa, Llama glama; identical to SEQ ID NO: 199

SEQ ID NO: 300 - protein, 8 aa, Llama glama
AQATYYPT

SEQ ID NO: 301 - protein, 10 aa, Llama glama
FGQGTKVELK

SEQ ID NO: 302 - protein, 114 aa, Llama glama; identical to SEQ ID NO: 248

SEQ ID NO: 303 - protein, 111 aa, Llama glama; identical to SEQ ID NO: 261

SEQ ID NO: 304 - DNA, 321 bp, Llama glama
tcctacgaactgactcagtcaccctcagtgacggtggcactgagacagacggccaagatcacctgtggcgggagccgcattggaagtaaaagtgttcagtggtaccagcagaagccgggccaggtccctgtgctggtcatctatgctgataacagacggccctcaaggatccctgagaggttctcaggctccaactctgggaacacggccaccctgataatcagcggggcccaggccgaggacgaggccgactactactgtcaggtgtgggacagcagtgctgctgtgttcggcggagggacccatctgaccgtcctcggt

SEQ ID NO: 305 - DNA, 372 bp, Llama glama
gaggtgcaggtgcaggagtcgggcccaggcctggtgacaccctcgcagacactctccctcacctgcactgtctctggtggctccattaaaaacaactattactactggaactggatccgccagccccccggaaaggggctggaatggatgggagccatagcttatagcggcagcacttactacagcccatccctccagagccgcacctccatctccagggacacgtccaagaaccagttcaccctgcagctgagctctgtgacccctgaagacacagccgtgtattactgtgccagagtgccgtcggtatttggccttccttatggcggcatgaactactggggcaaagggaccctggtcactgtctcctca

SEQ ID NO: 306 - DNA, 321 bp, Llama glama
gacatccagatgacccagtctccttcctccctgtctgcatctctgggagacagagtcaccatcacctgccaggccagtcagggcatcagcaagtacttagcctggtatcagcagaaaccagggcaagctcctaagctcctgatctatagtgcatccagactagaaaccggggtcccttccaggttcagcggcagtggatcagggacaactttcaccctcgtcatcagcggcctggaggctgaagatgccggaagttactactgtcagcagtattacagagcacccgctacgttcggccaggggaccaaggtggaactcaaa

SEQ ID NO: 307 - DNA, 348 bp, Llama glama
gaggtgcagctgcaggagtcgggcccaggcctggtgaagccctcgcagacgctctctctcacctgcactgtctctggtggctccatctcaaccagtggtactggttggagctggatccgccagcccccagggaaggggctggagtggatggggatcataggttatgatggcggcacttactacaacccatctctcaagagccgcacttccatctccagagacacgtccaagaaccagttctccctgcagttgacctctgtgacccctgaggacacggccgtgtattactgtgccagagggctggggccctggggccaggggatccaggtcaccgtgtcctcagccagt

SEQ ID NO: 308 - DNA, 336 bp, Llama glama
gcgaccatgctgacccagtccccaggctccctgtctgtcgtccctggagagtcagcctccatctcctgcaaggctagtcagagcctcgtacacactgatggaaagacctatttgtcttggctcctacagaagccgggccaatctccacagcgactgatctatcaagtttccacccgtggctctggggtcccagacagggtcactggcagcgggtcagggacagatttcaccctgaaaatcagcggggtgaaggctgaggatgctggagtgtattactgtgctcaaactacatattatcctccaacgttcggccaggggaccaaggtggaactcaaa

SEQ ID NO: 309 - DNA, 342 bp, Llama glama
gaggtccagctggttcagccaggggctgaggtgagacgccctggggcagcagtgaaggtctcctgcaaggcttctggatttgcgttgataagctcgtacatagactgggtgcggcaggcccctggacaagggcttgagtggatggggaggattgaccctgaagatggtggtacatcatatgcacaaaagttccagggccgagtcacggtcactgtcgacacggccaccagcacagcctacgtggagttgagcagtctgagatctgacgacacgggcgtctatttctgcatggtgggggattcaggctactggggccaggggacccaggtcaccgtgtcctca

SEQ ID NO: 310 - protein, 51 aa, Mus musculus
GNLKHKCFRKDLEQNETLESIMSTAESEEELKRYFYYLEGSKDALLCGFST

SEQ ID NO: 311 - protein, 51 aa, Artificial
  chimeric polypeptide
GNLKHKCFRNSLENNETLESIMSTAESEEELKRYFYYLEGSKDALLCGFST

SEQ ID NO: 312 - protein, 53 aa, Artificial
  chimeric polypeptide
GNLKHKCFRKDLEQNETLESIMSTAESEEEEDFRKYFYYLEGSKDALLCGFST

SEQ ID NO: 313 - protein, 51 aa, Artificial
  chimeric polypeptide
GNLKHKCFRKDLEQNETLESIMNTLESEEELKRYFYYLEGSKDALLCGFST

SEQ ID NO: 314 - protein, 51 aa, Artificial
  chimeric polypeptide
GNLKHKCFRKDLEQNETLESIMNTLESEEDFRKYFYYLEGSKDALLCGFST

SEQ ID NO: 315 - DNA, 231 bp, Artificial
  plasmid
ttactcgcggcccagccggccatggcccaggtgcagctgcaggagagcggggtcaccgtctaataagtctctgcggccgcaagcggtggaggcggttcaggcggaggtggatctggcggtggcggaagtgcacaggtccaagtgctcgagtaataaggcgcgcccgcacatcatcatcaccatcacggggccgcagaacaaaaactcatctcagaagaggatctgaatggg

SEQ ID NO: 316 - protein, 73 aa, Artificial
  chimeric polypeptide
LLAAQPAMAQVQLQESGVTVVSAAASGGGGSGGGGSGGGGSAQVQVLEGAPAHHHHHHGAAEQKLISEEDLNG

SEQ ID NO: 317 - DNA, 27 bp, Artificial
  primer
atgaaatacctattgcctacggcagcc

SEQ ID NO: 318 - DNA, 20 bp, Artificial
  primer
ccgctggattgttattactc

SEQ ID NO: 319 - DNA, 28 bp, Artificial
  primer
atgttgcggccgcggaggctgaggagac

SEQ ID NO: 320 - DNA, 28 bp, Artificial
  primer
atgttgcggccgcggaggctgaggacac

SEQ ID NO: 321 - DNA, 28 bp, Artificial
  primer
atgttgcggccgcggaggcgctgctaac

SEQ ID NO: 322 - DNA, 28 bp, Artificial
  primer
atgttgcggccgcggaggccgaggaaac

SEQ ID NO: 323 - DNA, 24 bp, Artificial
  primer
gagcggataacaatttcacacagg

SEQ ID NO: 324 - DNA, 37 bp, Artificial
  primer
acgtcagtggcgcgccggccttgggctgacccaggac

SEQ ID NO: 325 - DNA, 24 bp, Artificial
  primer
gagcggataacaatttcacacagg

SEQ ID NO: 326 - DNA, 31 bp, Artificial
  primer
acgtcagtggcgcgcccttggcatcagcccg

SEQ ID NO: 327 - DNA, 54 bp, Artificial
  primer
ggccgcaagcggtggaggcggttcaggcggaggtggatctggcggtggcggaag

SEQ ID NO: 328 - DNA, 55 bp, Artificial
  primer
tgcacttccgccaccgccagatccacctccgcctgaaccgcctccaccgcttgcg

SEQ ID NO: 329 - protein, 118 aa, Llama glama
QLQLVESGGGLVEPGGSLRLSCAASGFTFSNYYMSWVRQAPGKGPEWVSSIYSDLSTTYYADSVKGRFTISRDRAKNTLYLQMNSLKSEDTAVYYCARLSTVAGVDYWGKGTLVTVSS

SEQ ID NO: 330 - protein, 118 aa, Llama glama
QVQVQESGGGLVQPGGSLRLSCATSGFTFSNYYMSWVRQAPGKGPEWVSSIYSDLSTTYYADSVKGRFTISRDRAKNTLYLQMNSLKSEDTAVYYCARLSTVAGVDYWGKGTLVTVSS

SEQ ID NO: 331 - protein, 118 aa, Llama glama
QVQRQESGGGLVQPGGSLRLSCATSGFTFSNYYMSWVRQAPGKGPEWVSSIYSDLSTTYYADSVKGRFTISRDRAKNTLYLQMNSLKSEDTAVYYCARLSTVAGVDYWGKGTLVTVSS

SEQ ID NO: 332 - protein, 118 aa, Llama glama
QLQVVESGGGLVQPGGSLRLSCATSGFTFSNYYMSWVRQAPGKGPEWVSSIYSDLSTTYYADSVKGRFTISRDRAKNTLYLQMNSLKSEDTAVYYCARLSTVAGVDYWGKGTLVTVSS

SEQ ID NO: 333 - protein, 118 aa, Llama glama
QLQLVESGGGLVQPGGSLRLSCATSGFTFSNYYMSWVRQAPGKGPEWVSSIYSDLSTTYYADSVKGRFTISRDRAKNTLYLQMNSLKSEDTAVYYCARLSTVAGVDYWGKGTLVTVSS

SEQ ID NO: 334 - protein, 118 aa, Llama glama
QVQLVESGGGLVQPGGSLRLSCATSGFTFSNYYMSWVRQAPGKGPEWVSSIYSDLSTTYYADSVKGRFTISRDRAKNTLYLQMNSLKSEDTAVYYCARLSTVAGMDYWGKGTLVTVSS

SEQ ID NO: 335 - protein, 118 aa, Llama glama
ELQLVESGGGLVQPGGSLRLSCATSGFTFSNYYMSWVRQAPGKGPEWVSSIYSDLSTTYYADSVKGRFTISRDRAKNTLYLQMNSLKSEDTAVYYCARLSTVAGVDYWGKGTLVTVSS

SEQ ID NO: 336 - protein, 118 aa, Llama glama
EVQLQESGGGLVQPGGSLRLSCATSGFTFSNYYMSWVRQAPGKGPEWVSSIYSDLSTTYYADSVKGRFTISRDRAKNTLSLQMNSLKSEDTAVYYCARLSTVAGVDYWGKGTLVTVSS

SEQ ID NO: 337 - protein, 118 aa, Llama glama
EVQLVESGGGLVQPGGSLRLSCATSGFTFSNYYMSWVRQAPGKGPEWVSSIYSDLSTTYYADSVKGRFTISRDRAKNTLYLQMNSLKSEDTAVYYCARLSTVAGMDYWGKGTLVTVSS

SEQ ID NO: 338 - protein, 121 aa, Llama glama
EVQLVESGGGLVQPGGSLRLSCATSGFTFSNHYMSWVRQAPGKGLEWVSSIYSDGSDGGTTYYADSVKGRFTISRDRAKNTLSLQMNSLKSEDTAVYYCARLSTVAGMDYWGKGTLVTVSS

SEQ ID NO: 339 - protein, 118 aa, Llama glama
QVQVQESGGGLVQPGGSLRLSCATSGFTFSNYYMSWVRQAPGKGPEWVSSIYSDLSTTYYADSVKGRFTISRDRAKNTLYLQMNSLKSEDTAVYYCARLSTVAGMDYWGKGTLVTVSS

SEQ ID NO: 340 - protein, 118 aa, Llama glama
EVQVQESGGGLVQPGGSLRLSCATSGFTFSNYYMSWVRQAPGKGPEWVSSIYSDLSTTYYADSVKGRFTISRDRAKNTLYLQMNSLKSEDTAVYYCARLSTVAGVDYWGKGTLVTVSS

SEQ ID NO: 341 - protein, 126 aa, Llama glama
EVQVQESGPGLVKPSQTLSLTCTVSGGSITTDYSYWSWIRQPPGKGLEWMGAIAFSDTTYYSPSLKSRTSISRDTSKNQFTLQLSSVTPEDTAVYYCARLSTVAGMDYWGKGTLVTVSSASAAASG

SEQ ID NO: 342 - protein, 130 aa, Llama glama
EVQLVQPGAELRNPGASVKVSCKASGYTFTAYYIDWVRQAPGQGLEWMGRIDPEDGGTTYEQKFQGRVTFTADTSTSTVYVELSSLRSEDTAVYYCARSTEIPTIRYYGMDYWGKGTPVTVSSASAAASG

SEQ ID NO: 343 - protein, 123 aa, Llama glama
EVQLVQPGAELRNPGASVKVSCKASGYTFTSHYIDWVRQAPGQGLEWMGRIDPEDGGTTYEQKFQGRVTFTADTSTSTVYVELSSLRSEDTAVYYCARSTEIPTIRYYGMDYWGKGTPVTVSS

SEQ ID NO: 344 - protein, 113 aa, Llama glama
ELQVVESGGGLVPGGSLRLSCAASGFRVSVNAMSWVRQAPGKGLEWVSHIGSGGITGYADSVKGRFTISRDSAKNTLYLQMNSLKPEDTAVYYCTKRSSGYAWGQGTQVTVSS

SEQ ID NO: 345 - protein, 122 aa, Llama glama
EVQVQESGPGLVKPSQTLSLTCTVSGESITTNYYYWNWIRQPPGKALEWMGAIGDSGTTWYNPSLKSRTSISRDTSKNQFTLQLISVTPEDTAVYYCARGFYDGSSLDFASWGQGTQVTVSS

SEQ ID NO: 346 - protein, 122 aa, Llama glama
QVQLQESGPGLVKPSQTLSLTCTVSGGSITTNYYYWSWIRQPPGKGLEWMGAIAYSGSTYYSPSLKSRTSISRDTSKNQFTLQLSSVTPEDTAVYYCARGYYSGSSLDFGSWGQGTQVTVSS

SEQ ID NO: 347 - protein, 125 aa, Llama glama
QVQLQESGGGLVQPGGSLRLSCAASGFTFSWYAMSWVRQGPGKGLEWVSGINGGGGSTSYEDSVKGRFTISRDNAKNTLYLEMNSLKPEDTAVYYCARYSDGYSGSYTYTDMDYWGKGTLVSVSS

SEQ ID NO: 348 - protein, 125 aa, Llama glama
QVQLVESGGGLVQPGGSLRLSCAASGFTFSWYAMSWVRQGPGKGLEWVSAINGGGGSTSYEDSVKGRFTISRDNAKNTLYLEMNSLKPEDTAVYYCARYSDGYSGSYTYTDMDYWGKGTLVTVSS

SEQ ID NO: 349 - protein, 118 aa, Llama glama
EVQLVESEGGSVQPGDSLRLSCAASGFSFSNAGMSWVRQAPGKGLEWVSAIIPGGYAPYYADSVKGRFTISRDNAKNTVSLQMNSLKPEDTALYYCAKYGGNWDLEVWGQGTLVTVSS

SEQ ID NO: 350 - protein, 118 aa, Llama glama
QLQLVESGGGLQQPGGSLRLSCAASGFTFSSYAMGWVRQAPGKGLEWISIILNDGHRTYYTDSVKGRFTISRDNAKDTLSLQMNSLKPEDTAVYFCVKYVLNWAISDWGQGTQVTVSS

SEQ ID NO: 351 - protein, 124 aa, Llama glama
QLQVVESGGGLVQPGGSLRLSCAASGFTFDSYWMYWVRQAPGKGLEWLSAINPGGGMTYYADSVKGRFTIARDNAKNTVYLQMNSLKSEDTAVYYCAKELRGSYYYISSEYDYWGQGTQVTVSS

SEQ ID NO: 352 - protein, 122 aa, Llama glama
ELQLVESGGGLVQPGGSLRLSCAASGSTFSTAAMSWVRQAPGKGLEWVSAISSFGASTSYADSVKGRFTISRDIAKNTLYLQMNSLKPEDAAVYYCANVGLHPSWGFTSGFWGQGTQVTVSS

SEQ ID NO: 353 - protein, 125 aa, Llama glama
QVQLVESGGGLVQPGGSLRLSCAASGFTFSSYAMTWVRQAPGKGLEWVSTITSNGDSTSYADSVKGRFTISRDNAKNTLYLQMDSLKPEDTAVYYCAKLGDGSYWFYYYGMDYWGQGTQVTVTSS

SEQ ID NO: 354 - protein, 118 aa, Llama glama
QLQLVESGGGLVQPGGSLRLSCAASGGIFDVMGWYHQAPGKQRGLVASITPAGSTNYADSVKGRFTISRDNAKNTVYLQMNSLKPDDTGVYYCYADGIDPRYGGDEFWGQGTQVTVSS

SEQ ID NO: 355 - protein, 109 aa, Llama glama
SYELTQSPSVSVALRQTAKITCGGNNIGSKSAQWYQQKPGQAPVLVIYADSRRPSGIPERFSGSNSGNTATLTISGAQAEDEADYYCQVWDSSSNGAVFGGGTHLTVLG

SEQ ID NO: 356 - protein, 109 aa, Llama glama
SYELTQSPSVSVALRQTAKITCGGNNIGSKSAQWYQQKPGQAPVLVIYADYRRPSGIPERFSGSNSGNTATLTISGAQAEDEADYYCQVWDSSANAAVFGGGTHLTVLG

SEQ ID NO: 357 - protein, 109 aa, Llama glama
SYELTQSPSVSVALRQTAKITCGGNNIGSKSAQWYQQKPGQAPVLVIYADVRRPSEIPERFSGSNSGNTATLSVSGAQAEDEADYYCQVWDSSANAAVFGGGTHLTVLG

SEQ ID NO: 358 - protein, 109 aa, Llama glama
SYELTQSPSVSVALRQTAKITCGGNNIGSKSAQWYQQKPGQAPVLVIYADSRRPSGIPEKFSGSNSGNTATLTISGAQAEDEADYYCQVWDSSANAAVFGGGTHLTVLG

SEQ ID NO: 359 - protein, 109 aa, Llama glama
SYELTQSPSVSVALRQTAKITCGGNNIGSKSAQWYQQKPGQAPVLVIYADSRRPSGIPERFSGSNSGNTATLTISGAQAEDEADYYCQVWDSSANAAVFGGGTHLTVLG

SEQ ID NO: 360 - protein, 109 aa, Llama glama
SYELTQSPSVSVALRQTAKITCGGTNIGSKSAQWYQQKPGQAPMLVIYGDSRRPSGIPERFSGSNSGNTATLTISGAQAEDEADYYCQVWDSSANAAVFGGGTHLTVLG

SEQ ID NO: 361 - protein, 109 aa, Llama glama
SYELTQSPSVSVALRQAAKITCGGNDIGSKSAQWYQQKPGQAPVLVIYADSRRPSGIPERFSGSNSGNTATLTISGAQAEDEADYYCQVWDSSANAAVFGGGTHLTVLG

SEQ ID NO: 362 - protein, 107 aa, Llama glama
SYELTQSPSVSVALRQTAKITCGGNNIGSKSAQWYQQKPGQAPVLVIYADSRRPSGIPERFSGSNSGNTATLTISGAQAEDEADYYCQVWDSSAAVFGGGTHLTVLG

SEQ ID NO: 363 - protein, 109 aa, Llama glama
SYELTQSPSVSVALRQAAKITCGGDNMGSKSAQWYQQKPGQAPELVIYADNRRPSEIPERFSGSNSGDTATLTISGAQAEDEADYYCQVWDSGANAAVFGGGTHLTVLG

SEQ ID NO: 364 - protein, 109 aa, Llama glama
SYELTQSPSVSVALRQTAKITCGGNNIGDKSAQWYQQKPDQAPVLVIYADNRRPSEIPERFSGSNSGDTATLTISGAQAEDEADYYCQVWDVGANAAVFGGGTHLTVLG

SEQ ID NO: 365 - protein, 109 aa, Llama glama
SYELTQSPSVSVALRQTAKITCGGNDIESKSAQWYQQKPDQAPVLVIYADDRRPSEIPERFSGSNSGDTATLTISGAQAEDEADYYCQVWDSSANAAVFGGGTHLTVLG

SEQ ID NO: 366 - protein, 109 aa, Llama glama
SYELTQSPSVSVALRQTAKITCGGNNIGDKSAQWYQQKPDQAPVLVIYADNRRPSEIPERFSGSNSGDTATLTISGAQAEDEADYYCQVWDSGANAAVFGGGTHLTVLG

SEQ ID NO: 367 - protein, 109 aa, Llama glama
SYELTQSPSVSVALRQTAKITCGGNNIGSKSAQWYQQKPGQAPVLVIYADNRRPSEIPERFSGSNSGDTATLTISGAQAEDEADYYCQVWDSSANAAVFGGGTHLTVLG

SEQ ID NO: 368 - protein, 109 aa, Llama glama
SYELTQSPSVSVALRQTAKITCGGDNIGSKSAQWYQQKPGQAPVLVIYADSRRPSGIPERFSGSNSGNTATLTISGAQAEDEADYYCQVWDSSANAAVFGGGTHLTVLG

SEQ ID NO: 369 - protein, 108 aa, Llama glama
QSVLTQPSAVSVSLGQTARITCQGGSLGSYGANWHQQKPGQAPVLLIYGDNSRPSGIPERFSGSKSGGTATLTISGAQAEDEADYYCQSFDYSGNAVFGGGTHLTVLG

SEQ ID NO: 370 - protein, 112 aa, Llama glama
QAGLTQLPSVSGSPGQKITISCTGSSNNIGAGYSVQWFQQVPGSPPRLLIDGATNRDSGVPDRFSDSRSGSSASLTITGLQAEDEADYYCECYDNSLRGAVFGGGTHLTVLG

SEQ ID NO: 371 - protein, 106 aa, Llama glama
QAGLTQLPSVSGSPGQKITISCTGSSNNIGAGYSVQWFQQVPGSPPRLLIDGATNRDSGVPDRFSDSRSGSSASLTITGLQAEDEADYYWMLQQPQGVLCSAEGPI

SEQ ID NO: 372 - protein, 108 aa, Llama glama
LSYELTQSPSVSVALRQAAKITCGGNNIASKFVNWYQQKPGQAPILVISKDTNRPSGIPERFSGSNSGNTATLTVSGAQAEDEADYYCQVWDGSAAVFGGGTHLTVLG

SEQ ID NO: 373 - protein, 107 aa, Llama glama
LPVLTQPSAVSVSLGQTARITCQGGNVGSYYTSWYQQKPGQAPVLVIYKDSERPSGIPERFSGSSSGNTATLTVSGAQAEDEGDYYCQVWDSSAAVFGGGTHLTVLG

SEQ ID NO: 374 - protein, 111 aa, Llama glama
QAVVTQEPSLSVSPGGTVTLTCGLNSGSVTNGNYPGWYQQTPGQAPRALIYSTNSRHSGVPGRYSGSISGNKAALTITGAEPEDEADYYCALWQAAGTVVFGGGTHLTVLG

SEQ ID NO: 375 - protein, 111 aa, Llama glama
QAVVTQEPSLSVSPGGTVTLTCGLSSGSVTSSNYPVWYQQTPGQAPHLLIYNTNTRHSGVPSRFSGFISGNKAALTITGAQPEDEADYYCASYRRSGRAVFGGGTHLTVLG

SEQ ID NO: 376 - protein, 111 aa, Llama glama
QSALTQPPSVSGTLGKTVTISCTGTSSDIGGYNSVSWYQQLPGTAPKLLIYEVNKRASGIPDRFSGSKSGDTASLTISGLQSADEADYYCASYRNTRKSVFGGGTHLTVLG

SEQ ID NO: 377 - protein, 111 aa, Llama glama
QSVLTQPPSVSGSPGKTVTISCAGTSSDVGYGNYVSWYQQLPGMAPKLLIYNANKRASGITDRFSASKSGNTASLTISGLQSGDEAVYYCASYRYRDNAVFGGGTHLTVLG

SEQ ID NO: 378 - protein, 111 aa, Llama glama
LPVLTQPPSVSGTLGKTVTISCAGTSSDIGDYNYVSWYQQLPGTAPKLLIYKVSTRASGIPDRFSGSKSGNTASLTISGLQSEDEADYYCASYRTSNNVVFGGGTHLTVLG

SEQ ID NO: 379 - protein, 111 aa, Llama glama
SSALTQPPSVSGSPGKTVTISCAGTSSDVGYGNYVSWYQQLPGMAPKLLIHDVNKRASGITDRFSGSKSGNTASLTISGLQSEDEADYYCASYRRRNEVVFGGGTHLTVLG

SEQ ID NO: 380 - protein, 109 aa, Llama glama
SYELTQSSAVSLSLGQTARITCQGDNLGRYYVNWYQQKPGQAPVQVIYGNSNRPSGIPERFSGSSSGGTATLTISGAQAEDEADYYCQTYENSGVNLVFGGGTKLTVLG

SEQ ID NO: 381 - protein, 109 aa, Llama glama
SYELTQSPSVSVALRQTAKITCGGDNIGSKSAQWYQQKPGQAPVLVIYAGSRRPSGIPERFSGSNSGNTATLTVSGAQAEDEANYSCQVWDSSANASVFGGGTHLTVLG

SEQ ID NO: 382 - protein, 119 aa, Llama glama
QLQLVESGGGLVQPGGSLRLSCAASGFTFSSNAMTWVRQAPGKGLEWVSAISSTGNSITEADSVKGRFTISRDNAKNMLYLQMNSLKPEDTAVYYCAKLGGDTWYMEYWGQGTQVTVSS

SEQ ID NO: 383 - protein, 119 aa, Llama glama
ELQVVESGGGLVQPGGSLRLSCAASGFTFSSNAMTWVRQAPGKGLEWVSAISSTGNSITEADSVKGRFTISRDNAKNMLYLQMNSLKPEDTAVYYCAKLGGDTWYMEYWGQGTQVTVSS

SEQ ID NO: 384 - protein, 119 aa, Llama glama
QLQVVESGGGLVQPGGSLRLSCAASGFTFSSNAMTWVRQAPGKGLEWVSAISSTGNSITEADSVKGRFTISRDNAKNMLYLQMNSLKPEDTAVYYCAKLGGDTWYMEYWGQGTQVTVSS

SEQ ID NO: 385 - protein, 119 aa, Llama glama
QLQVVESGGGLVQPGGSLRLSCAASGFTFSRNAMTWVRQAPGKGLEWVSAISSTGNSITEADSVKGRFTISRDNAKNMLYLQMNSLKPEDTAVYYCAKLGGDTWYMEYWGQGTQVTVSS

SEQ ID NO: 386 - protein, 116 aa, Llama glama
QVQLVESGGGLVQPGGSLRLSCAASGFTFSSNAMTWVRQAPGKGLEWVSAISSTGNSITEADSVKGRFTISRDNAKNMLYLQMNSLKPEDTAVYYCAKLGWYMEYWGQGTQVTVSS

SEQ ID NO: 387 - protein, 119 aa, Llama glama
QVQLVESGGGLVQPGGSLRLSCAASGFTFSSNAMTWVRQAPGKGLEWVSAISSTGNSITEADSVKGRFTISRDNAKNMLYLQMNSLKPEDTAVYYCAKLGGDTWYMEYWGQGTQVTVSS

SEQ ID NO: 388 - protein, 119 aa, Llama glama
EVQLVESGGGLVQPGGSLRLSCAASGFTFSRNAMTWVRQAPGKGLEWVSAISSTGNSITEADSVKGRFTISRDNAKNMLYLQMNSLKPEDTAVYYCAKLGGDTWYMEYWGQGTQVTVSS

SEQ ID NO: 389 - protein, 119 aa, Llama glama
QVQLQESGGGLVQPGGSLRLSCAASGFTFSSNAMTWVRQAPGKGLEWVSAISSTGNSITEADSVKGRFTISRDNAKNMLYLQMNSLKPEDTAVYYCAKLGGDTWYMEYWGQGTQVTVSS

SEQ ID NO: 390 - protein, 119 aa, Llama glama
QVQVQESGGGLVQPGEFLRLSCAASGFTFSSNAMTWVRQAPGKGLEWVSAISSTGNSITEADSVKGRFTISRDNAKNMLYLQMNSLKPEDTAVYYCAKLGGDTWYMEYWGQGTQVTVSS

SEQ ID NO: 391 - protein, 124 aa, Llama glama
QLQVVESGGGLVQPGGSLRLSCAASGFTFSTYVMSWVRQAPGKGLEWVSGINSGGGSTSYADSVKGRFTISRDNAKNTLYLQMNSLKPEDTAVYYCAKVGDGSAWYYYYGMDYWGQGTQVTVSS

SEQ ID NO: 392 - protein, 119 aa, Llama glama
QVQLQESGGGLVQPGGSLRLSCATSGFTFSANAMTWVRQAPGKGLEWVSAISGSGASTSYADSVKGRFTISRDNAKNTLYLQMNNLKPEDTAVYHCAKLGGDTWYMEYWAKGTLVTVSS

SEQ ID NO: 393 - protein, 119 aa, Llama glama
EVQLQESGGGSVQPGGSLRLSCAASGFTFSANAMTWVRQAPGKGLEWVSAISGTGASTSYADSVKGRFTVSRDNAKNTLYLQVNNLKPEDTAVYRCAKLGGDTWYMEYWAKGTLVTVSS

SEQ ID NO: 394 - protein, 109 aa, Llama glama
SYELTQSPSVSVALRQTAKITCGGNGIGSKIVHWYQQKPGQAPVLVIYADVRRPSEIPERFSGSNSGDTATLTVSGAQAEDEADYYCQVWDSSANAAVFGGGTHLTVLG

SEQ ID NO: 395 - protein, 108 aa, Llama glama
SSALTQPSAVSVSLGQTARITCQGDNFGSIDPHWYQQKPGQDPVVVIYQYNNRPSGIPERFSGLKSGNTATLIISGAQAEDEADYYCQLWDSRAKAVFGGGTHLTVLG

SEQ ID NO: 396 - protein, 107 aa, Llama glama
SYELTQSPSVSVALRQAAKITCGGDNIASKYANWYQQKPGQAPVVVIYKDSNRPSGIPERFSGSNSGNTATLTVSGAQAEDEADYYCQVWDSGAAVFGGGTHLTVLG

SEQ ID NO: 397 - protein, 108 aa, Llama glama
SYELTQSPSVSVALRQTAKITCGGNNIGSKNAHWYQQKPGQAPVLVIYKDNERSSGTPERFSGSSSGDTATLTISGAQAEDEADYYCQVWDSSANAVFGGGTHLTVLG

SEQ ID NO: 398 - protein, 107 aa, Llama glama
SYELTQSPSVSVALRQAAKITCGGDNIASKYANWYQQKPGQAPVLVIYKDSNRPSGIPERFSGSNSGNTATLTVSGAQAEDEADYYCQVWDSSAAVFGGGTHLTVLG

SEQ ID NO: 399 - protein, 108 aa, Llama glama
SYELTQSPRVSVALRQTAKITCGGDNIGSKSAQWYQQKPGQAPVLVIYADSRRPSGIPERFSGSNSGNTATLTISGAQAEDEADYYCQVWDSSANAVFGGGTHLTVLG

SEQ ID NO: 400 - protein, 107 aa, Llama glama
SYELTQSPSVSVALRQAAKITCGGDNIGSKNAHWYQQKPGQAPVLVIYADSRRPSGIPERFSGSNSGNTATLVVSGAQAEDEADYYCQVWDSSAAVFGGGTHLTVLG

SEQ ID NO: 401 - protein, 108 aa, Llama glama
QSALTQPSALSVTLGQTAKITCQGGSLSNNYAHWYQQKPGQAPVMAIYDDDSRPSGIPERFSGSRSGGIATLTISGAQAEDEADYYCQVWDSSANAVFGGGTQLTVLG

SEQ ID NO: 402 - protein, 108 aa, Llama glama
SSALTQPSAVSVSLGQTARITCQGDNFGSSYPHWYQQKSGQVPVLVIYQYNDRPSGIPERFSGTKSGDTATLTISGAQAEDEADYYCQVWDSSANAVFGGGTHLTVLG

SEQ ID NO: 403 - protein, 108 aa, Llama glama
SYELTQSPSMSVALRQTAKITCGGNNIGSKSAQWYQQKPGQAPVLVIYADNRRPSGIPERFSGSNSGNTATLTISGAQAEDEADYYCQVWDSSANAVFGRGTHLTVLG

SEQ ID NO: 404 - protein, 107 aa, Llama glama
SYELTQSPSVSVALRQAAKITCDGDDIASKYANWYQQKPGQAPVMVIYKDNNRPSGIPERFSGSNSGNTATLTVSGAQAEDEADYYCQVWDSSAAVFGGGTHLTVLG

SEQ ID NO: 405 - protein, 107 aa, Llama glama
SYELTQSPSVSVALRQAAKITCGGDNIASKYANWYQQKPGQAPVLVIYKDSNRPSGIPERFSGSNSGNTATLTVNGAQAEDEADYYCQVWDNSAAVFGGGTHLTVLG

SEQ ID NO: 406 - protein, 109 aa, Llama glama
SSALTQPSAVSVSLGQTARITCQGGNLDGYYHAWYQQKPGQAPVQVIFGNNNRPSGIPERFSGSSSGGTTTLTISGAQAEDEADYYCQSYENSGYNIVFGGGTHLTVLG

SEQ ID NO: 407 - protein, 107 aa, Llama glama
LPVLTQPSALSVTLGQTAKITCQGDNLGSTYVHWYQHKPGQAPVLVIYVDSRRPSGIPERFSGSNSGNTATLTITGAQAEDEADYYCQVWDSSTAVFGGGTHLTVLG

SEQ ID NO: 408 - protein, 107 aa, Llama glama
SYELTQSPSVSVALRQTAKITCGGDNIGSKYVNWYQQKPSQTPVLVIYKDSNRPSGIPERFSGSNSGNTAALTISGAQAEDEADYYCQVWDGSAAVFGGGTHLSVLG

SEQ ID NO: 409 - protein, 115 aa, Llama glama
QVPLLESGGSLVPPGGSPTIPRATPGFPFHNYWMYWVPPAPGKALQWVSRFNFCGGSTYYTHSVKGIFTISRDNQKNTLYLQMNSLQPEDTALYYCRGGIVLHYWGQGTQVTVSS

SEQ ID NO: 410 - protein, 115 aa, Llama glama
QLQESGPGLVKPSQTLSLTCTVSGGSITTNYYYWSWIRHTPGKGLQWMGAIAYSGSTYYNPSLKSRTSISRDTFKNQFTLQLSSVTPEDTAVYYCARDLLGPSLLGQGTQVTVSS

SEQ ID NO: 411 - protein, 122 aa, Llama glama
AVQESGPGLVKPSQTLSLTCTVSGGSITTKYYYWNWIRQPPGKGLEWMGAIAYSGTTYYSPSLESRTSISRDTSKNQFTLQLSSVTPEDTAVYYCARGVYSGSYYYRYLEVWGRGTLVTVSS

SEQ ID NO: 412 - protein, 112 aa, Llama glama
GGGLVQPGGSLRLSCAASGFTFSNLGMHWVRQAPGKGLEWVSAINAGGSTTYYTDSVKGRFTISRDNAKNTLYLQMNSLKPEDTAVYHCAKLGGDTWYVDYWAKGTLVTVSS

SEQ ID NO: 413 - protein, 116 aa, Llama glama
QLQLVESGGGLVQPGGSLRLSCAASGFTFSSSWMYWVRQAPGKGLEWVSSIATVSLSTYYADSVKGRLTISRDNAKNTLYLQMDSLRSDDTAVYYCARNTATWDSWGQGTQVTVSS

SEQ ID NO: 414 - protein, 114 aa, Llama glama
GGGLVQPGGSLRLSCAASGFAFSSAWMLWVRHSPGTGLEWVSGINTQTGTTYYADSVKGRFTISRDNAKNTLSLQMNSLKLEDTAVYYCAIVVYTLAPDGYDYWGQGTQLTVSS

SEQ ID NO: 415 - protein, 113 aa, Llama glama
GGGLVQPGGSLILSCAASGFTFSTYAMNWVRQAPGKGPEWVSSIARGGGSAYYADSMKGRFAISRDDAKNTLYLQMNSLKPEDTAVYYCSTNGVDGNWAFDYWGQGTQVTISS

SEQ ID NO: 416 - protein, 114 aa, Llama glama
GGSLVQPGGSLKLSCAASGFTFSSSWMYWVPQAPGKGLEWVSTIDSDGTNTYYADSVKGRFTISRDNAKNTLYLQMNSLKSEDTAVYYCLKISDYGIYWGQGTWGQGTQVTVSS

SEQ ID NO: 417 - protein, 112 aa, Llama glama
SGPGLVKPSQTLSLTCTVSGGSITTNYYYWSWIRQSPGKGLEWMGAMTYTGSAYYSPSLKSRASISRDTFKNQFTLQLSSVTPEDTAVYYCARGGYSPYDYWGQGTQVTVSS

SEQ ID NO: 418 - protein, 117 aa, Llama glama
SGPGLVKPSQTLSLTCTVSGGSITASHYYWSWIRQPPGKGLEWMGTIRYSGITYYSPSLKSRTSISRDTSTNQFHLQLSSVTPEDTAVYYCARDSGSFYGFVKYDYWGQGTQVTVSS

SEQ ID NO: 419 - protein, 118 aa, Llama glama
SGPGLVKSSQTLSLTCTVSGGSITTIYHYWSWIRQPPGKGLEWMGAIHSSGSTYDSPSLKSRTSFSRDTSKNQFILQLSSVTPEDTAVYYCARGNTWGLRRGVRYDVWGQGTQVTVSS

SEQ ID NO: 420 - protein, 113 aa, Llama glama
GVGGGFVQPGGSLRLSCAASGFAFSGYAMSWVRQAPGEGLEWVAAIDSAGGNTTYVDSVKGRFTISRDNAKNTLYLQMNNLTYEDTGVYYCTRPPMGISPNYWGQGIQVTVSS

SEQ ID NO: 421 - protein, 115 aa, Llama glama
ESGGGLVQPGGSLRLSCATSGFTFSGYWMYWVRQAPGKGLEWVSSINTFSGTTIYADSVKGRFTISRDNAKNTLYLQINSLKPEDTALYYCARAYDLGYTVFRSWGQGTQVTVSS

SEQ ID NO: 422 - protein, 113 aa, Llama glama
GVGGGLVQPGGSLRLSCAGSGFTFSTAWMYWVRQAPGKGLEWVSGINTVNSDGTYYARSVKGRFTISRDNARNTVYLQMNALEPEDTALYYCARESVTTGWDWGQGTQVTVSS

SEQ ID NO: 423 - protein, 107 aa, Llama glama
PGGSLRLSCTASGFTFSSSFMNWVRQAPGKGLEWVSTINGRGIDRYYADSVKGRFGISRDNAKNTAYLQMNSLKLDDTAVYYCANLWPATVPDFGSWGQGTQVTVSS

SEQ ID NO: 424 - protein, 117 aa, Llama glama
PGAAAGVGGGLVQPGGSLRLSCVGSGFTFDDYVLNWVRQAPGKGLEWVSTIASGAGTTYYAGSVKGRFTISRDYYKNTLYLQMNSLTPDDTAVYHCTTGSWYRYNSWGQGTQVIVSS

SEQ ID NO: 425 - protein, 112 aa, Llama glama
GVGGGLVQPGGSLRLSCVGSGFTFDDYVLNWVRQAPGKGLEWVSTIASGAGTTYYAGSVKGRFTISRDYYKNTLYLQMNSLTPDDTAVYHCTTGSWYRYNSWGQGTQVIVSS

SEQ ID NO: 426 - protein, 119 aa, Llama glama
ELQLVESGGGLVQPGGSLTLSCAASGFTFSSNAMTWVRQAPGKGLEWVSAISSTGNSITEADSVKGRFTISRDNAKNMLYLQMNSLKPEDTAVYYCAKLGGDTWYMEYWGQGTQVTVSS

SEQ ID NO: 427 - protein, 119 aa, Llama glama
ELQLVESGGGLVQPGGSLRLSCAASGFTFSSNAMTWVRQAPGKGLEWVSAISSTGNSITEADSVKGRFTISRDNAKNMLYLQMNSLKPEDTAVYYCAKLGGDTWYMEYWGQGTQVTVSS

SEQ ID NO: 428 - protein, 119 aa, Llama glama
EVQVQESGGGLVQPGGSLRLSCAASGFTFSSNAMTWVRQAPGKGLEWVSAISSTGNSITEADSVKGRFTISRDNAKNMLYLQMNSLKPEDTAVYYCAKLGGDTWYMEYWGQGTQVTVSS

SEQ ID NO: 429 - protein, 119 aa, Llama glama
EVQLVESGGGLVQPGGSLRLSCAASGFTFSSNAMTWVRQAPGKGLEWVSAISSTGNSITEADSVKGRFTISRDNAKNMLYLQMNSLKPEDTAVYYCAKLGGDTWYMEYWGQGTQVTVSS

SEQ ID NO: 430 - protein, 119 aa, Llama glama; identical to SEQ ID NO: 429

SEQ ID NO: 431 - protein, 119 aa, Llama glama; identical to SEQ ID NO: 429

SEQ ID NO: 432 - protein, 119 aa, Llama glama
QVQLVESGGGLVQPGGSLRLSCAASGFTFSRNAMTWVRQAPGKGLEWVSAISSTGNSITEADSVKGRFTISRDNAKNMLYLQMNSLKPEDTAVYYCAKLGGDTWYMEYWGQGTQVTVSS

SEQ ID NO: 433 - protein, 119 aa, Llama glama; identical to SEQ ID NO: 387

SEQ ID NO: 434 - protein, 119 aa, Llama glama
QLQLVESGGGLVQPGGSLRLSCAASGFTFSSNAMTWVRQAPGKGLEWVSAISSTGNSITEADSMKGRFTISRDNAKNMLYLQMNSLKPEDTAVYYCAKLGGDTWYMEYWGRGTQVTVSS

SEQ ID NO: 435 - protein, 119 aa, Llama glama
QVQLVESGGGLVQPGGSLRLSCAASGFTFSANAMTWVRQAPGKGLEWVSAISGTGATTTYTDSVKGRFTISRDNAKNTLYLQMNNLKPEDTAVYHCAKLGGDTWYMEHWAKGTLVIVSS

SEQ ID NO: 436 - protein, 119 aa, Llama glama
QVQLVESGGGLVQPGGSLRLSCAASGFTFSSNAMSWVRQAPGKGLEWVSAISASGATTAYADSVRGRFTISRDNAKNTLYLQMNSLKPDDTAVYYCAKSEGATWYYAYWGQGTQVTVSS

SEQ ID NO: 437 - protein, 119 aa, Llama glama; identical to SEQ ID NO: 436

SEQ ID NO: 438 - protein, 119 aa, Llama glama
ELQLVESGGGLVRPGGSLRVSCAASGFTFSKNAMSWVRQAPGKGLEWVSAISGSAKTTTYADSVKGRFTISRDNAKNTLNLQMNSLNVEDTAVYYCAKSDSDTWYFGSWGQGTQVTVSS

SEQ ID NO: 439 - protein, 119 aa, Llama glama
QLQLVESGGGLVQPGGSLRLSCAASGFTFSKNAMSWVRQAPGKGLEWVSVISSTGNTTAHADSVKGRFTISRDNARNIMYLQMNRLKPEDTAVYYCAKLGGATWYMDSWGQGTQVTVSS

SEQ ID NO: 440 - protein, 119 aa, Llama glama
QLQVVESGGGLVQPGESLRLSCAASGFTFSKNAMSWVRQAPGKGLEWVSVISSTGNTTAHADSVKGRFTISRDNARNIMYLQMNRLKPEDTAVYYCAKLGGATWYMDSWGQGTQVTVSS

SEQ ID NO: 441 - protein, 119 aa, Llama glama
QVQLVESGGGLVQPGESLRLSCAASGFTFSKNAIAWVRQAPGKGLEWVSVISSTGNTTAHADSVKGRFTISRDNARNIMYLQMNSLKPEDTAVYYCAKLGGATWYMDSWGQGTQVTVSS

SEQ ID NO: 442 - protein, 30 aa, Llama glama
EVQLVESGGGLAQPGGSLRLSCRASGFAFG

SEQ ID NO: 443 - protein, 30 aa, Llama glama
EVQLVQPGAELRNPGASVKVSCKASGYTFT

SEQ ID NO: 444 - protein, 30 aa, Llama glama
EVQLVQPGHELKNPGASVKVSCKASGYIFT

SEQ ID NO: 445 - protein, 30 aa, Llama glama
EVQLVQPGTELRNPGSSVKVSCKASGYIFS

SEQ ID NO: 446 - protein, 30 aa, Llama glama
EVQLVQPGTELRNPGASVKVSCKTSGYTFT

SEQ ID NO: 447 - protein, 30 aa, Llama glama
EVQLQESGGALVQPGGSLRLSCAASGFTFS

SEQ ID NO: 448 - protein, 30 aa, Llama glama
QVQLQESGPGLVKPSQTLSLACTVSGGSIT

SEQ ID NO: 449 - protein, 30 aa, Llama glama
ELQLVESGGGLVQPGGSLRLSCAASGFTFD

SEQ ID NO: 450 - protein, 30 aa, Llama glama
QVQLVESGGGLVQPGGSLRLSCAASGFTFG

SEQ ID NO: 451 - protein, 30 aa, Llama glama
ELQLVESGGGLVEPGGSLRLSCAASGFTFS

SEQ ID NO: 452 - protein, 5 aa, Llama glama
SFWMY

SEQ ID NO: 453 - protein, 5 aa, Llama glama
RYYID

SEQ ID NO: 454 - protein, 5 aa, Llama glama
SSYID

SEQ ID NO: 455 - protein, 5 aa, Llama glama
NHYIH

SEQ ID NO: 456 - protein, 5 aa, Llama glama
SSYIN

SEQ ID NO: 457 - protein, 5 aa, Llama glama
KYYID

SEQ ID NO: 458 - protein, 5 aa, Llama glama
NYFID

SEQ ID NO: 459 - protein, 5 aa, Llama glama
GYYID

SEQ ID NO: 460 - protein, 5 aa, Llama glama
TYAMA

SEQ ID NO: 461 - protein, 5 aa, Llama glama
RSWMY

SEQ ID NO: 462 - protein, 7 aa, Llama glama
LNGGYWS

SEQ ID NO: 463 - protein, 5 aa, Llama glama
DYAMS

SEQ ID NO: 464 - protein, 5 aa, Llama glama
SYDMS

SEQ ID NO: 465 - protein, 5 aa, Llama glama
NYAMT

SEQ ID NO: 466 - protein, 14 aa, Llama glama
WVRQTPGKGLEWVS

SEQ ID NO: 467 - protein, 14 aa, Llama glama
WVRQVPGQGFEWMG

SEQ ID NO: 468 - protein, 14 aa, Llama glama
WLRQAPGQGPEWMG

SEQ ID NO: 469 - protein, 14 aa, Llama glama
WVRQAPGQGLEWVG

SEQ ID NO: 470 - protein, 14 aa, Llama glama
WVRQRPGEGLEWMG

SEQ ID NO: 471 - protein, 14 aa, Llama glama
WVRQAPGKGPEWVS

SEQ ID NO: 472 - protein, 17 aa, Llama glama
TVDAAGANTYYPDTVKG

SEQ ID NO: 473 - protein, 17 aa, Llama glama
RIDPEDGGTKVARKFQG

SEQ ID NO: 474 - protein, 17 aa, Llama glama
RMDPEDGTTKPAPKFQG

SEQ ID NO: 475 - protein, 17 aa, Llama glama
RIDPEDGGVKYAQRFRG

SEQ ID NO: 476 - protein, 17 aa, Llama glama
RIDPEDGRTKSRQNFQG

SEQ ID NO: 477 - protein, 17 aa, Llama glama
LIDPEDGATKYAQKFQG

SEQ ID NO: 478 - protein, 17 aa, Llama glama
SIDPEDGGTQTAQKFQG

SEQ ID NO: 479 - protein, 17 aa, Llama glama
RIDPEDGDTKYAQKFQG

SEQ ID NO: 480 - protein, 15 aa, Llama glama
FINKYGDGYADSVKG

SEQ ID NO: 481 - protein, 17 aa, Llama glama
AMNPGGGSTYYADSVKG

SEQ ID NO: 482 - protein, 16 aa, Llama glama
AIAYTGSTYISPSLKS

SEQ ID NO: 483 - protein, 17 aa, Llama glama
GTTRNGGRTYYAESMKG

SEQ ID NO: 484 - protein, 17 aa, Llama glama
AINKSGGSTYYADSVKG

SEQ ID NO: 485 - protein, 17 aa, Llama glama
AINSGGGSTYYGDAVKG

SEQ ID NO: 486 - protein, 32 aa, Llama glama
RFTISRDNAKNTLYLRMNSLKPEDTARYYCVT

SEQ ID NO: 487 - protein, 32 aa, Llama glama
RVTFTADTSASTVYLSLTNLRSDDTGVYYCAT

SEQ ID NO: 488 - protein, 32 aa, Llama glama
RVTFTADTSAHTAYMELTTLRVDDTAVYFCAI

SEQ ID NO: 489 - protein, 32 aa, Llama glama
RVTFTVDTSTTTAYVELHSLRTDDTGVYYCAF

SEQ ID NO: 490 - protein, 32 aa, Llama glama
RVTFTADTSTSTAYVELRNLRSEDTAVYYCVA

SEQ ID NO: 491 - protein, 32 aa, Llama glama
RVTFTTDTSTSTAYVELSSLRSEDTAVYYCLI

SEQ ID NO: 492 - protein, 32 aa, Llama glama
RVIFTVDTSTSTAYVEVSSLRPEDTGVYYCVI

SEQ ID NO: 493 - protein, 32 aa, Llama glama
GVTITADTSTTTAYLELSSLRSEDTAVYYCAL

SEQ ID NO: 494 - protein, 32 aa, Llama glama
RFTISRDNAKNTLTLQMNSLKPEDTAVYWCAK

SEQ ID NO: 495 - protein, 32 aa, Llama glama; identical to SEQ ID NO: 22

SEQ ID NO: 496 - protein, 32 aa, Llama glama
RTSISRDASENQFTLQLSAVTPEDSAVYYCAR

SEQ ID NO: 497 - protein, 32 aa, Llama glama
RFTISRDNDKNTLYLQMNSPKSEDTGVYYCAK

SEQ ID NO: 498 - protein, 32 aa, Llama glama
RFTISRDNAKNTLYLQMNSLKPEDTAVYYCAT

SEQ ID NO: 499 - protein, 32 aa, Llama glama
RFTISRDNAKNTLYLQINSLKPEDTAVYYCAK

SEQ ID NO: 500 - protein, 17 aa, Llama glama
DGAYDDPAIRGKIPLAS

SEQ ID NO: 501 - protein, 5 aa, Llama glama
RGRGS

SEQ ID NO: 502 - protein, 9 aa, Llama glama
DDGLGALNY

SEQ ID NO: 503 - protein, 7 aa, Llama glama
WSDDDDY

SEQ ID NO: 504 - protein, 8 aa, Llama glama
GAPDEYAY

SEQ ID NO: 505 - protein, 4 aa, Llama glama
FPRY

SEQ ID NO: 506 - protein, 4 aa, Llama glama
PWNY

SEQ ID NO: 507 - protein, 10 aa, Llama glama
SRGDHDDYDY

SEQ ID NO: 508 - protein, 5 aa, Llama glama
AGMST

SEQ ID NO: 509 - protein, 14 aa, Llama glama
HHYDSNWYYYGMDY

SEQ ID NO: 510 - protein, 11 aa, Llama glama
SDYALGSTFSS

SEQ ID NO: 511 - protein, 8 aa, Llama glama
MYGSSWSY

SEQ ID NO: 512 - protein, 9 aa, Llama glama
VGGLGYVDY

SEQ ID NO: 513 - protein, 11 aa, Llama glama
WGQGTLVTVSS

SEQ ID NO: 514 - protein, 11 aa, Llama glama
WGQGTRVTVSS

SEQ ID NO: 515 - protein, 11 aa, Llama glama
WGHGTQVTVSS

SEQ ID NO: 516 - protein, 11 aa, Llama glama
WGQGTQVTVSS

SEQ ID NO: 517 - protein, 11 aa, Llama glama
WGKGTLVTVSS

SEQ ID NO: 518 - protein, 23 aa, Llama glama
DIVMTQSPSSVTASVGEKVTITC

SEQ ID NO: 519 - protein, 23 aa, Llama glama
EIVLTQSPSSVTASAGEKVTINC

SEQ ID NO: 520 - protein, 23 aa, Llama glama
DVVLTQTPGSLSVVPGESASISC

SEQ ID NO: 521 - protein, 22 aa, Llama glama
SSELTQPSAVSVSLGQTARITC

SEQ ID NO: 522 - protein, 23 aa, Llama glama
EIVLTQTPGSLSVVPGESASISC

SEQ ID NO: 523 - protein, 23 aa, Llama glama
DLVLTQIPGSLSVVPGESASISC

SEQ ID NO: 524 - protein, 22 aa, Llama glama
SYELTQSSAVSVSLGQTARITC

SEQ ID NO: 525 - protein, 22 aa, Llama glama
NFMLTQLPSVSGSPGQKVTISC

SEQ ID NO: 526 - protein, 22 aa, Llama glama
SYELTQPPSVSESPGKTVTISC

SEQ ID NO: 527 - protein, 22 aa, Llama glama
SSALTQPPSVSGSPGKTVTISC

SEQ ID NO: 528 - protein, 22 aa, Llama glama
HSAVTQPPSVSGSPGKTVTISC

SEQ ID NO: 529 - protein, 22 aa, Llama glama
NFMLTQPPSVSVALRQTAKITC

SEQ ID NO: 530 - protein, 17 aa, Llama glama
KSSQSVVWASNLKTYLN

SEQ ID NO: 531 - protein, 16 aa, Llama glama
KASQSLVHTDGKTYLY

SEQ ID NO: 532 - protein, 11 aa, Llama glama
RGDSLERYGAN

SEQ ID NO: 533 - protein, 16 aa, Llama glama
KASQSLVQTDGKTYLS

SEQ ID NO: 534 - protein, 16 aa, Llama glama
KASQSLVHTDGNTYLS

SEQ ID NO: 535 - protein, 11 aa, Llama glama
QGGSLGSYGAN

SEQ ID NO: 536 - protein, 14 aa, Llama glama
TGSSSNIGGGYSVQ

SEQ ID NO: 537 - protein, 14 aa, Llama glama
AGTSSDVGYGDYVS

SEQ ID NO: 538 - protein, 11 aa, Llama glama
GGEDIRTKNVH

SEQ ID NO: 539 - protein, 15 aa, Llama glama
WYQQRPGQPPRLLIY

SEQ ID NO: 540 - protein, 15 aa, Llama glama
WYQQKPGQARVQVIY

SEQ ID NO: 541 - protein, 15 aa, Llama glama
WLLQKPGQRPQLLIY

SEQ ID NO: 542 - protein, 15 aa, Llama glama
WLLQKPGQRPQLLIT

SEQ ID NO: 543 - protein, 15 aa, Llama glama
WYQQKPGQAPVLLIY

SEQ ID NO: 544 - protein, 15 aa, Llama glama
WFQQLPGTPPKLLIY

SEQ ID NO: 545 - protein, 15 aa, Llama glama
WYQQLPGMAPSLLIY

SEQ ID NO: 546 - protein, 15 aa, Llama glama
WYQQLPGMAPKLLIS

SEQ ID NO: 547 - protein, 7 aa, Llama glama
YASTQQS

SEQ ID NO: 548 - protein, 7 aa, Llama glama
GDDIRPS

SEQ ID NO: 549 - protein, 7 aa, Llama glama
QVSNRVS

SEQ ID NO: 550 - protein, 7 aa, Llama glama
QVSNRNS

SEQ ID NO: 551 - protein, 7 aa, Llama glama
GDNSRPS

SEQ ID NO: 552 - protein, 7 aa, Llama glama
GNSNRAS

SEQ ID NO: 553 - protein, 7 aa, Llama glama
EVNKRAS

SEQ ID NO: 554 - protein, 7 aa, Llama glama
EVNKRAS

SEQ ID NO: 555 - protein, 7 aa, Llama glama
GVNKRAS

SEQ ID NO: 556 - protein, 32 aa, Llama glama
GVPDRFSGSGSTTDFTLTISNFQPEDAAVYYC

SEQ ID NO: 557 - protein, 32 aa, Llama glama
GIPERFSGSRLGDTATLTISGAQAEDEADYYC

SEQ ID NO: 558 - protein, 32 aa, Llama glama
GVPDRFTGSGSGTDFTLKISGVNAEDAGVYYC

SEQ ID NO: 559 - protein, 32 aa, Llama glama
GVPNRFTGSGSGTDFTLKISGVKPEDAAVYYC

SEQ ID NO: 560 - protein, 32 aa, Llama glama
GIPERFSGSKSGGTATLTISGAQAEDEADYYC

SEQ ID NO: 561 - protein, 32 aa, Llama glama
GVPDRFSGSRSGSSASLTITGLQAEDEADYYC

SEQ ID NO: 562 - protein, 32 aa, Llama glama
GIPERFSGSSSGGTATLTISGVQAEDEADYYC

SEQ ID NO: 563 - protein, 9 aa, Llama glama
QQVYRTPWT

SEQ ID NO: 564 - protein, 9 aa, Llama glama
QQAYKTPLT

SEQ ID NO: 565 - protein, 9 aa, Llama glama
AQATYSLYS

SEQ ID NO: 566 - protein, 11 aa, Llama glama
QSSDSSGYKND

SEQ ID NO: 567 - protein, 9 aa, Llama glama
AQATYYPYT

SEQ ID NO: 568 - protein, 8 aa, Llama glama
AQVTYAAS

SEQ ID NO: 569 - protein, 9 aa, Llama glama
AQAIYYPPS

SEQ ID NO: 570 - protein, 9 aa, Llama glama
AQVTRYPVT

SEQ ID NO: 571 - protein, 10 aa, Llama glama
QSFDYSGNAV

SEQ ID NO: 572 - protein, 12 aa, Llama glama
SSWDDSLSGHPV

SEQ ID NO: 573 - protein, 10 aa, Llama glama
ASYRSSVNVV

SEQ ID NO: 574 - protein, 10 aa, Llama glama
ASYRSANNAV

SEQ ID NO: 575 - protein, 10 aa, Llama glama
ASYKYSNNVV

SEQ ID NO: 576 - protein, 9 aa, Llama glama
QSVDSSNIV

SEQ ID NO: 577 - protein, 10 aa, Llama glama
FGQGTRLEIK

SEQ ID NO: 578 - protein, 10 aa, Llama glama
FGQGTKVELN

SEQ ID NO: 579 - protein, 11 aa, Llama glama
FSSRTHLTVLG

SEQ ID NO: 580 - protein, 10 aa, Llama glama
FGSGTRLEMK

SEQ ID NO: 581 - protein, 10 aa, Llama glama
FGGGTRLEIK

SEQ ID NO: 582 - protein, 11 aa, Llama glama
FGGGTHLTILG

SEQ ID NO: 583 - protein, 126 aa, Llama glama
EVQLVESGGGLAQPGGSLRLSCRASGFAFGSFWMYWVRQTPGKGLEWVSTVDAAGANTYYPDTVKGRFTISRDNAKNTLYLRMNSLKPEDTARYYCVTDGAYDDPAIRGKIPLASWGQGTLVTVSS

SEQ ID NO: 584 - protein, 114 aa, Llama glama
EVQLVQPGAELRNPGASVKVSCKASGYTFTRYYIDWVRQAPGQGLEWMGRIDPEDGGTKVARKFQGRVTFTADTSASTVYLSLTNLRSDDTGVYYCATRGRGSWGQGTRVTVSS

SEQ ID NO: 585 - protein, 118 aa, Llama glama
EVQLVQPGAELRNPGASVKVSCKASGYTFTSSYIDWVRQVPGQGFEWMGRMDPEDGTTKPAPKFQGRVTFTADTSAHTAYMELTTLRVDDTAVYFCAIDDGLGALNYWGHGTQVTVSS

SEQ ID NO: 586 - protein, 116 aa, Llama glama
EVQLVQPGHELKNPGASVKVSCKASGYIFTNHYIHWVRQAPGQGLEWMGRIDPEDGGVKYAQRFRGRVTFTVDTSTTTAYVELHSLRTDDTGVYYCAFWSDDDDYWGQGTQVTVSS

SEQ ID NO: 587 - protein, 117 aa, Llama glama
EVQLVQPGAELRNPGASVKVSCKASGYTFTSSYINWLRQAPGQGPEWMGRIDPEDGRTKSRQNFQGRVTFTADTSTSTAYVELRNLRSEDTAVYYCVAGAPDEYAYWGQGTQVTVSS

SEQ ID NO: 588 - protein, 113 aa, Llama glama
EVQLVQPGAELRNPGASVKVSCKASGYTFTKYYIDWVRQAPGQGLEWMGLIDPEDGATKYAQKFQGRVTFTTDTSTSTAYVELSSLRSEDTAVYYCLIFPRYWGQGTLVTVSS

SEQ ID NO: 589 - protein, 113 aa, Llama glama
EVQLVQPGTELRNPGSSVKVSCKASGYIFSNYFIDWVRQAPGQGLEWVGSIDPEDGGTQTAQKFQGRVIFTVDTSTSTAYVEVSSLRPEDTGVYYCVIPWNYWGQGTQVTVSS

SEQ ID NO: 590 - protein, 113 aa, Llama glama
EVQLVQPGTELRNPGASVKVSCKTSGYTFTGYYIDWVRQRPGEGLEWMGRIDPEDGDTKYAQKFQGGVTITADTSTTTAYLELSSLRSEDTAVYYCALGVDYWGQGTQVTVSS

SEQ ID NO: 591 - protein, 117 aa, Llama glama
EVQLVESGGGLVQPGGSLRLSCAASGFTFSTYAMAWVRQAPGKGPEWVSFINKYGDGYADSVKGRFTISRDNAKNTLTLQMNSLKPEDTAVYWCAKSRGDHDDYDYWGQGTQVTVSS

SEQ ID NO: 592 - protein, 114 aa, Llama glama
EVQLQESGGALVQPGGSLRLSCAASGFTFSRSWMYWVRQAPGKGLEWVSAMNPGGGSTYYADSVKGRFTISRDNAKNTLYLQMNSLKPEDTALYYCARAGMSTWGKGTLVTVSS

SEQ ID NO: 593 - protein, 124 aa, Llama glama
QVQLQESGPGLVKPSQTLSLACTVSGGSITLNGGYWSWIRQPPGKGLEWMGAIAYTGSTYISPSLKSRTSISRDASENQFTLQLSAVTPEDSAVYYCARHHYDSNWYYYGMDYWGKGTLVTVSS

SEQ ID NO: 594 - protein, 120 aa, Llama glama
ELQLVESGGGLVQPGGSLRLSCAASGFTFDDYAMSWVRQAPGKGLEWVSGTTRNGGRTYYAESMKGRFTISRDNDKNTLYLQMNSPKSEDTGVYYCAKSDYALGSTFSSWGQGTQVTVSS

SEQ ID NO: 595 - protein, 117 aa, Llama glama
QVQLVESGGGLVQPGGSLRLSCAASGFTFGSYDMSWVRQAPGKGPEWVSAINKSGGSTYYADSVKGRFTISRDNAKNTLYLQMNSLKPEDTAVYYCATMYGSSWSYWGQGTQVTVSS

SEQ ID NO: 596 - protein, 118 aa, Llama glama
ELQLVESGGGLVEPGGSLRLSCAASGFTFSNYAMTWVRQAPGKGLEWVSAINSGGGSTYYGDAVKGRFTISRDNAKNTLYLQINSLKPEDTAVYYCAKVGGLGYVDYWGQGTQVTVSS

SEQ ID NO: 597 - protein, 113 aa, Llama glama
DIVMTQSPSSVTASVGEKVTITCKSSQSVVWASNLKTYLNWYQQRPGQPPRLLIYYASTQQSGIPDRFSGSGSTTDFTLTISSVQPEDAAVYYCQQVYRTPWTFGQGTRLEIK

SEQ ID NO: 598 - protein, 113 aa, Llama glama
EIVLTQSPSSVTASAGEKVTINCKSSQSVLYSSNQKNYLAWYQQRLGQSPRLLIYWASTRESGVPDRFSGSGSTTDFTLTISNFQPEDAAVYYCQQAYKTPLTFGQGTKVELN

SEQ ID NO: 599 - protein, 112 aa, Llama glama
DVVLTQTPGSLSVVPGESASISCKASQSLVHTDGKTYLYWLLQKPGQSPQRLIYQVSNRGSGVPDRFTGSGSGTDFTLKISGVKAEDAGVYYCAQATYSLYSFGSGTRLEIK

SEQ ID NO: 600 - protein, 109 aa, Llama glama
SSELTQPSAVSVSLGQTARITCRGDSLERYGANWYQQKPGQARVQVIYGDDIRPSGIPERFSGSRLGDTATLTISGAQAEDEADYYCQSSDSSGYKNDFSSRTHLTVLG

SEQ ID NO: 601 - protein, 112 aa, Llama glama
EIVLTQTPGSLSVVPGESASISCKASQSLVHSDGKTYLYWLLQKPGQSPQRLIYQVSNRGSGVPDRFTGSGSGTDFTLKISGVKAEDAGVYYCAQATYYPYTFGSGTRLEIK

SEQ ID NO: 602 - protein, 111 aa, Llama glama
DLVLTQIPGSLSVVPGESASISCKASQSLVQTDGKTYLSWLLQKPGQRPQLLIYQVSNRVSGVPDRFTGSGSGTDFTLKISGVNAEDAGVYYCAQVTYAASFGSGTRLEMK

SEQ ID NO: 603 - protein, 112 aa, Llama glama
ATMLTQSPGSLSVVPGESASISCKASQSLVHTDGKTYLSWLLQKPGQRPQLLIYQVSNRGSGVPDRFTGSGSGTDFTLKISGVKAEDAGVYYCAQAIYYPPSFGSGTRLEIK

SEQ ID NO: 604 - protein, 112 aa, Llama glama
ATMLTQSPGSLSVVPGESASISCKASQSLVHTDGNTYLSWLLQKPGQRPQLLITQVSNRNSGVPNRFTGSGSGTDFTLKISGVKPEDAAVYYCAQVTRYPVTFGGGTRLEIK

SEQ ID NO: 605 - protein, 108 aa, Llama glama
SYELTQSSAVSVSLGQTARITCQGGSLGSYGANWYQQKPGQAPVLLIYGDNSRPSGIPERFSGSKSGGTATLTISGAQAEDEADYYCQSFDYSGNAVFGGGTHLTVLG

SEQ ID NO: 606 - protein, 113 aa, Llama glama
NFMLTQLPSVSGSPGQKVTISCTGSSSNIGGGYSVQWFQQLPGTPPKLLIYGNSNRASGVPDRFSGSRSGSSASLTITGLQAEDEADYYCSSWDDSLSGHPVFGGGTKLTVLG

SEQ ID NO: 607 - protein, 111 aa, Llama glama
SYELTQPPSVSESPGKTVTISCAGTSSDVGYGNYVSWYQQLPGMAPKLLLYEVNKRASGIADRFSGSKSGNTASLTISGLQSEDEADYYCASYRSSVNVVFGGGTHLTVLG

SEQ ID NO: 608 - protein, 111 aa, Llama glama
SSALTQPPSVSGSPGKTVTISCAGTSSDVGYGNYVSWYQQLPGMAPSLLIYEVNKRASGIADRFSGSKSGNTASLTISGLQSEDEADYYCASYRSANNAVFGGGTHLTILG

SEQ ID NO: 609 - protein, 111 aa, Llama glama
HSAVTQPPSVSGSPGKTVTISCAGTSSDVGYGDYVSWYQQLPGMAPKLLISGVNKRASGIADRFSGSKSGNTASLTISGLQSEDEADYYCASYKYSNNVVFGGGTHLTVLG

SEQ ID NO: 610 - protein, 107 aa, Llama glama
NFMLTQPPSVSVALRQTAKITCGGEDIRTKNVHWYQQKPGQAPVLVIYKDSERPSGIPERFSGSSSGGTATLTISGVQAEDEADYYCQSVDSSNIVFGGGTKLTVLG

SEQ ID NO: 611 - protein, 10 aa, Homo sapiens
VELFLADVEG

SEQ ID NO: 612 - DNA, 776 bp, Artificial
  plasmid
ggtaccgccacgatgggctggtcctgcatcatcctgtttctggtggccacggcccagccggccatggctgaattcggcgcgcccgacaagacacacacctgccctccttgtccagcccccgaactgctgggtgggcccagcgtgttcctgtttcctcctaaacccaaagacactctgatgattagtaggaccccagaagtcacttgcgtggtggttgacgtgtcacatgaagatcccgaggtcaagttcaattggtatgttgacggggtcgaagttcacaacgctaaaactaaaccaagagaggaacagtataactctacctaccgggtggtgagtgttctgactgtcctccatcaagactggctgaatggcaaagaatacaagtgtaaggtgagcaacaaagccctgcccgctcctatagagaaaacaatatccaaagccaaaggtcaacctcgcgagccacaggtgtacaccctcccaccaagccgcgatgaacttactaagaaccaagtctctcttacttgcctggttaaggggttctatccatccgacattgcagtcgagtgggagtctaatggacagcctgagaacaactacaaaaccacccctcctgttctggattctgacggatctttcttcctttattctaaactcaccgtggataaaagcaggtggcagcagggcaacgtgttcagctgttccgttatgcatgaggccctgcataaccattatacccagaagtctttgtccctcagtccaggaaagtgagcggccgc

SEQ ID NO: 613 - protein, 257 aa, Artificial
  chimeric polypeptide
GTATMGWSCIILFLVATAQPAMAEFGAPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAA

SEQ ID NO: 614 - protein, 107 aa, Llama glama
SYELTQSPSVSVALRQAAKITCGGDNIASKYVNWYQQKPGQAPVLVIYKDSSRPSGIPERFSGSNSGNTATLTVSGAQAEDEADYYCQVWDNSAAVFGGGTHLTVLG

SEQ ID NO: 615 - protein, 108 aa, Llama glama
QPVLNQPSALSVTLGQTAKITCQGGSLGSSYAHWYQQKPGQAPVLVIYADNRRPSGTPERFSGSNSGNTATLTISGAQAEDEADYYCQVWDSSANAVFGGGTHLTVLG

SEQ ID NO: 616 - protein, 251 aa, Artificial
  chimeric polypeptide
GVELFLADVEGGGSIEGRDMDPHGGCTCPQCPAPELPGGPSVFVFPPKPKDVLSISGRPEVTCVVVDVGKEDPEVNFNWYIDGVEVRTANTKPKEEQFNSTYRVVSVLPIQHQDWLTGKEFKCKVNNKALPAPIERTISKAKGQTREPQVYTLAPHREELAKDTVSVTCLVKGFYPADINVEWQRNGQPESEGTYANTPPQLDNDGTYFLYSKLSVGKNTWQRGETLTCVVMHEALHNHYTQKSISQSPGK

(19) United States
(12) Patent Application Publication
(10) Pub. No.: US 2016/0207996 A1
(43) Pub. Date: Jul. 21, 2016

(54) ANTIBODIES TO COMPLEX TARGETS

(71) Applicant: ARGEN-X N.V., Ah Breda (NL)

(72) Inventors: Peter Ulrichts, Breda (NL); Sebastian Van Der Woning, Breda (NL); Gitte De Boeck, Breda (NL); Erik Hofman, Breda (NL); Christophe Blanchetot, Breda (NL); Michael Saunders, Breda (NL); Johannes Joseph Wilhelmus De Haard, Breda (NL)

(21) Appl. No.: 14/913,984

(22) PCT Filed: Sep. 5, 2014
(86) PCT No.: PCT/EP2014/068980

§ 371 Date: Feb. 23, 2016

(30) Foreign Application Priority Data
Sep. 5, 2013 (GB) 1315851.4

(51) Int. Cl. C07K 16/28; C07K 16/00

(52) U.S. Cl. CPC C07K 16/28; C07K 16/00; C07K 2317/14; C07K 2317/22; C07K 2317/24; C07K 2317/92; C07K 2317/33; A61K 2039/53

(57) ABSTRACT

The present invention relates to antibodies and antigen binding fragments thereof derived from antibodies raised by DNA immunization of host animals, particularly camelids (e.g. llama). The antibodies and antigen binding fragments thereof bind to proteins which may be particularly large in size (at least 1115 amino acids in length), or have at least 8 transmembrane domains, or are naturally encoded by nucleotide sequences which are difficult to replicate in standard or common E. coli strains. The present invention also relates to antibodies and antigen binding fragments thereof which bind to ion channels, in particular the voltage-gated sodium channel Nav1.7. Methods of raising antibodies against particular protein targets by a process of DNA immunization are also provided.

### TECHNICAL FIELD

The present invention relates to antibodies and antigen binding fragments thereof derived from antibodies raised by DNA immunization of host animals, particularly camelids (e.g. llama). The antibodies and antigen binding fragments thereof bind to proteins which may be particularly large in size (at least 1115 amino acids in length), or have at least 8 transmembrane domains, or are naturally encoded by nucleotide sequences which are difficult to replicate in standard or common E. coli strains. The present invention also relates to antibodies and antigen binding fragments thereof which bind to ion channels, in particular the voltage-gated sodium channel Nav1.7. Methods of raising antibodies against particular protein targets by a process of DNA immunization are also provided.

### BACKGROUND

Antibodies exhibit specific binding activity for target antigens, and are valuable both as research tools and as therapeutic agents. Antibodies are produced by the immune system of an animal in response to exposure to an immunogen. Therefore, the administration to a host animal of a protein or peptide immunogen or antigen, can be used to raise antibodies against particular protein targets.

An alternative approach used to stimulate production of antibodies against a target protein or antigen in a host animal is DNA immunization, also referred to as DNA vaccination. DNA immunization involves the introduction of a nucleic acid molecule encoding one or more selected antigens into a host animal for the in vivo expression of the antigen. This approach has been used to generate antibodies against various proteins including the GPCRs: CXCR4; CCR3; CCR5; and Thyrotropin (Fujimoto et al. (2009) Human Antibodies, 18: 75-80; Costagliola et al. (1998) The Journal of Immunology, 160: 1458-1465), the plant potassium channel KAT1 (Gehwolf et al. (2007) FEBS Lett. 581(3): 448-452), and the membrane proteins: Endothelin-B; Mesothelin; HCV Host entry factor CD81; Ret tyrosine kinase and CD30 (Ducancel et al. (2013) mAbs 5:1, 56-69; Partha et al. (1999) Journal of Immunological Methods 231: 83-91; Nagata et al. (2003) Journal of Immunological Methods 280: 59-72). International patent application WO2010/070145 also describes DNA vaccination of camelids for the production of immunoglobulin sequences against cell-associated antigens. Nanobodies are described that bind to the ligand-operated ion channel P2X7 and the GPCRs CXCR4 and CXCR7.

Notwithstanding the approaches available for the production of antibodies against targets of interest, in some cases, the production of antibodies, particularly antibodies with certain preferred features and/or properties, has proved a significant challenge. This is the case for ion channels, which represent a particularly important class of protein targets.

Ion channels are transmembrane proteins, which play a key role in physiology and disease by modulating cellular functions such as electrical excitability, secretion, cell migration and gene transcription. Ion channels are typically formed as integral membrane proteins either by multisubunit protein assemblies or by the association of multiple domains within a single protein. The structure of the ion channel pore through the membrane is generally conserved for the ion channel family members. However, the opening and closing of the pore, known as the “gating process”, is controlled differently for the various ion channels.

Voltage-gated ion channels form a major sub-class within the ion channel family, and are gated by hyperpolarizations or depolarizations of the cell membrane. These channels are responsible for cellular excitability in cardiac and neuronal tissue. The voltage-gated ion channel proteins typically have six transmembrane helices (S1, S2, S3, S4, S5 and S6) spanning the membrane and three extracellular hydrophilic loops (E1, E2 and E3). The voltage gated potassium channels comprise a tetramer of the basic six transmembrane subunit. However, the sodium and calcium voltage-gated ion channels constitute a single protein with four linked domains (A, B, C and D), each domain having six transmembrane helices and three extracellular loops as shown in FIG. 1.

The voltage-gated sodium channels include nine different isoforms (Nav1.1 to 1.9). Nav1.7 is of particular interest as a therapeutic target because modulation of Nav1.7 activity has been found to affect pain in animal models. In humans, Nav1.7 is encoded by the gene SCN9A and is expressed in the peripheral nervous system i.e. in nociceptive dorsal root ganglions (DRG). Both gain-of-function and loss-of-function mutations of Nav1.7 result in pain-related abnormalities in humans.

Antibodies which bind to extracellular regions of ion channels have been produced using peptide immunization, and in some cases such antibodies are capable of modulating channel activity (reviewed in Naylor and Beech (2009) The Open Drug Discovery Journal. 1:36-42). Polyclonal antibodies, which bind to the E3 extracellular loop or “turret peptide”, have been identified as particularly effective blocking antibodies.

The success in developing monoclonal antibodies which bind ion channel targets and exhibit advantageous properties, for example agonistic or antagonistic activity, has been very limited. Yang et al. (Yang et al. (2012) PLoS ONE 7(4): e36379) describes a monoclonal antibody which specifically binds the potassium voltage-gated ion channel hKv1.3 and inhibits the channel current as measured by whole cell patch clamp. Gomez-Varela et al. (Gomez-Varela et al. (2007) Cancer Res. 67(15) 7343-9) describes a monoclonal antibody capable of binding to and blocking the ion flow through the voltage-gated potassium channel human Eag1.

US2011/0135662 describes the production of monoclonal antibodies which bind to extracellular loops of the voltage-gated sodium channel Nav1.7, for example antibodies designated 932, 983 and 1080. A number of these antibodies were found to partially inhibit Nav1.7 currents as measured by patch clamp analysis using HEK cells expressing human Nav1.7.

### SUMMARY OF THE INVENTION

The present invention is directed to the use of DNA immunization as a means to raise antibodies which bind to target proteins, wherein the target proteins are particularly long and/or complex in structure and/or wherein the target protein is naturally encoded by a nucleotide sequence which is difficult to replicate in standard or common E. coli strains. Typically, the antibodies raised by DNA immunization against said target proteins have different or superior properties to antibodies raised by other techniques.

In a first aspect, the present invention provides an antibody or antigen binding fragment thereof, which binds to a target protein, comprising at least one complementarity determining region (CDR) wherein:

(a) the target protein has a length of at least 1115 amino acids and the at least one CDR is derived from an antibody raised by immunization of a host animal with a DNA molecule comprising an open reading frame of at least 3345 nucleotides encoding:
(i) the full-length target protein;
(ii) a protein having at least 70% identity to the full-length target protein; or
(iii) a fragment of the full-length target protein;
   or
   (b) the target protein has at least 8 transmembrane domains and the at least one CDR is derived from an antibody raised by immunization of a host animal with a DNA molecule comprising an open reading frame encoding:
(i) the full-length target protein;
(ii) a protein having at least 70% identity to the full-length target protein; or
(iii) a fragment of the full-length target protein comprising at least 8 transmembrane domains;
   or
   (c) the target protein is naturally encoded by a nucleotide sequence which is difficult to replicate in a common E. coli strain and the at least one CDR is derived from an antibody raised by immunization of a host animal with a DNA molecule comprising an open reading frame, which is difficult to replicate in a common E. coli strain, encoding:
(i) the full-length target protein;
(ii) a protein having at least 70% identity to the full-length target protein; or
(iii) a fragment of the full-length target protein.

The present invention is also directed to antibodies, or antigen binding fragments thereof, which bind to the voltage-gated sodium channel human Nav1.7 and exhibit properties which are different, and generally superior, to Nav1.7 antibodies described in the prior art, in particular the Nav1.7 antibodies described in US2011/0135662. The superior properties of these antibodies are advantageous with regard to use in human therapy, particularly for the treatment of pain.

The Nav1.7 antibodies of the present invention are characterised by a binding affinity for human Nav1.7 which is higher than reference Nav1.7 antibodies previously described. The reference Nav1.7 antibodies are selected from Nav1.7 antibodies: CA167_00932 (referred to herein as “932” or “UCB_932”); CA167_00983 (referred to herein as “983” or “UCB_983”); and CA167_01080 (referred to herein as “1080” or “UCB_1080”), as shown in Table 3 of US2011/0135662, which is incorporated by reference herein in its entirety.

Therefore, in a second aspect of the invention, there is provided an antibody or antigen binding fragment thereof, which binds to the voltage-gated sodium channel human Nav1.7, said antibody or antigen binding fragment comprising at least one heavy chain variable domain (VH) and at least one light chain variable domain (VL), wherein said VH and VL domain, when tested as a mAb, exhibit an affinity of binding for an extracellular region of human Nav1.7, which is at least 10-fold, at least 20-fold, at least 50-fold, at least 100-fold higher than a reference antibody selected from the group consisting of: UCB_932; UCB_983; and UCB_1066, as described in US2011/0135662.

Preferred embodiments described herein exhibit a binding affinity for human Nav1.7 (as measured by standard techniques such as ELISA or BIACORE, as described elsewhere herein) which is (significantly) higher than the prior art Nav1.7 antibodies described. The Nav1.7 antibodies of the present invention typically exhibit superior binding affinity for the extracellular loops of human Nav1.7.

Therefore, in a third aspect of the invention, there is provided an antibody or antigen binding fragment thereof, which binds to the voltage-gated sodium channel human Nav1.7, said antibody or antigen binding fragment comprising at least one heavy chain variable domain (VH) and at least one light chain variable domain (VL), wherein said VH and VL domain, when tested as a mAb, exhibit an affinity of binding for an extracellular region of human Nav1.7, (EC₅₀ as measured by ELISA), of less than 2 nM, preferably less than 1 nM, preferably less than 0.4 nM. The highest affinity antibodies or antigen binding fragments may have an affinity of binding for an extracellular region of human Nav1.7, (EC₅₀ as measured by ELISA), of 0.05 nM. The ELISA analysis will typically be performed as described in Examples 3 and 8 herein.

In a fourth aspect of the invention, there is provided an antibody or antigen binding fragment thereof, which binds to the voltage gated sodium channel human Nav1.7, said antibody or antigen binding fragment comprising at least one heavy chain variable domain (VH) and at least one light chain variable domain (VL), wherein said VH and VL domain exhibit an off-rate (k_{off} measured by Biacore) for an extracellular region of human Nav1.7 of less than 5×10⁻³ s⁻¹, when tested as a mAb using the standard Biacore protocol described herein.

In preferred embodiments, the antibody or antigen binding fragment comprises at least one heavy chain variable domain (VH) and at least one light chain variable domain (VL), wherein said VH and VL domain exhibit an off-rate for an extracellular region of human Nav1.7 of less than 5×10⁻³ s⁻¹, less than 5×10⁻⁴ s⁻¹, less than 5×10⁻⁵ s⁻¹. In a further preferred embodiment, the Nav1.7 antibody or antibody binding fragment will exhibit an off-rate for an extracellular loop of human Nav1.7 in the range of from 0.03×10⁻⁴ s⁻¹, to 45×10⁻⁴ s⁻¹, when tested as a mAb.

The affinity of the antibody or antigen binding fragment for human Nav1.7, as measured by Biacore may be determined using a human Nav1.7 peptide construct, as described elsewhere herein, and shown in Table 4. For example, the off-rate may be determined by Biacore analysis using a hNav1.7 loop A3-llama Fc chimeric construct as represented by SEQ ID NO: 267 shown in Table 4. Alternatively, the off-rate may be determined by Biacore analysis using a hNav1.7 loop A3-GST chimeric construct as represented by SEQ ID NO: 272 shown in Table 4.

Preferred embodiments of the Nav1.7 antibodies described herein are capable of inhibiting the activity of the Nav1.7 sodium channel, particularly when tested in an in vitro patch clamp assay as described elsewhere herein, notably in Example 11. In certain embodiments, the antibodies of the invention may be capable of inhibiting Nav1.7 channel activity by at least 20%, at least 30%, at least 40%, at least 50%, at least 60%, at least 70%, at least 80% or at least 90%.

In non-limiting embodiments the invention provides the following antibodies, or antigen binding fragments thereof, which are defined by reference to specific structural characteristics, i.e. specified amino acid sequences of either the CDRs (one or more of SEQ ID NOs: 8-13, 16-21, 28-34, 100-109, 119-129, 141-149, 289, 291, 293, 452-465, 472-485, 500-512 (heavy chain CDRs) or SEQ ID NOs: 46-54, 62-68, 77-84, 161-172, 181-192, 204-215, 296, 298, 300, 530-538, 547-555, 563-576 (light chain CDRs)) or entire variable domains (one or more of SEQ ID NOs: 218-226, 236-247, 302, 583-596 (heavy chain variable domains) or SEQ ID NOs: 227-235, 248-259, 303, 597-610 (light chain variable domains)). All of these antibodies bind to the voltage-gated sodium channel human Nav1.7.

In particular embodiments, the antibodies defined by the following structural characteristics may additionally exhibit high human homology, as defined herein. The antibodies may be monoclonal antibodies produced by recombinant means. The CDRs of the following Nav1.7 antibodies may be camelid-derived, i.e. derived from conventional antibodies raised by immunisation of camelids (specifically llama). The invention also provides humanised or human germlined variants, affinity variants and variants containing conservative amino acid substitutions, as defined herein.

Embodiments of the Nav1.7 antibodies of the invention are now further described by reference to structural characteristics.

In one embodiment, there is provided an antibody or antigen binding fragment thereof, which binds to the voltage-gated sodium channel human Nav1.7, said antibody or antigen binding fragment comprising a heavy chain variable domain (VH) comprising a variable heavy chain CDR3 (HCDR3) selected from the group consisting of:

SEQ ID NO: 28 [gpgysgsyiegway], or sequence variant thereof,

SEQ ID NO: 29 [gpgysgkyiegway], or sequence variant thereof,

SEQ ID NO: 30 [rryglgseydy], or sequence variant thereof,

SEQ ID NO: 31 [ggvvawsydy], or sequence variant thereof,

SEQ ID NO: 32 [amvttsspwvktfga], or sequence variant thereof,

SEQ ID NO: 33 [glrvagsyftdfgt], or sequence variant thereof,

SEQ ID NO: 34 [drngeydy], or sequence variant thereof,

SEQ ID NO: 141 [vpsvfglpyggmny], or sequence variant thereof,

SEQ ID NO: 142 [glgp], or sequence variant thereof,

SEQ ID NO: 143 [dmdy], or sequence variant thereof,

SEQ ID NO: 144 [gldy], or sequence variant thereof,

SEQ ID NO: 145 [gdsgy], or sequence variant thereof,

SEQ ID NO: 146 [aggey], or sequence variant thereof,

SEQ ID NO: 147 [gvdy], or sequence variant thereof,

SEQ ID NO: 148 [tiegf], or sequence variant thereof,

SEQ ID NO: 149 [gsfga], or sequence variant thereof,

SEQ ID NO: 293 [eitl], or sequence variant thereof,

SEQ ID NO: 500 [dgayddpairgkiplas], or sequence variant thereof,

SEQ ID NO: 501 [rgrgs], or sequence variant thereof,

SEQ ID NO: 502 [ddglgalny], or sequence variant thereof,

SEQ ID NO: 503 [wsddddy], or sequence variant thereof,

SEQ ID NO: 504 [gapdeyay], or sequence variant thereof,

SEQ ID NO: 505 [fpry], or sequence variant thereof,

SEQ ID NO: 506 [pwny], or sequence variant thereof,

SEQ ID NO: 507 [srgdhddydy], or sequence variant thereof,

SEQ ID NO: 508 [agmst], or sequence variant thereof,

SEQ ID NO: 509 [hhydsnwyyygmdy], or sequence variant thereof,

SEQ ID NO: 510 [sdyalgstfss], or sequence variant thereof,

SEQ ID NO: 511 [mygsswsy], or sequence variant thereof,

SEQ ID NO: 512 [vgglgyvdy], or sequence variant thereof,

wherein the sequence variant comprises one, two or three amino acid substitutions (e.g. conservative substitutions, humanising substitutions or affinity variants) in the recited sequence.

The heavy chain variable domain of this antibody may alternatively or in addition comprise a variable heavy chain CDR2 (HCDR2) comprising a sequence selected from the group consisting of:

SEQ ID NO: 16 [aintgggstyyadsvkg], or sequence variant thereof,

SEQ ID NO: 17 [aiswnggstyyaesmkg], or sequence variant thereof,

SEQ ID NO: 18 [viaydgrtfyspslks], or sequence variant thereof,

SEQ ID NO: 19 [viaydgstyyspslks], or sequence variant thereof,

SEQ ID NO: 20 [viiydgstyyspslks], or sequence variant thereof,

SEQ ID NO: 21 [vidrggggtryadsvkg], or sequence variant thereof,

SEQ ID NO: 119 [aiaysgstyyspslqs], or sequence variant thereof,

SEQ ID NO: 120 [iigydggtyynpslks], or sequence variant thereof,

SEQ ID NO: 121 [ridpedggtkytqkfqg], or sequence variant thereof,

SEQ ID NO: 122 [ridpedggtkyaqkfqg], or sequence variant thereof,

SEQ ID NO: 123 [ridpedggtsyaqkfqg], or sequence variant thereof,

SEQ ID NO: 124 [ridpedgatkyapkfqg], or sequence variant thereof,

SEQ ID NO: 125 [ridpedggtsyaqkfqg], or sequence variant thereof,

SEQ ID NO: 126 [ridpedgdtkyaqkfqg], or sequence variant thereof,

SEQ ID NO: 127 [ridpedgetryahqfrd], or sequence variant thereof,

SEQ ID NO: 128 [dinsggeitayadsvkg], or sequence variant thereof,

SEQ ID NO: 129 [dinsggeitayadsvkg], or sequence variant thereof,

SEQ ID NO: 291 [virydgntrysptlks], or a sequence variant thereof,

SEQ ID NO: 472 [tvdaagantyypdtvkg], or a sequence variant thereof,

SEQ ID NO: 473 [ridpedggtkvarkfqg], or a sequence variant thereof,

SEQ ID NO: 474 [rmdpedgttkpapkfqg], or a sequence variant thereof,

SEQ ID NO: 475 [ridpedggvkyaqrfrg], or a sequence variant thereof,

SEQ ID NO: 476 [ridpedgrtksrqnfqg], or a sequence variant thereof,

SEQ ID NO: 477 [lidpedgatkyaqkfqg], or a sequence variant thereof,

SEQ ID NO: 478 [sidpedggtqt], or a sequence variant thereof,

SEQ ID NO: 479 [ridpedgdtkyaqkfqg], or a sequence variant thereof,

SEQ ID NO: 480 [finkygdgyadsvkg], or a sequence variant thereof,

SEQ ID NO: 481 [amnpgggstyyadsvkg], or a sequence variant thereof,

SEQ ID NO: 482 [aiaytgstyispslks], or a sequence variant thereof,

SEQ ID NO: 483 [gttrnggrtyyaesmkg], or a sequence variant thereof,

SEQ ID NO: 484 [ainksggstyyadsvkg], or a sequence variant thereof,

SEQ ID NO: 485 [ainsgggstyygdavkg], or a sequence variant thereof,

wherein the sequence variant comprises one, two or three amino acid substitutions (e.g. conservative substitutions, humanising substitutions or affinity variants) in the recited sequence.

The heavy chain variable domain of this antibody may alternatively or in addition comprise a variable heavy chain CDR1 (HCDR1) comprising a sequence selected from the group consisting of:

SEQ ID NO: 8 [sywmy], or sequence variant thereof,

SEQ ID NO: 9 [dyams], or sequence variant thereof,

SEQ ID NO: 10 [tsfyaws], or sequence variant thereof,

SEQ ID NO: 11 [nygws], or sequence variant thereof,

SEQ ID NO: 12 [nsyyass], or sequence variant thereof,

SEQ ID NO: 13 [nywmh], or sequence variant thereof,

SEQ ID NO: 100 [nnyyywn], or sequence variant thereof,

SEQ ID NO: 101 [tsgtgws], or sequence variant thereof,

SEQ ID NO: 102 [dyyih], or sequence variant thereof,

SEQ ID NO: 103 [sayiv], or sequence variant thereof,

SEQ ID NO: 104 [ssyid], or sequence variant thereof,

SEQ ID NO: 105 [ssdiq], or sequence variant thereof,

SEQ ID NO: 106 [ssyid], or sequence variant thereof,

SEQ ID NO: 107 [gyyid], or sequence variant thereof,

SEQ ID NO: 108 [sayid], or sequence variant thereof,

SEQ ID NO: 109 [fseyvms], or sequence variant thereof,

SEQ ID NO: 289 [dkssaws], or a sequence variant thereof,

SEQ ID NO: 452 [sfwmy], or a sequence variant thereof,

SEQ ID NO: 453 [ryyid], or a sequence variant thereof,

SEQ ID NO: 454 [ssyid], or a sequence variant thereof,

SEQ ID NO: 455 [nhyih], or a sequence variant thereof,

SEQ ID NO: 456 [ssyin], or a sequence variant thereof,

SEQ ID NO: 457 [kyyid], or a sequence variant thereof,

SEQ ID NO: 458 [nyfid], or a sequence variant thereof,

SEQ ID NO: 459 [gyyid], or a sequence variant thereof,

SEQ ID NO: 460 [tyama], or a sequence variant thereof,

SEQ ID NO: 461 [rswmy], or a sequence variant thereof,

SEQ ID NO: 462 [lnggyws], or a sequence variant thereof,

SEQ ID NO: 463 [dyams], or a sequence variant thereof,

SEQ ID NO: 464 [sydms], or a sequence variant thereof,

SEQ ID NO: 465 [nyamt], or a sequence variant thereof,

wherein the sequence variant comprises one, two or three amino acid substitutions (e.g. conservative substitutions, humanising substitutions or affinity variants) in the recited sequence.

The heavy chain variable domain may comprise any one of the listed variable heavy chain CDR3 sequences (HCDR3) in combination with any one of the variable heavy chain CDR2 sequences (HCDR2) and any one of the variable heavy chain CDR1 sequences (HCDR1). However, certain combinations of HCDR3 and HCDR2 and HCDR1 are particularly preferred, these being the “native” combinations which derive from a single common VH domain. These preferred combinations are listed in Tables 5, 10 and 26 and in preferred embodiments, the Nav1.7 antibody or antigen binding fragment thereof comprises a combination of variable heavy chain CDR3 (HCDR3), variable heavy chain CDR2 (HCDR2) and variable heavy chain CDR3 (HCDR3) selected from the group consisting of:

(i) HCDR3 comprising SEQ ID NO: 28; HCDR2 comprising SEQ ID NO: 16; HCDR1 comprising SEQ ID NO: 8;
(ii) HCDR3 comprising SEQ ID NO: 29; HCDR2 comprising SEQ ID NO: 16; HCDR1 comprising SEQ ID NO: 8;
(iii) HCDR3 comprising SEQ ID NO: 30; HCDR2 comprising SEQ ID NO: 17; HCDR1 comprising SEQ ID NO: 9;
(iv) HCDR3 comprising SEQ ID NO: 31; HCDR2 comprising SEQ ID NO: 18; HCDR1 comprising SEQ ID NO: 10;
(v) HCDR3 comprising SEQ ID NO: 32; HCDR2 comprising SEQ ID NO: 19; HCDR1 comprising SEQ ID NO: 11;
(vi) HCDR3 comprising SEQ ID NO: 33; HCDR2 comprising SEQ ID NO: 20; HCDR1 comprising SEQ ID NO: 12;
(vii) HCDR3 comprising SEQ ID NO: 34; HCDR2 comprising SEQ ID NO: 21; HCDR1 comprising SEQ ID NO: 13;
(viii) HCDR3 comprising SEQ ID NO: 141; HCDR2 comprising SEQ ID NO: 119; HCDR1 comprising SEQ ID NO: 100;
(ix) HCDR3 comprising SEQ ID NO: 142; HCDR2 comprising SEQ ID NO: 120; HCDR1 comprising SEQ ID NO: 101;
(x) HCDR3 comprising SEQ ID NO: 143; HCDR2 comprising SEQ ID NO: 121; HCDR1 comprising SEQ ID NO: 102;
(xi) HCDR3 comprising SEQ ID NO: 144; HCDR2 comprising SEQ ID NO: 122; HCDR1 comprising SEQ ID NO: 103;
(xii) HCDR3 comprising SEQ ID NO: 145; HCDR2 comprising SEQ ID NO: 123; HCDR1 comprising SEQ ID NO: 104;
(xiii) HCDR3 comprising SEQ ID NO: 146; HCDR2 comprising SEQ ID NO: 124; HCDR1 comprising SEQ ID NO: 105;
(xiv) HCDR3 comprising SEQ ID NO: 145; HCDR2 comprising SEQ ID NO: 125; HCDR1 comprising SEQ ID NO: 106;
(xv) HCDR3 comprising SEQ ID NO: 147; HCDR2 comprising SEQ ID NO: 126; HCDR1 comprising SEQ ID NO: 107;
(xvi) HCDR3 comprising SEQ ID NO: 148; HCDR2 comprising SEQ ID NO: 127; HCDR1 comprising SEQ ID NO: 108;
(xvii) HCDR3 comprising SEQ ID NO: 149; HCDR2 comprising SEQ ID NO: 128; HCDR1 comprising SEQ ID NO: 109;
(xviii) HCDR3 comprising SEQ ID NO: 149; HCDR2 comprising SEQ ID NO: 129; HCDR1 comprising SEQ ID NO: 109;
(xix) HCDR3 comprising SEQ ID NO: 293; HCDR2 comprising SEQ ID NO: 291; HCDR1 comprising SEQ ID NO: 289;
(xx) HCDR3 comprising SEQ ID NO: 500; HCDR2 comprising SEQ ID NO: 472; HCDR1 comprising SEQ ID NO: 452;
(xxi) HCDR3 comprising SEQ ID NO: 501; HCDR2 comprising SEQ ID NO: 473; HCDR1 comprising SEQ ID NO: 453;
(xxii) HCDR3 comprising SEQ ID NO: 502; HCDR2 comprising SEQ ID NO: 474; HCDR1 comprising SEQ ID NO: 454;
(xxiii) HCDR3 comprising SEQ ID NO: 503; HCDR2 comprising SEQ ID NO: 475; HCDR1 comprising SEQ ID NO: 455;
(xxiv) HCDR3 comprising SEQ ID NO: 504; HCDR2 comprising SEQ ID NO: 476; HCDR1 comprising SEQ ID NO: 456;
(xxv) HCDR3 comprising SEQ ID NO: 505; HCDR2 comprising SEQ ID NO: 477; HCDR1 comprising SEQ ID NO: 457;
(xxvi) HCDR3 comprising SEQ ID NO: 506; HCDR2 comprising SEQ ID NO: 478; HCDR1 comprising SEQ ID NO: 458;
(xxvii) HCDR3 comprising SEQ ID NO: 147; HCDR2 comprising SEQ ID NO: 479; HCDR1 comprising SEQ ID NO: 459;
(xxviii) HCDR3 comprising SEQ ID NO: 507; HCDR2 comprising SEQ ID NO: 480; HCDR1 comprising SEQ ID NO: 460;
(xxix) HCDR3 comprising SEQ ID NO: 508; HCDR2 comprising SEQ ID NO: 481; HCDR1 comprising SEQ ID NO: 461;
(xxx) HCDR3 comprising SEQ ID NO: 509; HCDR2 comprising SEQ ID NO: 482; HCDR1 comprising SEQ ID NO: 462;
(xxxi) HCDR3 comprising SEQ ID NO: 510; HCDR2 comprising SEQ ID NO: 483; HCDR1 comprising SEQ ID NO: 463;
(xxxii) HCDR3 comprising SEQ ID NO: 511; HCDR2 comprising SEQ ID NO: 484; HCDR1 comprising SEQ ID NO: 464; and
(xxxiii) HCDR3 comprising SEQ ID NO: 512; HCDR2 comprising SEQ ID NO: 485; HCDR1 comprising SEQ ID NO: 465.

In further embodiments, the antibody or antigen binding fragment thereof which binds to the voltage-gated sodium channel human Nav1.7 may alternatively or in addition comprise a light chain variable domain (VL), which is paired with the VH domain to form an antigen binding domain.

The light chain variable domain of the antibody may comprise a variable light chain CDR3 (LCDR3) comprising a sequence selected from the group consisting of:

SEQ ID NO: 77 [qqaynnpys], or sequence variant thereof,

SEQ ID NO: 78 [qqaysapys], or sequence variant thereof,

SEQ ID NO: 79 [qsgsssgnahav], or sequence variant thereof,

SEQ ID NO: 80 [qsahrsesav], or sequence variant thereof,

SEQ ID NO: 81 [qvwdsradaav], or sequence variant thereof,

SEQ ID NO: 82 [gcydsslstpv], or sequence variant thereof,

SEQ ID NO: 83 [qvwdssanaav], or sequence variant thereof,

SEQ ID NO: 84 [qqgysaplt], or sequence variant thereof,

SEQ ID NO: 204 [qvwdssaav], or sequence variant thereof,

SEQ ID NO: 205 [qqyyrapat], or sequence variant thereof,

SEQ ID NO: 206 [alsrvsgtygtv], or sequence variant thereof,

SEQ ID NO: 207 [aqdtyypys], or sequence variant thereof,

SEQ ID NO: 208 [aqttyyppt], or sequence variant thereof,

SEQ ID NO: 209 [qssdstdnav], or sequence variant thereof,

SEQ ID NO: 210 [aqhtyypps], or sequence variant thereof,

SEQ ID NO: 211 [aqttyfpia], or sequence variant thereof,

SEQ ID NO: 212 [aqttydpvt], or sequence variant thereof,

SEQ ID NO: 213 [qatyypls], or sequence variant thereof,

SEQ ID NO: 214 [aqatyvplg], or sequence variant thereof,

SEQ ID NO: 215 [aqatfkkit], or sequence variant thereof,

SEQ ID NO: 300 [aqatyypt], or sequence variant thereof,

SEQ ID NO: 563 [qqvyrtpwt], or sequence variant thereof,

SEQ ID NO: 564 [qqayktplt], or sequence variant thereof,

SEQ ID NO: 565 [aqatyslys], or sequence variant thereof,

SEQ ID NO: 566 [qssdssgyknd], or sequence variant thereof,

SEQ ID NO: 567 [aqatyypyt], or sequence variant thereof,

SEQ ID NO: 568 [aqvtyaas], or sequence variant thereof,

SEQ ID NO: 569 [aqaiyypps], or sequence variant thereof,

SEQ ID NO: 570 [aqvtrypvt], or sequence variant thereof,

SEQ ID NO: 571 [qsfdysgnav], or sequence variant thereof,

SEQ ID NO: 572 [sswddslsghpv], or sequence variant thereof,

SEQ ID NO: 573 [asyrssvnvv], or sequence variant thereof,

SEQ ID NO: 574 [asyrsannav], or sequence variant thereof,

SEQ ID NO: 575 [asykysnnvv], or sequence variant thereof,

SEQ ID NO: 576 [qsvdssniv], or sequence variant thereof,

wherein the sequence variant comprises one, two or three amino acid substitutions (e.g. conservative substitutions, humanising substitutions or affinity variants) in the recited sequence.

The antibody or antigen binding fragment may alternatively or in addition comprise a light chain variable domain CDR2 (LCDR2) comprising a sequence selected from the group consisting of:

SEQ ID NO: 62 [hastqes], or sequence variant thereof,

SEQ ID NO: 63 [yastqes], or sequence variant thereof,

SEQ ID NO: 64 [rdserps], or sequence variant thereof,

SEQ ID NO: 65 [adsrrps], or sequence variant thereof,

SEQ ID NO: 66 [svnkras], or sequence variant thereof,

SEQ ID NO: 67 [kdsnrps], or sequence variant thereof,

SEQ ID NO: 68 [wastres], or sequence variant thereof,

SEQ ID NO: 181 [adnrrps], or sequence variant thereof,

SEQ ID NO: 182 [sasrlet], or sequence variant thereof,

SEQ ID NO: 183 [ttnsrhs], or sequence variant thereof,

SEQ ID NO: 184 [rvsrrgs], or sequence variant thereof,

SEQ ID NO: 185 [qvstrgs], or sequence variant thereof,

SEQ ID NO: 186 [edserps], or sequence variant thereof,

SEQ ID NO: 187 [qvsnrgs], or sequence variant thereof,

SEQ ID NO: 188 [etsnrdp], or sequence variant thereof,

SEQ ID NO: 189 [qvsnrds], or sequence variant thereof,

SEQ ID NO: 190 [qvsnrgs], or sequence variant thereof,

SEQ ID NO: 191 [qvsrrds], or sequence variant thereof,

SEQ ID NO: 192 [qvsnras], or sequence variant thereof,

SEQ ID NO: 298 [qvsnrgs], or sequence variant thereof,

SEQ ID NO: 547 [yastqqs], or sequence variant thereof,

SEQ ID NO: 548 [gddirps], or sequence variant thereof,

SEQ ID NO: 549 [qvsnrvs], or sequence variant thereof,

SEQ ID NO: 550 [qvsnrns], or sequence variant thereof,

SEQ ID NO: 551 [gdnsrps], or sequence variant thereof,

SEQ ID NO: 552 [gnsnras], or sequence variant thereof,

SEQ ID NO: 553 [evnkras], or sequence variant thereof,

SEQ ID NO: 554 [gvnkras], or sequence variant thereof,

SEQ ID NO: 555 [kdserps], or sequence variant thereof,

wherein the sequence variant comprises one, two or three amino acid substitutions (e.g. conservative substitutions, humanising substitutions or affinity variants) in the recited sequence.

The antibody or antigen binding fragment may alternatively or in addition comprise a light chain variable domain CDR1 (LCDR1) comprising a sequence selected from the group consisting of:

SEQ ID NO: 46 [kssqsvvsgsnqksyln], or sequence variant thereof,

SEQ ID NO: 47 [kssqsvvsesnqrsyln], or sequence variant thereof,

SEQ ID NO: 48 [kssqsvvsgskqksyln], or sequence variant thereof,

SEQ ID NO: 49 [qgssfgssyah], or sequence variant thereof,

SEQ ID NO: 50 [qgtnlrssyvh], or sequence variant thereof,

SEQ ID NO: 51 [ggndigskstq], or sequence variant thereof,

SEQ ID NO: 52 [agtssdvgygnyvs], or sequence variant thereof,

SEQ ID NO: 53 [ggdniaskhah], or sequence variant thereof,

SEQ ID NO: 54 [kssqsvlyssnqknyla], or sequence variant thereof,

SEQ ID NO: 161 [ggsrigsksvq], or sequence variant thereof,

SEQ ID NO: 162 [qasqgiskyla], or sequence variant thereof,

SEQ ID NO: 163 [glssgsvtfgnyps], or sequence variant thereof,

SEQ ID NO: 164 [kasqslvhsdgmtyly], or sequence variant thereof,

SEQ ID NO: 165 [kasqslvhtdgktyls], or sequence variant thereof,

SEQ ID NO: 166 [qggdfrnyynn], or sequence variant thereof,

SEQ ID NO: 167 [kasqslvhsdgktyly], or sequence variant thereof,

SEQ ID NO: 168 [kagqslthpngktyls], or sequence variant thereof,

SEQ ID NO: 169 [ktsqslvhsdgktyly], or sequence variant thereof,

SEQ ID NO: 170 [ktsrslvhsdgktyls], or sequence variant thereof,

SEQ ID NO: 171 [kasqslvhsdgktyly], or sequence variant thereof,

SEQ ID NO: 172 [kanesivhpggktyly], or sequence variant thereof,

SEQ ID NO: 296 [kasqslvhsdgktyly], or sequence variant thereof,

SEQ ID NO: 530 [kssqsvvwasnlktyln], or sequence variant thereof,

SEQ ID NO: 531 [kasqslvhtdgktyly], or sequence variant thereof,

SEQ ID NO: 532 [rgdslerygan], or sequence variant thereof,

SEQ ID NO: 533 [kasqslvqtdgktyls], or sequence variant thereof,

SEQ ID NO: 534 [kasqslvhtdgntyls], or sequence variant thereof,

SEQ ID NO: 535 [qggslgsygan], or sequence variant thereof,

SEQ ID NO: 536 [tgsssnigggysvq], or sequence variant thereof,

SEQ ID NO: 537 [agtssdvgygdyvs], or sequence variant thereof,

SEQ ID NO: 538 [ggedirtknvh], or sequence variant thereof,

wherein the sequence variant comprises one, two or three amino acid substitutions (e.g. conservative substitutions, humanising substitutions or affinity variants) in the recited sequence.

The light chain variable domain may comprise any one of the listed variable light chain CDR3 sequences (LCDR3) in combination with any one of the variable light chain CDR2 sequences (LCDR2) and any one of the variable light chain CDR1 sequences (LCDR1). However, certain combinations of LCDR3 and LCDR2 and LCDR1 are particularly preferred, these being the “native” combinations which derive from a single common VL domain. These preferred combinations are listed in Tables 6, 11 and 27 and in preferred embodiments, the Nav1.7 antibody or antigen binding fragment thereof comprises a combination of variable light chain CDR3 (LCDR3), variable light chain CDR2 (LCDR2) and variable light chain CDR3 (LCDR3) selected from the group consisting of:

(i) LCDR3 comprising SEQ ID NO: 77; LCDR2 comprising SEQ ID NO: 62; LCDR1 comprising SEQ ID NO: 46;
(ii) LCDR3 comprising SEQ ID NO: 78; LCDR2 comprising SEQ ID NO: 63; LCDR1 comprising SEQ ID NO: 47;
(iii) LCDR3 comprising SEQ ID NO: 78; LCDR2 comprising SEQ ID NO: 63; LCDR1 comprising SEQ ID NO: 48;
(iv) LCDR3 comprising SEQ ID NO: 79; LCDR2 comprising SEQ ID NO: 64; LCDR1 comprising SEQ ID NO: 49;
(v) LCDR3 comprising SEQ ID NO: 80; LCDR2 comprising SEQ ID NO: 64; LCDR1 comprising SEQ ID NO: 50;
(vi) LCDR3 comprising SEQ ID NO: 81; LCDR2 comprising SEQ ID NO: 65; LCDR1 comprising SEQ ID NO: 51;
(vii) LCDR3 comprising SEQ ID NO: 82; LCDR2 comprising SEQ ID NO: 66; LCDR1 comprising SEQ ID NO: 52;
(viii) LCDR3 comprising SEQ ID NO: 83; LCDR2 comprising SEQ ID NO: 67; LCDR1 comprising SEQ ID NO: 53;
(ix) LCDR3 comprising SEQ ID NO: 84; LCDR2 comprising SEQ ID NO: 68; LCDR1 comprising SEQ ID NO: 54;
(x) LCDR3 comprising SEQ ID NO: 204; LCDR2 comprising SEQ ID NO: 181; LCDR1 comprising SEQ ID NO: 161;
(xi) LCDR3 comprising SEQ ID NO: 205; LCDR2 comprising SEQ ID NO: 182; LCDR1 comprising SEQ ID NO: 162;
(xii) LCDR3 comprising SEQ ID NO: 206; LCDR2 comprising SEQ ID NO: 183; LCDR1 comprising SEQ ID NO: 163;
(xiii) LCDR3 comprising SEQ ID NO: 207; LCDR2 comprising SEQ ID NO: 184; LCDR1 comprising SEQ ID NO: 164;
(xiv) LCDR3 comprising SEQ ID NO: 208; LCDR2 comprising SEQ ID NO: 185; LCDR1 comprising SEQ ID NO: 165;
(xv) LCDR3 comprising SEQ ID NO: 209; LCDR2 comprising SEQ ID NO: 186; LCDR1 comprising SEQ ID NO: 166;
(xvi) LCDR3 comprising SEQ ID NO: 210; LCDR2 comprising SEQ ID NO: 187; LCDR1 comprising SEQ ID NO: 167;
(xvii) LCDR3 comprising SEQ ID NO: 211; LCDR2 comprising SEQ ID NO: 188; LCDR1 comprising SEQ ID NO: 168;
(xviii) LCDR3 comprising SEQ ID NO: 212; LCDR2 comprising SEQ ID NO: 189; LCDR1 comprising SEQ ID NO: 169;
(xix) LCDR3 comprising SEQ ID NO: 213; LCDR2 comprising SEQ ID NO: 190; LCDR1 comprising SEQ ID NO: 170;
(xx) LCDR3 comprising SEQ ID NO: 214; LCDR2 comprising SEQ ID NO: 191; LCDR1 comprising SEQ ID NO: 171;
(xxi) LCDR3 comprising SEQ ID NO: 215; LCDR2 comprising SEQ ID NO: 192; LCDR1 comprising SEQ ID NO: 172;
(xxii) LCDR3 comprising SEQ ID NO: 300; LCDR2 comprising SEQ ID NO: 298; LCDR1 comprising SEQ ID NO: 296;
(xxiii) LCDR3 comprising SEQ ID NO: 563; LCDR2 comprising SEQ ID NO: 547; LCDR1 comprising SEQ ID NO: 530;
(xxiv) LCDR3 comprising SEQ ID NO: 564; LCDR2 comprising SEQ ID NO: 68; LCDR1 comprising SEQ ID NO: 54;
(xxv) LCDR3 comprising SEQ ID NO: 565; LCDR2 comprising SEQ ID NO: 187; LCDR1 comprising SEQ ID NO: 531;
(xxvi) LCDR3 comprising SEQ ID NO: 566; LCDR2 comprising SEQ ID NO: 548; LCDR1 comprising SEQ ID NO: 532;
(xxvii) LCDR3 comprising SEQ ID NO: 567; LCDR2 comprising SEQ ID NO: 187; LCDR1 comprising SEQ ID NO: 167;
(xxviii) LCDR3 comprising SEQ ID NO: 568; LCDR2 comprising SEQ ID NO: 549; LCDR1 comprising SEQ ID NO: 533;
(xxix) LCDR3 comprising SEQ ID NO: 569; LCDR2 comprising SEQ ID NO: 187; LCDR1 comprising SEQ ID NO: 165;
(xxx) LCDR3 comprising SEQ ID NO: 570; LCDR2 comprising SEQ ID NO: 550; LCDR1 comprising SEQ ID NO: 534;
(xxxi) LCDR3 comprising SEQ ID NO: 571; LCDR2 comprising SEQ ID NO: 551; LCDR1 comprising SEQ ID NO: 535;
(xxxii) LCDR3 comprising SEQ ID NO: 572; LCDR2 comprising SEQ ID NO: 552; LCDR1 comprising SEQ ID NO: 536;
(xxxiii) LCDR3 comprising SEQ ID NO: 573; LCDR2 comprising SEQ ID NO: 553; LCDR1 comprising SEQ ID NO: 52;
(xxxiv) LCDR3 comprising SEQ ID NO: 574; LCDR2 comprising SEQ ID NO: 553; LCDR1 comprising SEQ ID NO: 52;
(xxxv) LCDR3 comprising SEQ ID NO: 575; LCDR2 comprising SEQ ID NO: 554; LCDR1 comprising SEQ ID NO: 537; and
(xx) LCDR3 comprising SEQ ID NO: 576; LCDR2 comprising SEQ ID NO: 555; LCDR1 comprising SEQ ID NO: 538.

Any given Nav1.7 antibody or antigen binding fragment thereof comprising a VH domain paired with a VL domain to form a binding site for Nav1.7 antigen will comprise a combination of six CDRs: variable heavy chain CDR3 (HCDR3), variable heavy chain CDR2 (HCDR2), variable heavy chain CDR1 (HCDR1), variable light chain CDR3 (LCDR3), variable light chain CDR2 (LCDR2) and variable light chain CDR1 (LCDR1).

Although all combinations of six CDRs selected from the CDR sequence groups listed above are permissible, and within the scope of the invention, certain combinations of six CDRs are particularly preferred; these being the “native” combinations within a single Fab exhibiting high affinity binding to Nav1.7.

Preferred combinations of six CDRs include, but are not limited to, the combinations of variable heavy chain CDR3 (HCDR3), variable heavy chain CDR2 (HCDR2), variable heavy chain CDR1 (HCDR1), variable light chain CDR3 (LCDR3), variable light chain CDR2 (LCDR2) and variable light chain CDR1 (LCDR1) selected from the group consisting of:

(i) HCDR3 comprising SEQ ID NO: 28; HCDR2 comprising SEQ ID NO: 16; HCDR1 comprising SEQ ID NO: 8; LCDR3 comprising SEQ ID NO: 77; LCDR2 comprising SEQ ID NO: 62; LCDR1 comprising SEQ ID NO: 46;
(ii) HCDR3 comprising SEQ ID NO: 29; HCDR2 comprising SEQ ID NO: 16; HCDR1 comprising SEQ ID NO: 8; LCDR3 comprising SEQ ID NO: 78; LCDR2 comprising SEQ ID NO: 63; LCDR1 comprising SEQ ID NO: 47;
(iii) HCDR3 comprising SEQ ID NO: 29; HCDR2 comprising SEQ ID NO: 16; HCDR1 comprising SEQ ID NO: 8; LCDR3 comprising SEQ ID NO: 78; LCDR2 comprising SEQ ID NO: 63; LCDR1 comprising SEQ ID NO: 48;
(iv) HCDR3 comprising SEQ ID NO: 30; HCDR2 comprising SEQ ID NO: 17; HCDR1 comprising SEQ ID NO: 9; LCDR3 comprising SEQ ID NO: 79; LCDR2 comprising SEQ ID NO: 64; LCDR1 comprising SEQ ID NO: 49;
(v) HCDR3 comprising SEQ ID NO: 30; HCDR2 comprising SEQ ID NO: 17; HCDR1 comprising SEQ ID NO: 9; LCDR3 comprising SEQ ID NO: 80; LCDR2 comprising SEQ ID NO: 64; LCDR1 comprising SEQ ID NO: 50;
(vi) HCDR3 comprising SEQ ID NO: 31; HCDR2 comprising SEQ ID NO: 18; HCDR1 comprising SEQ ID NO: 10; LCDR3 comprising SEQ ID NO: 81; LCDR2 comprising SEQ ID NO: 65; LCDR1 comprising SEQ ID NO: 51;
(vii) HCDR3 comprising SEQ ID NO: 32; HCDR2 comprising SEQ ID NO: 19; HCDR1 comprising SEQ ID NO: 11; LCDR3 comprising SEQ ID NO: 82; LCDR2 comprising SEQ ID NO: 66; LCDR1 comprising SEQ ID NO: 52;
(viii) HCDR3 comprising SEQ ID NO: 33; HCDR2 comprising SEQ ID NO: 20; HCDR1 comprising SEQ ID NO: 12; LCDR3 comprising SEQ ID NO: 83; LCDR2 comprising SEQ ID NO: 67; LCDR1 comprising SEQ ID NO: 53;
(ix) HCDR3 comprising SEQ ID NO: 34; HCDR2 comprising SEQ ID NO: 21; HCDR1 comprising SEQ ID NO: 13; LCDR3 comprising SEQ ID NO: 84; LCDR2 comprising SEQ ID NO: 68; LCDR1 comprising SEQ ID NO: 54;
(x) HCDR3 comprising SEQ ID NO: 141; HCDR2 comprising SEQ ID NO: 119; HCDR1 comprising SEQ ID NO: 100; LCDR3 comprising SEQ ID NO: 204; LCDR2 comprising SEQ ID NO: 181; LCDR1 comprising SEQ ID NO: 161;
(xi) HCDR3 comprising SEQ ID NO: 142; HCDR2 comprising SEQ ID NO: 120; HCDR1 comprising SEQ ID NO: 101; LCDR3 comprising SEQ ID NO: 205; LCDR2 comprising SEQ ID NO: 182; LCDR1 comprising SEQ ID NO: 162;
(xii) HCDR3 comprising SEQ ID NO: 143; HCDR2 comprising SEQ ID NO: 121; HCDR1 comprising SEQ ID NO: 102; LCDR3 comprising SEQ ID NO: 206; LCDR2 comprising SEQ ID NO: 183; LCDR1 comprising SEQ ID NO: 163;
(xiii) HCDR3 comprising SEQ ID NO: 144; HCDR2 comprising SEQ ID NO: 122; HCDR1 comprising SEQ ID NO: 103; LCDR3 comprising SEQ ID NO: 207; LCDR2 comprising SEQ ID NO: 184; LCDR1 comprising SEQ ID NO: 164;
(xiv) HCDR3 comprising SEQ ID NO: 145; HCDR2 comprising SEQ ID NO: 123; HCDR1 comprising SEQ ID NO: 104; LCDR3 comprising SEQ ID NO: 208; LCDR2 comprising SEQ ID NO: 185; LCDR1 comprising SEQ ID NO: 165;
(xv) HCDR3 comprising SEQ ID NO: 146; HCDR2 comprising SEQ ID NO: 124; HCDR1 comprising SEQ ID NO: 105; LCDR3 comprising SEQ ID NO: 209; LCDR2 comprising SEQ ID NO: 186; LCDR1 comprising SEQ ID NO: 166;
(xvi) HCDR3 comprising SEQ ID NO: 145; HCDR2 comprising SEQ ID NO: 125; HCDR1 comprising SEQ ID NO: 106; LCDR3 comprising SEQ ID NO: 210; LCDR2 comprising SEQ ID NO: 187; LCDR1 comprising SEQ ID NO: 167;
(xvii) HCDR3 comprising SEQ ID NO: 145; HCDR2 comprising SEQ ID NO: 125; HCDR1 comprising SEQ ID NO: 106; LCDR3 comprising SEQ ID NO: 211; LCDR2 comprising SEQ ID NO: 188; LCDR1 comprising SEQ ID NO: 168;
(xviii) HCDR3 comprising SEQ ID NO: 147; HCDR2 comprising SEQ ID NO: 126; HCDR1 comprising SEQ ID NO: 107; LCDR3 comprising SEQ ID NO: 212; LCDR2 comprising SEQ ID NO: 189; LCDR1 comprising SEQ ID NO: 169;
(xix) HCDR3 comprising SEQ ID NO: 148; HCDR2 comprising SEQ ID NO: 127; HCDR1 comprising SEQ ID NO: 108; LCDR3 comprising SEQ ID NO: 213; LCDR2 comprising SEQ ID NO: 190; LCDR1 comprising SEQ ID NO: 170;
(xx) HCDR3 comprising SEQ ID NO: 149; HCDR2 comprising SEQ ID NO: 128; HCDR1 comprising SEQ ID NO: 109; LCDR3 comprising SEQ ID NO: 214; LCDR2 comprising SEQ ID NO: 191; LCDR1 comprising SEQ ID NO: 171;
(xxi) HCDR3 comprising SEQ ID NO: 149; HCDR2 comprising SEQ ID NO: 129; HCDR1 comprising SEQ ID NO: 109; LCDR3 comprising SEQ ID NO: 215; LCDR2 comprising SEQ ID NO: 192; LCDR1 comprising SEQ ID NO: 172;
(xxii) HCDR3 comprising SEQ ID NO: 293; HCDR2 comprising SEQ ID NO: 291; HCDR1 comprising SEQ ID NO: 289; LCDR3 comprising SEQ ID NO: 300; LCDR2 comprising SEQ ID NO: 298; LCDR1 comprising SEQ ID NO: 296;
(xxiii) HCDR3 comprising SEQ ID NO: 500; HCDR2 comprising SEQ ID NO: 472; HCDR1 comprising SEQ ID NO: 452; LCDR3 comprising SEQ ID NO: 563; LCDR2 comprising SEQ ID NO: 547; LCDR1 comprising SEQ ID NO: 530;
(xxiv) HCDR3 comprising SEQ ID NO: 501; HCDR2 comprising SEQ ID NO: 473; HCDR1 comprising SEQ ID NO: 453; LCDR3 comprising SEQ ID NO: 564; LCDR2 comprising SEQ ID NO: 68; LCDR1 comprising SEQ ID NO: 54;
(xxv) HCDR3 comprising SEQ ID NO: 502; HCDR2 comprising SEQ ID NO: 474; HCDR1 comprising SEQ ID NO: 454; LCDR3 comprising SEQ ID NO: 565; LCDR2 comprising SEQ ID NO: 187; LCDR1 comprising SEQ ID NO: 531;
(xxvi) HCDR3 comprising SEQ ID NO: 503; HCDR2 comprising SEQ ID NO: 475; HCDR1 comprising SEQ ID NO: 455; LCDR3 comprising SEQ ID NO: 566; LCDR2 comprising SEQ ID NO: 548; LCDR1 comprising SEQ ID NO: 532;
(xxvii) HCDR3 comprising SEQ ID NO: 504; HCDR2 comprising SEQ ID NO: 476; HCDR1 comprising SEQ ID NO: 456; LCDR3 comprising SEQ ID NO: 567; LCDR2 comprising SEQ ID NO: 187; LCDR1 comprising SEQ ID NO: 167;
(xxviii) HCDR3 comprising SEQ ID NO: 505; HCDR2 comprising SEQ ID NO: 477; HCDR1 comprising SEQ ID NO: 457; LCDR3 comprising SEQ ID NO: 568; LCDR2 comprising SEQ ID NO: 549; LCDR1 comprising SEQ ID NO: 533;
(xxix) HCDR3 comprising SEQ ID NO: 506; HCDR2 comprising SEQ ID NO: 478; HCDR1 comprising SEQ ID NO: 458; LCDR3 comprising SEQ ID NO: 569; LCDR2 comprising SEQ ID NO: 187; LCDR1 comprising SEQ ID NO: 165;
(xxx) HCDR3 comprising SEQ ID NO: 147; HCDR2 comprising SEQ ID NO: 479; HCDR1 comprising SEQ ID NO: 459; LCDR3 comprising SEQ ID NO: 570; LCDR2 comprising SEQ ID NO: 550; LCDR1 comprising SEQ ID NO: 534;
(xxxi) HCDR3 comprising SEQ ID NO: 507; HCDR2 comprising SEQ ID NO: 480; HCDR1 comprising SEQ ID NO: 460; LCDR3 comprising SEQ ID NO: 571; LCDR2 comprising SEQ ID NO: 551; LCDR1 comprising SEQ ID NO: 535;
(xxxii) HCDR3 comprising SEQ ID NO: 508; HCDR2 comprising SEQ ID NO: 481; HCDR1 comprising SEQ ID NO: 461; LCDR3 comprising SEQ ID NO: 572; LCDR2 comprising SEQ ID NO: 552; LCDR1 comprising SEQ ID NO: 536;
(xxxiii) HCDR3 comprising SEQ ID NO: 509; HCDR2 comprising SEQ ID NO: 482; HCDR1 comprising SEQ ID NO: 462; LCDR3 comprising SEQ ID NO: 573; LCDR2 comprising SEQ ID NO: 553; LCDR1 comprising SEQ ID NO: 52;
(xxxiv) HCDR3 comprising SEQ ID NO: 510; HCDR2 comprising SEQ ID NO: 483; HCDR1 comprising SEQ ID NO: 463; LCDR3 comprising SEQ ID NO: 574; LCDR2 comprising SEQ ID NO: 553; LCDR1 comprising SEQ ID NO: 52;
(xxxv) HCDR3 comprising SEQ ID NO: 511; HCDR2 comprising SEQ ID NO: 484; HCDR1 comprising SEQ ID NO: 464; LCDR3 comprising SEQ ID NO: 575; LCDR2 comprising SEQ ID NO: 554; LCDR1 comprising SEQ ID NO: 537; and
(xxxvi) HCDR3 comprising SEQ ID NO: 512; HCDR2 comprising SEQ ID NO: 485; HCDR1 comprising SEQ ID NO: 465; LCDR3 comprising SEQ ID NO: 576; LCDR2 comprising SEQ ID NO: 555; LCDR1 comprising SEQ ID NO: 538.

Further preferred Nav1.7 antibodies, exhibiting binding to the voltage-gated sodium channel human Nav1.7, include isolated antibodies or antigen binding fragments thereof, comprising a heavy chain variable domain having an amino acid sequence selected from the group consisting of: the amino acid sequences of SEQ ID NOs: 218-226, 236-247, 302 and 583-596, and amino acid sequences exhibiting at least 90%, 95%, 97%, 98% or 99% sequence identity to one of the recited sequences. Alternatively or in addition, the Nav1.7 antibodies may comprise a light chain variable domain having an amino acid sequence selected from the group consisting of: the amino acid sequences of SEQ ID NOs: 227-235, 248-259, 303 and 597-610, and amino acid sequences exhibiting at least 90%, 95%, 97%, 98% or 99% sequence identity to one of the recited sequences.

Although all possible pairings of VH domains and VL domains selected from the VH and VL domain sequence groups listed above are permissible, and within the scope of the invention, certain combinations of VH and VL are particularly preferred; these being the “native” combinations within a single Fab exhibiting binding to Nav1.7. Accordingly, preferred Nav1.7 antibodies, or antigen binding fragments thereof are those comprising a combination of a heavy chain variable domain (VH) and a light chain variable domain (VL), wherein the combination is selected from the group consisting of:

(i) VH comprising the amino acid sequence of SEQ ID NO: 218 and VL comprising the amino acid sequence of SEQ ID NO: 227;
(ii) VH comprising the amino acid sequence of SEQ ID NO: 219 and VL comprising the amino acid sequence of SEQ ID NO: 228;
(iii) VH comprising the amino acid sequence of SEQ ID NO: 220 and VL comprising the amino acid sequence of SEQ ID NO: 229;
(iv) VH comprising the amino acid sequence of SEQ ID NO: 221 and VL comprising the amino acid sequence of SEQ ID NO: 230;
(v) VH comprising the amino acid sequence of SEQ ID NO: 222 and VL comprising the amino acid sequence of SEQ ID NO: 231;
(vi) VH comprising the amino acid sequence of SEQ ID NO: 223 and VL comprising the amino acid sequence of SEQ ID NO: 232;
(vii) VH comprising the amino acid sequence of SEQ ID NO: 224 and VL comprising the amino acid sequence of SEQ ID NO: 233;
(viii) VH comprising the amino acid sequence of SEQ ID NO: 225 and VL comprising the amino acid sequence of SEQ ID NO: 234;
(ix) VH comprising the amino acid sequence of SEQ ID NO: 226 and VL comprising the amino acid sequence of SEQ ID NO: 235;
(x) VH comprising the amino acid sequence of SEQ ID NO: 236 and VL comprising the amino acid sequence of SEQ ID NO: 248;
(xi) VH comprising the amino acid sequence of SEQ ID NO: 237 and VL comprising the amino acid sequence of SEQ ID NO: 249;
(xii) VH comprising the amino acid sequence of SEQ ID NO: 238 and VL comprising the amino acid sequence of SEQ ID NO: 250;
(xiii) VH comprising the amino acid sequence of SEQ ID NO: 239 and VL comprising the amino acid sequence of SEQ ID NO: 251;
(xiv) VH comprising the amino acid sequence of SEQ ID NO: 240 and VL comprising the amino acid sequence of SEQ ID NO: 252;
(xv) VH comprising the amino acid sequence of SEQ ID NO: 241 and VL comprising the amino acid sequence of SEQ ID NO: 253;
(xvi) VH comprising the amino acid sequence of SEQ ID NO: 242 and VL comprising the amino acid sequence of SEQ ID NO: 254;
(xvii) VH comprising the amino acid sequence of SEQ ID NO: 243 and VL comprising the amino acid sequence of SEQ ID NO: 255;
(xviii) VH comprising the amino acid sequence of SEQ ID NO: 244 and VL comprising the amino acid sequence of SEQ ID NO: 256;
(xix) VH comprising the amino acid sequence of SEQ ID NO: 245 and VL comprising the amino acid sequence of SEQ ID NO: 257;
(xx) VH comprising the amino acid sequence of SEQ ID NO: 246 and VL comprising the amino acid sequence of SEQ ID NO: 258;
(xxi) VH comprising the amino acid sequence of SEQ ID NO: 247 and VL comprising the amino acid sequence of SEQ ID NO: 259; (xxii) VH comprising the amino acid sequence of SEQ ID NO: 302 and VL comprising the amino acid sequence of SEQ ID NO: 303;
(xxiii) VH comprising the amino acid sequence of SEQ ID NO: 583 and VL comprising the amino acid sequence of SEQ ID NO: 597;
(xxiv) VH comprising the amino acid sequence of SEQ ID NO: 584 and VL comprising the amino acid sequence of SEQ ID NO: 598;
(xxv) VH comprising the amino acid sequence of SEQ ID NO: 585 and VL comprising the amino acid sequence of SEQ ID NO: 599;
(xxvi) VH comprising the amino acid sequence of SEQ ID NO: 586 and VL comprising the amino acid sequence of SEQ ID NO: 600;
(xxvii) VH comprising the amino acid sequence of SEQ ID NO: 587 and VL comprising the amino acid sequence of SEQ ID NO: 601;
(xxviii) VH comprising the amino acid sequence of SEQ ID NO: 588 and VL comprising the amino acid sequence of SEQ ID NO: 602;
(xxix) VH comprising the amino acid sequence of SEQ ID NO: 589 and VL comprising the amino acid sequence of SEQ ID NO: 603;
(xxx) VH comprising the amino acid sequence of SEQ ID NO: 590 and VL comprising the amino acid sequence of SEQ ID NO: 604;
(xxxi) VH comprising the amino acid sequence of SEQ ID NO: 591 and VL comprising the amino acid sequence of SEQ ID NO: 605;
(xxxii) VH comprising the amino acid sequence of SEQ ID NO: 592 and VL comprising the amino acid sequence of SEQ ID NO: 606;
(xxxiii) VH comprising the amino acid sequence of SEQ ID NO: 593 and VL comprising the amino acid sequence of SEQ ID NO: 607;
(xxxiv) VH comprising the amino acid sequence of SEQ ID NO: 594 and VL comprising the amino acid sequence of SEQ ID NO: 608;
(xxxv) VH comprising the amino acid sequence of SEQ ID NO: 595 and VL comprising the amino acid sequence of SEQ ID NO: 609; and
(xxxvi) VH comprising the amino acid sequence of SEQ ID NO: 596 and VL comprising the amino acid sequence of SEQ ID NO: 610.

For each of the specific VH/VL combinations listed above, it is also permissible, and within the scope of the invention, to combine a variant VH domain having an amino acid sequence at least 90%, 92%, 95%, 97%, 98% or 99% identical to the recited VH domain sequence with a variant VL domain having an amino acid sequence at least 90%, 92%, 95%, 97%. 98% or 99% identical to the recited VL domain sequence.

Embodiments wherein the amino acid sequence of the VH domain exhibits less than 100% sequence identity with the sequences recited above may nevertheless comprise heavy chain CDRs which are identical to the HCDR1, HCDR2 and HCDR3 of the recited VH domain sequence whilst exhibiting amino acid sequence variation within the framework regions. Likewise, embodiments wherein the amino acid sequence of the VL domain exhibits less than 100% sequence identity with the sequences recited above may nevertheless comprise light chain CDRs which are identical to the LCDR1, LCDR2 and LCDR3 of the recited VL domain sequence whilst exhibiting amino acid sequence variation within the framework regions.

In the preceding paragraph, and elsewhere herein, the structure of the antibodies/antigen binding fragments is defined on the basis of % sequence identity with a recited reference sequence (with a given SEQ ID NO). In this context, % sequence identity between two amino acid sequences may be determined by comparing these two sequences aligned in an optimum manner and in which the amino acid sequence to be compared can comprise additions or deletions with respect to the reference sequence for an optimum alignment between these two sequences. The percentage of identity is calculated by determining the number of identical positions for which the amino acid residue is identical between the two sequences, by dividing this number of identical positions by the total number of positions in the comparison window and by multiplying the result obtained by 100 in order to obtain the percentage of identity between these two sequences. Typically, the comparison window will correspond to the full length of the sequence being compared. For example, it is possible to use the BLAST program, “BLAST 2 sequences” (Tatusova et al, “Blast 2 sequences—a new tool for comparing protein and nucleotide sequences”, FEMS Microbiol Lett. 174:247-250) available on the site http://www.ncbi.nlm.nih.gov/gorf/b12.html, the parameters used being those given by default (in particular for the parameters “open gap penalty”: 5, and “extension gap penalty”: 2; the matrix chosen being, for example, the matrix “BLOSUM 62” proposed by the program), the percentage of identity between the two sequences to be compared being calculated directly by the program.

### 8B6, 8A7 and 9A11

The most preferred Nav1.7 antibodies provided herein exhibit particularly advantageous properties. For example, the antibodies or antigen binding fragments thereof bind to human Nav1.7 with an affinity, as measured by ELISA (see for e.g., Example 8), of EC₅₀ less than 0.4 nM and/or effectively inhibit human Nav1.7 ion channel activity, by at least 40%, at least 50%, at least 60%, or at least 70%.

### 8B6

In a preferred embodiment, there is provided an antibody or antigen binding fragment thereof which binds to the voltage-gated sodium channel Nav1.7, said antibody or antigen binding fragment comprising a heavy chain variable (VH) domain comprising or consisting of an amino acid sequence selected from the group consisting of: the amino acid sequence of SEQ ID NO: 237, germlined variants and affinity variants thereof and amino acid sequences at least 90%, 95%, 97%, 98% or 99% identical thereto. Alternatively, or in addition, the antibody or antigen binding fragment thereof may comprise a light chain variable (VL) domain comprising or consisting of an amino acid sequence selected from the group consisting of: the amino acid sequence of SEQ ID NO: 249, germlined variants and affinity variants thereof and amino acid sequences at least 90%, 95%, 97%, 98% or 99% identical thereto.

Embodiments wherein the amino acid sequence of the VH domain exhibits less than 100% sequence identity with the sequence shown as SEQ ID NO: 237 may nevertheless comprise heavy chain CDRs which are identical to HCDR1, HCDR2 and HCDR3 of SEQ ID NO: 237 (SEQ ID NOs: 101, 120 and 142, respectively) whilst exhibiting amino acid sequence variation within the framework regions. Likewise, embodiments wherein the amino acid sequence of the VL domain exhibits less than 100% sequence identity with the sequence shown as SEQ ID NO: 249 may nevertheless comprise heavy chain CDRs which are identical to LCDR1, LCDR2 and LCDR3 of SEQ ID NO: 249 (SEQ ID NOs:162, 182 and 205, respectively) whilst exhibiting amino acid sequence variation within the framework regions.

### 8A7

In a preferred embodiment, there is provided an antibody or antigen binding fragment thereof which binds to the voltage-gated sodium channel Nav1.7, said antibody or antigen binding fragment comprising a heavy chain variable (VH) domain comprising or consisting of an amino acid sequence selected from the group consisting of: the amino acid sequence of SEQ ID NO: 236, germlined variants and affinity variants thereof and amino acid sequences at least 90%, 95%, 97%, 98% or 99% identical thereto. Alternatively, or in addition, the antibody or antigen binding fragment thereof may comprise a light chain variable (VL) domain comprising or consisting of an amino acid sequence selected from the group consisting of: the amino acid sequence of SEQ ID NO: 248, germlined variants and affinity variants thereof and amino acid sequences at least 90%, 95%, 97%, 98% or 99% identical thereto.

Embodiments wherein the amino acid sequence of the VH domain exhibits less than 100% sequence identity with the sequence shown as SEQ ID NO: 236 may nevertheless comprise heavy chain CDRs which are identical to HCDR1, HCDR2 and HCDR3 of SEQ ID NO: 236 (SEQ ID NOs: 100, 119 and 141, respectively) whilst exhibiting amino acid sequence variation within the framework regions. Likewise, embodiments wherein the amino acid sequence of the VL domain exhibits less than 100% sequence identity with the sequence shown as SEQ ID NO: 248 may nevertheless comprise heavy chain CDRs which are identical to LCDR1, LCDR2 and LCDR3 of SEQ ID NO: 248 (SEQ ID NOs:161, 181 and 204, respectively) whilst exhibiting amino acid sequence variation within the framework regions.

### 9A11

In a preferred embodiment, there is provided an antibody or antigen binding fragment thereof which binds to the voltage-gated sodium channel Nay 1.7, said antibody or antigen binding fragment comprising a heavy chain variable (VH) domain comprising or consisting of an amino acid sequence selected from the group consisting of: the amino acid sequence of SEQ ID NO: 240, germlined variants and affinity variants thereof and amino acid sequences at least 90%, 95%, 97%, 98% or 99% identical thereto. Alternatively, or in addition, the antibody or antigen binding fragment thereof may comprise a light chain variable (VL) domain comprising or consisting of an amino acid sequence selected from the group consisting of: the amino acid sequence of SEQ ID NO: 252, germlined variants and affinity variants thereof and amino acid sequences at least 90%, 95%, 97%, 98% or 99% identical thereto.

Embodiments wherein the amino acid sequence of the VH domain exhibits less than 100% sequence identity with the sequence shown as SEQ ID NO: 240 may nevertheless comprise heavy chain CDRs which are identical to HCDR1, HCDR2 and HCDR3 of SEQ ID NO: 240 (SEQ ID NOs: 104, 123 and 145, respectively) whilst exhibiting amino acid sequence variation within the framework regions. Likewise, embodiments wherein the amino acid sequence of the VL domain exhibits less than 100% sequence identity with the sequence shown as SEQ ID NO: 252 may nevertheless comprise heavy chain CDRs which are identical to LCDR1, LCDR2 and LCDR3 of SEQ ID NO: 252 (SEQ ID NOs:165, 185 and 208, respectively) whilst exhibiting amino acid sequence variation within the framework regions.

The Nav1.7 antibodies described herein are camelid-derived i.e. derived from an animal of the Camelidae family, for example llama (lama glama). The camelid-derived Nav1.7 antibodies may be isolated or recombinantly expressed monoclonal antibodies. Preferred embodiments may be a humanised (or germlined) monoclonal antibody (e.g. a humanised variant of a camelid-derived antibody), a chimeric antibody (e.g. a camelid-human chimeric antibody) or a humanised chimeric antibody (e.g. a chimeric antibody comprising humanised variants of camelid VH and VL domains and constant domains of a human antibody).

Camelid-derived Nav1.7 antibodies may comprise at least one hypervariable loop or complementarity determining region obtained from a VH domain or a VL domain of a species in the family Camelidae. In a particular embodiment, the Nav1.7 antibody, or antigen binding fragment thereof, may comprise a heavy chain variable domain (VH) and light chain variable domain (VL), wherein the VH and VL domains, or one or more CDRs thereof, are camelid-derived. In particular embodiments the antibody or antigen binding fragment thereof may comprise llama VH and/or VL domains, or human germlined variants of llama VH and VL domains. This antibody, or antigen binding fragment, may exhibit “high human homology”, as defined herein.

The camelid-derived Nav1.7 antibodies described herein typically exhibit VH and/or VL region amino acid sequences having at least 90% (e.g., 91%, 92%, 93%, 94%, 95%, 96%, 97%, 98%, or 99%) sequence identity to the closest matching human antibody germline sequence.

Further preferred embodiments of the invention include humanised (or human germlined) variants of the camelid-derived Nav1.7 antibodies.

In preferred embodiments, the VH domain and/or VL domain or at least one of the complementarity determining regions (CDRs) of the Nav1.7 antibodies described herein are derived from an antibody raised by immunization of a host animal with a DNA molecule comprising a nucleotide sequence encoding full-length Nav1.7, a protein having at least 70%, at least 80%, at least 90%, at least 95%, at least 98% identity to Nav1.7 or a fragment of full-length Nav1.7. Preferably, the host animal is a llama.

### Features/Properties of the Nav1.7 Antibodies

In the aforementioned aspects and embodiments, the Nav1.7 antibodies, or antigen binding fragments thereof, may each exhibit one or more, or any combination, of the following properties or features:

The antibody or antigen binding fragment may bind to Nav1.7 with superior affinity as compared with Nav1.7 antibodies of the prior art.

The antibody or antigen binding fragment may exhibit an affinity of binding which is at least 10-fold, at least 20-fold, at least 50-fold or at least 100-fold higher than a reference prior art antibody, wherein the reference antibody is selected from the group consisting of: UCB_932; UCB_983; and UCB_1066, as described in US2011/0135662.

The antibody or antigen binding fragment thereof may exhibit an EC₅₀ for binding to an extracellular region of human Nav1.7, of less than 2 nM, preferably less than 1 nM, preferably less than 0.4 nM, when tested by ELISA, preferably as described elsewhere herein (see for e.g., Example 8).

The antibody or antigen binding fragment with superior binding affinity for Nav1.7 may exhibit an off-rate for human Nav1.7, of less than 5×10⁻³ s⁻¹, less than 5×10⁻⁴ s⁻¹, less than 5×10⁻⁵ s⁻¹. and typically in the range of from 0.03 to 45×10⁻⁴ s⁻¹, when tested by BIACORE analysis, preferably as described elsewhere herein.

The affinity of the antibody or antigen binding fragment may be determined by testing the affinity of the corresponding monoclonal antibody (mAb) in an in vitro ELISA or BIACORE assay.

The affinity of the antibody or antigen binding fragment for human Nav1.7, as measured by Biacore may be determined using a human Nav1.7 peptide construct, as described elsewhere herein, and shown in Table 4. For example, the off-rate may be determined by Biacore analysis using a hNav1.7 loop A3-llama Fc chimeric construct as represented by SEQ ID NO: 267 shown in Table 4. Alternatively, the off-rate may be determined by Biacore analysis using a hNav1.7 loop A3-GST chimeric construct as represented by SEQ ID NO: 272 shown in Table 4.

The antibody or antigen binding fragment may bind to human Nav1.7 expressed on the surface of cells, particularly cells exhibiting low level expression of Nav1.7.

The antibody or antigen binding fragment may bind to an extracellular region of Nav1.7 selected from the following extracellular loops (see Table 3):
A3 as represented by SEQ ID NO: 262 (corresponding to amino acid residues 269 to 319 of SEQ ID NO:260);
B1 as represented by SEQ ID NO: 263 (corresponding to amino acid residues 753 to 765 of SEQ ID NO:260);
C1 as represented by SEQ ID NO: 264 (corresponding to amino acid residues 1201 to 1214 of SEQ ID NO:260);
D1 as represented by SEQ ID NO: 265 (corresponding to amino acid residues 1523 to 1536 of SEQ ID NO:260);
C3 as represented by SEQ ID NO: 266 (corresponding to amino acid residues 1334 to 1420 of SEQ ID NO:260);
B2 as represented by SEQ ID NO: 611 (corresponding to amino acid residues 809 to 818 of SEQ ID NO:260).

The antibody or antigen binding fragment may exhibit selective binding for only one extracellular loop of Nav1.7 wherein “selective binding” means that the antibody or antigen binding fragment thereof is capable of binding to one extracellular loop of Nav1.7, but not any other extracellular loop of Nav1.7. The antibody or antigen binding fragment may exhibit selective binding to an extracellular loop of Nav1.7 selected from A3, C1, B1, C1, D1, C3 or B2 (as characterised above), wherein selective binding means that the antibody does not bind to a second extracellular loop of Nav1.7 selected from A3, C1, B1, C1, D1, C3 or B2.

The antibody or antigen binding fragment may bind to human Nav1.7 with superior affinity and modulate the activity of Nav1.7. The antibody or antigen binding fragment may inhibit the activity of Nav1.7 i.e. reduce or completely block the flow of ions through the ion channel. Inhibition of channel activity may be tested by any techniques standard in the part, but is preferably determined using an in vitro patch clamp assay, as described elsewhere herein (see Example 10).

The antibody or antigen binding fragment may inhibit Nav1.7 channel activity by at least 20%, at least 30%, at least 40%, at least 50%, at least 60%, at least 70%, at least 80%, or at least 90% as compared with channel activity measured in the absence of antibody or antigen binding fragment or as compared with channel activity measured in the presence of a suitable control.

The antibody or antigen binding fragment may be capable of binding Nav1.7 in both native form (e.g. Nav1.7 expressed on the surface of a cell, such as a Nav1.7 expressing cell line) and denatured form and/or be capable of immunoprecipitating Nav1.7, for example according to an immunoprecipitation protocol as described elsewhere herein (see Example 10).

The antibody or antigen binding fragment may exhibit cross-reactivity for human Nav1.7 and species homologues thereof, for example Nav1.7 of primate, mouse and/or rat origin. Alternatively, the antibody or antigen binding fragment may exhibit specific binding to human Nav1.7.

The antibody or antigen binding fragment thereof may exhibit selective binding to human Nav1.7 such that there is no (detectable) binding of the antibody or antigen binding fragment to other voltage-gated sodium channels, particularly Nav1.2 and Nav1.5. In preferred embodiments, the antibodies or antigen binding fragments do not bind human Nav1.2 and/or human Nav1.5. The antibody or antigen binding fragment may exhibit selective binding to human Nav1.7 such that there is no (detectable) binding to any other ion channels within the ion channel protein family.

The antibody or antigen binding fragment may provide very high production yields (>4 g/L) in recombinant antibody expression systems, such as for example the CHK1SV cell line (proprietary to BioWa/Lonza), as compared to a 1-2 g/L historical average for therapeutic antibody products, resulting in a substantial reduction in production costs.

The antibody may exhibit one or more effector functions selected from antibody-dependent cell-mediated cytotoxicity (ADCC), complement dependent cytotoxicity (CDC) and antibody-dependent cell-mediated phagocytosis (ADCP) against cells expressing Nav1.7 protein on the cell surface. Alternatively, the antibody may not possess any effector function.

In further aspects, the invention also provides polynucleotide molecules which encode the above-listed antibodies and antigen binding fragments, particularly antibodies and antigen binding fragments which bind to human Nav1.7, in addition to expression vectors comprising the polynucleotides, host cells containing the vectors and methods of recombinant expression/production of the antibodies described herein.

In a still further aspect, the invention provides a pharmaceutical composition comprising any one of the antibodies described above, particularly the Nav1.7 antibodies, and a pharmaceutically acceptable carrier or excipient.

A still further aspect of the invention concerns methods of medical treatment using the above-listed Nav1.7 antibodies, particularly in the prophylaxis and/or treatment of pain.

The present invention also relates to methods for raising antibodies against particular protein targets, for example ion channels, using DNA immunization.

Therefore, in a further aspect, the present invention provides a method of raising an antibody which binds a target protein wherein

(a) the target protein has a length of at least 1115 amino acids, and the method comprises immunizing a host animal with a DNA molecule comprising an open reading frame of at least 3345 nucleotides encoding:
(i) the full-length target protein;
(ii) a protein having at least 70% identity to the full-length target protein; or
(iii) a fragment of the full-length target protein;

Or

(b) the target protein has at least 8 transmembrane domains and the method comprises immunizing a host animal with a DNA molecule comprising an open reading frame encoding:
(i) the full-length target protein;
(ii) a protein having at least 70% identity to the full-length target protein; or
(iii) a fragment of the full-length target protein having at least 8 transmembrane domains;

### Or

(c) the target protein is naturally encoded by a nucleotide sequence which is difficult to replicate in a common E. coli strain and the method comprises immunizing a host animal with a DNA molecule comprising an open reading frame, which is difficult to replicate in a common E. coli strain, encoding:
(i) the full-length target protein;
(ii) a protein having at least 70% identity to the full-length target protein; or
(iii) a fragment of the full-length target protein.

### BRIEF DESCRIPTION OF THE DRAWINGS

The invention will be further understood with reference to the following experimental examples and the accompanying Figures in which:

FIG. 1. Diagrammatic representation of a multi-domain voltage-gated ion channel of the calcium or sodium family.

FIG. 2. The amino acid and nucleotide sequences of human Nav1.7, as encoded by SCN9A.

FIG. 3. shows the binding affinity for human Nav1.7 extracellular loop A3 of Nav1.7 antibodies raised by peptide immunization, as measured by ELISA.

FIG. 4. shows immunoprecipitation of full-length Nav1.7 from a cell lysate using Nav1.7 antibodies raised by peptide immunization.

FIG. 5. shows the EcoRI and HindIII restriction digest analysis of pCMV6-hNav1.7 plasmid extracted from XL-10 Gold cells (“Gold”) or Copycutter cells (CC).

FIG. 6. shows the specificity of binding to either Nav1.7 extracellular loop A3, extracellular loop B1-C1-D1 or extracellular loop C3 for Nav1.7 antibodies derived by DNA immunization.

FIG. 7. shows the binding affinity for human Nav1.7 extracellular loop A3 (A), extracellular loop B1-C1-D1 (B) or extracellular loop C3 (C) of Nav1.7 antibodies raised by DNA immunization, as measured by ELISA.

FIG. 8. shows the binding of Nav1.7 antibodies raised by DNA immunization to (A) Nav1.7 extracellular loop A3 as compared with Nav1.5 extracellular loop A3, (B) Nav1.7 extracellular loop B1-C1-D1 as compared with Nav1.5 extracellular loop B1-C1-D1, and (C) Nav1.7 extracellular loop A3 as compared with Nav1.2 extracellular loop A3.

FIG. 9. Immunoprecipitation results for Nav1.7 antibodies raised by DNA Immunization.

FIG. 10. Patch clamp analysis for Nav1.7 antibodies raised by DNA Immunization.

FIG. 11. Rodent cross-reactivity for Nav1.7 mAbs derived from DNA immunization.

FIG. 12. shows an alignment between the sequences of human Nav1.7 loop A3, mouse Nav1.7 loop A3 and various chimeric human-mouse loop A3 sequences (A) and also the binding affinity of Nav1.7 mAb 8A7 for human Nav1.7 extracellular loop A3 and the various human-mouse chimeric loop A3 sequences as measured by ELISA (B).

FIG. 13. shows the results of functional characterization of mAb 8A7 using manual patch clamp electrophysiology. (A) Voltage/current density curve. (B) Peak current density analysis on hNav1.7 expressing cells. (C) Peak current density analysis on hNav1.5 expressing cells.

FIG. 14. shows an exemplary immune response in a llama immunized with DNA encoding the GCGR, wherein the DNA immunizations were boosted optionally with cells expressing the GCGR.

FIG. 15. shows the scFv vector compared with the Fab vector. In the scFv display vector the VH and VL are coupled by a GC linker. There is only the gene 3 leader sequence. In the Fab vector, pCB3, the VHCH1 and VLCL both have their own leader sequence and ribosomal binding site (RBS). The orientation is also different: in the scFv vector, the VH is fused to gene 3 whereas in the Fab vector the VL is fused to gene 3.

FIG. 16. shows the nucleotide and amino acid sequences of the scFv vector (SEQ ID NOs: 315 and 316, respectively).

FIG. 17. shows the results of an ELISA of monoclonal phage 1C3 comparing scFv display with Fab display. Left panel: phage ELISA on Nt-GCGR coating. Nt-GCGR was coated at 1 ug/ml. Right panel: Phage ELISA on GCGR-lipoparticles. GCGR-lipoparticles were coated at 5 Units per well.

FIG. 18. shows enrichment of GCGR binders following selection on GCGR lipoparticles and the GCGR N terminal domain using Fab and scFv library screening.

FIG. 19. shows the results of an scFv binding ELISA testing specific binding to the N terminal domain of GCGR.

FIG. 20. shows an alignment of VH and VL sequences from scFv clones identified following one round of selection on GCGR lipoparticles and a second round of selection on Nt-GCGR.

FIG. 21 shows enrichment of GCGR binders following selection on GCGR lipoparticles and the GCGR N terminal domain using Fab and scFv library screening.

FIG. 22. shows enrichment of GCGR binders following selection on GCGR lipoparticles and counter-selection using null lipoparticles.

FIG. 23. shows an alignment of VH and VL sequences from scFv clones identified following three rounds of selection on GCGR lipoparticles with a round of counter-selection on null lipoparticles.

FIG. 24. shows the results of an scFv binding ELISA. Plates were coated with the N-terminal domain of GCGR, with GCGR lipoparticles, or with negative control CXCR4 lipoparticles. Periplasmic extracts were added to the lipoparticles in 2 different volumes (20 μl and 60 μl). Anti-Myc 9E10 was used as detection antibody. As negative control, binding to the null lipoparticles was included.

FIG. 25. shows the results of a mAb binding ELISA. Binding to GCGR- and CXCR4-lipoparticles (negative control) (A) and binding to Nt-GCGR-hFc (B). Lipoparticles were coated at 10 U/well. Nt-GCGR was coated at 1 μg/ml. mAbs were allowed to bind to the coated target at 1 μg/ml.

FIG. 26. shows mAb binding to GCGR overexpressing CHO cells analyzed by FACS. Binding of mAbs to CHO-GCGR and CHO-CXCR4 (negative control). Binding of benchmark Fab A9 compared to family 19 mAbs. (top left). Binding of family 19 mAb to CHO-GCGR cells.

FIG. 27. shows binding of mAbs tested on HEK293E GCGR-expressing cells by flow cytometry. Curves from left to right show: (i) binding curve to irrelevant transfected HEK293E cells; (ii) binding curve to HEK293EΔNt cells; (iii) binding curve to HEK293E cells expressing full GCGR; (iv) binding curve to HEK293Ecells expressing full GCGR, but competed with excess of Nt domain. (Upper left) benchmark A9, (upper right) mAb 6B3, (lower left) mAB 6C6 and (lower right). mAb 6A3 were tested at a concentration of 1 μg/ml.

FIG. 27. shows enrichment of GCGR binders following selection on GCGR lipoparticles and counter-selection using null lipoparticles and Nt-GCGR. Lipoparticles were directly coated to the plate (20 or 2 U/well). Counter selection on null-lipoparticles (100 U/well) was performed in all 3 selection rounds, counter selection with 50 μg/ml Nt-GCGR was performed in round 2 and round 3. Diagnosis on N-terminal GCGR domain and null lipoparticles was included.

FIG. 29. shows a sequence alignment of VH domains identified following counter-selection on Nt-GCGR.

FIG. 30. shows the results of GCGR lipoparticle ELISA. 10 U/well GCGR lipoparticle and 10 U/well irrelevant control lipoparticle (CXCR4) were coated and 22 μl periplasmic extract was added. Mouse anti-Myc 9E10 was used for detection of scFv binding.

FIG. 31. shows binding of mAb/scFv-Fc on GCGR overexpressing cells by FACS. mAbs were tested at a concentration of 1 μg/ml.

FIG. 32. shows the nucleotide and amino acid sequences of the scFv-Fc fusion vector (SEQ ID NOs: 612 and 613, respectively). Depicted sequence was cloned in the pUPE vector by KpnI-NotI restriction cloning.

### DEFINITIONS

“DNA Immunization”—As used herein, the term “DNA immunization” (also referred to as DNA Vaccination or Nucleic Acid Immunization) means the introduction of a nucleic acid molecule encoding one or more selected antigens into a host animal for the in vivo expression of the antigen. The nucleic acid molecule can be introduced directly into a recipient host animal to stimulate an immune response, such as by injection, inhalation, oral, intranasal and mucosal administration, or can be introduced ex vivo into cells which have been removed from the host. In the latter case, the transformed cells containing the nucleic acid are reintroduced into the recipient host animal for expression of the antigen in vivo.
“Antibody” or “Immunoglobulin”—As used herein, the term “immunoglobulin” includes a polypeptide having a combination of two heavy and two light chains whether or not it possesses any relevant specific immunoreactivity. “Antibodies” refers to such assemblies which have significant known specific immunoreactive activity to an antigen of interest (e.g. the voltage gated sodium channel Nav1.7). The term “Nav1.7 antibodies” is used herein to refer to antibodies which exhibit immunological specificity for Nav1.7 protein, including human Nav1.7 and species homologues thereof. Antibodies and immunoglobulins comprise light and heavy chains, with or without an interchain covalent linkage between them. Basic immunoglobulin structures in vertebrate systems are relatively well understood.

The generic term “immunoglobulin” comprises five distinct classes of antibody that can be distinguished biochemically. All five classes of antibodies are within the scope of the present invention, the following discussion will generally be directed to the IgG class of immunoglobulin molecules. With regard to IgG, immunoglobulins comprise two identical light polypeptide chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable region.

The light chains of an antibody are classified as either kappa or lambda (κ,λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA, IgD or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the instant disclosure and, accordingly, are within the scope of the instant invention.

As indicated above, the variable region of an antibody allows the antibody to selectively recognize and specifically bind epitopes on antigens. That is, the VL domain and VH domain of an antibody combine to form the variable region that defines a three dimensional antigen binding site. This quaternary antibody structure forms the antigen binding site present at the end of each arm of the Y. More specifically, the antigen binding site is defined by three complementary determining regions (CDRs) on each of the VH and VL chains.

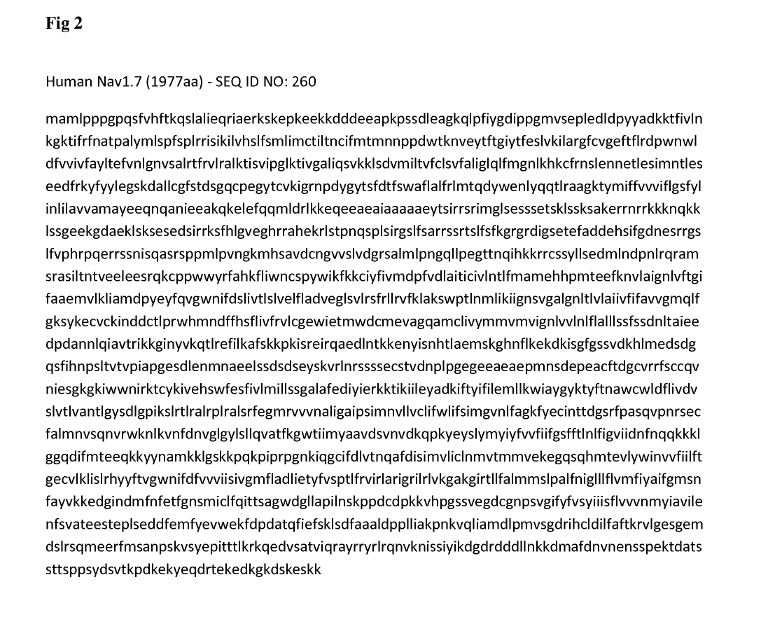

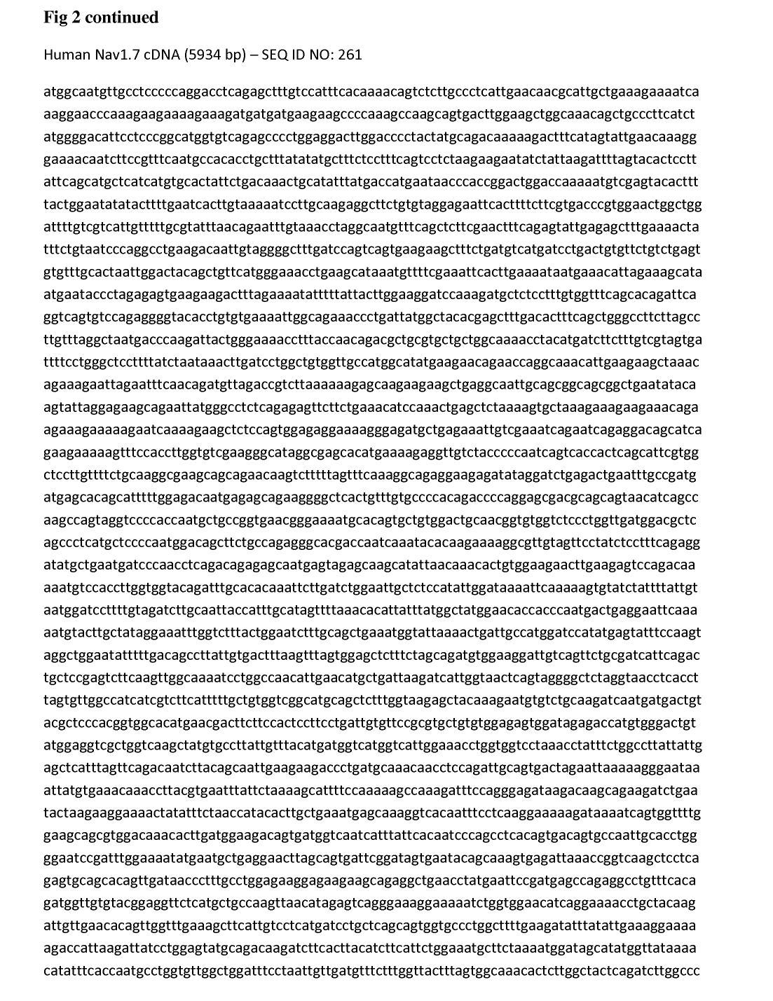

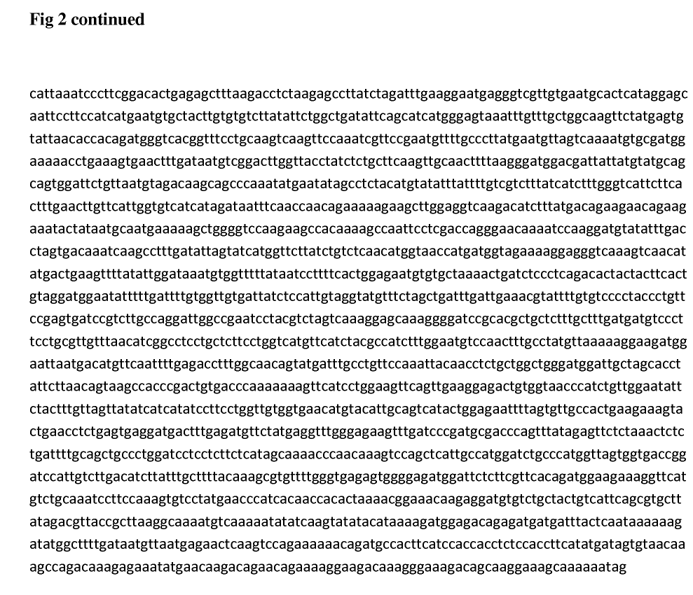

“Nav1.7”, “Nav1.7 protein” or “Nav1.7 antigen”—As used herein, the terms Nav1.7, Nav1.7 protein and Nav1.7 antigen are used interchangeably and refer to the voltage gated sodium channel protein encoded in humans by the gene SCN9A. The terms are broad enough to encompass all species homologues of the protein including human, rat and mouse proteins. The amino acid sequence of the full-length human Nav1.7 protein is represented by SEQ ID NO: 260, and the encoding nucleotide sequence is represented by SEQ ID NO: 261 (see FIG. 2). These sequences correspond to the sequences deposited in the SwissProt database as human protein SCN9A, accession number Q15858.
“Binding Site”—As used herein, the term “binding site” comprises a region of a polypeptide which is responsible for selectively binding to a target antigen of interest (e.g. Nav1.7). Binding domains comprise at least one binding site. Exemplary binding domains include an antibody variable domain. The antibody molecules of the invention may comprise a single binding site or multiple (e.g., two, three or four) binding sites.
“Derived From”—As used herein the term “derived from” a designated protein (e.g. a camelid antibody or antigen-binding fragment thereof) refers to the origin of the polypeptide or amino acid sequence. In one embodiment, the polypeptide or amino acid sequence which is derived from a particular starting polypeptide is a CDR sequence or sequence related thereto. In one embodiment, the amino acid sequence which is derived from a particular starting polypeptide is not contiguous. For example, in one embodiment, one, two, three, four, five, or six CDRs are derived from a starting antibody. In one embodiment, the polypeptide or amino acid sequence which is derived from a particular starting polypeptide or amino acid sequence has an amino acid sequence that is essentially identical to that of the starting sequence, or a portion thereof wherein the portion consists of at least 3-5 amino acids, at least 5-10 amino acids, at least 10-20 amino acids, at least 20-30 amino acids, or at least 30-50 amino acids, or which is otherwise identifiable to one of ordinary skill in the art as having its origin in the starting sequence. In one embodiment, the one or more CDR sequences derived from the starting antibody are altered to produce variant CDR sequences, e.g. affinity variants, wherein the variant CDR sequences maintain target antigen binding activity.
“Camelid-Derived”—In certain preferred embodiments, the antibodies of the invention comprise framework amino acid sequences and/or CDR amino acid sequences derived from a camelid conventional antibody raised by active immunisation, particularly by DNA immunization, of a camelid. However, antibodies of the invention comprising camelid-derived amino acid sequences may be engineered to comprise framework and/or constant region sequences derived from a human amino acid sequence (i.e. a human antibody) or other non-camelid mammalian species. For example, a human or non-human primate framework region, heavy chain portion, and/or hinge portion may be included in the subject Nav1.7 antibodies. In one embodiment, one or more non-camelid amino acids may be present in the framework region of a “camelid-derived” antibody, e.g., a camelid framework amino acid sequence may comprise one or more amino acid mutations in which the corresponding human or non-human primate amino acid residue is present. Moreover, camelid-derived VH and VL domains, or humanised variants thereof, may be linked to the constant domains of human antibodies to produce a chimeric molecule, as described elsewhere herein.
“Conservative amino acid substitution”—A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide may be replaced with another amino acid residue from the same side chain family. In another embodiment, a string of amino acids can be replaced with a structurally similar string that differs in order and/or composition of side chain family members.
“Heavy chain portion”—As used herein, the term “heavy chain portion” includes amino acid sequences derived from the constant domains of an immunoglobulin heavy chain. A polypeptide comprising a heavy chain portion comprises at least one of: a CH1 domain, a hinge (e.g., upper, middle, and/or lower hinge region) domain, a CH2 domain, a CH3 domain, or a variant or fragment thereof. In one embodiment, an antibody or antigen binding fragment of the invention may comprise the Fc portion of an immunoglobulin heavy chain (e.g., a hinge portion, a CH2 domain, and a CH3 domain). In another embodiment, an antibody or antigen binding fragment of the invention may lack at least a portion of a constant domain (e.g., all or part of a CH2 domain). In certain embodiments, at least one, and preferably all, of the constant domains are derived from a human immunoglobulin heavy chain. For example, in one preferred embodiment, the heavy chain portion comprises a fully human hinge domain. In other preferred embodiments, the heavy chain portion comprising a fully human Fc portion (e.g., hinge, CH2 and CH3 domain sequences from a human immunoglobulin).

In certain embodiments, the constituent constant domains of the heavy chain portion are from different immunoglobulin molecules. For example, a heavy chain portion of a polypeptide may comprise a CH2 domain derived from an IgG1 molecule and a hinge region derived from an IgG3 or IgG4 molecule. In other embodiments, the constant domains are chimeric domains comprising portions of different immunoglobulin molecules. For example, a hinge may comprise a first portion from an IgG1 molecule and a second portion from an IgG3 or IgG4 molecule. As set forth above, it will be understood by one of ordinary skill in the art that the constant domains of the heavy chain portion may be modified such that they vary in amino acid sequence from the naturally occurring (wild-type) immunoglobulin molecule. That is, the polypeptides of the invention disclosed herein may comprise alterations or modifications to one or more of the heavy chain constant domains (CH1, hinge, CH2 or CH3) and/or to the light chain constant region domain (CL). Exemplary modifications include additions, deletions or substitutions of one or more amino acids in one or more domains.

“Chimeric”—A “chimeric” protein comprises a first amino acid sequence linked to a second amino acid sequence with which it is not naturally linked in nature. The amino acid sequences may normally exist in separate proteins that are brought together in the fusion polypeptide or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship. Exemplary chimeric antibodies of the invention include fusion proteins comprising camelid-derived VH and VL domains, or humanised variants thereof, fused to the constant domains of a human antibody, e.g. human IgG1, IgG2, IgG3 or IgG4.
“Variable region” or “variable domain”—The terms “variable region” and “variable domain” are used herein interchangeably and are intended to have equivalent meaning. The term “variable” refers to the fact that certain portions of the variable domains VH and VL differ extensively in sequence among antibodies and are used in the binding and specificity of each particular antibody for its target antigen. However, the variability is not evenly distributed throughout the variable domains of antibodies. It is concentrated in three segments called “hypervariable loops” in each of the VL domain and the VH domain which form part of the antigen binding site. The first, second and third hypervariable loops of the VLambda light chain domain are referred to herein as L1(λ), L2(λ) and L3(λ) and may be defined as comprising residues 24-33 (L1(λ), consisting of 9, 10 or 11 amino acid residues), 49-53 (L2(λ), consisting of 3 residues) and 90-96 (L3(λ), consisting of 5 residues) in the VL domain (Morea et al., Methods 20:267-279 (2000)). The first, second and third hypervariable loops of the VKappa light chain domain are referred to herein as L1(κ), L2(κ) and L3(κ) and may be defined as comprising residues 25-33 (L1(K), consisting of 6, 7, 8, 11, 12 or 13 residues), 49-53 (L2(K), consisting of 3 residues) and 90-97 (L3(K), consisting of 6 residues) in the VL domain (Morea et al., Methods 20:267-279 (2000)). The first, second and third hypervariable loops of the VH domain are referred to herein as H1, H2 and H3 and may be defined as comprising residues 25-33 (H1, consisting of 7, 8 or 9 residues), 52-56 (H2, consisting of 3 or 4 residues) and 91-105 (H3, highly variable in length) in the VH domain (Morea et al., Methods 20:267-279 (2000)).

Unless otherwise indicated, the terms L1, L2 and L3 respectively refer to the first, second and third hypervariable loops of a VL domain, and encompass hypervariable loops obtained from both Vkappa and Vlambda isotypes. The terms H1, H2 and H3 respectively refer to the first, second and third hypervariable loops of the VH domain, and encompass hypervariable loops obtained from any of the known heavy chain isotypes, including γ, ε, δ, α or μ.

The hypervariable loops L1, L2, L3, H1, H2 and H3 may each comprise part of a “complementarity determining region” or “CDR”, as defined below. The terms “hypervariable loop” and “complementarity determining region” are not strictly synonymous, since the hypervariable loops (HVs) are defined on the basis of structure, whereas complementarity determining regions (CDRs) are defined based on sequence variability (Kabat et al., Sequences of Proteins of Immunological Interest, 5th Ed. Public Health Service, National Institutes of Health, Bethesda, Md., 1983) and the limits of the HVs and the CDRs may be different in some VH and VL domains.

The CDRs of the VL and VH domains can typically be defined as comprising the following amino acids: residues 24-34 (LCDR1), 50-56 (LCDR2) and 89-97 (LCDR3) in the light chain variable domain, and residues 31-35 or 31-35b (HCDR1), 50-65 (HCDR2) and 95-102 (HCDR3) in the heavy chain variable domain; (Kabat et al., Sequences of Proteins of Immunological Interest, 5th Ed. Public Health Service, National Institutes of Health, Bethesda, Md. (1991)). Thus, the HVs may be comprised within the corresponding CDRs and references herein to the “hypervariable loops” of VH and VL domains should be interpreted as also encompassing the corresponding CDRs, and vice versa, unless otherwise indicated.

The more highly conserved portions of variable domains are called the framework region (FR), as defined below. The variable domains of native heavy and light chains each comprise four FRs (FR1, FR2, FR3 and FR4, respectively), largely adopting a 3-sheet configuration, connected by the three hypervariable loops. The hypervariable loops in each chain are held together in close proximity by the FRs and, with the hypervariable loops from the other chain, contribute to the formation of the antigen-binding site of antibodies. Structural analysis of antibodies revealed the relationship between the sequence and the shape of the binding site formed by the complementarity determining regions (Chothia et al., J. Mol. Biol. 227: 799-817 (1992)); Tramontano et al., J. Mol. Biol, 215:175-182 (1990)). Despite their high sequence variability, five of the six loops adopt just a small repertoire of main-chain conformations, called “canonical structures”. These conformations are first of all determined by the length of the loops and secondly by the presence of key residues at certain positions in the loops and in the framework regions that determine the conformation through their packing, hydrogen bonding or the ability to assume unusual main-chain conformations.

“CDR”—As used herein, the term “CDR” or “complementarity determining region” means the non-contiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat based on sequence comparisons.

TABLE 1
CDR definitions

| | CDR Definitions | | |
|---|---|---|---|
| | Kabat¹ | Chothia² | MacCallum³ |
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

“Framework region”—The term “framework region” or “FR region” as used herein, includes the amino acid residues that are part of the variable region, but are not part of the CDRs (e.g., using the Kabat definition of CDRs). Therefore, a variable region framework is between about 100-120 amino acids in length but includes only those amino acids outside of the CDRs. For the specific example of a heavy chain variable domain and for the CDRs as defined by Kabat et al., framework region 1 corresponds to the domain of the variable region encompassing amino acids 1-30; framework region 2 corresponds to the domain of the variable region encompassing amino acids 36-49; framework region 3 corresponds to the domain of the variable region encompassing amino acids 66-94, and framework region 4 corresponds to the domain of the variable region from amino acids 103 to the end of the variable region. The framework regions for the light chain are similarly separated by each of the light claim variable region CDRs. Similarly, using the definition of CDRs by Chothia et al. or McCallum et al. the framework region boundaries are separated by the respective CDR termini as described above. In preferred embodiments the CDRs are as defined by Kabat.

In naturally occurring antibodies, the six CDRs present on each monomeric antibody are short, non-contiguous sequences of amino acids that are specifically positioned to form the antigen binding site as the antibody assumes its three dimensional configuration in an aqueous environment. The remainder of the heavy and light variable domains show less inter-molecular variability in amino acid sequence and are termed the framework regions. The framework regions largely adopt a 3-sheet conformation and the CDRs form loops which connect, and in some cases form part of, the 3-sheet structure. Thus, these framework regions act to form a scaffold that provides for positioning the six CDRs in correct orientation by inter-chain, non-covalent interactions. The antigen binding site formed by the positioned CDRs defines a surface complementary to the epitope on the immunoreactive antigen. This complementary surface promotes the non-covalent binding of the antibody to the immunoreactive antigen epitope. The position of CDRs can be readily identified by one of ordinary skill in the art.

“Hinge region”—As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux K. H. et al. J. Immunol. 161:4083-90 1998). Antibodies of the invention comprising a “fully human” hinge region may contain one of the hinge region sequences shown in Table 2 below.

TABLE 2
human hinge sequences

| IgG | Upper hinge | Middle hinge | Lower hinge |
|---|---|---|---|
| IgG1 | EPKSCDKTHT | CPPCP | APELLGGP |
| | (SEQ ID NO: 273) | (SEQ ID NO: 274) | (SEQ ID NO: 275) |
| IgG3 | ELKTPLGDTTHT | CPRCP (EPKSCDTPPPCPRCP)₃ | APELLGGP |
| | (SEQ ID NO: 276) | (SEQ ID NO: 277) | (SEQ ID NO: 278) |
| IgG4 | ESKYGPP | CPSCP | APEFLGGP |
| | (SEQ ID NO: 279) | (SEQ ID NO: 280) | (SEQ ID NO: 281) |
| IgG42 | ERK | CCVECPPPCP | APPVAGP |
| | (SEQ ID NO: 282) | (SEQ ID NO: 283) | (SEQ ID NO: 284) |

“CH2 domain”—As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system, Kabat E A et al. Sequences of Proteins of Immunological Interest. Bethesda, US Department of Health and Human Services, NIH. 1991). The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.
“Fragment”—The term “fragment”, as used in the context of antibodies of the invention, refers to a part or portion of an antibody or antibody chain comprising fewer amino acid residues than an intact or complete antibody or antibody chain. The term “antigen-binding fragment” refers to a polypeptide fragment of an immunoglobulin or antibody that binds antigen or competes with intact antibody (i.e., with the intact antibody from which they were derived) for antigen binding (i.e., specific binding to Nav1.7). As used herein, the term “fragment” of an antibody molecule includes antigen-binding fragments of antibodies, for example, an antibody light chain variable domain (VL), an antibody heavy chain variable domain (VH), a single chain antibody (scFv), a F(ab′)2 fragment, a Fab fragment, an Fd fragment, an Fv fragment, and a single domain antibody fragment (DAb). Fragments can be obtained, e.g., via chemical or enzymatic treatment of an intact or complete antibody or antibody chain or by recombinant means.
“Valency”—As used herein the term “valency” refers to the number of potential target binding sites in a polypeptide. Each target binding site specifically binds one target molecule or specific site on a target molecule. When a polypeptide comprises more than one target binding site, each target binding site may specifically bind the same or different molecules (e.g., may bind to different ligands or different antigens, or different epitopes on the same antigen).
“Specificity”—The term “specificity” refers to the ability to bind (e.g., immunoreact with) a given target, e.g., Nav1.7. A polypeptide may be monospecific and contain one or more binding sites which specifically bind a target or a polypeptide may be multispecific and contain two or more binding sites which specifically bind the same or different targets.
“Synthetic”—As used herein the term “synthetic” with respect to polypeptides includes polypeptides which comprise an amino acid sequence that is not naturally occurring. For example, non-naturally occurring polypeptides which are modified forms of naturally occurring polypeptides (e.g., comprising a mutation such as an addition, substitution or deletion) or which comprise a first amino acid sequence (which may or may not be naturally occurring) that is linked in a linear sequence of amino acids to a second amino acid sequence (which may or may not be naturally occurring) to which it is not naturally linked in nature.
“Engineered”—As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques). Preferably, the antibodies of the invention are engineered, including for example, humanized and/or chimeric antibodies, and antibodies which have been engineered to improve one or more properties, such as antigen binding, stability/half-life or effector function.
“Modified antibody”—As used herein, the term “modified antibody” includes synthetic forms of antibodies which are altered such that they are not naturally occurring, e.g., antibodies that comprise at least two heavy chain portions but not two complete heavy chains (such as, domain deleted antibodies or minibodies); multispecific forms of antibodies (e.g., bispecific, trispecific, etc.) altered to bind to two or more different antigens or to different epitopes on a single antigen); heavy chain molecules joined to scFv molecules and the like. scFv molecules are known in the art and are described, e.g., in U.S. Pat. No. 5,892,019. In addition, the term “modified antibody” includes multivalent forms of antibodies (e.g., trivalent, tetravalent, etc., antibodies that bind to three or more copies of the same antigen). In another embodiment, a modified antibody of the invention is a fusion protein comprising at least one heavy chain portion lacking a CH2 domain and comprising a binding domain of a polypeptide comprising the binding portion of one member of a receptor ligand pair.

The term “modified antibody” may also be used herein to refer to amino acid sequence variants of the antibodies of the invention as structurally defined herein. It will be understood by one of ordinary skill in the art that an antibody may be modified to produce a variant antibody which varies in amino acid sequence in comparison to the antibody from which it was derived. For example, nucleotide or amino acid substitutions leading to conservative substitutions or changes at “nonessential” amino acid residues may be made (e.g., in CDR and/or framework residues). Amino acid substitutions can include replacement of one or more amino acids with a naturally occurring or non-natural amino acid.

“Humanising substitutions”—As used herein, the term “humanising substitutions” refers to amino acid substitutions in which the amino acid residue present at a particular position in the VH or VL domain of an antibody (for example a camelid-derived Nav1.7 antibody) is replaced with an amino acid residue which occurs at an equivalent position in a reference human VH or VL domain. The reference human VH or VL domain may be a VH or VL domain encoded by the human germline. Humanising substitutions may be made in the framework regions and/or the CDRs of the antibodies, defined herein.
“Humanised variants”—As used herein the term “humanised variant” refers to a variant antibody which contains one or more “humanising substitutions” compared to a reference antibody, wherein a portion of the reference antibody (e.g. the VH domain and/or the VL domain or parts thereof containing at least one CDR) has an amino acid derived from a non-human species, and the “humanising substitutions” occur within the amino acid sequence derived from a non-human species.
“Germlined variants”—The term “germlined variant” is used herein to refer specifically to “humanised variants” in which the “humanising substitutions” result in replacement of one or more amino acid residues present at a particular position (s) in the VH or VL domain of an antibody (for example a camelid-derived Nav1.7 antibody) with an amino acid residue which occurs at an equivalent position in a reference human VH or VL domain encoded by the human germline. It is typical that for any given “germlined variant”, the replacement amino acid residues substituted into the germlined variant are taken exclusively, or predominantly, from a single human germline-encoded VH or VL domain. The terms “humanised variant” and “germlined variant” are often used interchangeably herein. Introduction of one or more “humanising substitutions” into a camelid-derived (e.g. llama derived) VH or VL domain results in production of a “humanised variant” of the camelid (llama)-derived VH or VL domain. If the amino acid residues substituted in are derived predominantly or exclusively from a single human germline-encoded VH or VL domain sequence, then the result may be a “human germlined variant” of the camelid (llama)-derived VH or VL domain.
“Affinity variants”—As used herein, the term “affinity variant” refers to a variant antibody which exhibits one or more changes in amino acid sequence compared to a reference antibody, wherein the affinity variant exhibits an altered affinity for the target antigen in comparison to the reference antibody. For example, affinity variants will exhibit a changed affinity for Nav1.7, as compared to the reference Nav1.7 antibody. Preferably the affinity variant will exhibit improved affinity for the target antigen, e.g. Nav1.7, as compared to the reference antibody. Affinity variants typically exhibit one or more changes in amino acid sequence in the CDRs, as compared to the reference antibody. Such substitutions may result in replacement of the original amino acid present at a given position in the CDRs with a different amino acid residue, which may be a naturally occurring amino acid residue or a non-naturally occurring amino acid residue. The amino acid substitutions may be conservative or non-conservative.
“High human homology”—An antibody comprising a heavy chain variable domain (VH) and a light chain variable domain (VL) will be considered as having high human homology if the VH domains and the VL domains, taken together, exhibit at least 90% amino acid sequence identity to the closest matching human germline VH and VL sequences. Antibodies having high human homology may include antibodies comprising VH and VL domains of native non-human antibodies which exhibit sufficiently high % sequence identity to human germline sequences, including for example antibodies comprising VH and VL domains of camelid conventional antibodies, as well as engineered, especially humanised or germlined, variants of such antibodies and also “fully human” antibodies.

In one embodiment the VH domain of the antibody with high human homology may exhibit an amino acid sequence identity or sequence homology of 80% or greater with one or more human VH domains across the framework regions FR1, FR2, FR3 and FR4. In other embodiments the amino acid sequence identity or sequence homology between the VH domain of the polypeptide of the invention and the closest matching human germline VH domain sequence may be 85% or greater, 90% or greater, 95% or greater, 97% or greater, or up to 99% or even 100%.

In one embodiment the VH domain of the antibody with high human homology may contain one or more (e.g. 1 to 10) amino acid sequence mis-matches across the framework regions FR1, FR2, FR3 and FR4, in comparison to the closest matched human VH sequence.

In another embodiment the VL domain of the antibody with high human homology may exhibit a sequence identity or sequence homology of 80% or greater with one or more human VL domains across the framework regions FR1, FR2, FR3 and FR4. In other embodiments the amino acid sequence identity or sequence homology between the VL domain of the polypeptide of the invention and the closest matching human germline VL domain sequence may be 85% or greater 90% or greater, 95% or greater, 97% or greater, or up to 99% or even 100%.

In one embodiment the VL domain of the antibody with high human homology may contain one or more (e.g. 1 to 10) amino acid sequence mis-matches across the framework regions FR1, FR2, FR3 and FR4, in comparison to the closest matched human VL sequence.

Before analyzing the percentage sequence identity between the antibody with high human homology and human germline VH and VL, the canonical folds may be determined, which allows the identification of the family of human germline segments with the identical combination of canonical folds for H1 and H2 or L1 and L2 (and L3). Subsequently the human germline family member that has the highest degree of sequence homology with the variable region of the antibody of interest is chosen for scoring the sequence homology. The determination of Chothia canonical classes of hypervariable loops L1, L2, L3, H1 and H2 can be performed with the bioinformatics tools publicly available on webpage www.bioinf.org.uk/abs/chothia.html.page. The output of the program shows the key residue requirements in a datafile. In these datafiles, the key residue positions are shown with the allowed amino acids at each position. The sequence of the variable region of the antibody of interest is given as input and is first aligned with a consensus antibody sequence to assign the Kabat numbering scheme. The analysis of the canonical folds uses a set of key residue templates derived by an automated method developed by Martin and Thornton (Martin et al., J. Mol. Biol. 263:800-815 (1996)).

With the particular human germline V segment known, which uses the same combination of canonical folds for H1 and H2 or L1 and L2 (and L3), the best matching family member in terms of sequence homology can be determined. With bioinformatics tools the percentage sequence identity between the VH and VL domain framework amino acid sequences of the antibody of interest and corresponding sequences encoded by the human germline can be determined, but actually manual alignment of the sequences can be applied as well. Human immunoglobulin sequences can be identified from several protein data bases, such as VBase (http://vbase.mrc-cpe.cam.ac.uk/) or the Pluckthun/Honegger database (http://www.bioc.unizh.ch/antibody/Sequences/Germlines. To compare the human sequences to the V regions of VH or VL domains in an antibody of interest a sequence alignment algorithm such as available via websites like www.expasy.ch/tools/#align can be used, but also manual alignment with the limited set of sequences can be performed. Human germline light and heavy chain sequences of the families with the same combinations of canonical folds and with the highest degree of homology with the framework regions 1, 2, and 3 of each chain are selected and compared with the variable region of interest; also the FR4 is checked against the human germline JH and JK or JL regions.

Note that in the calculation of overall percent sequence homology the residues of FR1, FR2 and FR3 are evaluated using the closest match sequence from the human germline family with the identical combination of canonical folds. Only residues different from the closest match or other members of the same family with the same combination of canonical folds are scored (NB—excluding any primer-encoded differences). However, for the purposes of humanization, residues in framework regions identical to members of other human germline families, which do not have the same combination of canonical folds, can be considered “human”, despite the fact that these are scored “negative” according to the stringent conditions described above. This assumption is based on the “mix and match” approach for humanization, in which each of FR1, FR2, FR3 and FR4 is separately compared to its closest matching human germline sequence and the humanized molecule therefore contains a combination of different FRs as was done by Qu and colleagues (Qu et al., Clin. Cancer Res. 5:3095-3100 (1999)) and Ono and colleagues (Ono et al., Mol. Immunol. 36:387-395 (1999)). The boundaries of the individual framework regions may be assigned using the IMGT numbering scheme, which is an adaptation of the numbering scheme of Chothia (Lefranc et al., NAR 27: 209-212 (1999); imgt.cines.fr).

Antibodies with high human homology may comprise hypervariable loops or CDRs having human or human-like canonical folds, as discussed in detail below.

In one embodiment at least one hypervariable loop or CDR in either the VH domain or the VL domain of the antibody with high human homology may be obtained or derived from a VH or VL domain of a non-human antibody, for example a conventional antibody from a species of Camelidae, yet exhibit a predicted or actual canonical fold structure which is substantially identical to a canonical fold structure which occurs in human antibodies.

It is well established in the art that although the primary amino acid sequences of hypervariable loops present in both VH domains and VL domains encoded by the human germline are, by definition, highly variable, all hypervariable loops, except CDR H3 of the VH domain, adopt only a few distinct structural conformations, termed canonical folds (Chothia et al., J. Mol. Biol. 196:901-917 (1987); Tramontano et al. Proteins 6:382-94 (1989)), which depend on both the length of the hypervariable loop and presence of the so-called canonical amino acid residues (Chothia et al., J. Mol. Biol. 196:901-917 (1987)). Actual canonical structures of the hypervariable loops in intact VH or VL domains can be determined by structural analysis (e.g. X-ray crystallography), but it is also possible to predict canonical structure on the basis of key amino acid residues which are characteristic of a particular structure (discussed further below). In essence, the specific pattern of residues that determines each canonical structure forms a “signature” which enables the canonical structure to be recognised in hypervariable loops of a VH or VL domain of unknown structure; canonical structures can therefore be predicted on the basis of primary amino acid sequence alone.

The predicted canonical fold structures for the hypervariable loops of any given VH or VL sequence in an antibody with high human homology can be analysed using algorithms which are publicly available from www.bioinf.org.uk/abs/chothia.html,

www.biochem.ucl.ac.uk/˜martin/antibodies.html and
www.bioc.unizh.ch/antibody/Sequences/Germlines/Vbase_hVk.html. These tools permit query VH or VL sequences to be aligned against human VH or VL domain sequences of known canonical structure, and a prediction of canonical structure made for the hypervariable loops of the query sequence.
In the case of the VH domain, H1 and H2 loops may be scored as having a canonical fold structure “substantially identical” to a canonical fold structure known to occur in human antibodies if at least the first, and preferable both, of the following criteria are fulfilled:
1. An identical length, determined by the number of residues, to the closest matching human canonical structural class.
2. At least 33% identity, preferably at least 50% identity with the key amino acid residues described for the corresponding human H1 and H2 canonical structural classes.
(note for the purposes of the foregoing analysis the H1 and H2 loops are treated separately and each compared against its closest matching human canonical structural class)

The foregoing analysis relies on prediction of the canonical structure of the H1 and H2 loops of the antibody of interest. If the actual structures of the H1 and H2 loops in the antibody of interest are known, for example based on X-ray crystallography, then the H1 and H2 loops in the antibody of interest may also be scored as having a canonical fold structure “substantially identical” to a canonical fold structure known to occur in human antibodies if the length of the loop differs from that of the closest matching human canonical structural class (typically by ±1 or ±2 amino acids) but the actual structure of the H1 and H2 loops in the antibody of interest matches the structure of a human canonical fold.

Key amino acid residues found in the human canonical structural classes for the first and second hypervariable loops of human VH domains (H1 and H2) are described by Chothia et al., J. Mol. Biol. 227:799-817 (1992), the contents of which are incorporated herein in their entirety by reference. In particular, Table 3 on page 802 of Chothia et al., which is specifically incorporated herein by reference, lists preferred amino acid residues at key sites for H1 canonical structures found in the human germline, whereas Table 4 on page 803, also specifically incorporated by reference, lists preferred amino acid residues at key sites for CDR H2 canonical structures found in the human germline.

In one embodiment, both H1 and H2 in the VH domain of the antibody with high human homology exhibit a predicted or actual canonical fold structure which is substantially identical to a canonical fold structure which occurs in human antibodies.

Antibodies with high human homology may comprise a VH domain in which the hypervariable loops H1 and H2 form a combination of canonical fold structures which is identical to a combination of canonical structures known to occur in at least one human germline VH domain. It has been observed that only certain combinations of canonical fold structures at H1 and H2 actually occur in VH domains encoded by the human germline. In an embodiment H1 and H2 in the VH domain of the antibody with high human homology may be obtained from a VH domain of a non-human species, e.g. a Camelidae species, yet form a combination of predicted or actual canonical fold structures which is identical to a combination of canonical fold structures known to occur in a human germline or somatically mutated VH domain. In non-limiting embodiments H1 and H2 in the VH domain of the antibody with high human homology may be obtained from a VH domain of a non-human species, e.g. a Camelidae species, and form one of the following canonical fold combinations: 1-1, 1-2, 1-3, 1-6, 1-4, 2-1, 3-1 and 3-5.

An antibody with high human homology may contain a VH domain which exhibits both high sequence identity/sequence homology with human VH, and which contains hypervariable loops exhibiting structural homology with human VH.

It may be advantageous for the canonical folds present at H1 and H2 in the VH domain of the antibody with high human homology, and the combination thereof, to be “correct” for the human VH germline sequence which represents the closest match with the VH domain of the antibody with high human homology in terms of overall primary amino acid sequence identity. By way of example, if the closest sequence match is with a human germline VH3 domain, then it may be advantageous for H1 and H2 to form a combination of canonical folds which also occurs naturally in a human VH3 domain. This may be particularly important in the case of antibodies with high human homology which are derived from non-human species, e.g. antibodies containing VH and VL domains which are derived from camelid conventional antibodies, especially antibodies containing humanised camelid VH and VL domains.

Thus, in one embodiment the VH domain of a Nav1.7 antibody with high human homology may exhibit a sequence identity or sequence homology of 80% or greater, 85% or greater, 90% or greater, 95% or greater, 97% or greater, or up to 99% or even 100% with a human VH domain across the framework regions FR1, FR2, FR3 and FR4, and in addition H1 and H2 in the same antibody are obtained from a non-human VH domain (e.g. derived from a Camelidae species), but form a combination of predicted or actual canonical fold structures which is the same as a canonical fold combination known to occur naturally in the same human VH domain.

In other embodiments, L1 and L2 in the VL domain of the antibody with high human homology are each obtained from a VL domain of a non-human species (e.g. a camelid-derived VL domain), and each exhibits a predicted or actual canonical fold structure which is substantially identical to a canonical fold structure which occurs in human antibodies.

As with the VH domains, the hypervariable loops of VL domains of both VLambda and VKappa types can adopt a limited number of conformations or canonical structures, determined in part by length and also by the presence of key amino acid residues at certain canonical positions.

Within an antibody of interest having high human homology, L1, L2 and L3 loops obtained from a VL domain of a non-human species, e.g. a Camelidae species, may be scored as having a canonical fold structure “substantially identical” to a canonical fold structure known to occur in human antibodies if at least the first, and preferable both, of the following criteria are fulfilled:

1. An identical length, determined by the number of residues, to the closest matching human structural class.
2. At least 33% identity, preferably at least 50% identity with the key amino acid residues described for the corresponding human L1 or L2 canonical structural classes, from either the VLambda or the VKappa repertoire.
(note for the purposes of the foregoing analysis the L1 and L2 loops are treated separately and each compared against its closest matching human canonical structural class)

The foregoing analysis relies on prediction of the canonical structure of the L1, L2 and L3 loops in the VL domain of the antibody of interest. If the actual structure of the L1, L2 and L3 loops is known, for example based on X-ray crystallography, then L1, L2 or L3 loops derived from the antibody of interest may also be scored as having a canonical fold structure “substantially identical” to a canonical fold structure known to occur in human antibodies if the length of the loop differs from that of the closest matching human canonical structural class (typically by ±1 or ±2 amino acids) but the actual structure of the Camelidae loops matches a human canonical fold.

Key amino acid residues found in the human canonical structural classes for the CDRs of human VLambda and VKappa domains are described by Morea et al. Methods, 20: 267-279 (2000) and Martin et al., J. Mol. Biol., 263:800-815 (1996). The structural repertoire of the human VKappa domain is also described by Tomlinson et al. EMBO J. 14:4628-4638 (1995), and that of the VLambda domain by Williams et al. J. Mol. Biol., 264:220-232 (1996). The contents of all these documents are to be incorporated herein by reference.

L1 and L2 in the VL domain of an antibody with high human homology may form a combination of predicted or actual canonical fold structures, which is identical to a combination of canonical fold structures known to occur in a human germline VL domain. In non-limiting embodiments L1 and L2 in the VLambda domain of an antibody with high human homology (e.g. an antibody containing a camelid-derived VL domain or a humanised variant thereof) may form one of the following canonical fold combinations: 11-7, 13-7(A,B,C), 14-7(A,B), 12-11, 14-11 and 12-12 (as defined in Williams et al. J. Mol. Biol. 264:220-32 (1996) and as shown on http://www.bioc.uzh.ch/antibody/Sequences/Germlines/VBase_hVL.html). In non-limiting embodiments L1 and L2 in the Vkappa domain may form one of the following canonical fold combinations: 2-1, 3-1, 4-1 and 6-1 (as defined in Tomlinson et al. EMBO J. 14:4628-38 (1995) and as shown on http://www.bioc.uzh.ch/antibody/Sequences/Germlines/VBase_hVK.html).

In a further embodiment, all three of L1, L2 and L3 in the VL domain of an antibody with high human homology may exhibit a substantially human structure. It is preferred that the VL domain of the antibody with high human homology exhibits both high sequence identity/sequence homology with human VL, and also that the hypervariable loops in the VL domain exhibit structural homology with human VL.

In one embodiment, the VL domain of a Nav1.7 antibody with high human homology may exhibit a sequence identity of 80% or greater, 85% or greater, 90% or greater, 95% or greater, 97% or greater, or up to 99% or even 100% with a human VL domain across the framework regions FR1, FR2, FR3 and FR4, and in addition hypervariable loop L1 and hypervariable loop L2 may form a combination of predicted or actual canonical fold structures which is the same as a canonical fold combination known to occur naturally in the same human VL domain.

It is, of course, envisaged that VH domains exhibiting high sequence identity/sequence homology with human VH, and also structural homology with hypervariable loops of human VH will be combined with VL domains exhibiting high sequence identity/sequence homology with human VL, and also structural homology with hypervariable loops of human VL to provide antibodies with high human homology containing VH/VL pairings (e.g camelid-derived VH/VL pairings) with maximal sequence and structural homology to human-encoded VH/VL pairings.

“scFv” or “scFv fragment”—An scFv or scFv fragment means a single chain variable fragment. An scFv is a fusion protein of a VH domain and a VL domain of an antibody connected via a linker.

### DETAILED DESCRIPTION

As summarised above, the invention relates at least in part, to antibodies or antigen binding fragments thereof, derived from antibodies raised by DNA immunization.

Immunization with a nucleic acid, typically DNA, involves the delivery to a host animal of a nucleic acid “immunogen” comprising a nucleotide sequence, which encodes a target antigen of interest. The cells of the host typically take up the nucleic acid that has been introduced and express the encoded antigen by normal cellular mechanisms. DNA immunization provides an alternative means for raising antibodies against particular targets, as compared with immunization using purified protein or peptide antigens or whole cells expressing target antigens.

The problem with immunization techniques based around direct delivery of the protein antigen of interest is that sufficiently high level recombinant expression of the antigen of interest is required. This can be particularly difficult if antigen expression creates a lethal phenotype in the expression system used. It can also be difficult to achieve high level recombinant expression if the nucleic acid, for example the DNA encoding the antigen, is inherently unstable in the expression system and/or cannot be replicated successfully in a standard or common E. coli strain. This may be the case in particular for large protein targets encoded by long nucleotide sequences. For membrane-associated proteins, immunization strategies may involve delivery of whole cells expressing the target antigen. However, this approach can be unsuccessful if the expression level of the membrane protein is low and/or the immune response of the host animal is directed against cell-associated antigens other than the target of interest.

For large protein targets, it is sometimes preferable or necessary to immunize with a peptide corresponding to a fragment taken from the full-length protein target or antigen. However, using this approach, the peptide may be presented to the immune system of the host animal in a non-native configuration and therefore the antibodies raised may exhibit sub-optimal properties when tested for binding activity and/or functional activity against the full-length target protein. This is particularly the case for proteins with transmembrane spanning domains such as GPCRs and ion channels, which can adopt a complex three-dimensional structure relative to the membrane.

The present inventors have found DNA immunization to be a particularly effective approach for the production of antibodies which bind proteins, wherein said target proteins are particularly long and/or have several transmembrane domains and/or are naturally encoded by a nucleotide sequence which is difficult to replicate successfully in standard or common E. coli strains. In the context of the present invention, DNA immunization is typically used to raise antibodies against target proteins wherein antibodies against said target proteins raised by techniques other than DNA immunization do not exhibit desirable properties such as high affinity binding and/or agonistic or antagonistic properties.

Therefore, in a first aspect, the present invention provides an antibody or antigen binding fragment, which binds to a target protein, comprising at least one complementarity determining region (CDR) derived from an antibody raised by DNA immunization of a host animal.

In certain embodiments, the target protein to which the antibody or antigen binding fragment binds is a protein having a length of at least 1115 amino acids, at least 1300 amino acids, at least 1500 amino acids, or at least 1900 amino acids. The target protein may have a maximum length of 3000 amino acids, 2400 amino acids or 2100 amino acids. In certain embodiments, the target protein may have a length in the range 1115-2500 amino acids, preferably 1700-2400 amino acids, preferably 1791-2347 amino acids.

The target protein may be naturally encoded by a nucleotide sequence of at least 3345, at least 3800, at least 4000, at least 4500, at least 5000, at least 5500 nucleotides or at least 5931 nucleotides in length. The target protein may be naturally encoded by a nucleotide sequence having a maximum length of 10,000, 7500, 7100, or 6500 nucleotides. In certain embodiments, the target protein may be naturally encoded by a nucleotide sequence having a length in the range 3345-7500 nucleotides, preferably 4000-7500 nucleotides, preferably 5000-7100 nucleotides, more preferably 5373-7041 nucleotides.

The term “naturally encoded by a nucleotide sequence” should be interpreted in its broadest sense and may be taken to mean that the native gene/genomic DNA encoding the full-length protein has a nucleotide length as defined, or that the pre-mRNA or mature mRNA transcript generated following transcription of the native gene has a nucleotide length as defined. The length of the native gene/genomic sequence may be calculated so as to exclude residues upstream and/or downstream of the start and stop codons, respectively, for example, upstream regulatory elements and/or the native promoter. The naturally-encoding nucleotide sequence may also be calculated using the length of the complementary DNA (cDNA) sequence i.e. to exclude intronic sequences located in the native gene.

Alternatively or in addition, the target protein to which the antibody or antigen binding fragment binds may be a membrane protein having at least 8, at least 10, at least 12, at least 14, at least 18 or at least 20 transmembrane domains. As used herein, a transmembrane domain is intended to mean a domain of a protein, which spans the width of any cellular membrane including but not limited to the outer cell membrane, the nuclear membrane, the membrane surrounding organelles such as mitochondria and chloroplasts. The target protein may be a membrane protein having up to 24 transmembrane domains.

Alternatively or in addition, the target protein to which the antibody or antigen binding fragment binds may be a protein naturally encoded by a nucleotide sequence which is difficult to replicate in a common E. coli strain. As noted above, the term “naturally encoded by a nucleotide sequence” should be interpreted in its broadest sense and may be taken to mean that the native gene/genomic DNA is difficult to replicate in a standard or common E. coli strain, or may be taken to mean that the cDNA sequence is difficult to replicate in a standard or common E. coli strain. The pre-mRNA or mature mRNA transcript generated following transcription of the native gene may be difficult to replicate in a standard or common E. coli strain.

The target protein may be naturally encoded by a nucleotide sequence which is inherently unstable or difficult to replicate in standard or common E. coli strains. In certain embodiments, the nucleotide sequence is difficult to replicate in the standard E. coli strain XL10-Gold (as supplied by Stratagene). E-coli XL10-Gold cells are tetracycline and chloramphenicol resistant and have a genotype and background as follows: TetrD(mcrA)183 D(mcrCB-hsdSMR-mrr)173 endA1 supE44 thi-1 recA1 gyrA96 relA1 lac Hte [F′ proAB lacIqZDM15 Tn10 (Tetr) Amy Camr] (Genes listed signify mutant alleles. Genes on the F′ episome, however, are wild-type unless indicated otherwise). E. coli XL10-Gold cells are defined by the manufacturer as follows:

XL10-Gold ultracompetent cells were created for transformation of large DNA molecules with high efficiency. These cells exhibit the Hte phenotype, which increases the transformation efficiency of ligated and large DNA molecules. XL10-Gold ultracompetent cells are ideal for constructing plasmid DNA libraries because they decrease size bias and produce larger, more complex plasmid libraries. XL10-Gold cells are deficient in all known restriction systems [D(mcrA)183 D(mcrCB-hsdSMR-mrr)173]. The strain is endonuclease deficient (endA), greatly improving the quality of miniprep DNA, and recombination deficient (recA), helping to ensure insert stability. The lacIqZDM15 gene on the F′ episome allows blue-white screening for recombinant plasmids.

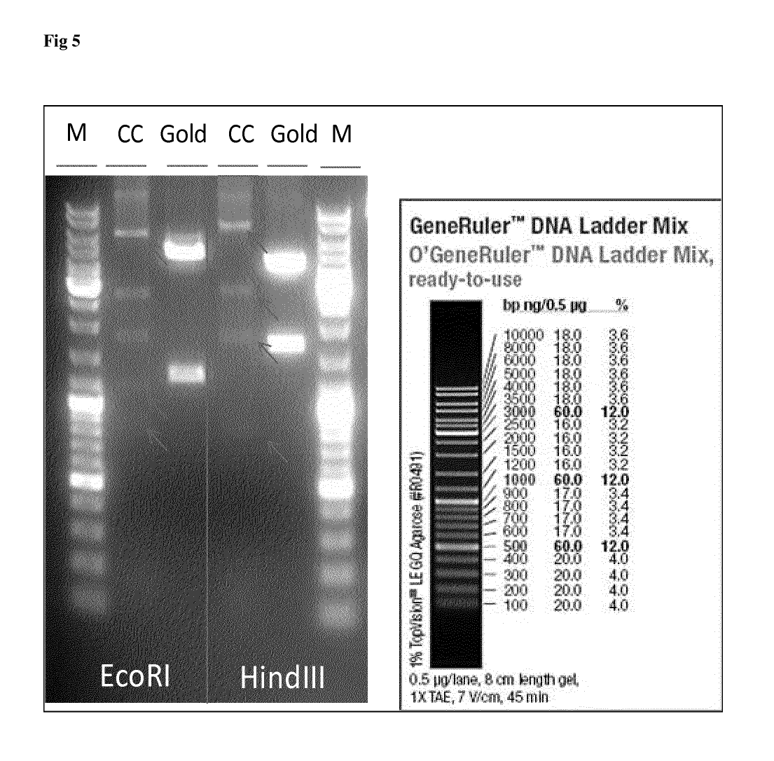

A nucleotide sequence which is “difficult to replicate” may be defined herein as a nucleotide sequence, which when transferred to E. coli XL10-Gold and, optionally subjected to a minipreparation protocol, does not replicate successfully. Successful replication may be determined by restriction enzyme digest followed by DNA gel electrophoresis and analysis of the resultant DNA fragment pattern, as described elsewhere herein and shown in FIG. 5.

The nucleotide sequence encoding the target protein may be “inherently unstable” in standard or common E. coli strains, particularly E. coli strain XL-10 Gold (from Stratagene). The term “inherently unstable” means that the nucleotide sequence undergoes recombination leading to disruption of the coding sequence.

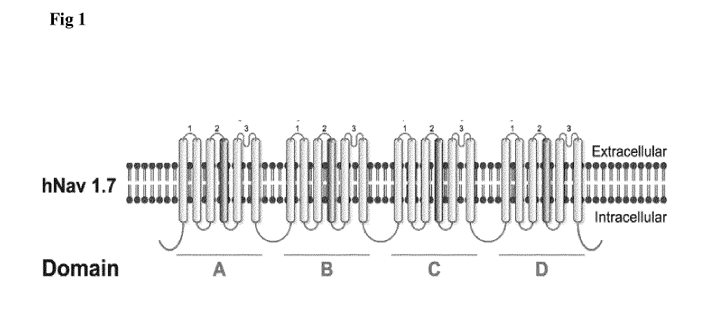

In certain embodiments, the target protein to which the antibody or antigen binding fragment binds is selected from the class of ion channel proteins, preferably the multi-domain voltage-gated calcium and sodium channels, more preferably the voltage-gated sodium channels. In a particularly preferred embodiment, the antibodies or antigen binding fragments of the invention bind specifically to the voltage gated sodium channel human Nav1.7. The multi-domain voltage gated calcium and sodium ion channels are typically naturally encoded by nucleotide sequences of at least 5373 nucleotides, and typically nucleotide sequences in the range 5373-7041 nucleotides. In addition, the voltage-gated calcium and sodium channels typically have 24 transmembrane domains (four domains each with six transmembrane domains, as shown in FIG. 1). It is particularly advantageous to raise antibodies which bind large voltage-gated ion channels, such as the sodium and calcium channels, by DNA immunization, because the immune system of the host animal can be exposed to the full-length protein in its native conformation.

DNA immunization and DNA vaccination protocols have been described in the prior art and any suitable protocol may be adopted for raising antibodies according to the present invention. The DNA immunogen may be delivered to the host animal using any standard gene delivery protocol including but not limited to needle-mediated injection, needle-free jet injection, by a ballistic method or by topical application of the DNA into the skin in patches. Any suitable route of administration may be used including but not limited to intradermal intravenous, intramuscular, intrasplenic and intrahepatic.

The protocol may also include means to stimulate uptake of the DNA by cells of the host animal and/or means to promote integration of the DNA or the encoding nucleotide sequence into the genome of the animal so as to facilitate expression of the protein by cells of the host. For example, delivery of the DNA immunogen may be accompanied by or followed by in vivo electroporation.

It would be understood by one of skill in the art that several factors influence the outcome of DNA immunization including the location of immunization or site of delivery of the DNA, the form of the immunogen, the presence/absence of adjuvants or co administration of biological adjuvants such as cytokines and co administration of other co-stimulatory molecules. In certain embodiments, the DNA molecule may be delivered with an appropriate adjuvant or co-stimulatory molecule.

In accordance with the present invention, the host animal is immunized with a DNA molecule comprising an open reading frame encoding (i) the full-length target protein, (ii) a protein having at least 70% identity to the full-length target protein or (iii) a fragment of the full-length target protein.

For embodiments wherein the target protein to which the antibody or antigen binding fragment binds has a length of at least 1115 amino acids, the open reading frame has a length of at least 3345 nucleotides. Furthermore, in all embodiments where the open reading frame encodes the full-length target protein, the open reading frame comprised within the DNA molecule will typically be equivalent in length to the naturally-encoding nucleotide sequence of the target protein. For example, an antibody which binds a protein having a length of 1977 amino acids, which is naturally-encoded by a nucleotide sequence of 5931 nucleotides in length, may be raised by immunization of a host animal with a DNA molecule comprising an open reading frame of 5931 nucleotides in length encoding the full-length target protein.

Irrespective of the target protein, the DNA molecule may comprise an open reading frame encoding a protein having at least 70% identity, at least 80% identity, at least 90% identity, at least 95% identity, at least 98% identity or at least 99% identity to the full-length target protein. As described above, percentage identity between optimally aligned sequences may be determined using alignment tools available to the skilled person. In the context of the present invention, the percentage identity of the comparator protein is calculated relative to the full-length target protein. In embodiments wherein the protein encoded by the open reading frame comprised within the DNA molecule used for immunization is not the target protein, but is a protein having at least 70% identity thereto, the protein may be a chimeric protein, for example a chimeric ion channel protein, in which different regions or segments of the protein are derived from protein homologues taken from different species. For example, if the DNA immunization is to be carried out in a camelid host, for example a llama, the open reading frame may encode a chimeric or hybrid protein which has one or more regions corresponding to the camelid (llama) protein and one or more non-native regions corresponding to the same protein derived from a different species, for example, human. This approach may allow for the generation of antibodies with binding specificities for particular regions of a target protein i.e. the non-native regions derived from the species other than the host species, whilst still allowing a full-length protein in native conformation to be presented to the host animal's immune system. In embodiments wherein a chimeric protein is encoded by the open reading frame comprised within the DNA molecule, the “non-native” regions of the protein may be 100% identical to the corresponding regions of the target protein, for example the corresponding human protein, and the sequence divergence as compared with the target protein may occur in the regions of the protein derived from the host species.

The DNA molecule may also comprise an open reading frame encoding a fragment of the full-length target protein. For embodiments, wherein the target protein to which the antibody or antigen binding fragment binds has a length of at least 1115 amino acids, the open reading frame encoding the fragment has a length of at least 3345 nucleotides. The fragment therefore has a minimum length of 1115 amino acids. A fragment is defined with reference to the full-length protein and may be reduced in length by one or more amino acids wherein the deleted amino acids are taken from the N terminus, the C terminus or an internal portion of the full-length protein. The fragment of the target protein may have a length of at least 1115 amino acids, at least 1300 amino acids, at least 1500 amino acids, or at least 1900 amino acids and/or be shorter than the full-length target protein by one or more amino acids, for example 10, 20, 30, 40, 50, 100, 200, 500 amino acids.

For embodiments, wherein the target protein to which the antibody or antigen binding fragment binds has a length of at least 1115 amino acids and wherein the DNA molecule comprises an open reading frame encoding a protein having at least 70% identity, at least 80% identity, at least 90% identity, at least 95% identity, at least 98% identity or at least 99% identity to the full-length target protein, or wherein the DNA molecule comprises an open reading frame encoding a fragment of the full-length target protein, the encoding open reading frame is still required to have a length of at least 3345, at least 3800, at least 4000, at least 4500, at least 5000, at least 5500 nucleotides or at least 5931 nucleotides. The nucleotide sequence may have a maximum length of 10,000, 7500, 7100 or 6500 nucleotides, and in certain embodiments may have a length in the range 3345-7500 nucleotides, preferably 4000-7500 nucleotides, preferably 5000-7100 nucleotides, more preferably 5373-7041 nucleotides.

For embodiments wherein the target protein has at least 8 transmembrane domains, the DNA molecule may comprise an open reading frame encoding a fragment of the full-length protein, wherein the fragment is reduced in length, as compared with the full-length target protein, by one or more amino acids, for example, 10, 20, 30, 40, 50, 100, 200, 500 amino acids. The fragment of the full-length target protein may have at least 8, at least 9, at least 10, at least 11, or at least 12 transmembrane domains.

The DNA molecule may comprise one or more suitable regulatory sequences (such as promoters, enhancers, terminators) operably linked to the nucleotide sequence encoding the protein or fragment thereof thereby allowing for expression of the protein or fragment thereof in the cells of the host. As used herein, the term “operably linked” should be taken to mean that the elements are in a functional relationship with each other. For example, a promoter is operably linked to a nucleotide sequence encoding a target protein if said promoter is able to initiate or control transcription of the nucleotide sequence. The DNA molecule may alternatively or in addition, include other elements, for example genes encoding recombinase enzymes to facilitate integration of the DNA molecule into the genomic DNA of the host cells.

The DNA molecule may take any form suitable for transformation of the intended host animal including naked DNA, plasmid DNA, cosmid DNA, expression vector DNA, viral vector DNA. Suitable DNA molecules are known to those skilled in the art in the field of DNA immunization/DNA vaccination and include various commercially available expression vectors and plasmids. The DNA molecule may be formulated in a suitable fashion for administration to the host animal, for example, packaged within a liposome, reconstituted in an appropriate buffer and/or formulated with an appropriate carrier.

The DNA molecule used for immunization may be generated using any standard molecular biology techniques known to those in the art.

The antibody or antigen binding fragment thereof comprises at least one complementarity determining region (CDR) derived from an antibody raised by DNA immunization. In one embodiment, the antibody or antigen binding fragment comprises at least one heavy chain variable domain (VH) wherein the VH domain CDR1 and/or the VH domain CDR2 and/or the VH domain CDR3 is derived from an antibody raised by DNA immunization of a host animal. In a further embodiment, the antibody or antigen binding fragment comprises at least one heavy chain variable domain (VH) derived from an antibody raised by DNA immunization of a host animal. The antibody or antigen binding fragment may alternatively or in addition comprise at least one light chain variable domain (VL) and wherein the VL domain CDR1 and/or the VL domain CDR2 and/or the VL domain CDR3 is derived from an antibody raised by DNA immunization of a host animal. In certain embodiments, the antibody or antigen binding fragment comprises at least one light chain variable domain (VL) derived from an antibody raised by DNA immunization of a host animal. The antibody or antigen binding fragment of this first aspect of the invention may be any antibody or antigen binding fragment of the type described elsewhere herein including but not limited to chimeric antibodies, humanised antibodies and antibodies exhibiting high human homology.

### Nav1.7 Antibodies and Binding Affinity

In a further aspect, the present invention relates to antibodies and antigen binding fragments thereof which bind to the voltage-dated sodium channel Nav1.7, preferably human Nav1.7, with a greater or higher affinity than Nav1.7 antibodies described in the prior art, particularly the Nav1.7 antibodies described in US2011/0135662, particularly CA167_00932 (referred to herein as “932” or “UCB_932”); CA167_00983 (referred to herein as “983” or “UCB_983”); and CA167_01080 (referred to herein as “1080” or “UCB_1080”).

In preferred embodiments, the antibodies and antigen binding fragments of the present invention exhibit an affinity of binding for an extracellular region of human Nav1.7, which is at least 10-fold, at least 20-fold, at least 50-fold or at least 100-fold higher than a reference antibody selected from the group consisting of: UCB_932; UCB_983; and UCB_1066, as described in US2011/0135662.

As used herein, the term “affinity” or “binding affinity” should be understood based on the usual meaning in the art in the context of antibody binding, and reflects the strength and/or stability of binding between an antigen and a binding site on an antibody or antigen binding fragment thereof.

The binding affinity of an antibody or antigen binding fragment thereof for its respective antigen can be determined experimentally using techniques known in the art. For example, BIACORE instruments measure affinity based on the immobilization of a target protein or antigen on a biosensor chip while the antibody or antibody fragment is passed over the immobilized target under specific flow conditions. These experiments yield kₒₙ and k_{off} measurements, which can be translated into K_{D} values, wherein K_{D} is the equilibrium constant for the dissociation of an antigen with an antibody or fragment thereof. The lesser the K_{D} value, the stronger the binding interaction between an antibody and its target antigen. Affinity may also be measured using radioimmunoassay and enzyme immunoassays, such as ELISA, as described elsewhere herein. These experiments can be used to establish an EC₅₀ value, which may also be used as a measure of binding affinity, wherein the lower the value, the greater the binding strength of the interaction between the antibody and its antigen.

The Nav1.7 antibodies or antigen binding fragments thereof of the invention may exhibit an affinity of binding for an extracellular region of human Nav1.7 (EC₅₀ as measured by ELISA), of less than 2 nM, preferably less than 1 nM, preferably less than 0.4 nM. The highest affinity antibodies or antigen binding fragments may have an affinity of binding for an extracellular region of human Nav1.7, (EC₅₀ as measured by ELISA), of 0.05 nM. Therefore the Nav1.7 antibodies or antigen binding fragments thereof of the invention may exhibit an affinity of binding for an extracellular region of human Nav1.7 (EC₅₀ as measured by ELISA) of in the range of from 0.05 nM to 2 nM, 0.05 to 1 nM, or in the range of from 0.05 to 0.4 nM. The ELISA protocol used to determine the EC₅₀ value for the Nav1.7 antibodies described herein may be the protocol described in Examples 3 and 8.

Alternatively, or in addition, the Nav1.7 antibodies or antigen binding fragments thereof may exhibit an off-rate (k_{off} measured by Biacore) for an extracellular region of human Nav1.7 of less than 5×10⁻³ s⁻¹, less than 5×10⁻⁴ s⁻¹ or less than 5×10⁻⁵ s⁻¹. The Nav1.7 antibodies or antigen binding fragments thereof may exhibit an off-rate for an extracellular region of human Nav1.7 in the range of from 0.03×10⁻⁴ s⁻¹ to 45×10⁻⁴ s⁻¹.

The affinity of the antibody or antigen binding fragment for human Nav1.7, as measured by Biacore may be determined using a human Nav1.7 peptide construct, as described elsewhere herein, and shown in Table 4. For example, the off-rate may be determined by Biacore analysis using a hNav1.7 loop A3-llama Fc chimeric construct as represented by SEQ ID NO: 267 shown in Table 4. Alternatively, the off-rate may be determined by Biacore analysis using a hNav1.7 loop A3-GST chimeric construct as represented by SEQ ID NO: 272 shown in Table 4.

The affinity of the antibody or antigen binding fragment may be determined by testing the affinity of the corresponding monoclonal antibody (mAb) in an in vitro ELISA assay, for example, according to the protocol as described elsewhere herein in Examples 3 and 8.

For embodiments wherein the affinity of binding for an extracellular region of Nav1.7, as measured by ELISA, is EC₅₀ less than 2 nM, the antibody or antigen binding fragment may comprise a heavy chain variable domain (VH) comprising a variable heavy chain CDR3 (HCDR3) selected from the group consisting of: SEQ ID NO: 141 to 149, optionally comprising a heavy chain variable CDR2 (HCDR2) selected from the group consisting of: SEQ ID NO: 119 to 129, and optionally comprising a heavy chain variable CDR1 (HCDR1) selected from the group consisting of: SEQ ID NO: 100 to 109.

Alternatively or in addition, the antibody or antigen binding fragment which exhibits an affinity of binding for an extracellular region of Nav1.7, as measured by EC₅₀, of less than 2 nM, may comprise a light chain variable domain (VL) comprising a variable light chain CDR3 (LCDR3) selected from the group consisting of: SEQ ID NO: 204 to 215, optionally comprising a light chain variable CDR2 (LCDR2) selected from the group consisting of: SEQ ID NO: 181 to 192, and optionally comprising a light chain variable CDR1 (LCDR1) selected from the group consisting of SEQ ID NO: 161 to 172.

The heavy chain variable domain may comprise any one of the listed variable heavy chain CDR3 sequences (HCDR3) in combination with any one of the variable heavy chain CDR2 sequences (HCDR2) and any one of the variable heavy chain CDR1 sequences (HCDR1). However, certain combinations of HCDR3 and HCDR2 and HCDR1 are particularly preferred, these being the “native” combinations which derive from a single common VH domain. These preferred combinations are listed in Table 10. Further, the light chain variable domain may comprise any one of the listed variable light chain CDR3 sequences (LCDR3) in combination with any one of the variable light chain CDR2 sequences (LCDR2) and any one of the variable light chain CDR1 sequences (LCDR1). However, certain combinations of LCDR3 and LCDR2 and LCDR1 are particularly preferred, these being the “native” combinations which derive from a single common VL domain. These preferred combinations are listed in Table 11.

Any given Nav1.7 antibody or antigen binding fragment thereof comprising a VH domain paired with a VL domain to form a binding site for Nav1.7 antigen will comprise a combination of six CDRs: variable heavy chain CDR3 (HCDR3), variable heavy chain CDR2 (HCDR2), variable heavy chain CDR1 (HCDR1), variable light chain CDR3 (LCDR3), variable light chain CDR2 (LCDR2) and variable light chain CDR1 (LCDR1).

Although all combinations of six CDRs selected from the CDR sequence groups listed above are permissible, and within the scope of the invention, certain combinations of six CDRs are particularly preferred; these being the “native” combinations within a single Fab exhibiting high affinity binding to Nav1.7.

Preferred combinations of six CDRs include, but are not limited to, the combinations of variable heavy chain CDR3 (HCDR3), variable heavy chain CDR2 (HCDR2), variable heavy chain CDR1 (HCDR1), variable light chain CDR3 (LCDR3), variable light chain CDR2 (LCDR2) and variable light chain CDR1 (LCDR1) selected from the group consisting of:

(i) HCDR3 comprising SEQ ID NO: 141; HCDR2 comprising SEQ ID NO: 119; HCDR1 comprising SEQ ID NO: 100; LCDR3 comprising SEQ ID NO: 204; LCDR2 comprising SEQ ID NO: 181; LCDR1 comprising SEQ ID NO: 161;
(ii) HCDR3 comprising SEQ ID NO: 142; HCDR2 comprising SEQ ID NO: 120; HCDR1 comprising SEQ ID NO: 101; LCDR3 comprising SEQ ID NO: 205; LCDR2 comprising SEQ ID NO: 182; LCDR1 comprising SEQ ID NO: 162;
(iii) HCDR3 comprising SEQ ID NO: 143; HCDR2 comprising SEQ ID NO: 121; HCDR1 comprising SEQ ID NO: 102; LCDR3 comprising SEQ ID NO: 206; LCDR2 comprising SEQ ID NO: 183; LCDR1 comprising SEQ ID NO: 163;
(iv) HCDR3 comprising SEQ ID NO: 144; HCDR2 comprising SEQ ID NO: 122; HCDR1 comprising SEQ ID NO: 103; LCDR3 comprising SEQ ID NO: 207; LCDR2 comprising SEQ ID NO: 184; LCDR1 comprising SEQ ID NO: 164;
(v) HCDR3 comprising SEQ ID NO: 145; HCDR2 comprising SEQ ID NO: 123; HCDR1 comprising SEQ ID NO: 104; LCDR3 comprising SEQ ID NO: 208; LCDR2 comprising SEQ ID NO: 185; LCDR1 comprising SEQ ID NO: 165;
(vi) HCDR3 comprising SEQ ID NO: 146; HCDR2 comprising SEQ ID NO: 124; HCDR1 comprising SEQ ID NO: 105; LCDR3 comprising SEQ ID NO: 209; LCDR2 comprising SEQ ID NO: 186; LCDR1 comprising SEQ ID NO: 166;
(vii) HCDR3 comprising SEQ ID NO: 145; HCDR2 comprising SEQ ID NO: 125; HCDR1 comprising SEQ ID NO: 106; LCDR3 comprising SEQ ID NO: 210; LCDR2 comprising SEQ ID NO: 187; LCDR1 comprising SEQ ID NO: 167;
(viii) HCDR3 comprising SEQ ID NO: 145; HCDR2 comprising SEQ ID NO: 125; HCDR1 comprising SEQ ID NO: 106; LCDR3 comprising SEQ ID NO: 211; LCDR2 comprising SEQ ID NO: 188; LCDR1 comprising SEQ ID NO: 168;
(ix) HCDR3 comprising SEQ ID NO: 147; HCDR2 comprising SEQ ID NO: 126; HCDR1 comprising SEQ ID NO: 107; LCDR3 comprising SEQ ID NO: 212; LCDR2 comprising SEQ ID NO: 189; LCDR1 comprising SEQ ID NO: 169;
(x) HCDR3 comprising SEQ ID NO: 148; HCDR2 comprising SEQ ID NO: 127; HCDR1 comprising SEQ ID NO: 108; LCDR3 comprising SEQ ID NO: 213; LCDR2 comprising SEQ ID NO: 190; LCDR1 comprising SEQ ID NO: 170;
(xi) HCDR3 comprising SEQ ID NO: 149; HCDR2 comprising SEQ ID NO: 128; HCDR1 comprising SEQ ID NO: 109; LCDR3 comprising SEQ ID NO: 214; LCDR2 comprising SEQ ID NO: 191; LCDR1 comprising SEQ ID NO: 171; and
(xii) HCDR3 comprising SEQ ID NO: 149; HCDR2 comprising SEQ ID NO: 129; HCDR1 comprising SEQ ID NO: 109; LCDR3 comprising SEQ ID NO: 215; LCDR2 comprising SEQ ID NO: 192; LCDR1 comprising SEQ ID NO: 172.

Alternatively, or in addition, the Nav1.7 antibodies or antigen binding fragments thereof may exhibit an off-rate (k_{off} measured by Biacore) for an extracellular region of human Nav1.7 of less than 5×10⁻³ s⁻¹, less than 5×10⁻⁴ s⁻¹, or less than 5×10⁻⁵ s⁻¹, The Nav1.7 antibodies or antigen binding fragments thereof may exhibit an off-rate for an extracellular region of human Nav1.7 in the range of from 0.03×10⁻⁴ s⁻¹, to 45×10⁴ s.

The affinity of the antibody or antigen binding fragment may be determined by testing the affinity of the corresponding Fab fragment in a BIACORE assay, for example, according to the protocols as described elsewhere herein. The affinity of the antibody or antigen binding fragment for human Nav1.7, as measured by Biacore may be determined using a human Nav1.7 peptide construct, as described elsewhere herein, and shown in Table 4. For example, the off-rate may be determined by Biacore analysis using a hNav1.7 loop A3-llama Fc chimeric construct as represented by SEQ ID NO: 267 shown in Table 4. Alternatively, the off-rate may be determined by Biacore analysis using a hNav1.7 loop A3-GST chimeric construct as represented by SEQ ID NO: 272 shown in Table 4.

For embodiments wherein the Nav1.7 antibodies or antigen binding fragments thereof exhibit an off-rate for an extracellular region of human Nav1.7 of less than 5×10⁻³ s⁻¹, the antibody or antigen binding fragment may comprise a heavy chain variable domain (VH) comprising a variable heavy chain CDR3 (HCDR3) selected from the group consisting of: SEQ ID NO: 28 to 34, optionally comprising a heavy chain variable CDR2 (HCDR2) selected from the group consisting of: SEQ ID NO: 16 to 21, and optionally comprising a heavy chain variable CDR1 (HCDR1) selected from the group consisting of: SEQ ID NO: 8 to 13.

Alternatively or in addition, the antibody or antigen binding fragment which binds to A3 of Nav1.7 may comprise a light chain variable domain (VL) comprising a variable light chain CDR3 (LCDR3) selected from the group consisting of: SEQ ID NO: 77 to 84, optionally comprising a light chain variable CDR2 (LCDR2) selected from the group consisting of: SEQ ID NO: 62 to 68, and optionally comprising a light chain variable CDR1 (LCDR1) selected from the group consisting of SEQ ID NO: 46 to 54.

The heavy chain variable domain may comprise any one of the listed variable heavy chain CDR3 sequences (HCDR3) in combination with any one of the variable heavy chain CDR2 sequences (HCDR2) and any one of the variable heavy chain CDR1 sequences (HCDR1). However, certain combinations of HCDR3 and HCDR2 and HCDR1 are particularly preferred, these being the “native” combinations which derive from a single common VH domain. These preferred combinations are listed in Table 5. Further, the light chain variable domain may comprise any one of the listed variable light chain CDR3 sequences (LCDR3) in combination with any one of the variable light chain CDR2 sequences (LCDR2) and any one of the variable light chain CDR1 sequences (LCDR1). However, certain combinations of LCDR3 and LCDR2 and LCDR1 are particularly preferred, these being the “native” combinations which derive from a single common VL domain. These preferred combinations are listed in Table 6.

Any given Nav1.7 antibody or antigen binding fragment thereof comprising a VH domain paired with a VL domain to form a binding site for Nav1.7 antigen will comprise a combination of six CDRs: variable heavy chain CDR3 (HCDR3), variable heavy chain CDR2 (HCDR2), variable heavy chain CDR1 (HCDR1), variable light chain CDR3 (LCDR3), variable light chain CDR2 (LCDR2) and variable light chain CDR1 (LCDR1).

Although all combinations of six CDRs selected from the CDR sequence groups listed above are permissible, and within the scope of the invention, certain combinations of six CDRs are particularly preferred; these being the “native” combinations within a single Fab exhibiting high affinity binding to Nav1.7.

Preferred combinations of six CDRs include, but are not limited to, the combinations of variable heavy chain CDR3 (HCDR3), variable heavy chain CDR2 (HCDR2), variable heavy chain CDR1 (HCDR1), variable light chain CDR3 (LCDR3), variable light chain CDR2 (LCDR2) and variable light chain CDR1 (LCDR1) selected from the group consisting of:

(i) HCDR3 comprising SEQ ID NO: 28; HCDR2 comprising SEQ ID NO: 16; HCDR1 comprising SEQ ID NO: 8; LCDR3 comprising SEQ ID NO: 77; LCDR2 comprising SEQ ID NO: 62; LCDR1 comprising SEQ ID NO: 46;
(ii) HCDR3 comprising SEQ ID NO: 29; HCDR2 comprising SEQ ID NO: 16; HCDR1 comprising SEQ ID NO: 8; LCDR3 comprising SEQ ID NO: 78; LCDR2 comprising SEQ ID NO: 63; LCDR1 comprising SEQ ID NO: 47;
(iii) HCDR3 comprising SEQ ID NO: 29; HCDR2 comprising SEQ ID NO: 16; HCDR1 comprising SEQ ID NO: 8; LCDR3 comprising SEQ ID NO: 78; LCDR2 comprising SEQ ID NO: 63; LCDR1 comprising SEQ ID NO: 48;
(iv) HCDR3 comprising SEQ ID NO: 30; HCDR2 comprising SEQ ID NO: 17; HCDR1 comprising SEQ ID NO: 9; LCDR3 comprising SEQ ID NO: 79; LCDR2 comprising SEQ ID NO: 64; LCDR1 (v) HCDR3 comprising SEQ ID NO: 30; HCDR2 comprising SEQ ID NO: 17; HCDR1 comprising SEQ ID NO: 9; LCDR3 comprising SEQ ID NO: 80; LCDR2 comprising SEQ ID NO: 64; LCDR1 comprising SEQ ID NO: 50;
(vi) HCDR3 comprising SEQ ID NO: 31; HCDR2 comprising SEQ ID NO: 18; HCDR1 comprising SEQ ID NO: 10; LCDR3 comprising SEQ ID NO: 81; LCDR2 comprising SEQ ID NO: 65; LCDR1 comprising SEQ ID NO: 51;
(vii) HCDR3 comprising SEQ ID NO: 32; HCDR2 comprising SEQ ID NO: 19; HCDR1 comprising SEQ ID NO: 11; LCDR3 comprising SEQ ID NO: 82; LCDR2 comprising SEQ ID NO: 66; LCDR1 comprising SEQ ID NO: 52;
(viii) HCDR3 comprising SEQ ID NO: 33; HCDR2 comprising SEQ ID NO: 20; HCDR1 comprising SEQ ID NO: 12; LCDR3 comprising SEQ ID NO: 83; LCDR2 comprising SEQ ID NO: 67; LCDR1 comprising SEQ ID NO: 53; and
(ix) HCDR3 comprising SEQ ID NO: 34; HCDR2 comprising SEQ ID NO: 21; HCDR1 comprising SEQ ID NO: 13; LCDR3 comprising SEQ ID NO: 84; LCDR2 comprising SEQ ID NO: 68; LCDR1 comprising SEQ ID NO: 54.

### Nav1.7 Structure and Binding

As discussed elsewhere herein and depicted in FIG. 1, Nav1.7 is a transmembrane protein consisting of four domains (A, B, C and D), each domain having six transmembrane helices (S1, S2, S3, S4, S5 and S6) and three extracellular hydrophilic loops (E1, E2 and E3). The E1, E2 and E3 extracellular loops are distinct within each of domains A, B, C and D of Nav1.7, and are therefore identified herein as extracellular loops A1, A2 and A3 (corresponding to E1, E2 and E3 of domain A), B1, B2 and B3 (corresponding to E1, E2 and E3 of domain B), C1, C2 and C3 (corresponding to E1, E2 and E3 of domain C) and D1, D2 and D3 (corresponding to E1, E2 and E3 of domain D).

The Nav1.7 antibodies and antigen binding fragments of the present invention typically bind to an extracellular region of the Nav1.7 ion channel, wherein an extracellular region may encompass an extracellular part of one of the transmembrane domains and/or one of the extracellular loops from any one of the four domains. Preferably, the Nav1.7 antibodies and antigen binding fragments bind one of the Nav1.7 extracellular loops.

In certain embodiments, the antibody or antigen binding fragment binds to an extracellular region which is poorly conserved and/or exhibits a high degree of sequence diversity between different members of the voltage-gated sodium channel family. For example, the sequence of the A3 extracellular loop is poorly conserved between Nav1.7 and other sodium channels, therefore antibodies that recognise an epitope within or including a region of the A3 extracellular loop are unlikely to exhibit binding to other members of the voltage-gated sodium channel family.

In preferred embodiments, the Nav1.7 antibodies of the present invention bind to an extracellular loop of Nav1.7 selected from (see Table 3):
A3 as represented by SEQ ID NO: 262 (corresponding to amino acid residues 269 to 319 of SEQ ID NO:260);
B1 as represented by SEQ ID NO: 263 (corresponding to amino acid residues 753 to 765 of SEQ ID NO:260);
C1 as represented by SEQ ID NO: 264 (corresponding to amino acid residues 1201 to 1214 of SEQ ID NO:260);
D1 as represented by SEQ ID NO: 265 (corresponding to amino acid residues 1523 to 1536 of SEQ ID NO:260);
C3 as represented by SEQ ID NO: 266 (corresponding to amino acid residues 1334 to 1420 of SEQ ID NO:260); and
B2 as represented by SEQ ID NO: 611 (corresponding to amino acid residues 809 to 818 of SEQ ID NO:260).

TABLE 3
Extracellular loops of Nav1.7

| Extra- | X-X aa of | | |
|---|---|---|---|
| cellular | SEQ ID NO: | | |
| loop | 260 | Sequence | SEQ ID NO: |
| A3 | 269-319 | GNLKHKCFRNSLENNETLESIMNTLESEEDFRKYFY | 262 |
| | | YLEGSKDALLCGFST | |
| B1 | 753-765 | EHHPMTEEFKNVL | 263 |
| C1 | 1201-1214 | EDIYIERKKTIKII | 264 |
| D1 | 1523-1536 | VEKEGQSQHMTEVL | 265 |
| C3 | 1334-1420 | GKFYECINTTDGSRFPASQVPNRSECFALMNVSQ | 266 |
| | | NVRWKNLKVNFDNVGLGYLSLLQVATFKGWTII | |
| | | MYAAVDSVNVDKQPKYEYSL | |
| B2 | 809-818 | VELFLADVEG | 611 |

In certain embodiments, the Nav1.7 antibodies or antigen binding fragments will exhibit “selective binding” to one of the extracellular loops selected from the group above, wherein selective binding means that the antibody binds to the extracellular loop, to the exclusion of other extracellular loops selected from the group consisting of A3, B1, C1, D1 C3 and B2 identified above, or to the exclusion of any other extracellular region within Nav1.7.

### Extracellular Loop A3 Binders

In preferred embodiments, the antibody or antigen binding fragment thereof binds to extracellular loop A3 of Nav1.7. In embodiments wherein the antibody or antigen binding fragment thereof binds to A3, the antibody or antigen binding fragment may comprise a heavy chain variable domain (VH) comprising a variable heavy chain CDR3 (HCDR3) selected from the group consisting of: SEQ ID NO: 28 to 34, 141 to 142, 500, 501 and 503, optionally comprising a heavy chain variable CDR2 (HCDR2) selected from the group consisting of: SEQ ID NO: 16 to 21, 119 to 120, 472, 473 and 475, and optionally comprising a heavy chain variable CDR1 (HCDR1) selected from the group consisting of: SEQ ID NO: 8 to 13, 100 to 101, 452, 453 and 455.

Alternatively or in addition, the antibody or antigen binding fragment which binds to A3 of Nav1.7 may comprise a light chain variable domain (VL) comprising a variable light chain CDR3 (LCDR3) selected from the group consisting of: SEQ ID NO: 77 to 84, 204 to 205, 563, 564 and 566, optionally comprising a light chain variable CDR2 (LCDR2) selected from the group consisting of: SEQ ID NO: 62 to 68, 181 to 182, 547 and 548, and optionally comprising a light chain variable CDR1 (LCDR1) selected from the group consisting of SEQ ID NO: 46 to 54, 161 to 162, 530 and 532.

The heavy chain variable domain may comprise any one of the listed variable heavy chain CDR3 sequences (HCDR3) in combination with any one of the variable heavy chain CDR2 sequences (HCDR2) and any one of the variable heavy chain CDR1 sequences (HCDR1). However, certain combinations of HCDR3 and HCDR2 and HCDR1 are particularly preferred, these being the “native” combinations which derive from a single common VH domain. These preferred combinations are listed in Tables 5, 10 and 26. Further, the light chain variable domain may comprise any one of the listed variable light chain CDR3 sequences (LCDR3) in combination with any one of the variable light chain CDR2 sequences (LCDR2) and any one of the variable light chain CDR1 sequences (LCDR1). However, certain combinations of LCDR3 and LCDR2 and LCDR1 are particularly preferred, these being the “native” combinations which derive from a single common VL domain. These preferred combinations are listed in Tables 6, 11 and 27.

Any given Nav1.7 antibody or antigen binding fragment thereof comprising a VH domain paired with a VL domain to form a binding site for Nav1.7 antigen will comprise a combination of six CDRs: variable heavy chain CDR3 (HCDR3), variable heavy chain CDR2 (HCDR2), variable heavy chain CDR1 (HCDR1), variable light chain CDR3 (LCDR3), variable light chain CDR2 (LCDR2) and variable light chain CDR1 (LCDR1).

Although all combinations of six CDRs selected from the CDR sequence groups listed above are permissible, and within the scope of the invention, certain combinations of six CDRs are particularly preferred; these being the “native” combinations within a single Fab exhibiting high affinity binding to Nav1.7.

Preferred combinations of six CDRs include, but are not limited to, the combinations of variable heavy chain CDR3 (HCDR3), variable heavy chain CDR2 (HCDR2), variable heavy chain CDR1 (HCDR1), variable light chain CDR3 (LCDR3), variable light chain CDR2 (LCDR2) and variable light chain CDR1 (LCDR1) selected from the group consisting of:

(i) HCDR3 comprising SEQ ID NO: 28; HCDR2 comprising SEQ ID NO: 16; HCDR1 comprising SEQ ID NO: 8; LCDR3 comprising SEQ ID NO: 77; LCDR2 comprising SEQ ID NO: 62; LCDR1 comprising SEQ ID NO: 46;
(ii) HCDR3 comprising SEQ ID NO: 29; HCDR2 comprising SEQ ID NO: 16; HCDR1 comprising SEQ ID NO: 8; LCDR3 comprising SEQ ID NO: 78; LCDR2 comprising SEQ ID NO: 63; LCDR1 comprising SEQ ID NO: 47;
(iii) HCDR3 comprising SEQ ID NO: 29; HCDR2 comprising SEQ ID NO: 16; HCDR1 comprising SEQ ID NO: 8; LCDR3 comprising SEQ ID NO: 78; LCDR2 comprising SEQ ID NO: 63; LCDR1 comprising SEQ ID NO: 48;
(iv) HCDR3 comprising SEQ ID NO: 30; HCDR2 comprising SEQ ID NO: 17; HCDR1 comprising SEQ ID NO: 9; LCDR3 comprising SEQ ID NO: 79; LCDR2 comprising SEQ ID NO: 64; LCDR1 comprising SEQ ID NO: 49;
(v) HCDR3 comprising SEQ ID NO: 30; HCDR2 comprising SEQ ID NO: 17; HCDR1 comprising SEQ ID NO: 9; LCDR3 comprising SEQ ID NO: 80; LCDR2 comprising SEQ ID NO: 64; LCDR1 comprising SEQ ID NO: 50;
(vi) HCDR3 comprising SEQ ID NO: 31; HCDR2 comprising SEQ ID NO: 18; HCDR1 comprising SEQ ID NO: 10; LCDR3 comprising SEQ ID NO: 81; LCDR2 comprising SEQ ID NO: 65; LCDR1 comprising SEQ ID NO: 51;
(vii) HCDR3 comprising SEQ ID NO: 32; HCDR2 comprising SEQ ID NO: 19; HCDR1 comprising SEQ ID NO: 11; LCDR3 comprising SEQ ID NO: 82; LCDR2 comprising SEQ ID NO: 66; LCDR1 comprising SEQ ID NO: 52;
(viii) HCDR3 comprising SEQ ID NO: 33; HCDR2 comprising SEQ ID NO: 20; HCDR1 comprising SEQ ID NO: 12; LCDR3 comprising SEQ ID NO: 83; LCDR2 comprising SEQ ID NO: 67; LCDR1 comprising SEQ ID NO: 53;
(ix) HCDR3 comprising SEQ ID NO: 34; HCDR2 comprising SEQ ID NO: 21; HCDR1 comprising SEQ ID NO: 13; LCDR3 comprising SEQ ID NO: 84; LCDR2 comprising SEQ ID NO: 68; LCDR1 comprising SEQ ID NO: 54;
(x) HCDR3 comprising SEQ ID NO: 141; HCDR2 comprising SEQ ID NO: 119; HCDR1 comprising SEQ ID NO: 100; LCDR3 comprising SEQ ID NO: 204; LCDR2 comprising SEQ ID NO: 181; LCDR1 comprising SEQ ID NO: 161;
(xi) HCDR3 comprising SEQ ID NO: 142; HCDR2 comprising SEQ ID NO: 120; HCDR1 comprising SEQ ID NO: 101; LCDR3 comprising SEQ ID NO: 205; LCDR2 comprising SEQ ID NO: 182; LCDR1 comprising SEQ ID NO: 162;
(xii) HCDR3 comprising SEQ ID NO: 500; HCDR2 comprising SEQ ID NO: 472; HCDR1 comprising SEQ ID NO: 452; LCDR3 comprising SEQ ID NO: 563; LCDR2 comprising SEQ ID NO: 547; LCDR1 comprising SEQ ID NO: 530;
(xiii) HCDR3 comprising SEQ ID NO: 501; HCDR2 comprising SEQ ID NO: 473; HCDR1 comprising SEQ ID NO: 453; LCDR3 comprising SEQ ID NO: 564; LCDR2 comprising SEQ ID NO: 68; LCDR1 comprising SEQ ID NO: 54; and
(xiv) HCDR3 comprising SEQ ID NO: 503; HCDR2 comprising SEQ ID NO: 475; HCDR1 comprising SEQ ID NO: 455; LCDR3 comprising SEQ ID NO: 566; LCDR2 comprising SEQ ID NO: 548; LCDR1 comprising SEQ ID NO: 532;

In preferred embodiments, the antibodies or antigen binding fragments described herein which bind to the extracellular loop A3 of human Nav1.7 exhibit an affinity of binding for an extracellular region of human Nav1.7, which is at least 10-fold, at least 20-fold, at least 50-fold or at least 100-fold higher than the reference antibody UCB_932 as described in US2011/0135662.

### Extracellular Loop B1, C1 and D1 Binders

In preferred embodiments, the antibody or antigen binding fragment thereof binds to an extracellular loop selected from B1, C1 or D1 of Nav1.7. In embodiments wherein the antibody or antigen binding fragment thereof binds to B1, C1 or D1, the antibody or antigen binding fragment may comprise a heavy chain variable domain (VH) comprising a variable heavy chain CDR3 (HCDR3) selected from the group consisting of: SEQ ID NO: 143 to 148, 502 and 504 to 507, optionally comprising a heavy chain variable CDR2 (HCDR2) selected from the group consisting of: SEQ ID NO: 121 to 127, 474 and 476-480, and optionally comprising a heavy chain variable CDR1 (HCDR1) selected from the group consisting of: SEQ ID NO: 102 to 108, 454 and 456-460.

Alternatively or in addition, the antibody or antigen binding fragment which binds to B1, C1 or D1 of Nav1.7 may comprise a light chain variable domain (VL) comprising a variable light chain CDR3 (LCDR3) selected from the group consisting of: SEQ ID NO: 206 to 213, 565 and 567 to 571, optionally comprising a light chain variable CDR2 (LCDR2) selected from the group consisting of: SEQ ID NO: 183 to 190 and 549-551, and optionally comprising a light chain variable CDR1 (LCDR1) selected from the group consisting of SEQ ID NO: 163 to 170, 531 and 533 to 535.

The heavy chain variable domain may comprise any one of the listed variable heavy chain CDR3 sequences (HCDR3) in combination with any one of the variable heavy chain CDR2 sequences (HCDR2) and any one of the variable heavy chain CDR1 sequences (HCDR1). However, certain combinations of HCDR3 and HCDR2 and HCDR1 are particularly preferred, these being the “native” combinations which derive from a single common VH domain. These preferred combinations are listed in Tables 10 and 26. Further, the light chain variable domain may comprise any one of the listed variable light chain CDR3 sequences (LCDR3) in combination with any one of the variable light chain CDR2 sequences (LCDR2) and any one of the variable light chain CDR1 sequences (LCDR1). However, certain combinations of LCDR3 and LCDR2 and LCDR1 are particularly preferred, these being the “native” combinations which derive from a single common VL domain. These preferred combinations are listed in Tables 11 and 27.

Any given Nav1.7 antibody or antigen binding fragment thereof comprising a VH domain paired with a VL domain to form a binding site for Nav1.7 antigen will comprise a combination of six CDRs: variable heavy chain CDR3 (HCDR3), variable heavy chain CDR2 (HCDR2), variable heavy chain CDR1 (HCDR1), variable light chain CDR3 (LCDR3), variable light chain CDR2 (LCDR2) and variable light chain CDR1 (LCDR1).

Although all combinations of six CDRs selected from the CDR sequence groups listed above are permissible, and within the scope of the invention, certain combinations of six CDRs are particularly preferred; these being the “native” combinations within a single Fab exhibiting high affinity binding to Nav1.7.

Preferred combinations of six CDRs include, but are not limited to, the combinations of variable heavy chain CDR3 (HCDR3), variable heavy chain CDR2 (HCDR2), variable heavy chain CDR1 (HCDR1), variable light chain CDR3 (LCDR3), variable light chain CDR2 (LCDR2) and variable light chain CDR1 (LCDR1) selected from the group consisting of:

(i) HCDR3 comprising SEQ ID NO: 143; HCDR2 comprising SEQ ID NO: 121; HCDR1 comprising SEQ ID NO: 102; LCDR3 comprising SEQ ID NO: 206; LCDR2 comprising SEQ ID NO: 183; LCDR1 comprising SEQ ID NO: 163;
(ii) HCDR3 comprising SEQ ID NO: 144; HCDR2 comprising SEQ ID NO: 122; HCDR1 comprising SEQ ID NO: 103; LCDR3 comprising SEQ ID NO: 207; LCDR2 comprising SEQ ID NO: 184; LCDR1 comprising SEQ ID NO: 164;
(iii) HCDR3 comprising SEQ ID NO: 145; HCDR2 comprising SEQ ID NO: 123; HCDR1 comprising SEQ ID NO: 104; LCDR3 comprising SEQ ID NO: 208; LCDR2 comprising SEQ ID NO: 185; LCDR1 comprising SEQ ID NO: 165;
(iv) HCDR3 comprising SEQ ID NO: 146; HCDR2 comprising SEQ ID NO: 124; HCDR1 comprising SEQ ID NO: 105; LCDR3 comprising SEQ ID NO: 209; LCDR2 comprising SEQ ID NO: 186; LCDR1 comprising SEQ ID NO: 166;
(v) HCDR3 comprising SEQ ID NO: 145; HCDR2 comprising SEQ ID NO: 125; HCDR1 comprising SEQ ID NO: 106; LCDR3 comprising SEQ ID NO: 210; LCDR2 comprising SEQ ID NO: 187; LCDR1 comprising SEQ ID NO: 167;
(vi) HCDR3 comprising SEQ ID NO: 145; HCDR2 comprising SEQ ID NO: 125; HCDR1 comprising SEQ ID NO: 106; LCDR3 comprising SEQ ID NO: 211; LCDR2 comprising SEQ ID NO: 188; LCDR1 comprising SEQ ID NO: 168;
(vii) HCDR3 comprising SEQ ID NO: 147; HCDR2 comprising SEQ ID NO: 126; HCDR1 comprising SEQ ID NO: 107; LCDR3 comprising SEQ ID NO: 212; LCDR2 comprising SEQ ID NO: 189; LCDR1 comprising SEQ ID NO: 169;
(viii) HCDR3 comprising SEQ ID NO: 148; HCDR2 comprising SEQ ID NO: 127; HCDR1 comprising SEQ ID NO: 108; LCDR3 comprising SEQ ID NO: 213; LCDR2 comprising SEQ ID NO: 190; LCDR1 comprising SEQ ID NO: 170;
(vi) HCDR3 comprising SEQ ID NO: 502; HCDR2 comprising SEQ ID NO: 474; HCDR1 comprising SEQ ID NO: 454; LCDR3 comprising SEQ ID NO: 565; LCDR2 comprising SEQ ID NO: 187; LCDR1 comprising SEQ ID NO: 531;
(vi) HCDR3 comprising SEQ ID NO: 504; HCDR2 comprising SEQ ID NO: 476; HCDR1 comprising SEQ ID NO: 456; LCDR3 comprising SEQ ID NO: 567; LCDR2 comprising SEQ ID NO: 187; LCDR1 comprising SEQ ID NO: 167;
(vi) HCDR3 comprising SEQ ID NO: 505; HCDR2 comprising SEQ ID NO: 477; HCDR1 comprising SEQ ID NO: 457; LCDR3 comprising SEQ ID NO: 568; LCDR2 comprising SEQ ID NO: 549; LCDR1 comprising SEQ ID NO: 533;
(vi) HCDR3 comprising SEQ ID NO: 506; HCDR2 comprising SEQ ID NO: 478; HCDR1 comprising SEQ ID NO: 458; LCDR3 comprising SEQ ID NO: 569; LCDR2 comprising SEQ ID NO: 187; LCDR1 comprising SEQ ID NO: 165;
(vi) HCDR3 comprising SEQ ID NO: 147; HCDR2 comprising SEQ ID NO: 479; HCDR1 comprising SEQ ID NO: 459; LCDR3 comprising SEQ ID NO: 570; LCDR2 comprising SEQ ID NO: 550; LCDR1 comprising SEQ ID NO: 534; and
(vi) HCDR3 comprising SEQ ID NO: 507; HCDR2 comprising SEQ ID NO: 480; HCDR1 comprising SEQ ID NO: 460; LCDR3 comprising SEQ ID NO: 571; LCDR2 comprising SEQ ID NO: 551; LCDR1 comprising SEQ ID NO: 535.

### Extracellular Loop C3 Binders

In preferred embodiments, the antibody or antigen binding fragment thereof binds to the extracellular loop C3 of Nav1.7. In embodiments wherein the antibody or antigen binding fragment thereof binds to C3, the antibody or antigen binding fragment may comprise a heavy chain variable domain (VH) comprising a variable heavy chain CDR3 (HCDR3) consisting of: SEQ ID NO: 149, optionally comprising a heavy chain variable CDR2 (HCDR2) selected from the group consisting of: SEQ ID NO: 128 and 129, and optionally comprising a heavy chain variable CDR1 (HCDR1) consisting of: SEQ ID NO: 109.

Alternatively or in addition, the antibody or antigen binding fragment which binds to C3 of Nav1.7 may comprise a light chain variable domain (VL) comprising a variable light chain CDR3 (LCDR3) selected from the group consisting of: SEQ ID NO: 214 and 215, optionally comprising a light chain variable CDR2 (LCDR2) selected from the group consisting of: SEQ ID NO: 191 and 192, and optionally comprising a light chain variable CDR1 (LCDR1) selected from the group consisting of SEQ ID NO: 171 and 172.

The heavy chain variable domain may comprise any one of the listed variable heavy chain CDR3 sequences (HCDR3) in combination with any one of the variable heavy chain CDR2 sequences (HCDR2) and any one of the variable heavy chain CDR1 sequences (HCDR1). However, certain combinations of HCDR3 and HCDR2 and HCDR1 are particularly preferred, these being the “native” combinations which derive from a single common VH domain. These preferred combinations are listed in Table 10. Further, the light chain variable domain may comprise any one of the listed variable light chain CDR3 sequences (LCDR3) in combination with any one of the variable light chain CDR2 sequences (LCDR2) and any one of the variable light chain CDR1 sequences (LCDR1). However, certain combinations of LCDR3 and LCDR2 and LCDR1 are particularly preferred, these being the “native” combinations which derive from a single common VL domain. These preferred combinations are listed in Table 11.

Any given Nav1.7 antibody or antigen binding fragment thereof comprising a VH domain paired with a VL domain to form a binding site for Nav1.7 antigen will comprise a combination of six CDRs: variable heavy chain CDR3 (HCDR3), variable heavy chain CDR2 (HCDR2), variable heavy chain CDR1 (HCDR1), variable light chain CDR3 (LCDR3), variable light chain CDR2 (LCDR2) and variable light chain CDR1 (LCDR1).

Although all combinations of six CDRs selected from the CDR sequence groups listed above are permissible, and within the scope of the invention, certain combinations of six CDRs are particularly preferred; these being the “native” combinations within a single Fab exhibiting high affinity binding to Nav1.7.

Preferred combinations of six CDRs include, but are not limited to, the combinations of variable heavy chain CDR3 (HCDR3), variable heavy chain CDR2 (HCDR2), variable heavy chain CDR1 (HCDR1), variable light chain CDR3 (LCDR3), variable light chain CDR2 (LCDR2) and variable light chain CDR1 (LCDR1) selected from the group consisting of:

(i) HCDR3 comprising SEQ ID NO: 149; HCDR2 comprising SEQ ID NO: 128; HCDR1 comprising SEQ ID NO: 109; LCDR3 comprising SEQ ID NO: 214; LCDR2 comprising SEQ ID NO: 191; LCDR1 comprising SEQ ID NO: 171; and
(ii) HCDR3 comprising SEQ ID NO: 149; HCDR2 comprising SEQ ID NO: 129; HCDR1 comprising SEQ ID NO: 109; LCDR3 comprising SEQ ID NO: 215; LCDR2 comprising SEQ ID NO: 192; LCDR1 comprising SEQ ID NO: 172.

In preferred embodiments, the antibodies or antigen binding fragments described herein which bind to the extracellular loop C3 of human Nav1.7 exhibit an affinity of binding for an extracellular region of human Nav1.7, which is at least 10-fold, at least 20-fold, at least 50-fold or at least 100-fold higher than the reference antibody UCB_1066 as described in US2011/0135662.

### Extracellular Loop B2 Binders

In preferred embodiments, the antibody or antigen binding fragment thereof binds to the extracellular loop B2 of Nav1.7. In embodiments wherein the antibody or antigen binding fragment thereof binds to B2, the antibody or antigen binding fragment may comprise a heavy chain variable domain (VH) comprising a variable heavy chain CDR3 (HCDR3) selected from the group consisting of: SEQ ID NO: 508 to 512, optionally comprising a heavy chain variable CDR2 (HCDR2) selected from the group consisting of: SEQ ID NO: 481 to 485, and optionally comprising a heavy chain variable CDR1 (HCDR1) selected from the group consisting of: SEQ ID NO: 461 to 465.

Alternatively or in addition, the antibody or antigen binding fragment which binds to B2 of Nav1.7 may comprise a light chain variable domain (VL) comprising a variable light chain CDR3 (LCDR3) selected from the group consisting of: SEQ ID NO: 572 to 576, optionally comprising a light chain variable CDR2 (LCDR2) selected from the group consisting of: SEQ ID NO: 552 to 555, and optionally comprising a light chain variable CDR1 (LCDR1) selected from the group consisting of SEQ ID NO: 536 to 538.

The heavy chain variable domain may comprise any one of the listed variable heavy chain CDR3 sequences (HCDR3) in combination with any one of the variable heavy chain CDR2 sequences (HCDR2) and any one of the variable heavy chain CDR1 sequences (HCDR1). However, certain combinations of HCDR3 and HCDR2 and HCDR1 are particularly preferred, these being the “native” combinations which derive from a single common VH domain. These preferred combinations are listed in Table 26. Further, the light chain variable domain may comprise any one of the listed variable light chain CDR3 sequences (LCDR3) in combination with any one of the variable light chain CDR2 sequences (LCDR2) and any one of the variable light chain CDR1 sequences (LCDR1). However, certain combinations of LCDR3 and LCDR2 and LCDR1 are particularly preferred, these being the “native” combinations which derive from a single common VL domain. These preferred combinations are listed in Table 27.

Any given Nav1.7 antibody or antigen binding fragment thereof comprising a VH domain paired with a VL domain to form a binding site for Nav1.7 antigen will comprise a combination of six CDRs: variable heavy chain CDR3 (HCDR3), variable heavy chain CDR2 (HCDR2), variable heavy chain CDR1 (HCDR1), variable light chain CDR3 (LCDR3), variable light chain CDR2 (LCDR2) and variable light chain CDR1 (LCDR1).

Although all combinations of six CDRs selected from the CDR sequence groups listed above are permissible, and within the scope of the invention, certain combinations of six CDRs are particularly preferred; these being the “native” combinations within a single Fab exhibiting high affinity binding to Nav1.7.

Preferred combinations of six CDRs include, but are not limited to, the combinations of variable heavy chain CDR3 (HCDR3), variable heavy chain CDR2 (HCDR2), variable heavy chain CDR1 (HCDR1), variable light chain CDR3 (LCDR3), variable light chain CDR2 (LCDR2) and variable light chain CDR1 (LCDR1) selected from the group consisting of:

(i) HCDR3 comprising SEQ ID NO: 508; HCDR2 comprising SEQ ID NO: 481; HCDR1 comprising SEQ ID NO: 461; LCDR3 comprising SEQ ID NO: 572; LCDR2 comprising SEQ ID NO: 552; LCDR1 comprising SEQ ID NO: 536;
(ii) HCDR3 comprising SEQ ID NO: 509; HCDR2 comprising SEQ ID NO: 482; HCDR1 comprising SEQ ID NO: 462; LCDR3 comprising SEQ ID NO: 573; LCDR2 comprising SEQ ID NO: 553; LCDR1 comprising SEQ ID NO: 52;
(iii) HCDR3 comprising SEQ ID NO: 510; HCDR2 comprising SEQ ID NO: 483; HCDR1 comprising SEQ ID NO: 463; LCDR3 comprising SEQ ID NO: 574; LCDR2 comprising SEQ ID NO: 553; LCDR1 comprising SEQ ID NO: 52;
(iv) HCDR3 comprising SEQ ID NO: 511; HCDR2 comprising SEQ ID NO: 484; HCDR1 comprising SEQ ID NO: 464; LCDR3 comprising SEQ ID NO: 575; LCDR2 comprising SEQ ID NO: 554; LCDR1 comprising SEQ ID NO: 537; and
(v) HCDR3 comprising SEQ ID NO: 512; HCDR2 comprising SEQ ID NO: 485; HCDR1 comprising SEQ ID NO: 465; LCDR3 comprising SEQ ID NO: 576; LCDR2 comprising SEQ ID NO: 555; LCDR1 comprising SEQ ID NO: 538.

The Nav1.7 antibodies of the present invention may exhibit selective binding to human Nav1.7 or may bind to human Nav1.7 and exhibit cross-reactivity with Nav1.7 homologues from other species, for example Nav1.7 of primate, mouse and/or rat origin. Nav1.7 antibodies exhibiting cross-reactivity may be beneficial in terms of drug development, for the reason that the antibodies can be tested in animal models of disease.

### Modulation of Nav1.7 Channel Activity

In preferred embodiments, the Nav1.7 antibodies or antigen binding fragments of the present invention are capable of binding to the Nav1.7 ion channel and modulating its activity. Modulation of ion channel activity should be taken to mean an increase or decrease in ion channel activity, wherein changes in activity may be measured for example, by detecting a change in ion channel currents or by detecting an effect at the level of cellular or physiological functions regulated by ion channel activity. The Nav1.7 antibodies of the invention may therefore act as agonist or antagonists of ion channel activity, wherein an agonist increases activity and an antagonist decreases activity. In preferred embodiments, the Nav1.7 antibodies are antagonists or “inhibitors” of ion channel activity.

As described elsewhere herein, voltage-gated ion channels such as Nav1.7 are gated by changes in membrane polarization. These channels typically exist in one of three conformational states: an “open” state, a “closed” state or an “inactivated” state, the state being dependent on the surrounding membrane potential. It is only possible for ions to flow through the channel pore when the channel is in the open state. The Nav1.7 antibodies and antigen binding fragments of the present invention which act as antagonists or inhibitors of ion channel activity may do so via any means which restricts or blocks the flow of ions through the pore including but not limited to stabilization of the closed or inactivated state. In preferred embodiments, the Nav1.7 antibodies inhibit Nav1.7 activity by stabilizing the inactivated state of the channel.

A decrease in ion channel activity corresponds to a decrease in the flow of ions through the pore of the channel. This may be measured by a decrease in the amplitude of current through a patch clamp assay. The decrease may be a partial decrease in the flow of ions or may be a total block of ion flow. The Nav1.7 antibodies of the present invention may decrease channel activity, as measured by a decrease in the flow of ions or a decrease in the amplitude of current through a patch clamp assay, by at least 40%, at least 50%, at least 60%, at least 70% or at least 80%, as compared with the activity measured in the absence of antibody or in the presence of a suitable control.

Techniques for measuring ion channel activity are known in the art. For the Nav1.7 antibodies of the present invention, the inhibition of channel activity may be measured in an in vitro patch clamp assay, as described elsewhere herein. The patch clamp assay may be used to assess the inhibitory effect of a compound, including an antibody of the invention, on the various channel states (open, closed, inactivated). Therefore, an inhibitory or antagonizing effect may be measured or determined at the level of the transition of the closed channel state to the open state or the inactivated channel state to the open state. In the context of the present invention, a reduction or decrease in Nav1.7 ion channel activity may reflect a reduction or decrease in ion channel activity at the level of the transition from the closed state to the open state, the inactivated state to the open state or both, preferably the inactivated state to the open state.

### Cross-Reactivity

As noted above, the Nav1.7 antibodies or antigen binding fragments of the present invention may bind to Nav1.7 homologues from different species, or may bind to Nav1.7 of human origin only. In preferred embodiments, the Nav1.7 antibodies or antigen binding fragments of the present invention will not exhibit any detectable binding to the voltage-gated sodium channel human Nav1.2. Alternatively or in addition, the Nav1.7 antibodies or antigen binding fragment of the present invention will not exhibit any detectable binding to the voltage-gated sodium channel human Nav1.5. Nav1.5 is an ion channel expressed by cardiac cells and plays a key role in controlling the excitability and thus contractility of heart tissue. A Nav1.7 antibody which does not bind Nav1.5 is therefore desirable from a therapeutic point of view for the reason that it would be less likely to trigger side-effects such as heart arrthymias.

In certain embodiments, the Nav1.7 antibodies and antigen binding fragments as described herein will not exhibit (detectable) binding to any other ion channel proteins, for example when measured using a standard binding assay, such as described elsewhere herein.

The Nav1.7 antibodies and antigen binding fragments described herein may also exhibit an affinity of binding to human Nav1.7, which is greater than the binding affinity of the antibody or antigen binding fragment for other voltage-gated sodium channels. For example, the antibodies of the invention may exhibit at least five-fold, at least ten-fold, at least twenty-fold greater affinity for human Nav1.7 as compared with any other member of the voltage-gated sodium channel family, preferably human Nav1.2, or preferably human Nav1.5.

### Camelid-Derived Antibodies

In certain aspects of the invention, the antibodies or antigen binding fragments thereof described herein may comprise at least one hypervariable loop or complementarity determining region obtained from a VH domain or a VL domain of a species in the family Camelidae. In particular, the antibody or antigen binding fragment may comprise VH and/or VL domains, or CDRs thereof, obtained by active immunisation of outbred camelids, e.g. llamas, with a Nav1.7 antigen or a DNA molecule comprising a nucleotide sequence encoding the Nav1.7 protein or fragment thereof.

By “hypervariable loop or complementarity determining region obtained from a VH domain or a VL domain of a species in the family Camelidae” is meant that that hypervariable loop (HV) or CDR has an amino acid sequence which is identical, or substantially identical, to the amino acid sequence of a hypervariable loop or CDR which is encoded by a Camelidae immunoglobulin gene. In this context “immunoglobulin gene” includes germline genes, immunoglobulin genes which have undergone rearrangement, and also somatically mutated genes. Thus, the amino acid sequence of the HV or CDR obtained from a VH or VL domain of a Camelidae species may be identical to the amino acid sequence of a HV or CDR present in a mature Camelidae conventional antibody. The term “obtained from” in this context implies a structural relationship, in the sense that the HVs or CDRs of the Nav1.7 antibody embody an amino acid sequence (or minor variants thereof) which was originally encoded by a Camelidae immunoglobulin gene. However, this does not necessarily imply a particular relationship in terms of the production process used to prepare the Nav1.7 antibody.

Camelid-derived Nav1.7 antibodies may be derived from any camelid species, including inter alia, llama, dromedary, alpaca, vicuna, guanaco or camel.

Nav1.7 antibodies comprising camelid-derived VH and VL domains, or CDRs thereof, are typically recombinantly expressed polypeptides, and may be chimeric polypeptides. The term “chimeric polypeptide” refers to an artificial (non-naturally occurring) polypeptide which is created by juxtaposition of two or more peptide fragments which do not otherwise occur contiguously. Included within this definition are “species” chimeric polypeptides created by juxtaposition of peptide fragments encoded by two or more species, e.g. camelid and human.

Camelid-derived CDRs may comprise one of the CDR sequences shown as SEQ ID Nos: 28-34, 141-149, 293 or 500-512 (heavy chain CDR3), or SEQ ID Nos: 16-21, 119-129, 291 or 472-485 (heavy chain CDR2) or SEQ ID Nos: 8-13, 100-109, 289 or 452-465 (heavy chain CDR1) or one of the CDR sequences shown as SEQ ID NOs: 77-84, 204-215, 300 or 563-576 (light chain CDR3), or SEQ ID Nos: 62-68, 181-192, 298 or 547-555 (light chain CDR2) or SEQ ID Nos:46-54, 161-172, 296 or 530-538 (light chain CDR1).

In one embodiment the entire VH domain and/or the entire VL domain may be obtained from a species in the family Camelidae. In specific embodiments, the camelid-derived VH domain may comprise the amino acid sequence shown as SEQ ID NOs: 218-226, 236-247, 302 or 583-596, whereas the camelid-derived VL domain may comprise the amino acid sequence show as SEQ ID Nos: 227-235, 248-259, 303 or 597-610. The camelid-derived VH domain and/or the camelid-derived VL domain may then be subject to protein engineering, in which one or more amino acid substitutions, insertions or deletions are introduced into the camelid amino acid sequence. These engineered changes preferably include amino acid substitutions relative to the camelid sequence. Such changes include “humanisation” or “germlining” wherein one or more amino acid residues in a camelid-encoded VH or VL domain are replaced with equivalent residues from a homologous human-encoded VH or VL domain. In certain embodiments, the camelid-derived VH domain may exhibit at least 90%, 95%, 97%, 98% or 99% identity with the amino acid sequence shown as SEQ ID NOs: 218-226, 236-247, 302 or 583-596. Alternatively, or in addition, the camelid-derived VL domain may exhibit at least 90%, 95%, 97%, 98% or 99% identity with the amino acid sequence shown as SEQ ID Nos: 227-235, 248-259, 303 or 597-610.

Isolated camelid VH and VL domains obtained by active immunisation of a camelid (e.g. llama) with a human Nav1.7 antigen or a DNA molecule comprising a nucleotide sequence encoding a Nav1.7 antigen can be used as a basis for engineering antigen binding polypeptides according to the invention. Starting from intact camelid VH and VL domains, it is possible to engineer one or more amino acid substitutions, insertions or deletions which depart from the starting camelid sequence. In certain embodiments, such substitutions, insertions or deletions may be present in the framework regions of the VH domain and/or the VL domain. The purpose of such changes in primary amino acid sequence may be to reduce presumably unfavourable properties (e.g. immunogenicity in a human host (so-called humanization), sites of potential product heterogeneity and or instability (glycosylation, deamidation, isomerisation, etc.) or to enhance some other favourable property of the molecule (e.g. solubility, stability, bioavailability etc.). In other embodiments, changes in primary amino acid sequence can be engineered in one or more of the hypervariable loops (or CDRs) of a Camelidae VH and/or VL domain obtained by active immunisation. Such changes may be introduced in order to enhance antigen binding affinity and/or specificity, or to reduce presumably unfavourable properties, e.g. immunogenicity in a human host (so-called humanization), sites of potential product heterogeneity and or instability, glycosylation, deamidation, isomerisation, etc., or to enhance some other favourable property of the molecule, e.g. solubility, stability, bioavailability, etc.

Thus, in one embodiment, the invention provides a variant Nav1.7 antibody which contains at least one amino acid substitution in at least one framework or CDR region of either the VH domain or the VL domain in comparison to a camelid-derived VH or VL domain, examples of which include but are not limited to the camelid VH domains comprising the amino acid sequences shown as SEQ ID NO: 218-226, 236-247, 302 or 583-596, and the camelid VL domains comprising the amino acid sequences show as SEQ ID NO: 227-235, 248-259, 303 or 597-610.

In other embodiments, there are provided “chimeric” antibody molecules comprising camelid-derived VH and VL domains (or engineered variants thereof) and one or more constant domains from a non-camelid antibody, for example human-encoded constant domains (or engineered variants thereof). In such embodiments it is preferred that both the VH domain and the VL domain are obtained from the same species of camelid, for example both VH and VL may be from Lama glama or both VH and VL may be from Lama pacos (prior to introduction of engineered amino acid sequence variation). In such embodiments both the VH and the VL domain may be derived from a single animal, particularly a single animal which has been actively immunised with a Nav1.7 antigen.

As an alternative to engineering changes in the primary amino acid sequence of Camelidae VH and/or VL domains, individual camelid-derived hypervariable loops or CDRs, or combinations thereof, can be isolated from camelid VH/VL domains and transferred to an alternative (i.e. non-Camelidae) framework, e.g. a human VH/VL framework, by CDR grafting. In particular, non-limiting, embodiments the camelid-derived CDRs may be selected from CDRs having the amino acid sequences shown as SEQ ID Nos: 28-34, 141-149, 293 or 500-512 (heavy chain CDR3), or SEQ ID Nos: 16-21, 119-129, 291 or 472-485 (heavy chain CDR2) or SEQ ID Nos: 8-13, 100-109, 289 or 452-465 (heavy chain CDR1) or one of the CDR sequences shown as SEQ ID NOs: 77-84, 204-215, 300 or 563-576 (light chain CDR3), or SEQ ID Nos: 62-68, 181-192, 298 or 547-555 (light chain CDR2) or SEQ ID Nos:46-54, 161-172, 296 or 530-538 (light chain CDR1).

Nav1.7 antibodies comprising camelid-derived VH and VL domains, or CDRs thereof, can take various different embodiments in which both a VH domain and a VL domain are present. The term “antibody” herein is used in the broadest sense and encompasses, but is not limited to, monoclonal antibodies (including full length monoclonal antibodies), polyclonal antibodies, multispecific antibodies (e.g., bispecific antibodies), so long as they exhibit the appropriate immunological specificity for a Nav1.7 protein. The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes) on the antigen, each monoclonal antibody is directed against a single determinant or epitope on the antigen.

“Antibody fragments” comprise a portion of a full length antibody, generally the antigen binding or variable domain thereof. Examples of antibody fragments include Fab, Fab′, F(ab′)2, bispecific Fab's, and Fv fragments, diabodies, linear antibodies, single-chain antibody molecules, a single chain variable fragment (scFv) and multispecific antibodies formed from antibody fragments (see Holliger and Hudson, Nature Biotechnol. 23:1126-36 (2005), the contents of which are incorporated herein by reference).

In non-limiting embodiments, Nav1.7 antibodies comprising camelid-derived VH and VL domains, or CDRs thereof, may comprise CH1 domains and/or CL domains, the amino acid sequence of which is fully or substantially human. Where the antigen binding polypeptide of the invention is an antibody intended for human therapeutic use, it is typical for the entire constant region of the antibody, or at least a part thereof, to have fully or substantially human amino acid sequence. Therefore, one or more or any combination of the CH1 domain, hinge region, CH2 domain, CH3 domain and CL domain (and CH4 domain if present) may be fully or substantially human with respect to its amino acid sequence.

Advantageously, the CH1 domain, hinge region, CH2 domain, CH3 domain and CL domain (and CH4 domain if present) may all have fully or substantially human amino acid sequence. In the context of the constant region of a humanised or chimeric antibody, or an antibody fragment, the term “substantially human” refers to an amino acid sequence identity of at least 90%, or at least 92%, or at least 95%, or at least 97%, or at least 99% with a human constant region. The term “human amino acid sequence” in this context refers to an amino acid sequence which is encoded by a human immunoglobulin gene, which includes germline, rearranged and somatically mutated genes. The invention also contemplates polypeptides comprising constant domains of “human” sequence which have been altered, by one or more amino acid additions, deletions or substitutions with respect to the human sequence, excepting those embodiments where the presence of a “fully human” hinge region is expressly required.

The presence of a “fully human” hinge region in the Nav1.7 antibodies of the invention may be beneficial both to minimise immunogenicity and to optimise stability of the antibody.

As discussed elsewhere herein, it is contemplated that one or more amino acid substitutions, insertions or deletions may be made within the constant region of the heavy and/or the light chain, particularly within the Fc region. Amino acid substitutions may result in replacement of the substituted amino acid with a different naturally occurring amino acid, or with a non-natural or modified amino acid. Other structural modifications are also permitted, such as for example changes in glycosylation pattern (e.g. by addition or deletion of N- or O-linked glycosylation sites). Depending on the intended use of the antibody, it may be desirable to modify the antibody of the invention with respect to its binding properties to Fc receptors, for example to modulate effector function. For example cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp. Med. 176:1191-1195 (1992) and Shopes, B. J. Immunol. 148:2918-2922 (1992). The invention also contemplates immunoconjugates comprising an antibody as described herein conjugated to a cytotoxic agent such as a chemotherapeutic agent, toxin (e.g., an enzymatically active toxin of bacterial, fungal, plant or animal origin, or fragments thereof), or a radioactive isotope (i.e., a radioconjugate). Fc regions may also be engineered for half-life extension, as described by Chan and Carter, Nature Reviews: Immunology, Vol. 10, pp 301-316, 2010, incorporated herein by reference.

In yet another embodiment, the Fc region is modified to increase the ability of the antibody to mediate antibody dependent cellular cytotoxicity (ADCC) and/or to increase the affinity of the antibody for an Fcγ receptor by modifying one or more amino acids. In alternative embodiments, the Fc region may be engineered such that there is no effector function. A Nav1.7 antibody having no Fc effector function may be particularly useful as a Nav1.7 channel blocking agent. In certain embodiments, the antibodies of the invention may have an Fc region derived from naturally-occurring IgG isotypes having reduced effector function, for example IgG4. Fc regions derived from IgG4 may be further modified to increase therapeutic utility, for example by the introduction of modifications that minimise the exchange of arms between IgG4 molecules in vivo.

In still another embodiment, the glycosylation of an antibody is modified. For example, an aglycoslated antibody can be made (i.e., the antibody lacks glycosylation). Glycosylation can be altered to, for example, increase the affinity of the antibody for the target antigen. Such carbohydrate modifications can be accomplished by; for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen.

Also envisaged are variant Nav1.7 antibodies having an altered type of glycosylation, such as a hypofucosylated antibody having reduced amounts of fucosyl residues or a fully or partially de-fucosylated antibody (as described by Natsume et al., Drug Design Development and Therapy, Vol. 3, pp 7-16, 2009) or an antibody having increased bisecting GlcNac structures. Such altered glycosylation patterns have been demonstrated to increase the ADCC activity of antibodies, producing typically 10-fold enhancement of ADCC relative to an equivalent antibody comprising a “native” human Fc domain. Such carbohydrate modifications can be accomplished by, for example, expressing the antibody in a host cell with altered glycosylation enzymatic machinery (as described by Yamane-Ohnuki and Satoh, mAbs 1:3, 230-236, 2009). Examples of non-fucosylated antibodies with enhanced ADCC function are those produced using the Potelligent™ technology of BioWa Inc.

The invention can, in certain embodiments, encompass chimeric Camelidae/human antibodies, and in particular chimeric antibodies in which the VH and VL domains are of fully camelid sequence (e.g. Llama or alpaca) and the remainder of the antibody is of fully human sequence. Nav1.7 antibodies can include antibodies comprising “humanised” or “germlined” variants of camelid-derived VH and VL domains, or CDRs thereof, and camelid/human chimeric antibodies, in which the VH and VL domains contain one or more amino acid substitutions in the framework regions in comparison to camelid VH and VL domains obtained by active immunisation of a camelid with a Nav1.7 antigen or a DNA molecule comprising a nucleotide sequence encoding a Nav1.7 antigen or a fragment thereof. Such “humanisation” increases the % sequence identity with human germline VH or VL domains by replacing mis-matched amino acid residues in a starting Camelidae VH or VL domain with the equivalent residue found in a human germline-encoded VH or VL domain.

Nav1.7 antibodies may also be CDR-grafted antibodies in which CDRs (or hypervariable loops) derived from a camelid antibody, or otherwise encoded by a camelid gene, are grafted onto a human VH and VL framework, with the remainder of the antibody also being of fully human origin. Such CDR-grafted Nav1.7 antibodies may contain CDRs having the amino acid sequences shown as SEQ ID Nos: 28-34, 141-149, 293 or 500-512 (heavy chain CDR3), or SEQ ID Nos: 16-21, 119-129, 291 or 472-485 (heavy chain CDR2) or SEQ ID Nos: 8-13, 100-109, 289 or 452-465 (heavy chain CDR1) or one of the CDR sequences shown as SEQ ID NOs: 77-84, 204-215, 300 or 563-576 (light chain CDR3), or SEQ ID Nos: 62-68, 181-192, 298 or 547-555 (light chain CDR2) or SEQ ID Nos:46-54, 161-172, 296 or 530-538 (light chain CDR1).

Humanised, chimeric and CDR-grafted Nav1.7 antibodies as described above, particularly antibodies comprising hypervariable loops or CDRs derived from active immunisation of camelids, can be readily produced using conventional recombinant DNA manipulation and expression techniques, making use of prokaryotic and eukaryotic host cells engineered to produce the polypeptide of interest and including but not limited to bacterial cells, yeast cells, mammalian cells, insect cells, plant cells, some of them as described herein and illustrated in the accompanying examples.

Camelid-derived Nav1.7 antibodies include variants wherein the hypervariable loop(s) or CDR(s) of the VH domain and/or the VL domain are obtained from a conventional camelid antibody raised against human Nav1.7, but wherein at least one of said (camelid-derived) hypervariable loops or CDRs has been engineered to include one or more amino acid substitutions, additions or deletions relative to the camelid-encoded sequence. Such changes include “humanisation” of the hypervariable loops/CDRs. Camelid-derived HVs/CDRs which have been engineered in this manner may still exhibit an amino acid sequence which is “substantially identical” to the amino acid sequence of a camelid-encoded HV/CDR. In this context, “substantial identity” may permit no more than one, or no more than two amino acid sequence mis-matches with the camelid-encoded HV/CDR. Particular embodiments of the Nav1.7 antibody may contain humanised variants of the CDR sequences shown as SEQ ID Nos: 28-34, 141-149, 293 or 500-512 (heavy chain CDR3), or SEQ ID Nos: 16-21, 119-129, 291 or 472-485 (heavy chain CDR2) or SEQ ID Nos: 8-13, 100-109, 289 or 452-465 (heavy chain CDR1) or one of the CDR sequences shown as SEQ ID NOs: 77-84, 204-215, 300 or 563-576 (light chain CDR3), or SEQ ID Nos: 62-68, 181-192, 298 or 547-555 (light chain CDR2) or SEQ ID Nos:46-54, 161-172, 296 or 530-538 (light chain CDR1).

The camelid-derived Nav1.7 antibodies provided herein may be of any isotype. Antibodies intended for human therapeutic use will typically be of the IgA, IgD, IgE IgG, IgM type, often of the IgG type, in which case they can belong to any of the four sub-classes IgG1, IgG2a and b, IgG3 or IgG4. Within each of these sub-classes it is permitted to make one or more amino acid substitutions, insertions or deletions within the Fc portion, or to make other structural modifications, for example to enhance or reduce Fc-dependent functionalities.

In preferred embodiments, the camelid-derived Nav1.7 antibodies described herein comprise a VH domain and/or a VL domain or at least one CDR obtained by immunization of an outbred camelid with a DNA molecule comprising a nucleotide sequence encoding a Nav1.7 protein or a fragment thereof.

Camelid-derived CDRs obtained by DNA immunization may comprise one of the CDR sequences shown as SEQ ID Nos: 28-34, 141-149, 293 or 500-512 (heavy chain CDR3), or SEQ ID Nos: 16-21, 119-129, 291 or 472-485 (heavy chain CDR2) or SEQ ID Nos: 8-13, 100-109, 289 or 452-465 (heavy chain CDR1) or one of the CDR sequences shown as SEQ ID NOs: 77-84, 204-215, 300 or 563-576 (light chain CDR3), or SEQ ID Nos: 62-68, 181-192, 298 or 547-555 (light chain CDR2) or SEQ ID Nos:46-54, 161-172, 296 or 530-538 (light chain CDR1).

The preferred combinations of heavy chain CDRs, light chain CDRs and preferred combinations of heavy and light chain CDRs selected from the groups recited above are described elsewhere herein (see also Tables 4, 5, 9, 10, 26 and 27) and are equally applicable to camelid-derived Nav1.7 antibodies obtained by DNA immunization.

### Preferred Nav1.7 Antibodies

The Nav1.7 antibodies or antigen binding fragments of the present invention exhibit a combination of the properties/features described above. In preferred embodiments, the combination of properties/features may be selected from:
(i) an affinity of binding for an extracellular region of human Nav1.7, (EC₅₀ as measured by ELISA), of less than 0.4 nM; and/or
(ii) inhibition of Nav1.7 ion channel activity by at least 40%; and/or
(iii) binding to the A3 extracellular loop of Nav1.7 as represented by SEQ ID NO: 262 (corresponding to amino acid residues 269 to 319 of SEQ ID NO:260); and/or
(iv) inability to bind the voltage-gated sodium channel human Nav1.5; and/or
(v) binding to rodent Nav1.7; and/or
(vi) high human homology; and/or
(vii) derived from an antibody raised by DNA immunization, preferably immunization of a camelid.

In preferred embodiments, the Nav1.7 antibodies or antigen binding fragments of the present invention have properties (i) and (ii) as defined above. In preferred embodiments, the Nav1.7 antibodies or antigen binding fragments of the present invention have properties (i), (ii) and (iii) as defined above. In preferred embodiments, the Nav1.7 antibodies or antigen binding fragments of the present invention have properties (i) (ii), (iii) and (iv) as defined above. In preferred embodiments, the Nav1.7 antibodies or antigen binding fragments of the present invention have properties (i) to (vii) as defined above. In any preferred embodiment, wherein the antibody or antigen binding fragment has at least property (iii) as defined above, the antibody may exhibit an affinity of binding for an extracellular region of human Nav1.7, which is at least 10-fold higher, at least 20-fold higher, at least 50-fold higher, at least 100-fold higher than a reference antibody selected from the group consisting of: UCB_932; UCB_983; and UCB_1066, as described in US2011/0135662, preferably UCB_932.

Alternatively, or in addition, the preferred Nav1.7 antibodies of the present invention may have at least one complementarity determining region selected from SEQ ID NOs: 100, 101, 119, 120, 141, 142, 161, 162, 181, 182, 204, 205.

Alternatively, or in addition, the preferred Nav1.7 antibodies or antigen binding fragments may comprise a heavy chain variable domain (VH) comprising a variable heavy chain CDR3 (HCDR3) consisting of: SEQ ID NO: 141 or 142, optionally comprising a heavy chain variable CDR2 (HCDR2) consisting of: SEQ ID NO: 119 or 120, and optionally comprising a heavy chain variable CDR1 (HCDR1) consisting of: SEQ ID NO: 100 or 101. Alternatively or in addition, the antibody or antigen binding fragment may comprise a light chain variable domain (VL) comprising a variable light chain CDR3 (LCDR3) consisting of: SEQ ID NO: 204 or 205, optionally comprising a light chain variable CDR2 (LCDR2) consisting of: SEQ ID NO: 181 or 182, and optionally comprising a light chain variable CDR1 (LCDR1) consisting of SEQ ID NO: 161 or 162.

The heavy chain variable domain may comprise either of the listed variable heavy chain CDR3 sequences (HCDR3) in combination with either of the variable heavy chain CDR2 sequences (HCDR2) and either of the variable heavy chain CDR1 sequences (HCDR1). However, certain combinations of HCDR3 and HCDR2 and HCDR1 are particularly preferred, these being the “native” combinations which derive from a single common VH domain. These preferred combinations are listed in Table 10. Further, the light chain variable domain may comprise either of the listed variable light chain CDR3 sequences (LCDR3) in combination with either of the variable light chain CDR2 sequences (LCDR2) and either of the variable light chain CDR1 sequences (LCDR1). However, certain combinations of LCDR3 and LCDR2 and LCDR1 are particularly preferred, these being the “native” combinations which derive from a single common VL domain. These preferred combinations are listed in Table 11.

Any given Nav1.7 antibody or antigen binding fragment thereof comprising a VH domain paired with a VL domain to form a binding site for Nav1.7 antigen will comprise a combination of six CDRs: variable heavy chain CDR3 (HCDR3), variable heavy chain CDR2 (HCDR2), variable heavy chain CDR1 (HCDR1), variable light chain CDR3 (LCDR3), variable light chain CDR2 (LCDR2) and variable light chain CDR1 (LCDR1).

Although all combinations of six CDRs selected from the CDR sequences listed above are permissible, and within the scope of the invention, certain combinations of six CDRs are particularly preferred; these being the “native” combinations within a single Fab exhibiting high affinity binding to Nav1.7.

Preferred combinations of six CDRs include, but are not limited to, the combinations of variable heavy chain CDR3 (HCDR3), variable heavy chain CDR2 (HCDR2), variable heavy chain CDR1 (HCDR1), variable light chain CDR3 (LCDR3), variable light chain CDR2 (LCDR2) and variable light chain CDR1 (LCDR1) selected from:

(i) HCDR3 comprising SEQ ID NO: 141; HCDR2 comprising SEQ ID NO: 119; HCDR1 comprising SEQ ID NO: 100; LCDR3 comprising SEQ ID NO: 204; LCDR2 comprising SEQ ID NO: 181; LCDR1 comprising SEQ ID NO: 161; and
(ii) HCDR3 comprising SEQ ID NO: 142; HCDR2 comprising SEQ ID NO: 120; HCDR1 comprising SEQ ID NO: 101; LCDR3 comprising SEQ ID NO: 205; LCDR2 comprising SEQ ID NO: 182; LCDR1 comprising SEQ ID NO: 162.

### Cross-Competing Antibodies

The present invention also includes monoclonal antibodies or antigen-binding fragments thereof that “cross-compete” with the antibodies or antigen binding fragments disclosed herein. Cross-competing antibodies are those that bind Nav1.7 at site(s) that are identical to, or overlapping with, the site(s) at which the present Nav1.7 antibodies bind. Competing monoclonal antibodies or antigen-binding fragments thereof can be identified, for example, via an antibody competition assay. For example, an extracellular loop region from Nav1.7 can be bound to a solid support. Then, an antibody or antigen binding fragment thereof of the present invention and a monoclonal antibody or antigen-binding fragment thereof suspected of being able to compete with such invention antibody are added. One of the two molecules is labelled. If the labelled compound and the unlabeled compound bind to separate and discrete sites on Nav1.7, the labelled compound will bind to the same level whether or not the suspected competing compound is present. However, if the sites of interaction are identical or overlapping, the unlabeled compound will compete, and the amount of labelled compound bound to the antigen will be lowered. If the unlabeled compound is present in excess, very little, if any, labelled compound will bind. For purposes of the present invention, competing monoclonal antibodies or antigen-binding fragments thereof are those that decrease the binding of the present antibody compounds to Nav1.7 by about 50%, about 60%, about 70%, about 80%, about 85%, about 90%, about 95%, or about 99%. Details of procedures for carrying out such competition assays are well known in the art and can be found, for example, in Harlow and Lane (1988) Antibodies, A Laboratory Manual, Cold Spring Harbor Laboratory Press, Cold Spring Harbor, N.Y., pages 567-569, ISBN 0-87969-314-2. Such assays can be made quantitative by using purified antibodies. A standard curve is established by titrating one antibody against itself, i.e., the same antibody is used for both the label and the competitor. The capacity of an unlabeled competing monoclonal antibody or antigen-binding fragment thereof to inhibit the binding of the labelled molecule to the plate is titrated. The results are plotted, and the concentrations necessary to achieve the desired degree of binding inhibition are compared.

### Polynucleotides Encoding Nav1.7 Antibodies

The invention also provides a polynucleotide molecules encoding the antibodies of the invention, including the Nav1.7 antibodies of the invention, also expression vectors containing a nucleotide sequences which encode the antibodies of the invention operably linked to regulatory sequences which permit expression of the antigen binding polypeptide in a host cell or cell-free expression system, and a host cell or cell-free expression system containing this expression vector.

In particular embodiments, the polynucleotide encoding the Nav1.7 antibody of the invention may comprise one or more of the polynucleotide sequences shown as SEQ ID NOs: 304-309, which sequences encode VH or VL domains of Nav1.7 antibodies, or a variant sequence which encodes a functional VH or VL domain of a Nav1.7 antibody, wherein said variant sequence exhibits at least 80%, 85%, 90%, 95%, 97% or 99% sequence identity when optimally aligned to one of SEQ ID NOs: 304-309.

In this context, % sequence identity between two polynucleotide sequences may be determined by comparing these two sequences aligned in an optimum manner and in which the polynucleotide sequence to be compared can comprise additions or deletions with respect to the reference sequence for an optimum alignment between these two sequences. The percentage of identity is calculated by determining the number of identical positions for which the nucleotide residue is identical between the two sequences, by dividing this number of identical positions by the total number of positions in the comparison window and by multiplying the result obtained by 100 in order to obtain the percentage of identity between these two sequences. For example, it is possible to use the BLAST program, “BLAST 2 sequences” (Tatusova et al, “Blast 2 sequences—a new tool for comparing protein and nucleotide sequences”, FEMS Microbiol Lett. 174:247-250) available on the site http://www.ncbi.nlm.nih.gov/gorf/b12.html, the parameters used being those given by default (in particular for the parameters “open gap penalty”: 5, and “extension gap penalty”: 2; the matrix chosen being, for example, the matrix “BLOSUM 62” proposed by the program), the percentage of identity between the two sequences to be compared being calculated directly by the program.

Polynucleotide molecules encoding the antibodies of the invention include, for example, recombinant DNA molecules. The terms “nucleic acid”, “polynucleotide” or a “polynucleotide molecule” as used herein interchangeably and refer to any DNA or RNA molecule, either single- or double-stranded and, if single-stranded, the molecule of its complementary sequence. In discussing nucleic acid molecules, a sequence or structure of a particular nucleic acid molecule may be described herein according to the normal convention of providing the sequence in the 5′ to 3′ direction. In some embodiments of the invention, nucleic acids or polynucleotides are “isolated.” This term, when applied to a nucleic acid molecule, refers to a nucleic acid molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or non-human host organism. When applied to RNA, the term “isolated polynucleotide” refers primarily to an RNA molecule encoded by an isolated DNA molecule as defined above. Alternatively, the term may refer to an RNA molecule that has been purified/separated from other nucleic acids with which it would be associated in its natural state (i.e., in cells or tissues). An isolated polynucleotide (either DNA or RNA) may further represent a molecule produced directly by biological or synthetic means and separated from other components present during its production.

For recombinant production of an antibody according to the invention, a recombinant polynucleotide encoding it may be prepared (using standard molecular biology techniques) and inserted into a replicable vector for expression in a chosen host cell, or a cell-free expression system. Suitable host cells may be prokaryote, yeast, or higher eukaryote cells, specifically mammalian cells. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen. Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); mouse myeloma cells SP2/0-AG14 (ATCC CRL 1581; ATCC CRL 8287) or NS0 (HPA culture collections no. 85110503); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2), as well as DSM's PERC-6 cell line. Expression vectors suitable for use in each of these host cells are also generally known in the art.

It should be noted that the term “host cell” generally refers to a cultured cell line. Whole human beings into which an expression vector encoding an antigen binding polypeptide according to the invention has been introduced are explicitly excluded from the definition of a “host cell”.

### Antibody Production

In an important aspect, the invention also provides a method of producing antibodies of the invention which comprises culturing a host cell (or cell free expression system) containing polynucleotide (e.g. an expression vector) encoding the antibody under conditions which permit expression of the antibody, and recovering the expressed antibody. This recombinant expression process can be used for large scale production of antibodies, including Nav1,7 antibodies according to the invention, including monoclonal antibodies intended for human therapeutic use. Suitable vectors, cell lines and production processes for large scale manufacture of recombinant antibodies suitable for in vivo therapeutic use are generally available in the art and will be well known to the skilled person.

### Therapeutic Utility of Nav1.7 Antibodies

The Nav1.7 antibodies provided herein can be used as medicaments, particularly for use in the treatment or prophylaxis of a pathological disorder that is mediated by Nav1.7 or that is associated with an increased level of Nav1.7, or that is associated with an increase or decrease in Nav1.7 channel activity.

The term “treating” or “treatment” means slowing, interrupting, arresting, controlling, stopping, reducing severity of a symptom, disorder, condition or disease, but does not necessarily involve a total elimination of all disease-related symptoms, conditions or disorders. The term “prophylaxis” means preventing the onset of a disorder, condition or disease or preventing the onset of symptoms associated with a disorder, condition or disease.

Studies from humans and animal models have identified Nav1.7 as a protein which plays a role in sensing pain. Gain-of-function mutations in the gene encoding Nav1.7, SCN9A, have been identified as the cause of erythromelalgia, an autosomal dominant disorder associated with bouts of burning pain, and also paroxysmal extreme pain disorder (Dib-Hajj et al. 2008). Furthermore, a loss-of-function mutation in human Nav1.7 has been linked to a disorder associated with insensitivity to pain (Cox et al., 2006).

The Nav1.7 antibodies or antigen binding fragments described herein may be used for the treatment or prophylaxis of pain, including neuropathic pain, somatic pain, visceral pain, nociceptive pain, acute pain, chronic pain, breakthrough pain and/or inflammatory pain.

In certain embodiments, the antibodies or antigen binding fragments may be used to treat neuropathic pain including but not limited to painful diabetic neuropathy (PDN), post-herpetic neuropathy (PHN), or trigeminal neuralgia (TN), or for the treatment or prophylaxis of neuropathic pain caused by or related to spinal cord injuries, multiple sclerosis, phantom limb pain, post-stroke pain, HIV-associated pain, chronic back pain, osteoarthritis, rheumatoid arthritis, inflammation and/or cancer.

For human therapeutic use the Nav1.7 antibodies described herein may be administered to a human subject in need of treatment in an “effective amount”. The term “effective amount” refers to the amount or dose of a Nav1.7 antibody which, upon single or multiple dose administration to a human patient, provides therapeutic efficacy in the treatment of disease. Therapeutically effective amounts of the Nav1.7 antibody can comprise an amount in the range of from about 0.1 mg/kg to about 20 mg/kg per single dose. The amount of antibody administered at any given time point may be varied so that optimal amounts of Nav1.7 antibody, whether employed alone or in combination with any other therapeutic agent, are administered during the course of treatment.

It is also contemplated to administer the Nav1.7 antibodies described herein, or pharmaceutical compositions comprising such antibodies, in combination with any other pain treatment, as a combination therapy.

### Pharmaceutical Compositions

The scope of the invention includes pharmaceutical compositions, containing one or a combination of Nav1.7 antibodies of the invention, or antigen-binding fragments thereof, formulated with one or more a pharmaceutically acceptable carriers or excipients. Such compositions may include one or a combination of (e.g., two or more different) Nav1.7 antibodies. Techniques for formulating monoclonal antibodies for human therapeutic use are well known in the art and are reviewed, for example, in Wang et al., Journal of Pharmaceutical Sciences, Vol. 96, pp 1-26, 2007.

### Methods of Obtaining Antibodies by DNA Immunization

In a further aspect, the present invention provides a method of raising an antibody which binds a target protein wherein the target protein has a length of at least 1115 amino acids and/or has at least 8 transmembrane domains and/or is naturally encoded by a nucleotide sequence which is difficult to replicate in a common E. coli strain.

Features of the target protein to which the antibody binds and the DNA used for immunization, have already been described in the context of the other aspects of the invention detailed above. However, all embodiments relating to the target protein and the DNA molecule used for DNA immunization are equally applicable to the methods of the present invention described below.

In the methods of the invention, the host animal is immunized with a DNA molecule in order to induce a humoral immune response against the antigen(s) encoded by the nucleotide sequence(s) included in the DNA molecule. As noted above, DNA immunization and DNA vaccination protocols have been described in the prior art and any suitable protocol may be adopted for raising antibodies in the context of methods of the present invention. The immunization protocol must be effective so as to induce an adequate antibody titre in the host animal. Therefore, the method may involve one or multiple immunizations. For example, the method may involve 2, 3, 4, 5 or 6 separate administrations of DNA to the host animal. Wherein the method requires multiple immunizations, the immunizations may be at different sites within the animal, and/or may be at suitable time intervals, for example, at intervals of 3, 7, 14, 21 or 28 days. In certain embodiments, immunizations may be performed on a weekly basis, or on alternate weeks, until an adequate antibody titre is generated.

The methods of the invention may include a step of “boosting” the host animal with a composition comprising the target protein or a fragment thereof. A suitable composition for use in boosting the host animal's immune response may comprise cells expressing the protein or the fragment thereof. The composition may be administered to the host animal by any means known to those skilled in the art, and may optionally include a suitable adjuvant.

Non-human host animals suitable for DNA immunization according to the methods of the present invention include but are not limited to mouse, rat, rabbit, goat, hamster, chicken, monkey or an animal from the Camelidae family. In preferred embodiments, the host animal is a camelid, preferably a llama or alpaca.

In certain embodiments, the method may further comprise the steps of (i) separating or isolating antibodies from the immunized host animal and (ii) identifying antibodies having binding specificity for the target protein. Suitable methods for harvesting sera and identifying polyclonal and/or monoclonal antibodies with binding specificity for the target antigen are known in the art.

Alternatively or in addition, the method of the invention may further comprise step(s) to isolate material from the host animal wherein the material is used as a source of antibody producing cells containing nucleotide sequences from which antigen binding fragments immunoreactive with the target protein can be identified. Such material may include peripheral blood monocytes (PBMCs), peripheral blood lymphocytes (PBLs), peripheral lymph nodes, spleen and/or bone marrow.

The method may further comprise a step of constructing a library or collection of immunoglobulin sequences derived from the immunized host, in order to screen for antibodies or antigen binding fragments thereof immunoreactive with the target protein of interest. Methods for library construction are known in the art, for example following the immunization of camelids with target antigens, as described in WO2010/001251 incorporated herein by reference in its entirety.

In preferred embodiments, the methods of the present invention involve DNA immunization of a host animal, preferably a camelid host, and following DNA immunization, include the steps of:
harvesting blood, sera, lymphoid tissue, PBMCs or PBLs from the host animal;
isolating nucleic acid and amplifying regions encoding VH and/or VL domains of camelid conventional antibodies; each gene segment containing a sequence of nucleotides encoding a VH domain or a sequence of nucleotides encoding a VL domain of a camelid conventional antibody; and
cloning the gene segments into expression vectors, such that each expression vector contains at least a gene segment encoding a VH domain and/or a gene segment encoding a VL domain.

Preferably the methods involve harvesting peripheral blood lymphocytes from which nucleic acid can be isolated. The nucleic acid from which the VH and VL domain sequences derive may be total RNA, mRNA or genomic DNA isolated from the cells within the sample taken from the host. The expression vectors may be any suitable vectors used for library construction, and are preferably phage or phagemid vectors allowing selection of target specific antibody fragments using phage display based selection methods.

The methods of the present invention may also include screening steps to select and isolate antibody fragments which bind the target protein. For example, libraries of phage expressing VH and VL domains may be selected by a single round or multiple rounds of panning on a suitable source of the target antigen of interest, for example, an immobilized form of the target antigen.

In certain embodiments, nucleic acids encoding VH and VL domains are cloned into expression vectors, preferably phage or phagemid vectors, such that each vector comprises a nucleotide sequence encoding a single chain variable fragment (scFv) of a conventional antibody. In this way, libraries of phage expressing scFv fragments can be constructed and used for screening against a suitable source of the target antigen of interest. In preferred embodiments, the nucleotide sequences encoding the scFv fragments are cloned into expression vectors in operable linkage with a sequence of nucleotides encoding one or more more constant domains of a human antibody, thereby producing a library of expression vectors encoding chimeric antigen binding polypeptides comprising scFv fragments fused to one or more constant domains of a human antibody, preferably the Fc region of a human IgG. Such chimeric antigen binding polypeptides can be screened for immunoreactivity with any suitable source of target antigen.

It has been found that screening using scFv libraries and in particular, chimeric antigen binding polypeptides comprising an scFv fragment fused to an Fc domain, is particularly advantageous for identifying antigen binding polypeptides immunoreactive with complex target proteins as defined elsewhere herein. This is particularly the case when screening is carried out using the full-length target protein, for example cells overexpressing the full-length target protein of interest or membranes or lipoparticles derived therefrom. When screening is carried out using cells, membranes and/or lipoparticles carrying membrane-associated target proteins such as ion channels and GPCRs, the density of antigen presentation to the library is typically low as compared with screening using a recombinantly produced target protein. The membrane environment also results in background problems during phage panning.

Screening using scFv phage libraries, as compared with libraries of Fab-expressing phage, is advantageous because scFv display on phage is more efficient than Fab display. The low density of target antigen or protein is therefore compensated for by the higher density of phage-displayed scFv fragments. As such, the present invention encompasses the identification of antibodies immunoreactive with complex target proteins as defined elsewherein herein using libraries of phage expressing scFv fragments, optionally wherein the fragments are fused to one or more constant domains of a human antibody. In preferred embodiments, the scFv libraries are screened using lipoparticles carrying the target protein of interest.

Once nucleotide sequences encoding VH and/or VL antibody fragments immunoreactive with the target protein or antigen of interest have been identified, the sequences can be used to produce further antibodies or antigen binding fragments thereof which bind to the target. Therefore, the methods of the present invention may include the further step(s) of preparing a recombinant antibody or antigen binding fragment thereof by:
determining the nucleotide sequence encoding at least one hypervariable loop or CDR of the VH and/or the VL domain immunoreactive with the target protein; and
expressing an antibody or antigen binding fragment thereof which binds to said target protein, said antibody or antigen binding fragment thereof comprising a VH and a VL domain, wherein at least one hypervariable loop or CDR of the VH or VL domain has an amino acid sequence encoded by the nucleotide sequence determined in the preceding step.

Alternatively, the methods of the present invention may include the further step(s) of preparing a recombinant antibody or antigen binding fragment by:
isolating nucleic acid encoding at least one hypervariable loop or CDR of the VH and/or the VL domain immunoreactive with the target antigen,
preparing a recombinant polynucleotide comprising a nucleotide sequence encoding hypervariable loop(s) or CDR(s) having amino acid sequence(s) identical or substantially identical to the hypervariable loop(s) or CDR(s) encoded by the nucleic acid isolated in the preceding step, which recombinant polynucleotide encodes an antibody or antigen binding fragment thereof comprising a VH domain and a VL domain that specifically binds to said target antigen, and
expressing said antibody or antigen binding fragment thereof from the recombinant polynucleotide of the preceding step.

The present invention also extends to antibody producing cells, antibodies and/or antigen binding fragments obtainable or obtained from the methods described herein.

### INCORPORATION BY REFERENCE

Various publications are cited in the foregoing description and throughout the following examples, each of which is incorporated by reference herein in its entirety.

### EXAMPLES

The invention will be further understood with reference to the following non-limiting experimental examples.

### Nav1.7 Antibodies Generated by Peptide Immunization

### Example 1

### Immunization of Llama

Immunizations of llamas and harvesting of peripheral blood lymphocytes (PBLs) as well as the subsequent extraction of RNA and amplification of antibody fragments were performed as described by De Haard and colleagues (De Haard H, et al., J. Bact. 187:4531-4541, 2005; Basilico C., et al., J. Clin. Invest. 124:3172-86, 2014). Two llamas were immunized six times in a weekly interval with hNav1.7-loopA3-llama Fc fusion (SEQ ID NO: 267, see Table 4) by intramuscular injections in the neck divided over two spots. For the first 2 immunizations 100 g antigen was used, whilst for the subsequent immunizations 50 g antigen was used. Antigen was mixed with Freund's Incomplete Adjuvant prior to immunization.

Four days after the last immunization, 400 ml blood was collected for extraction of total RNA from the PBLs using a Ficoll-Paque gradient to isolate PBLs and the method described by Chomczynski P, et al., Anal. Biochem. 162: 156-159, 1987 to prepare the RNA. On average, a few 100 μg was extracted and aliquoted, prior to use for random primed cDNA synthesis and subsequent PCR amplification of the llama VHCH1, VλCλ and VκCκ gene segments.

### Example 2

### Library Construction, Selection and Screening

Independent VλCλ and VκCκ libraries were constructed using a single-step PCR, in which 25 cycles with tagged primers was done (De Haard H., et al., J. Biol. Chem. 274: 18218-30, 1999). The VHCH1 libraries were built using a two step PCR, in which 25 cycles with non tagged primers was done followed by 10 cycles using tagged version of these primers. The sizes of the individual libraries were between 10⁸ and 10⁹ cfu. Next the antibody fragments were re-cloned to form Fab-libraries. The final libraries were between 1×10⁸ and 4×10⁹ cfu. Quality control of the libraries was routinely performed using PCR.

Three rounds of selections were done on directly coated hNav1.7-loopA3, hNav1.7-LoopB1-C1-D1 or hNav1.7-LoopC3 (as represented by SEQ ID NOs. 267, 268 and 269, see Table 4) using standard protocols. Elutions were done with trypsin or triethylamine. An aliquot of the eluted phages was used to infect TG1 bacteria, which were subsequently plated on an Agar Plate containing ampicillin and 2% glucose.

TABLE 4
Constructs used for peptide immunization, screening and functional testing of Nav1.7 antibodies

| Constructs | Amino Acid Sequences | SEQ ID NO: |
|---|---|---|
| hNav1.7-loopA3-Ilfc | GNLKHKCFRNSLENNETLESIMNTLESEEDFRKYFYYLEGSKDALLCGFSTIEGRDMDPHGGCTCPQCPAPELPGGPSVFVFPPKPKDVLSIS | 267 |
| | GRPEVTCVVVDVGKEDPEVNFNWYIDGVEVRTANTKPKEEQFNSTYRVVSVLPIQHQDWLTGKEFKCKVNNKALPAPIERTISKAKGQTRE | |
| | PQVYTLAPHREELAKDTVSVTCLVKGFYPADINVEWQRNGQPESEGTYANTPPQLDNDGTYFLYSKLSVGKNTWQRGETLTCVVMHEAL | |
| | HNHYTQKSISQSPGK | |
| hNav1.7-loopB1-C1-D1-Ilfc | GGSCGSEHHPMTEEFKNVLGGSGGSEDIYIERKKTIKIIGGSGGSVEKEGQSQHMTEVLGGSCGSIEGRDMDPHGGCTCPQCPAPELPGG | 268 |
| | PSVFVFPPKPKDVLSISGRPEVTCVVVDVGKEDPEVNFNWYIDGVEVRTANTKPKEEQFNSTYRVVSVLPIQHQDWLTGKEFKCKVNNKAL | |
| | PAPIERTISKAKGQTREPQVYTLAPHREELAKDTVSVTCLVKGFYPADINVEWQRNGQPESEGTYANTPPQLDNDGTYFLYSKLSVGKNTW | |
| | QRGETLTCVVMHEALHNHYTQKSISQSPGK | |
| hNav1.7-loopC3-Ilfc | GKFYECINTTDGSRFPASQVPNRSECFALMNVSQNVRWKNLKVNFDNVGLGYLSLLQVATFKGWTIIMYAAVDSVNVDKQPKYEYSLIEGR | 269 |
| | DMDPHGGCTCPQCPAPELPGGPSVFVFPPKPKDVLSISGRPEVTCVVVDVGKEDPEVNFNWYIDGVEVRTANTKPKEEQFNSTYRVVSVL | |
| | PIQHQDWLTGKEFKCKVNNKALPAPIERTISKAKGQTREPQVYTLAPHREELAKDTVSVTCLVKGFYPADINVEWQRNGQPESEGTYANTP | |
| | PQLDNDGTYFLYSKLSVGKNTWQRGETLTCVVMHEALHNHYTQKSISQSPGK | |
| hNav1.5-loopA3-IlFc | GNLRHKCVRNFTALNGTNGSVEADGLVWESLDLYLSDPENYLLKNGTSDVLLCGNSSIEGRDMDPHGGCTCPQCPAPELPGGPSVFVFPP | 270 |
| | KPKDVLSISGRPEVTCVVVDVGKEDPEVNFNWYIDGVEVRTANTKPKEEQFNSTYRVVSVLPIQHQDWLTGKEFKCKVNNKALPAPIERTIS | |
| | KAKGQTREPQVYTLAPHREELAKDTVSVTCLVKGFYPADINVEWQRNGQPESEGTYANTPPQLDNDGTYFLYSKLSVGKNTWQRGETLTC | |
| | VVMHEALHNHYTQKSISQSPGK | |
| GST-tagged hNav1.2-loopA3 sequence | GST-tag- | 271 |
| | NLRNKCLQWPPDNSSFEINITSFFNNSLDGNGTTFNRTVSIFNWDEYIEDKSHFYFLEGQNDALLCGNSSDAGQCPEGYICVKAGRNPNY | |
| GST-tagged hNav1.7-loopA3 sequences | GST-tag-GNLKHKCFRNSLENNETLESIMNTLESEEDFRKYFYYLEGSKDALLCGFSTDSGQCPEGYTCVKIGRNPDY | 272 |
| RatB1-C1-D1-IlFc | GGSCGSEHHPMTEEFKNVLGGSGGSEDIYIEKKKTIKIIGGSGGSVEKEGQTEYMDYVLGGSCGSIEGRDMDPHGGCTCPQCPAPELPGGP | 285 |
| | SVFVFPPKPKDVLSISGRPEVTCVVVDVGKEDPEVNFNWYIDGVEVRTANTKPKEEQFNSTYRVVSVLPIQHQDWLTGKEFKCKVNNKALP | |
| | APIERTISKAKGQTREPQVYTLAPHREELAKDTVSVTCLVKGFYPADINVEWQRNGQPESEGTYANTPPQLDNDGTYFLYSKLSVGKNTWQ | |
| | RGETLTCVVMHEALHNHYTQKSISQSPGK | |
| ratA3-IlFc | GNLKHKCFRKELEENETLESIMNTAESEEELKKYFYYLEGSKDALLCGFSTIEGRDMDPHGGCTCPQCPAPELPGGPSVFVFPPKPKDVLSISG | 286 |
| | RPEVTCVVVDVGKEDPEVNFNWYIDGVEVRTANTKPKEEQFNSTYRVVSVLPIQHQDWLTGKEFKCKVNNKALPAPIERTISKAKGQTREP | |
| | QVYTLAPHREELAKDTVSVTCLVKGFYPADINVEWQRNGQPESEGTYANTPPQLDNDGTYFLYSKLSVGKNTWQRGETLTCVVMHEALH | |
| | NHYTQKSISQSPGK | |
| hNav1.5-B1-C1-D1-IlFc | GGSCGSEHYNMTSEFEEMLGGSGGSEDIYLEERKTIKVLGGSGGSVETDDQSPEKINILGGSCGSIEGRDMDPHGGCTCPQCPAPELPGGP | 287 |
| | SVFVFPPKPKDVLSISGRPEVTCVVVDVGKEDPEVNFNWYIDGVEVRTANTKPKEEQFNSTYRVVSVLPIQHQDWLTGKEFKCKVNNKALP | |
| | APIERTISKAKGQTREPQVYTLAPHREELAKDTVSVTCLVKGFYPADINVEWQRNGQPESEGTYANTPPQLDNDGTYFLYSKLSVGKNTWQ | |
| | RGETLTCVVMHEALHNHYTQKSISQSPGK | |

Letters in italics: Nav1.7/Nav1.5/Nav1.2 sequence;
Letters in bold: linker sequences;
Underlined sequence: Ilama Fc

Individual colonies were isolated from all the libraries and Fab was produced in the periplasm in 96-deep well plates containing 1 ml 2TY+Amp+1 mM IPTG according to standard protocols. After O/N induction at 26° C., the pellet was frozen (O/N at −20° C.) and thawed in PBS. After a final centrifugation to pellet the bacterial debris, the PBS containing the Fabs (periplasmic fractions, peris) was collected and screened.

Loop-specific Fabs were identified. The VH and VL of interesting loop-specific Fab clones were fused to the constant domains of human IgG1 or to human CCK respectively and produced as bivalent chimeric monoclonal antibodies using the system described in patent application WO2009/145606. The chimeric llama-human monoclonal antibodies were purified using protein A resin.

TABLE 5
Framework regions and CDR sequences for VH domains of peptide-raised Nav1.7 antibodies

| | | SEQ | | SEQ | | SEQ | | SEQ | | SEQ | | SEQ | | SEQ |
|---|---|---|---|---|---|---|---|---|---|---|---|---|---|---|
| | | ID | | ID | | ID | | ID | | ID | | ID | | ID |
| Clone | FR1 | NO. | CDR1 | NO. | FR2 | NO. | CDR2 | NO. | FR3 | NO. | CDR3 | NO. | FR4 | NO. |
| 3F11 | qvqlvesggg | 1 | sywmy | 8 | wvrqapg | 14 | aintgggst | 16 | rftisrdnakntlylqm | 22 | gpgysgs | 28 | wgqgt | 35 |
| | lvqpggslrls | | | | kglewvs | | yyadsvkg | | nslkpedtalyycar | | yiegway | | qvtvss | |
| | caasgftfs | | | | | | | | | | | | | |
| 2F12 | qvqlvesggg | 1 | sywmy | 8 | wvrqapg | 14 | aintgggst | 16 | rftisrdnakntlylqm | 22 | gpgysgk | 29 | wgqgt | 35 |
| | lvqpggslrls | | | | kglewvs | | yyadsvkg | | nslkpedtalyycar | | yiegway | | qvtvss | |
| | caasgftfs | | | | | | | | | | | | | |
| 3E1 | evqvqesgg | 2 | sywmy | 8 | wvrqapg | 14 | aintgggst | 16 | rftisrdnakntlylqm | 22 | gpgysgk | 29 | wgqgt | 35 |
| | glvqpggslrl | | | | kglewvs | | yyadsvkg | | nslkpedtalyycar | | yiegway | | qvtvss | |
| | scaasgftfs | | | | | | | | | | | | | |
| 4E8 | evqlqesggg | 3 | dyams | 9 | wvrqapg | 14 | aiswnggs | 17 | rftisrdnakntlylqm | 23 | rryglgse | 30 | wgqgt | 35 |
| | lvqpggslrls | | | | kglewvs | | tyyaesmkg | | nslkskdtavyycar | | ydy | | qvtvss | |
| | caasgftfd | | | | | | | | | | | | | |
| 4F6 | qlqlvesgggl | 4 | dyams | 9 | wvrqapg | 14 | aiswnggs | 17 | rftisrdnakntlylqm | 23 | rryglgse | 30 | wgqgt | 35 |
| | vqpggslrlsc | | | | kglewvs | | tyyaesmkg | | nslkskdtavyycar | | ydy | | qvtvss | |
| | aasgftfd | | | | | | | | | | | | | |
| 4F7 | qvqlqesgp | 5 | tsfyaws | 10 | wirqppgk | 15 | viaydgrtf | 18 | rthisrdtsknqfslqlss | 24 | ggvvaws | 31 | wgqgt | 35 |
| | glvkpsqtlslt | | | | glewmg | | yspslks | | vtpedtavyycar | | ydy | | qvtvss | |
| | ctvsggsit | | | | | | | | | | | | | |
| 4H6 | qvqlqesgp | 5 | nygws | 11 | wirqppgk | 15 | viaydgsty | 19 | rtsisrdtsknqfslqlss | 25 | amvtts | 32 | wgqgt | 35 |
| | glvkpsqtlslt | | | | glewmg | | yspslks | | vtpedtavyycas | | spwvkt | | qvtvss | |
| | ctvsggsit | | | | | | | | | | fga | | | |
| 2D11 | evqlqesgpg | 6 | nsyyass | 12 | wirqppgk | 15 | viiydgsty | 20 | rtsisrdtsknqfslqlss | 26 | glrvagsy | 33 | wgqgt | 35 |
| | lvkpsqtlsltc | | | | glewmg | | yspslks | | vtpedtavyycar | | ftdfgt | | qvtvss | |
| | tvsggsit | | | | | | | | | | | | | |
| 6H8 | evqlvesggg | 7 | nywmh | 13 | wvrqapg | 14 | vidrggggt | 21 | rftisrdnakntlylrmn | 27 | drngeydy | 34 | wgrgt | 36 |
| | lvqpggtlrls | | | | kglewvs | | ryadsvkg | | slkpedtalyyctr | | | | qvtvss | |
| | caasgftfs | | | | | | | | | | | | | |

TABLE 6
Framework regions and CDR sequences for VL domains of peptide-raised Nav1.7 antibodies

| | | SEQ | | SEQ | | SEQ | | SEQ | | SEQ | | SEQ | | SEQ |
|---|---|---|---|---|---|---|---|---|---|---|---|---|---|---|
| | | ID | | ID | | ID | | ID | | ID | | ID | | ID |
| Clone | FR1 | NO. | CDR1 | NO. | FR2 | NO. | CDR2 | NO. | FR3 | NO. | CDR3 | NO. | FR4 | NO. |
| 3F11 | divmtqsp | 37 | kssq | 46 | wyqqrpgqs | 55 | hastqes | 62 | gipdrfsgsgsttdftltis | 69 | qqay | 77 | fgsgtrleik | 85 |
| | tsvta | | svvs | | prlliy | | | | svqpedaavyyc | | nnpys | | | |
| | svgek | | gsnq | | | | | | | | | | | |
| | vtinc | | ksyln | | | | | | | | | | | |
| 2F12 | divmtqsp | 38 | kssq | 47 | wyqqrpgqs | 55 | yastqes | 63 | glpdrfsgsgsttdftltis | 70 | qqay | 78 | fgsgtrleik | 85 |
| | rsvtas | | svvse | | prlliy | | | | svqpedaavyyc | | sapys | | | |
| | vgekv | | snqr | | | | | | | | | | | |
| | finc | | syln | | | | | | | | | | | |
| 3E1 | divmtqsp | 39 | kssq | 48 | wyqqrpgqs | 55 | yastqes | 63 | gipdrfsgsgsttdftltis | 69 | qqay | 78 | fgsgtrleik | 85 |
| | ssvta | | svvsg | | prlliy | | | | svqpedaavyyc | | sapys | | | |
| | svgekvt | | skqk | | | | | | | | | | | |
| | inc | | syln | | | | | | | | | | | |
| 4E8 | nfmltqps | 40 | qgs | 49 | wyqqmpgq | 56 | rdserps | 64 | gipdrfsgsnsggtatlti | 71 | qsgs | 79 | fgggthltvlg | 86 |
| | avsvslg | | sfgs | | apilviy | | | | sgaqaedeadyyc | | av | | ssgnah | |
| | qtaritc | | syah | | | | | | | | | | | |
| 4F6 | hsavtqps | 41 | qgtn | 50 | wyqqmpgq | 57 | rdserps | 64 | giperfsgsssggtatlai | 72 | qsah | 80 | fgggthltvlg | 86 |
| | avsvslg | | lrss | | apvlviy | | | | sgaqaedeadyyc | | rsesav | | | |
| | qtaritc | | yvh | | | | | | | | | | | |
| 4F7 | syeltqs | 42 | ggnd | 51 | wyqqkagq | 58 | adsrrps | 65 | giperfsgsnsgntatlti | 73 | qvwd | 81 | fgggthltvlg | 86 |
| | psvsval | | igsk | | apvlviy | | | | sgaqaedeadyyc | | av | | srada | |
| | rqtakitc | | stq | | | | | | | | | | | |
| 4H6 | qagltqpp | 43 | agts | 52 | wyqqlpgm | 59 | svnkras | 66 | giadrfsgsksgntaslti | 74 | gcyd | 82 | fgggtkltvlg | 87 |
| | svsgspgk | | sdvg | | apkllly | | | | sglqsedeadyyc | | sslstpv | | | |
| | tvtisc | | ygn | | | | | | | | | | | |
| | | | yvs | | | | | | | | | | | |
| 2D11 | syeltqs | 44 | ggdn | 53 | wyqqkpgqs | 60 | kdsnrps | 67 | giperfsgsnsgntatltv | 75 | qvwd | 83 | fgggthltvlg | 86 |
| | psvsvalr | | iask | | pvlviy | | | | sgaqaedeadyyc | | av | | ssana | |
| | qaak | | hah | | | | | | | | | | | |
| | itc | | | | | | | | | | | | | |
| 6H8 | diqmtqsp | 45 | kssq | 54 | wyqqrlgqs | 61 | wastres | 68 | gvpdrfsgsgsttdftlti | 76 | qqgy | 84 | fgqgtkvelk | 88 |
| | ssvtasag | | svlys | | prlliy | | | | nnfqpedaavyyc | | saplt | | | |
| | ekvtinc | | snqk | | | | | | | | | | | |
| | | | nyla | | | | | | | | | | | |

TABLE 7
Variable Domain Sequences for peptide-raised Nav1.7 antibodies

| | | | SEQ ID |
|---|---|---|---|
| Clone | VH | | NO. |
| 3F11 | qvqlvesggglvqpggslrlscaasgftfssywmywvrqapgkglewvsaintgggstyy | | 218 |
| | adsvkgrftisrdnakntlylqmnslkpedtalyycargpgysgsyiegwaywgqgtqvt | | |
| | vss | | |
| 2F12 | qvqlvesggglvqpggslrlscaasgftfssywmywvrqapgkglewvsaintgggstyy | | 219 |
| | adsvkgrftisrdnakntlylqmnslkpedtalyycargpgysgkyiegwaywgqgtqvt | | |
| | vss | | |
| 3E1 | evqvqesggglvqpggslrlscaasgftfssywmywvrqapgkglewvsaintgggsty | | 220 |
| | yadsvkgrftisrdnakntlylqmnslkpedtalyycargpgysgkyiegwaywgqgtq | | |
| | vtvss | | |
| 4E8 | evqlqesggglvqpggslrlscaasgftfddyamswvrqapgkglewvsaiswnggsty | | 221 |
| | yaesmkgrftisrdnakntlylqmnslkskdtavyycarrryglgseydywgqgtqvtvss | | |
| 4F6 | qlqlvesggglvqpggslrlscaasgftfddyamswvrqapgkglewvsaiswnggstyy | | 222 |
| | aesmkgrftisrdnakntlylqmnslkskdtavyycarrryglgseydywgqgtqvtvss | | |
| 4F7 | qvqlqesgpglvkpsqtlsltctvsggsittsfyawswirqppgkglewmgviaydgrtfy | | 223 |
| | spslksrthisrdtsknqfslqlssvtpedtavyycarggvvawsydywgqgtqvtvss | | |
| 4H6 | qvqlqesgpglvkpsqtlsltctvsggsitnygwswirqppgkglewmgviaydgstyys | | 224 |
| | pslksrtsisrdtsknqfslqlssvtpedtavyycasamvttsspwvktfgawgqgtqvtv | | |
| | ss | | |
| 2D11 | evqlqesgpglvkpsqtlsltctvsggsitnsyyasswirqppgkglewmgviiydgstyy | | 225 |
| | spslksrtsisrdtsknqfslqlssvtpedtavyycarglrvagsyftdfgtwgqgtqvtvss | | |
| 6H8 | evqlvesggglyqpggtlrlscaasgftfsnywmhwvrqapgkglewvsvidrggggtry | | 226 |
| | adsvkgrftisrdnakntlylrmnslkpedtalyyctrdrngeydywgrgtqvtvss | | |
| | | | SEQ ID |
| | Clone | VL | NO. |
| | 3F11 | divmtqsptsvtasvgekvtinckssqsvvsgsnqksylnwyqqrpgqsprlli | 227 |
| | | yhastqesgipdrfsgsgsttdftltissvqpedaavyycqqaynnpysfgsgtr | |
| | | leik | |
| | 2F12 | divmtqsprsvtasvgekvfinckssqsvvsesnqrsylnwyqqrpgqsprlli | 228 |
| | | yyastqesglpdrfsgsgsttdftltissvqpedaavyycqqaysapysfgsgtrl | |
| | | eik | |
| | 3E1 | divmtqspssvtasvgekvtinckssqsvvsgskqksylnwyqqrpgqsprlli | 229 |
| | | yyastqesgipdrfsgsgsttdftltissvqpedaavyycqqaysapysfgsgtrl | |
| | | eik | |
| | 4E8 | nfmltqpsavsvslgqtaritcqgssfgssyahwyqqmpgqapilviyrdser | 230 |
| | | psgipdrfsgsnsggtatltisgaqaedeadyycqsgsssgnahavfgggthltv | |
| | | lg | |
| | 4F6 | hsavtqpsavsvslgqtaritcqgtnlrssyvhwyqqmpgqapvlvivrdser | 231 |
| | | psgiperfsgsssggtatlaisgaqaedeadyycqsahrsesavfgggthltvlg | |
| | 4F7 | syeltqspsvsvalrqtakitcggndigskstqwyqqkagqapylviyadsrrps | 232 |
| | | giperfsgsnsgntatltisgaqaedeadyycqvwdsradaavfgggthltvlg | |
| | 4H6 | qagltqppsvsgspgktvtiscagtssdvgygnyvswyqqlpgmapklllysv | 233 |
| | | nkrasgiadrfsgsksgntasltisglqsedeadyycgcydsslstpvfgggtkltv | |
| | | lg | |
| | 2D11 | syeltqspsvsvalrqaakitcggdniaskhahwyqqkpgqspylviykdsnr | 234 |
| | | psgiperfsgsnsgntatltvsgaqaedeadyycqvwdssanaavfgggthlt | |
| | | vlg | |
| | 6H8 | diqmtqspssvtasagekvtinckssqsvlyssnqknylawyqqrlgqsprlliy | 235 |
| | | wastresgvpdrfsgsgsttdftltinnfqpedaavyycqqgysapltfgqgtkv | |
| | | elk | |

### Example 3

### Binding Affinity for Nav1.7 Extracellular Loops

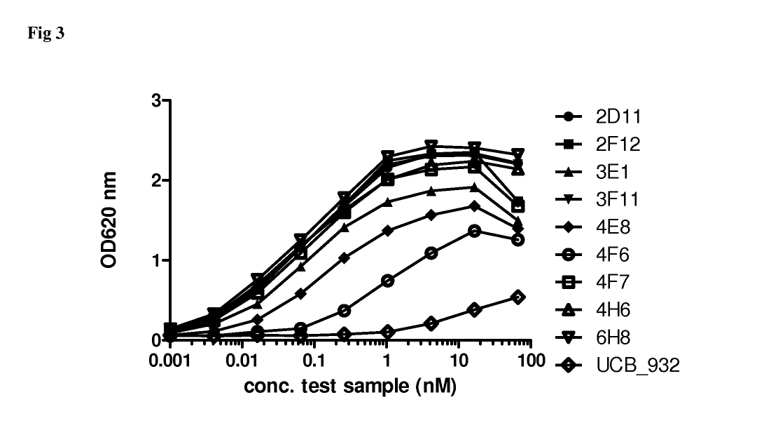

mAbs originating from the loopA3-llama Fc immunization were assayed for their affinity for hNav1.7-loopA3. Therefore, a Maxisorp plate was coated with 10 ng/well of loopA3-llamaFc chimera and incubated overnight at 4° C. The next day, the plate was blocked with PBS+1% casein for 2 hours at RT. Subsequently a concentration gradient of antibodies was applied (range 0.25 pM-66 nM) for another hour. Antibody binding to loopA3 was detected using a HRP-conjugated anti-human Fc antibody (Abcam, Ab7499) (incubation 1 hour at RT, dilution 1/5000 in PBS+0.1% casein), followed by TMB addition and OD620 nm measurement. EC50 values were determined using GraphPad Prism software. Results are shown in FIG. 3 and Table 8. For comparison purposes a known hNav1.7-loopA3 binding monoclonal antibody, UCB_932 (US2011/0135662), was tested. All mAbs have a much improved affinity for hNav1.7-loopA3 compared to the reference mAb and EC50 values range between 57 pM and 874 pM except for the UCB_932 whose EC50 was too low to be determined.

TABLE 8
Affinity expressed as EC₅₀ (pM) for hNav1.7 loopA3 for individual
Loop-immunization-derived antibodies as measured in a binding ELISA

| | 2D11 | 2F12 | 3E1 | 3F11 | 4E8 | 4F6 | 4F7 | 4H6 | 6H8 | UCB_932 |
|---|---|---|---|---|---|---|---|---|---|---|
| EC50 | 71 | 57 | 63 | 72 | 129 | 874 | 57 | 67 | 66 | nd |
| (pM) | | | | | | | | | | |

In a further experiment, affinity of the loopA3-binding mAbs was determined using Biacore Surface Plasmon Resonance (SPR) analysis. A CM5 chip was coated with ˜100 RU of Nav1.7 loopA3-llama Fc or Nav1.7-loopA3-GST ((SEQ ID NO: 272, see Table 4). As a negative control, GCGR-Nt-llama Fc was used (data not shown). Immobilization was performed in accordance with the method provided by Biacore/GE and using the NHS/EDC kit (Biacore AB). Antibodies were diluted in HEPES-buffered saline (0.1M HEPES, 1.5M NaCl, 30 mM EDTA, 0.5% v/v surfactant P20) and binding of each mAb was measured at 6 different concentrations (0.3125-10 μg/ml). After binding of the mAb to loopA3, dissociation was monitored for a period of 10 minutes. Off-rate and K_{D} value were calculated using the Fit kinetics application of the BIAevalution software and are summarized in Table 9.

TABLE 9
Overview of off-rates and K_{D} values
of selected anti- Nav1.7-loopA3 mAbs

| | |
|---|---|
| hNav1.7-loopA3-llFc | hNav1.7-loopA3-GST |

| | Off-rate | Kd | Off-rate | Kd |
|---|---|---|---|---|
| Clone | (10⁻⁴ s⁻¹) | (10⁻¹⁰ M) | (10⁻⁴ s⁻¹) | (10⁻¹⁰ M) |
| 3F11 | 0.156 | 0.253 | 0.177 | 0.374 |
| 2F12 | 0.192 | 0.241 | 0.108 | 0.169 |
| 3E1 | 4.1 | 2 | 3.56 | 2.16 |
| 4F6 | 42.2 | 51 | 25.4 | 33.5 |
| 4E8 | 4.22 | 42.8 | 6.3 | 452 |
| 4F7 | 0.268 | 0.84 | 0.184 | 0.825 |
| 4H6 | 0.448 | 1.2 | 0.154 | 0.505 |
| 2D11 | 0.126 | 0.375 | 0.213 | 0.879 |
| 6H8 | 0.0358 | 0.0181 | 0.0069 | 0.00609 |

### Example 4

### Nav1.7 Immune Precipitation

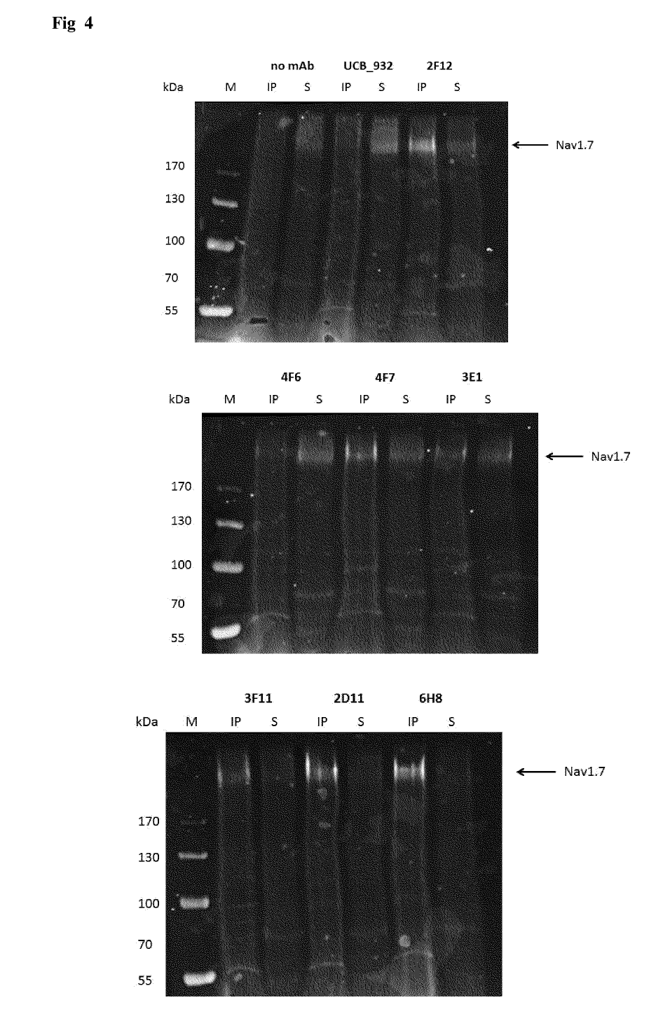

Inducible hNav1.7-HEK293 cells (BPS Bioscience, CA; cat. nr. 60507) were induced for 24 hrs with 1 μg/ml doxycycline and 3 mM Na-butyrate. Cells were harvested, washed with PBS and lysed in RIPA buffer (Sigma, cat. nr. R0278) at a density of 1×10⁷ cells per ml.
Aliquots of the cleared lysate (40 μl each) were incubated with 2 μg of each mAb, for 2 hours at 4° C. in a rotator. Protein A beads (GE Healthcare, cat nr. 17-5138-01) were added (15 μl bead volume) and the lysate was further incubated overnight. After adding extra 180 μl RIPA buffer, the beads were pelleted by centrifugation, the supernatant was stored for analysis, and the beads were washed extensively with RIPA buffer. The bead pellets were resuspended in 30 μl SDS-PAGE sample buffer and boiled. In parallel, 30 ul samples of the supernatants were supplemented with SDS-PAGE sample buffer and boiled. The samples were size-separated on 6% SDS-polyacrylamide gels, blotted to nitrocellulose membranes and stained with a mouse anti-hNav1.7 antibody (Millipore, clone N68/6). After secondary staining with donkey anti-mouse IR800CW (Li-Cor), the blots were imaged in a Li-Cor Odyssey IR imaging system at 700 and 800 nm.
Corresponding immune precipitate (IP) and supernatant (S) from the same mAb are loaded in adjacent positions on the SDS-PAGE gels. A PAGE-ruler prestained protein marker (M) (Fermentas) was used for reference. As shown in FIG. 4, a number of mAbs are able to precipitate Nav1.7 from the cell lysate, as shown by the presence of Nav1.7 in the precipitate fraction. These mAbs include 2F12, 4F7, 3E1, 3F11, 2D11 and 6H8. No Nav1.7 was found in the precipitate fraction of UCB_932, or when no mAb was used.

### Nav1.7 Antibodies Generated by DNA Immunization

### Example 5

### Immunization of Llama

Four llamas were immunized by DNA vaccination followed by a cell boost. Therefore, llamas were injected with 1 ml pCMV6-hNav1.7 expression vector (Origene, SC309017, concentration 2 mg/ml) at 4 different sites (hip and shoulders, left and right) aiming at 4 different draining lymph nodes. At each of the 4 sites, 250 μl plasmid was injected intradermally at 8 spots. Directly thereafter the electric pulse was given at each spot. The pulses are at 450V with a resistance below 3,000 ohm. This procedure was executed 4 times in a two-weekly interval. Four weeks after the last DNA immunization, llamas were boosted with 2.10⁷ hNav1.7-expressing HEK293 cells. Cells were injected subcutaneously, 1 ml at each of the four sites where the DNA injections have been taken place.

In total 8 mg of DNA per animal was used, and the appropriate E. coli strains capable of replicating the pCMV6-hNav1.7 plasmid were tested. Therefore, 10 ng vector was transformed in a common E. coli strain (XL-10 Gold, Agilent) and in the Copycutter E. coli strain (Epicentre, C400CH10). Miniprep DNA preparation was done of some resulting bacterial clones and successful replication of the pCMV6-hNav1.7 plasmid was verified using DNA restriction enzyme digest analysis. A representative analysis of both transformations (either in XL-10 Gold (“Gold”) or in Copycutter E. coli cells (“CC”)) is shown in FIG. 5. Herein, a restriction digest analysis was done using restriction enzymes EcoRI or HindIII followed by a DNA gel electrophoresis. The EcoRI digest should result in band sizes of 815 bp, 1127 bp, 1740 bp, 2667 bp, 5869 bp and the HindIII digest in expected band sizes of 811 bp, 1764 bp, 2822 bp, 6821 bp respectively. FIG. 5 demonstrates that transformation of pCMV6-hNav1.7 in XL-10 gold cells does not lead to the correct DNA fragment pattern whereas the transformation in Copycutter cells does. This suggests the DNA instability generated by the Nav1.7 cDNA can be limited by using the Copycutter E. coli strain and not by using others. Similar DNA instability was observed using NEB-5alpha or TOP10 E. coli strains even though these strains are known to be very good strains for the conservation of plasmid DNA. As a result, all pCMV6-hNav1.7 DNA used for immunization was made using the Copycutter E. coli strain and plasmid preps were done according to manufacturer's instructions. Prior to immunization, DNA was checked for the correct insert sequence and restriction digest analysis was performed to verify plasmid integrity. In addition, DNA was assayed for functionality using manual patch clamp electrophysiology.

Four days after the last immunization, 400 ml blood was collected for extraction of total RNA from the PBLs using a Ficoll-Paque gradient to isolate PBLs and the method described by Chomczynski P., et al., Anal. Biochem. 162: 156-9, 1987 to prepare the RNA. On average, a few 100 g was extracted and aliquoted, prior to use for random primed cDNA synthesis and subsequent PCR amplification of the llama VHCH1, VλCλ and VκCκ gene segments.

### Example 6

### Library Construction, Selection and Screening

Fab fragments binding to human hNav1.7-loopA3, hNav1.7-LoopB1-C1-D1 or hNav1.7-LoopC3 (see Table 4) were selected, and antibodies were generated as described in Example 2.

TABLE 10
Framework regions and CDR sequences for VH domains of Nav1.7
antibodies raised by DNA immunization

| | | SEQ | | SEQ | | SEQ | | SEQ | | SEQ | | SEQ | | SEQ |
|---|---|---|---|---|---|---|---|---|---|---|---|---|---|---|
| | | ID | | ID | | ID | | ID | | ID | | ID | | ID |
| Clone | FR1 | NO. | CDR1 | NO. | FR2 | NO. | CDR2 | NO. | FR3 | NO. | CDR3 | NO. | FR4 | NO. |
| 8A7 | evqv | 89 | nnyy | 100 | wirqppgk | 110 | aiaysgstyysp | 119 | rtsisrdtsknqftlqlssvtp | 130 | vpsv | 141 | wgkgtlvt | 150 |
| | qesg | | ywn | | glewmg | | slqs | | edtavyycar | | fglp | | vss | |
| | pglvt | | | | | | | | | | ygg | | | |
| | psqtl | | | | | | | | | | mny | | | |
| | sltctv | | | | | | | | | | | | | |
| | sggs | | | | | | | | | | | | | |
| | ik | | | | | | | | | | | | | |
| 8B6 | evql | 90 | tsgt | 101 | wirqppgk | 110 | iigydggtyynp | 120 | rtsisrdtsknqfslqltsvtp | 131 | glgp | 142 | wgqgiqvt | 151 |
| | qesg | | gws | | glewmg | | slks | | edtavyycar | | | | vss | |
| | pglv | | | | | | | | | | | | | |
| | kpsq | | | | | | | | | | | | | |
| | tlsl | | | | | | | | | | | | | |
| | tctv | | | | | | | | | | | | | |
| | sgg | | | | | | | | | | | | | |
| | sis | | | | | | | | | | | | | |
| 9A1 | evql | 91 | dyyih | 102 | wvrqapg | 111 | ridpedggtkyt | 121 | rvtftadtststvyleldslrse | 132 | dmdy | 143 | wgkgtlvt | 150 |
| | vqpg | | | | qglewmg | | qkfqg | | dtavyycvg | | | | vss | |
| | aelrn | | | | | | | | | | | | | |
| | pgas | | | | | | | | | | | | | |
| | vkvsc | | | | | | | | | | | | | |
| | kasg | | | | | | | | | | | | | |
| | dtft | | | | | | | | | | | | | |
| 9A6 | evql | 92 | sayiv | 103 | wvrqapg | 112 | ridpedggtky | 122 | rvtftadtstntayvelsslrs | 133 | gldy | 144 | wgkgtlvt | 150 |
| | vqpg | | | | eglewmg | | aqkfqg | | ddtavyycta | | | | vss | |
| | aelr | | | | | | | | | | | | | |
| | npga | | | | | | | | | | | | | |
| | svkv | | | | | | | | | | | | | |
| | scka | | | | | | | | | | | | | |
| | sgytyt | | | | | | | | | | | | | |
| 9A11 | evql | 93 | ssyid | 104 | wvrqapg | 111 | ridpedggtsya | 123 | rvtvtvdtatstayvelsslrs | 134 | gdsgy | 145 | wgqgtqvt | 152 |
| | vqpg | | | | qglewmg | | qkfqg | | ddtgvyfcmv | | | | vss | |
| | aevr | | | | | | | | | | | | | |
| | rpga | | | | | | | | | | | | | |
| | avkv | | | | | | | | | | | | | |
| | sckas | | | | | | | | | | | | | |
| | gfali | | | | | | | | | | | | | |
| 9C3 | evql | 94 | ssdiq | 105 | wvrqapa | 113 | ridpedgatky | 124 | rvtftadtftrtayvelsnlrs | 135 | aggey | 146 | wgkgtivt | 150 |
| | vqpg | | | | qglewmg | | apkfqg | | ddtavyycmt | | | | vss | |
| | aelr | | | | | | | | | | | | | |
| | npgt | | | | | | | | | | | | | |
| | svkv | | | | | | | | | | | | | |
| | sckt | | | | | | | | | | | | | |
| | sgyy | | | | | | | | | | | | | |
| | ft | | | | | | | | | | | | | |
| 9C12 | evql | 95 | ssyid | 106 | wvrqapg | 114 | ridpedggtsya | 125 | rvtvtvdtatstvyvelsslrs | 136 | gdsgy | 145 | wgqgtqvt | 152 |
| | vqpg | | | | qglewmg | | qkfqg | | ddtgvyfcmv | | | | vss | |
| | aevr | | | | | | | | | | | | | |
| | rpga | | | | | | | | | | | | | |
| | avkv | | | | | | | | | | | | | |
| | sckas | | | | | | | | | | | | | |
| | gfali | | | | | | | | | | | | | |
| 9F10 | evql | 95 | ssyid | 106 | wvrqapg | 114 | ridpedggtsya | 125 | rvtvtvdtatstvyvelsslrs | 136 | gdsgy | 145 | wgqgtqvt | 152 |
| | vqpg | | | | qglewmg | | qkfqg | | ddtgvyfcmv | | | | vss | |
| | asgf | | | | | | | | | | | | | |
| | aliae | | | | | | | | | | | | | |
| | vrrpg | | | | | | | | | | | | | |
| | aavk | | | | | | | | | | | | | |
| | vsck | | | | | | | | | | | | | |
| 9G6 | evql | 96 | gyyid | 107 | wvrqvpg | 115 | ridpedgdtky | 126 | rvtitadtstttaymelsslrs | 137 | gvdy | 147 | wgqgtqvt | 152 |
| | vqpg | | | | eglewmg | | aqkfqg | | edtavyycsl | | | | vss | |
| | aelt | | | | | | | | | | | | | |
| | kpga | | | | | | | | | | | | | |
| | svkv | | | | | | | | | | | | | |
| | scnt | | | | | | | | | | | | | |
| | sgytft | | | | | | | | | | | | | |
| 9H4 | evql | 97 | sayid | 108 | wvrqapg | 116 | ridpedgetrya | 127 | rvtftadtststvyaelnslrs | 138 | tiegf | 148 | wgqgtqvt | 152 |
| | vqpg | | | | qglewmg | | hqfrd | | edtavyycai | | | | vss | |
| | aelr | | | | | | | | | | | | | |
| | npga | | | | | | | | | | | | | |
| | svkvs | | | | | | | | | | | | | |
| | ckas | | | | | | | | | | | | | |
| | gyift | | | | | | | | | | | | | |
| 10B6 | qlql | 98 | fsey | 109 | wvrqapg | 117 | dinsggeitaya | 128 | rftvsrdntkntvylqmnsl | 139 | gsfga | 149 | wgqgtqvt | 152 |
| | vesg | | vms | | kglewvs | | dsvkg | | kpedtavyycaq | | | | vss | |
| | gglv | | | | | | | | | | | | | |
| | qpgg | | | | | | | | | | | | | |
| | slrls | | | | | | | | | | | | | |
| | caas | | | | | | | | | | | | | |
| | gftfs | | | | | | | | | | | | | |
| 10C4 | evql | 99 | fsey | 109 | wvrqapg | 118 | dinsggeitaya | 129 | rftvsrdntkntvylqmnsl | 140 | gsfga | 149 | wgqgtqvt | 152 |
| | vesg | | vms | | kglewvs | | dsvkg | | kpedtavyycaq | | | | vss | |
| | gglv | | | | | | | | | | | | | |
| | qpgg | | | | | | | | | | | | | |
| | slrls | | | | | | | | | | | | | |
| | caas | | | | | | | | | | | | | |
| | gftfs | | | | | | | | | | | | | |
| 7B9 | evqv | 288 | dkss | 289 | wirqppgk | 290 | virydgntrysp | 291 | rttisrdtsknqislqltsvtp | 292 | eitl | 293 | wgqgtqvt | 294 |
| | qesg | | aws | | glewmg | | tlks | | ddtavyycsr | | | | vss | |
| | pglv | | | | | | | | | | | | | |
| | kps | | | | | | | | | | | | | |
| | qtls | | | | | | | | | | | | | |
| | ltctv | | | | | | | | | | | | | |
| | sga | | | | | | | | | | | | | |
| | sia | | | | | | | | | | | | | |

TABLE 11
Framework regions and CDR sequences for VL domains of Nav1.7
antibodies raised by DNA immunization

| | | SEQ | | SEQ | | SEQ | | SEQ |
|---|---|---|---|---|---|---|---|---|
| | | ID | | ID | | ID | | ID |
| Clone | FR1 | NO. | CDR1 | NO. | FR2 | NO. | CDR2 | NO. |
| 8A7 | syeltqspsvtvalrq | 153 | ggsrigsksvq | 161 | wyqqkpgqvp | 173 | adnrrps | 181 |
| | takitc | | | | vlviy | | | |
| 8B6 | diqmtqspsslsasl | 154 | qasqgiskyla | 162 | wyqqkpgqap | 174 | sasrlet | 182 |
| | gdrvtitc | | | | klliy | | | |
| 9A1 | qtyvtqepslsyspg | 155 | glssgsvtfgn | 163 | wyqqtpgqap | 175 | ttnsrhs | 183 |
| | gtvtltc | | yps | | rtliy | | | |
| 9A6 | atmltqspgslsvvp | 156 | kasqslvhsd | 164 | wlqqkpgqsp | 176 | rvsrrgs | 184 |
| | gesasisc | | gmtyly | | qrliy | | | |
| 9A11 | atmltqspgslsvvp | 156 | kasqslyhtd | 165 | wllqkpgqspq | 177 | qvstrgs | 185 |
| | gesasisc | | gktyls | | rliy | | | |
| 9C3 | qpvlnqpsalsyslg | 157 | qggdfrnyy | 166 | wyqqkpgqap | 178 | edserps | 186 |
| | qtaritc | | nn | | vlviy | | | |
| 9C12 | atmltqspaslsvvp | 158 | kasqslvhsd | 167 | wllqkpgqspq | 177 | qvsnrgs | 187 |
| | gesasisc | | gktyly | | rliy | | | |
| 9F10 | atmltqspgslsvgp | 159 | kagqslthpn | 168 | wllqkpgqsprr | 179 | etsnrdp | 188 |
| | gesasisc | | gktyls | | liy | | | |
| 9G6 | atmltqspgslsvvp | 156 | ktsqslvhsd | 169 | wllqkpgqspq | 177 | qvsnrds | 189 |
| | gesasisc | | gktyly | | rliy | | | |
| 9H4 | atmltqspgslsvvp | 156 | ktsrslvhsdg | 170 | wllqkpgqspq | 177 | qvsnrgs | 190 |
| | gesasisc | | ktyls | | rliy | | | |
| 10C4 | atmltqspgslsvvp | 156 | kasqslvhsd | 171 | wlrqkpgqspq | 180 | qvsrrds | 191 |
| | gesasisc | | gktyly | | rliy | | | |
| 10B6 | atmltqspgslsvvp | 160 | kanesivhpg | 172 | wllqkpgqspq | 177 | qvsnras | 192 |
| | gesatiac | | gktyly | | rliy | | | |
| 7B9 | atmltqspgslsvvp | 295 | kasqslvhsd | 296 | wllqkpgqspq | 297 | qvsnrgs | 298 |
| | gesasisc | | gktyly | | rliy | | | |

| | | SEQ | | SEQ | | SEQ |
|---|---|---|---|---|---|---|
| | | ID | | ID | | ID |
| Clone | FR3 | NO. | CDR3 | NO. | FR4 | NO. |
| 8A7 | riperfsgsnsgntatliisgaqaed | 193 | qvwdssaav | 204 | fgggthltvlg | 216 |
| | eadyyc | | | | | |
| 8B6 | gvpsrfsgsgsgttftivisgleaeda | 194 | qqyyrapat | 205 | fgqgtkvelk | 217 |
| | gsyyc | | | | | |
| 9A1 | gvpsrfsgsisgnkatltisgaqped | 195 | alsrvsgtygtv | 206 | fgggthltvlg | 216 |
| | eadfyc | | | | | |
| 9A6 | gvpdrftgsgsgtdftlnisdmkae | 196 | aqdtyypys | 207 | fgsgtsveik | 218 |
| | dagvyyc | | | | | |
| 9A11 | gvpdrvtgsgsgtdftlkisgvkae | 197 | aqttyyppt | 208 | fgqgtkvelk | 217 |
| | dagvyyc | | | | | |
| 9C3 | giperfsgsssgdtviltvsrvqadd | 198 | qssdstdnav | 209 | fgggthltvlg | 216 |
| | eadyyc | | | | | |
| 9C12 | gvpdrftgsgsgtdftlkisgvkaed | 199 | aqhtyypps | 210 | fgsgtrleik | 219 |
| | agvyyc | | | | | |
| 9F10 | gvpdrftgsgsgtdftlrivgvtved | 200 | aqttyfpia | 211 | fgqgtkvelk | 217 |
| | agvyyc | | | | | |
| 9G6 | gvpdrftgsgsgtdftlkisgvkaed | 199 | aqttydpvt | 212 | fgqgtkvelk | 217 |
| | agvyyc | | | | | |
| 9H4 | gvpdrftgsgsgtdftlkisgvkaed | 201 | qatyypls | 213 | fgsgtrveik | 220 |
| | agvyyca | | | | | |
| 10C4 | gvpdrftgsgsgtdftlkisgvkaed | 202 | aqatyvplg | 214 | fgsgtrleik | 219 |
| | aglyyc | | | | | |
| 10B6 | gvpdrftgsgsgtdftlkisglkaed | 203 | aqatfkkit | 215 | fgqgtkvelk | 217 |
| | agvyyc | | | | | |
| 7B9 | gvpdrftgsgsgtdftlkisgvkaed | 299 | aqatyypt | 300 | fgqgtkvelk | 301 |
| | agvyyc | | | | | |

TABLE 12
Variable Domain Sequences for Nav1.7 antibodies raised
by DNA Immunization

| | | SEQ |
|---|---|---|
| | | ID |
| Clone | VH | NO. |
| 8A7 | evqvqesgpglvtpsqtlsltctvsggsiknnyyywnwirqppgkglewmgaiaysgstyy | 236 |
| | spslqsrtsisrdtsknqftlqlssvtpedtavyycarypsvfglpyggmnywgkgtlvtvss | |
| 8B6 | evqlqesgpglvkpsqtlsltctvsggsistsgtgwswirqppgkglewmgiigydggtyyn | 237 |
| | pslksrtsisrdtsknqfslqltsvtpedtavyycarglgpwgqgiqvtvss | |
| 9A1 | evqlvqpgaelrnpgasvkvsckasgdtftdyyihwvrqapgqglewmgridpedggtk | 238 |
| | ytqkfqgrvtftadtststvyleldslrsedtavyycvgdmdywgkgtlvtvss | |
| 9A6 | evqlvqpgaelrnpgasvkvsckasgytytsayivwvrqapgeglewmgridpedggtky | 239 |
| | aqkfqgrvtftadtstntayvelsslrsddtavyyctagldywgkgtlvtvss | |
| 9A11 | evqlvqpgaevrrpgaavkvsckasgfalissyidwvrqapgqglewmgridpedggtsy | 240 |
| | aqkfqgrvtvtvdtatstayvelsslrsddtgvyfcmygdsgywgqgtqvtvss | |
| 9C3 | evqlvqpgaelrnpgtsvkvscktsgyyftssdiqwvrqapaqglewmgridpedgatky | 241 |
| | apkfqgrvtftadtftrtayvelsnlrsddtavyycmtaggeywgkgtlvtvss | |
| 9C12 | evqlvqpgaevrrpgaavkvsckasgfalissyidwvrqapgqglewmgridpedggtsy | 242 |
| | aqkfqgrvtvtvdtatstvyvelsslrsddtgvyfcmygdsgywgqgtqvtvss | |
| 9F10 | evqlvqpgaevrrpgaavkvsckasgfalissyidwvrqapgqglewmgridpedggtsy | 243 |
| | aqkfqgrvtvtvdtatstvyvelsslrsddtgvyfcmygdsgywgqgtqvtvss | |
| 9G6 | evqlvqpgaeltkpgasvkvscntsgytftgyyidwvrqvpgeglewmgridpedgdtky | 244 |
| | aqkfqgrvtitadtstttaymelsslrsedtavyycslgvdywgqgtqvtvss | |
| 9H4 | evqlvqpgaelrnpgasvkvsckasgyiftsayidwvrqapgqglewmgridpedgetry | 245 |
| | ahqfrdrvtftadtststvyaelnslrsedtavyycaitiegfwgqgtqvtvss | |
| 10C4 | evqlvesggglvqpggslrlscaasgftfsfseyvmswvrqapgkglewvsdinsggeitay | 246 |
| | adsvkgrftvsrdntkntvylqmnslkpedtavyycaqgsfgawgqgtqvtvss | |
| 10B6 | qlqlvesggglvqpggslrlscaasgftfsfseyvmswvrqapgkglewvsdinsggeitaya | 247 |
| | dsvkgrftvsrdntkntvylqmnslkpedtavyycaqgsfgawgqgtqvtvss | |
| 7B9 | evqvqesgpglvkpsqtlsltctvsgasiadkssawswirqppgkglewmgvirydgntry | 302 |
| | sptlksrttisrdtsknqislqltsvtpddtavyycsreitlwgqgtqvtvss | |

| | | SEQ |
|---|---|---|
| | | ID |
| Clone | VL | NO. |
| 8A7 | syeltqspsytvalrqtakitcggsrigsksvqwyqqkpgqvpvlviyadnrrpsriper | 248 |
| | fsgsnsgntatliisgaqaedeadyycqvwdssaavfgggthltvlg | |
| 8B6 | diqmtqspsslsaslgdrvtitcqasqgiskylawyqqkpgqapklliysasrletgvps | 249 |
| | rfsgsgsgttftivisgleaedagsyycqqyyrapatfgqgtkvelk | |
| 9A1 | qtvvtqepslsvspggtvtltcglssgsvtfgnypswyqqtpgqaprtliyttnsrhsgv | 250 |
| | psrfsgsisgnkatltisgaqpedeadfycalsrvsgtygtvfgggthltvlg | |
| 9A6 | atmltqspgslsvvpgesasisckasqslvhsdgmtylywlqqkpgqspqrliyrvsrrg | 251 |
| | sgypdrftgsgsgtdftlnisdmkaedagvyycaqdtyypysfgsgtsveik | |
| 9A11 | atmltqspgslsvvpgesasisckasqslvhtdgktylswllqkpgqspqrliyqvstrg | 252 |
| | sgypdrvtgsgsgtdftlkisgvkaedagvyycaqttyypptfgqgtkvelk | |
| 9C3 | qpvlnqpsalsvslgqtaritcqggdfrnyynnwyqqkpgqapvlviyedserpsgiper | 253 |
| | fsgsssgdtviltvsrvqaddeadyycqssdstdnavfgggthltvlg | |
| 9C12 | atmltqspaslsvvpgesasisckasqslvhsdgktylywllqkpgqspqrliyqvsnrg | 254 |
| | sgypdrftgsgsgtdftlkisgvkaedagvyycaqhtyyppsfgsgtrleik | |
| 9F10 | atmltqspgslsvgpgesasisckagqslthpngktylswllqkpgqsprrliyetsnrd | 255 |
| | pgypdrftgsgsgtdftlrivgvtvedagvyycaqttyfpiafgqgtkvelk | |
| 9G6 | atmltqspgslsvvpgesasiscktsqslvhsdgktylywllqkpgqspqrliyqvsnrd | 256 |
| | sgypdrftgsgsgtdftlkisgvkaedagvyycaqttydpvtfgqgtkvelk | |
| 9H4 | atmltqspgslsvvpgesasiscktsrslvhsdgktylswllqkpgqspqrliyqvsnrg | 257 |
| | sgypdrftgsgsgtdftlkisgvkaedagvyycaqatyyplsfgsgtrveik | |
| 10C4 | atmltqspgslsvvpgesatiackanesivhpggktylywllqkpgqspqrliyqvsnra | 258 |
| | sgypdrftgsgsgtdftlkisglkaedagvyycaqatfkkitfgqgtkvelk | |
| 10B6 | atmltqspgslsvvpgesasisckasqslvhsdgktylywlrqkpgqspqrliyqvsrrd | 259 |
| | sgypdrftgsgsgtdftlkisgvkaedaglyycaqatyvplgfgsgtrleik | |
| 7B9 | atmltqspgslsvvpgesasisckasqslvhsdgktylywllqkpgqspqrliyqvsnrg | 303 |
| | sgvpdrftgsgsgtdftlkisgvkaedagvyyc aqatyypt fgqgtkvelk | |

### Example 7

### Specificity of Nav1.7 Antibody Binding for Particular Extracellular Loops

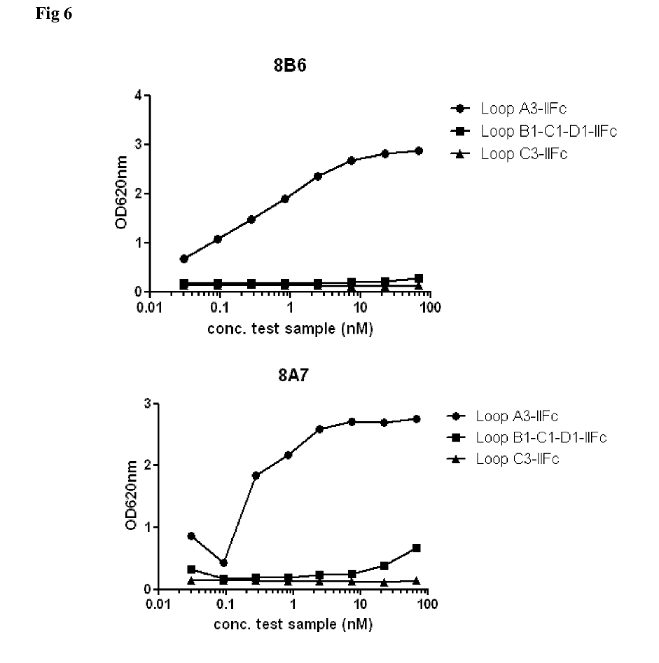

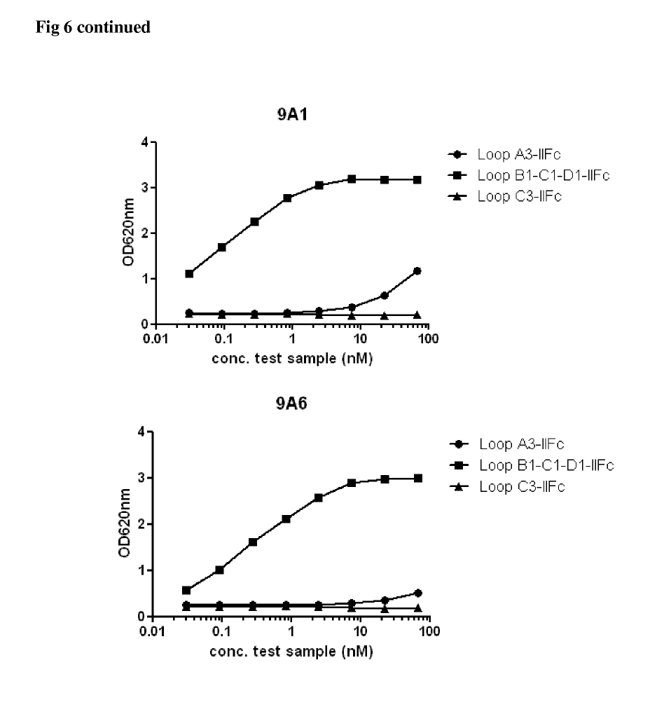

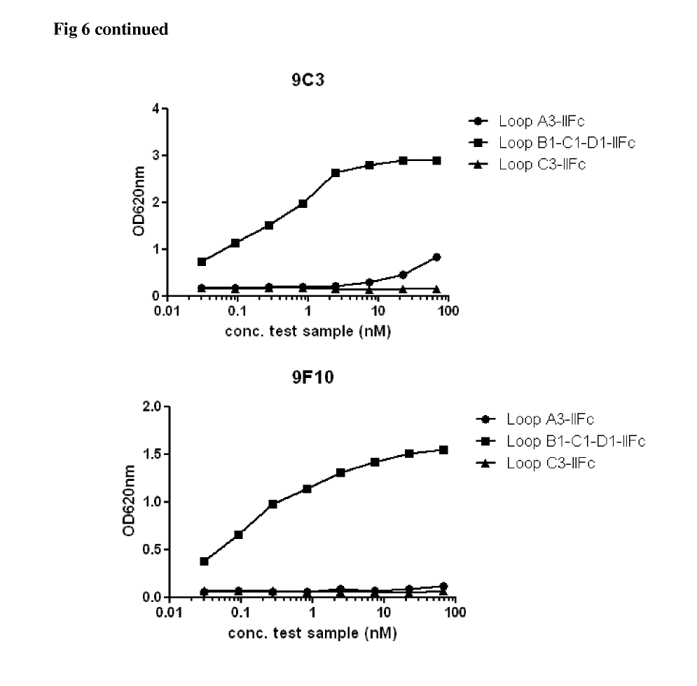

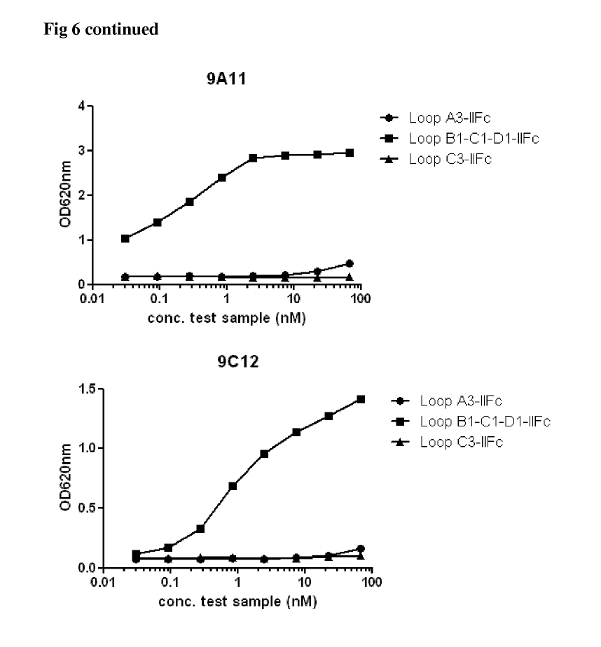

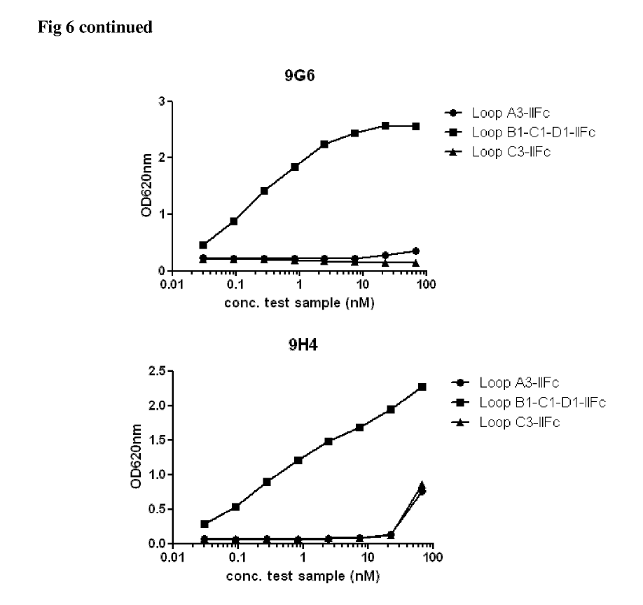

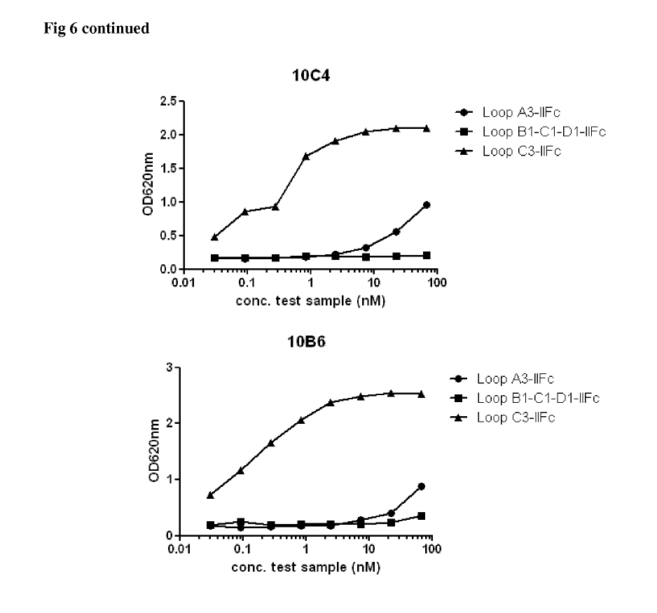

For all DNA-immunization derived mAbs the binding affinity for the various hNav1.7-loop-llama Fc chimeras was tested in a binding ELISA. Therefore, a Maxisorp plate (Nunc) was coated with 20 ng/well of hNav1.7-loopA3-llama Fc, hNav1.7-loopB1-C1-D1-llama Fc or hNav1.7-loopC3-llama Fc. Remainder of the ELISA setup is as described above. FIG. 6 shows that all DNA-derived mAbs are highly specific for the particular loop on which they were selected. Only at very high concentration minor cross-reactivity is observed for some clones.

### Example 8

### Binding Affinity for Nav1.7 Extracellular Loops

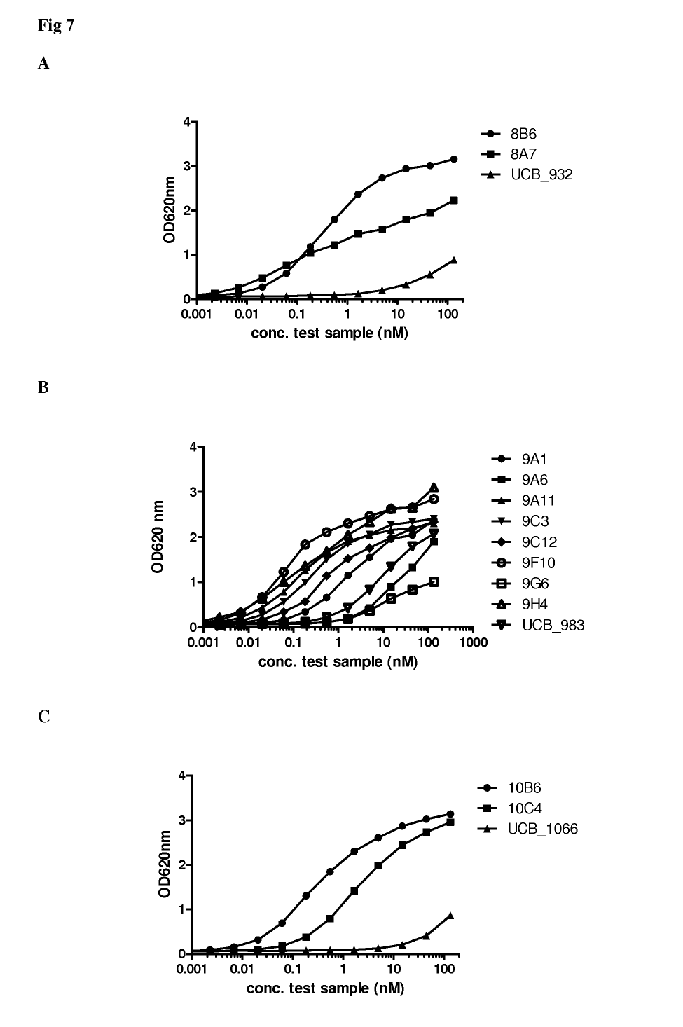

In a next experiment, the binding affinity of the loop-specific mAbs was determined and compared to reference mAbs as described in US2011/0135662 (for loop A3: UCB_932, for loopB1-C1-D1: UCB_983, for loopC3: UCB_1066; US2011/0135662). Affinities were determined using an ELISA with a loop-coating of 10 ng/well (loopA3-llama Fc), 20 ng/well (loop-B1-C1-D1-llama Fc) or 50 ng/well (loopC3-llama Fc). The remainder of the experimental setup was identical to abovementioned binding ELISAs. Tables 13 to 15 summarize EC₅₀ values (nM) as determined using GraphPad Prism software and results are shown in FIG. 7. Most of the mAbs show pM affinity for the respective hNav1.7 extracellular loop they are binding. In addition, these hNav1.7 antibodies display significantly improved affinity over the reference antibodies (see FIG. 7). The EC₅₀ for the reference mAbs could not be determined (nd) from the ELISA data available because the binding was too weak.

TABLE 13
EC₅₀ values as determined using a binding ELISA for
loopA3-binding mAbs derived from DNA immunization.

| | 8B6 | 8A7 | UCB_932 |
|---|---|---|---|
| EC50 (nM) | 0.37 | 0.23 | nd |

TABLE 14
EC₅₀ values as determined using a binding ELISA for
loopA3-binding mAbs derived from DNA immunization.

| | 9A1 | 9A6 | 9A11 | 9C3 | 9C12 | 9F10 | 9G6 | 9H4 | UCB_983 |
|---|---|---|---|---|---|---|---|---|---|
| EC50 | 0.33 | nd | 0.091 | 0.17 | 0.22 | 0.081 | nd | 0.19 | nd |
| (nM) | | | | | | | | | |

TABLE 15
EC₅₀ values as determined using a binding ELISA for
loopC3-binding mAbs derived from DNA immunization.

| | 10B6 | 10C4 | UCB_1066 |
|---|---|---|---|
| EC50 (nM) | 0.33 | 1.57 | nd |

### Example 9

### Specificity of Nav1.7 Antibodies Versus Nav1.5 and Nav1.2

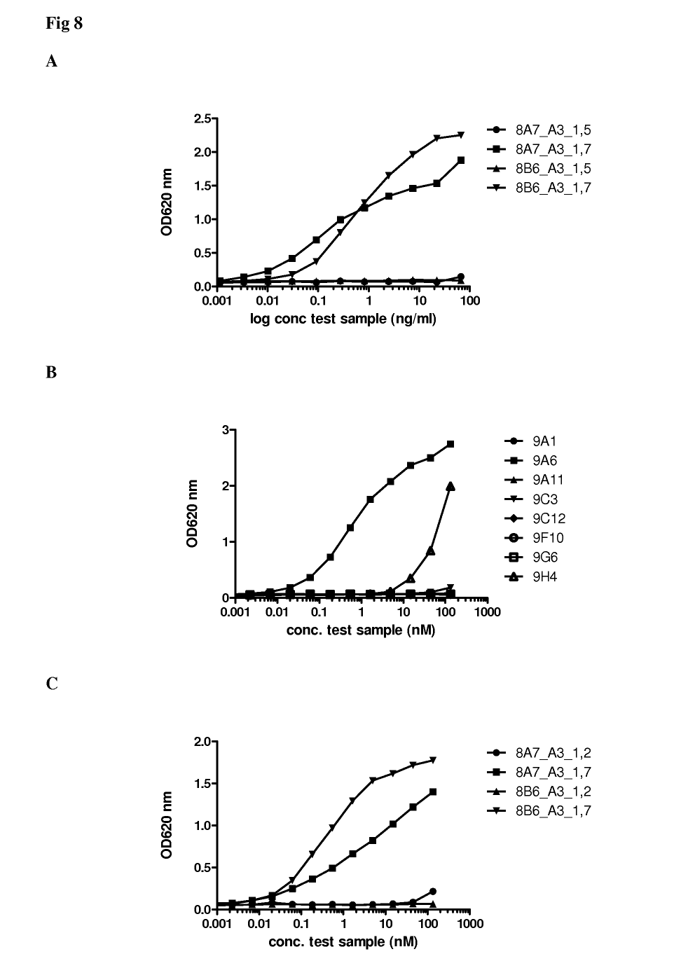

The therapeutic potency of an anti-hNav1.7 antibody will highly depend on the lack of binding to other Nay family members. Of particular interest is hNav1.5, which is expressed on cardiac muscle cells. As such, cross-reactive binding of hNav1.7 antibodies to hNav1.5 could lead to heart failure. To assay the specificity of the mAbs derived from DNA immunization for hNav1.7, the corresponding loopA3 and loopB1-C1-D1 sequences of hNav1.5 were cloned as llamaFc chimera and cross-reactivity was determined using a binding ELISA (FIG. 8). All clones, except clone 9A6 and 9H4, show no cross-reactivity to hNav1.5. In addition, cross reactivity for hNav1.2 was tested for the loopA3-binding clones 8A7 and 8B6 using hNav1.2 and hNav1.7 derived monomeric, GST-tagged loopA3 peptides (Abnova, H00006326-Q01 (hNav1.2) and H00006335-Q01 (hNav1.7)). Again, no cross-reactive binding was observed.

### Example 10

### Immune Precipitation of hNav1.7 from Cell Lysate with DNA Immunization-Derived mAbs

Inducible hNav1.7-HEK293 cells (BPS Bioscience, CA; cat. nr. 60507) were induced for 24 hrs with 1 jag/ml doxycycline and 3 mM Na-butyrate. Cells were harvested, washed with PBS and lysed in RIPA buffer (Sigma, cat. nr. R0278) at a density of 1×10⁷ cells per ml.
Aliquots of the cleared lysate (40 μl each) were incubated with 2 g of each mAb, for 2 hours at 4° C. in a rotator. Protein A beads (GE Healthcare, cat nr. 17-5138-01) were added (15 μl bead volume) and the lysate was further incubated overnight. After adding extra 180 μl RIPA buffer, the beads were pelleted by centrifugation, the supernatant was stored for analysis, and the beads were washed extensively with RIPA buffer. The bead pellets were resuspended in 30 jl SDS-PAGE sample buffer and boiled. In parallel, 30 ul samples of the supernatants were supplemented with SDS-PAGE sample buffer and boiled. The samples were size-separated on 6% SDS-polyacrylamide gels, blotted to nitrocellulose membranes and stained with a mouse anti-hNav1.7 antibody (Millipore, clone N68/6). After secondary staining with donkey anti-mouse IR800CW (Li-Cor), the blots were imaged in a Li-Cor Odyssey IR imaging system at 700 and 800 nm.
The results are shown in FIG. 9. Corresponding immune precipitate (IP) and supernatant (S) from the same mAb are loaded in adjacent positions on the SDS-PAGE gels. As positive controls (+), 10 μl of hNav1.7-HEK293 cell lysate was loaded on the gels. A PAGE-ruler prestained protein marker (M) (Fermentas) was used for reference. As shown in FIG. 9, a number of mAbs are able to precipitate Nav1.7 from the cell lysate, as shown by the presence of Nav1.7 in the precipitate fraction. These mAbs include 8B6 and 9A1. When no mAb was used in the immune precipitation, no Nav1.7 was found in the precipitate fraction.

### Example 11

### Functional Testing of Nav1.7 Antibodies

### Solutions and Antibodies:

Extracellular solution was composed of 108.75 mM Choline-CL, 36.25 mM NaCl, 4 mM KCl, 1 mM MgCl₂, 2 mM CaCl₂, 10 mM Hepes and 10 mM Glucose, pH was adjusted to 7.4 with NaOH.
Intracellular solution was composed of 120 mM CsF, 15 mM NaCl, 10 mM EGTA, 10 mM HEPES, pH was adjusted to 7.25 with CsOH.
Antibodies were dialysed to extracellular solution and stored at 4° C. until testing.

### Cell Culture Conditions:

HEK293/hNav1.7 cells (K1.6) were cultured in Eagle's Minimum Essential Medium (EMEM) (LONZA cat. BE12-125F; 500 mL), supplemented with Fetal Bovine Serum (Euroclone cat. ECS 0180 L; 50 mL), Penicillin/Streptomycin (Lonza cat. DE17-602E; 5 mL of 100× Solution), Ultraglutamine-1 (Lonza cat. BE17-605E/U1; 5 mL of 200 mM Solution), and neomycin (Invivogen, cat. ant-gn-5; 2.5 mL of 100 mg/mL stock solution).

### Experimental Protocol:

48 or 72 hours before experiment, 10⁷ or 5.10⁶ cells, respectively were seeded onto T225 flasks. Just before the experiments cells were washed twice with D-PBS w/o Ca²⁺/Mg²⁺ (Euroclone cat. No.: ECB4004L) and detached from the flask with trypsin-EDTA (Sigma, cat. No: T4174 diluted 1/10). Cells were then re-suspended in the suspension solution: 25 mL EX-CELL ACF CHO medium (Sigma, cat. No: C5467); 0.625 mL HEPES (Lonza, cat. No: BE17-737E); 0.25 mL of 100× Penicillin/Streptomycin (LONZA, cat. No: DE17-602E), 0.1 mL of Soybean trypsin inhibitor 10 mg/mL (Sigma, cat. No: T6522) and placed on the QPatch 16×.

### Patch Clamp Analysis:

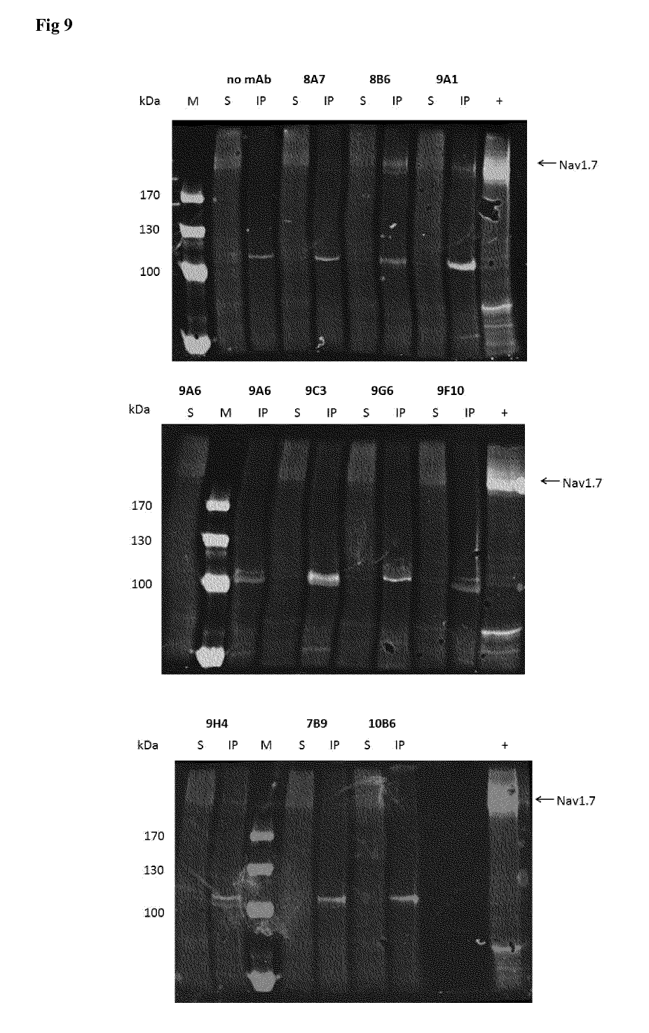

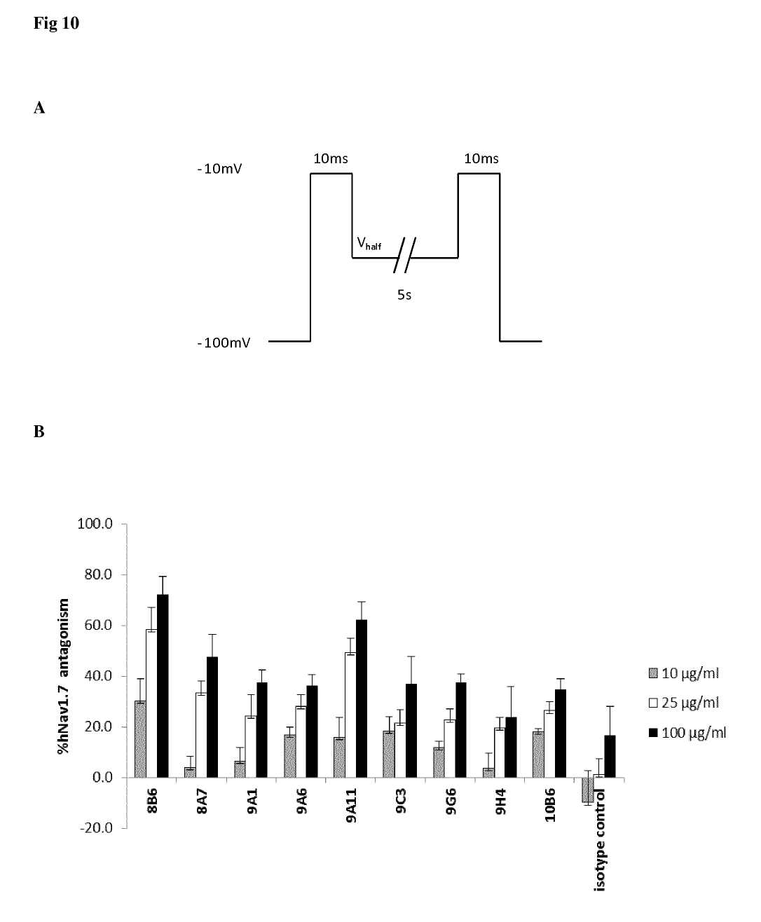

Standard whole-cell voltage clamp experiments were performed at room temperature.
For voltage clamp experiments on Nav1.7, data were sampled at 25 KHz. After establishment of the seal and the passage in the whole cell configuration, the Nav1.7 expressing HEK293 cells were held at −100 mV and the current was evoked using the voltage protocol illustrated in FIG. 10A, applied every 15 sec. in the absence of any compound (vehicle period) and in the presence of increasing concentrations of the antibody under investigation (10, 25 and 100 μg/ml). This voltage protocol allows assessing the potential effect of the compound under investigation on both the closed and the inactivated states of Nav1.7 (P01 and P02, respectively).
The output is defined as the maximal inward current evoked by the first and the second depolarization step (I_{P01} and I_{P02} respectively). For data acquisition the Sophion proprietary software was used.
No significant inhibition on the P01 protocol could be observed for the tested mAbs. However, as shown in FIG. 10B and summarized in Table 16, the antibodies had a clear antagonizing effect on the P02 protocol compared to the isotype control (anti-CD70-hIgG1 mAb), suggesting a stabilization effect on the inactivated form of hNav1.7.

TABLE 16
% inhibition of inward current following P02 voltage protocol

| | | | | | | | | | | isotype |
|---|---|---|---|---|---|---|---|---|---|---|
| | 8B6 | 8A7 | 9A1 | 9A6 | 9A11 | 9C3 | 9G6 | 9H4 | 10B6 | control |
| 10 μg/ml | 30.2 | 4.0 | 6.4 | 16.9 | 16.0 | 18.3 | 11.8 | 3.8 | 18.1 | −9.9 |
| 25 μg/ml | 58.4 | 33.3 | 24.3 | 28.0 | 49.4 | 21.6 | 22.8 | 19.8 | 26.3 | 1.2 |
| 100 μg/ml | 72.2 | 47.6 | 37.5 | 36.2 | 62.3 | 36.9 | 37.5 | 23.9 | 34.7 | 16.6 |

### Example 12

### Cross-Reactivity with Rodent hNav1.7

Further characterisation of antagonistic potency of the hNav1.7 binding mAbs could be done using various in vivo and in vitro rodent systems. Determination of the rodent cross-reactivity of the mAbs derived from DNA immunization would identify the usefulness of such assays. Corresponding loopA3 and loopB1-C1-D1 sequences of rat hNav1.7 were cloned as llamaFc chimera and cross-reactivity was assayed in a binding ELISA (FIG. 11). All but one tested antibodies (9A6) show cross-reactivity for the corresponding rat chimera. Most clones bind with similar affinity to the rat orthologue sequence (i.e. 8B6, 9A11), although some show reduced affinity (i.e. 8A7, 9A1).

TABLE 17
Nucleotide sequences encoding the VL and VH domains of Nav1.7 antibodies

| Clone | Nucleotide Sequence | SEQ ID NO: |
|---|---|---|
| >8A7_VL | TCCTACGAACTGACTCAGTCACCCTCAGTGACGGTGGCACTGAGACAGACGGCCAAGATC | 304 |
| | ACCTGTGGCGGGAGCCGCATTGGAAGTAAAAGTGTTCAGTGGTACCAGCAGAAGCCGGGC | |
| | CAGGTCCCTGTGCTGGTCATCTATGCTGATAACAGACGGCCCTCAAGGATCCCTGAGAGG | |
| | TTCTCAGGCTCCAACTCTGGGAACACGGCCACCCTGATAATCAGCGGGGCCCAGGCCGAG | |
| | GACGAGGCCGACTACTACTGTCAGGTGTGGGACAGCAGTGCTGCTGTGTTCGGCGGAGGG | |
| | ACCCATCTGACCGTCCTCGGT | |
| >8A7_VH | GAGGTGCAGGTGCAGGAGTCGGGCCCAGGCCTGGTGACACCCTCGCAGACACTCTCCCTC | 305 |
| | ACCTGCACTGTCTCTGGTGGCTCCATTAAAAACAACTATTACTACTGGAACTGGATCCGC | |
| | CAGCCCCCCGGAAAGGGGCTGGAATGGATGGGAGCCATAGCTTATAGCGGCAGCACTTAC | |
| | TACAGCCCATCCCTCCAGAGCCGCACCTCCATCTCCAGGGACACGTCCAAGAACCAGTTC | |
| | ACCCTGCAGCTGAGCTCTGTGACCCCTGAAGACACAGCCGTGTATTACTGTGCCAGAGTG | |
| | CCGTCGGTATTTGGCCTTCCTTATGGCGGCATGAACTACTGGGGCAAAGGGACCCTGGTC | |
| | ACTGTCTCCTCA | |
| >8B6_VL | GACATCCAGATGACCCAGTCTCCTTCCTCCCTGTCTGCATCTCTGGGAGACAGAGTCACC | 306 |
| | ATCACCTGCCAGGCCAGTCAGGGCATCAGCAAGTACTTAGCCTGGTATCAGCAGAAACCA | |
| | GGGCAAGCTCCTAAGCTCCTGATCTATAGTGCATCCAGACTAGAAACCGGGGTCCCTTCC | |
| | AGGTTCAGCGGCAGTGGATCAGGGACAACTTTCACCCTCGTCATCAGCGGCCTGGAGGCT | |
| | GAAGATGCCGGAAGTTACTACTGTCAGCAGTATTACAGAGCACCCGCTACGTTCGGCCAG | |
| | GGGACCAAGGTGGAACTCAAA | |
| >8B6_VH | GAGGTGCAGCTGCAGGAGTCGGGCCCAGGCCTGGTGAAGCCCTCGCAGACGCTCTCTCTC | 307 |
| | ACCTGCACTGTCTCTGGTGGCTCCATCTCAACCAGTGGTACTGGTTGGAGCTGGATCCGC | |
| | CAGCCCCCAGGGAAGGGGCTGGAGTGGATGGGGATCATAGGTTATGATGGCGGCACTTAC | |
| | TACAACCCATCTCTCAAGAGCCGCACTTCCATCTCCAGAGACACGTCCAAGAACCAGTTC | |
| | TCCCTGCAGTTGACCTCTGTGACCCCTGAGGACACGGCCGTGTATTACTGTGCCAGAGGG | |
| | CTGGGGCCCTGGGGCCAGGGGATCCAGGTCACCGTGTCCTCAGCCAGT | |
| >9A11_VL | GCGACCATGCTGACCCAGTCCCCAGGCTCCCTGTCTGTCGTCCCTGGAGAGTCAGCCTCC | 308 |
| | ATCTCCTGCAAGGCTAGTCAGAGCCTCGTACACACTGATGGAAAGACCTATTTGTCTTGG | |
| | CTCCTACAGAAGCCGGGCCAATCTCCACAGCGACTGATCTATCAAGTTTCCACCCGTGGC | |
| | TCTGGGGTCCCAGACAGGGTCACTGGCAGCGGGTCAGGGACAGATTTCACCCTGAAAATC | |
| | AGCGGGGTGAAGGCTGAGGATGCTGGAGTGTATTACTGTGCTCAAACTACATATTATCCT | |
| | CCAACGTTCGGCCAGGGGACCAAGGTGGAACTCAAA | |
| >9A11_VH | GAGGTCCAGCTGGTTCAGCCAGGGGCTGAGGTGAGACGCCCTGGGGCAGCAGTGAAGGTC | 309 |
| | TCCTGCAAGGCTTCTGGATTTGCGTTGATAAGCTCGTACATAGACTGGGTGCGGCAGGCC | |
| | CCTGGACAAGGGCTTGAGTGGATGGGGAGGATTGACCCTGAAGATGGTGGTACATCATAT | |
| | GCACAAAAGTTCCAGGGCCGAGTCACGGTCACTGTCGACACGGCCACCAGCACAGCCTAC | |
| | GTGGAGTTGAGCAGTCTGAGATCTGACGACACGGGCGTCTATTTCTGCATGGTGGGGGAT | |
| | TCAGGCTACTGGGGCCAGGGGACCCAGGTCACCGTGTCCTCA | |

### Example 13

### Determination of 8A7 Binding Epitope Using Mouse-Human Loop A3 Chimeras

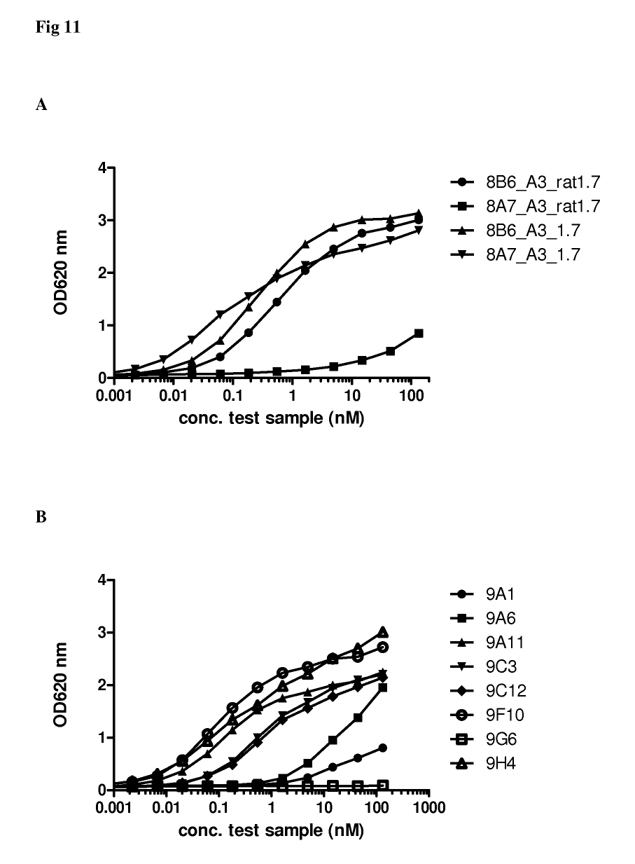

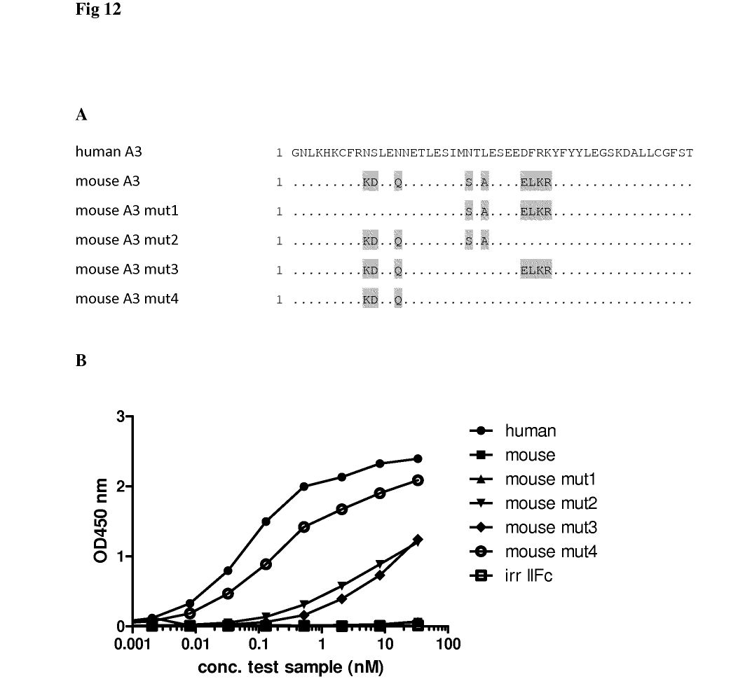

The binding epitope of the hNav1.7 loopA3 binding mAb 8A7 was determined. To this end, various mouse-human-loopA3 chimeras were constructed, changing distinct regions in the mouse loopA3 sequence into the corresponding human sequence. These constructs were designed using standard recombinant DNA and PCR methodologies based on the previously described mouse A3-llamaFc construct. The sequence alignment of all mouse-human chimeras can be found in FIG. 12A and the sequences are shown in Table 18 below.

TABLE 18
Human and mouse Nav1.7 loop A3 sequences with
mutant chimeric sequences

| | | SEQ |
|---|---|---|
| A3 loop | Sequence | ID NO |
| Human A3 | GNLKHKCFRNSLENNETLESIMNTLESEEDFRKYFYYLEGSKDALLCGFST | 262 |
| Mouse A3 | GNLKHKCFRKDLEQNETLESIMSTAESEEELKRYFYYLEGSKDALLCGFST | 310 |
| Mouse A3 | GNLKHKCFRNSLENNETLESIMSTAESEEELKRYFYYLEGSKDALLCGFST | 311 |
| mut1 | | |
| Mouse A3 | GNLKHKCFRKDLEQNETLESIMSTAESEEEEDFRKYFYYLEGSKDALLCGFST | 312 |
| mut2 | | |
| Mouse A3 | GNLKHKCFRKDLEQNETLESIMNTLESEEELKRYFYYLEGSKDALLCGFST | 313 |
| mut3 | | |
| Mouse A3 | GNLKHKCFRKDLEQNETLESIMNTLESEEDFRKYFYYLEGSKDALLCGFST | 314 |
| mut4 | | |

Binding of mAb 8A7 to the various mouse-human loop chimeras was assayed in a binding ELISA using a similar approach as described above. The results are shown in FIG. 12B. 8A7 did not show any binding to the wild-type mouse A3 loop or the mouse A3 mut1 loop, whilst partial binding was observed using A3 mutants 2 and 3. Combining the amino acid substitutions present in mutants 2 and 3 (see mutant 4) significantly increased the affinity of 8A7 for the target sequence, resulting in a similar binding profile as compared to the binding affinity seen with the human A3 loop sequence. This indicates that hNav1.7 residues ²⁹¹NTLESEEDFRK³⁰¹ are essential components of the 8A7 epitope.

### Example 14

### Further Functional Testing of the 8A7 Antibody Using Manual Patch Clamp Electrophysiology

The hNav1.7 antagonizing potency of mAb 8A7 was characterised using manual patch clamp electrophysiology.

### Solutions and Antibodies

Internal solution (in mM): 140 CsF, 10 NaCl, 10 HEPES and 1 EGTA, 15 Glucose (adjusted to pH 7.38 with CsOH). External bathing solution (in mM): 140 NaCl, 3 KCl, 1 MgCl₂, 1 CaCl₂, 10 HEPES and 20 Glucose (pH=7.4), adjusted with NaOH. Antibodies were dialyzed to extracellular solution and stored at −20° C. until testing.

### Cell Culture Conditions

HEK293/hNav1.7 cells were maintained in DMEM-F12 medium (Gibco-Life technologies) including 10% FBS, 1.0 g/l Glucose, 1% Penicillin/Streptomycin (PAA Laboratories GmbH) and Img/ml Geneticin.
HEK293/hNav1.5 cells were maintained in DMEM (Gibco-Life Technologies) including 10% FBS, 1.0 g/l Glucose, 1% Penicillin/Streptomycin (PAA Laboratories GmbH).

### Experimental Protocol

For patch clamp recordings, stably transfected HEK293 cells expressing hNav1.7 were used within 1-3 days after splitting. Whole-cell voltage clamp recordings were performed using glass electrodes with tip resistances of 1.5 to 2.0 MQ, manufactured with a DMZ puller (Zeitz Instruments GmbH, Martinsried, Germany), filled with internal solution. The monoclonal antibody 8A7 or the isotype control were diluted to a final concentration of 25 μg/ml. Incubation of the respective antibody lasted from 10 min to 70 min during patch clamp recordings. Membrane currents were acquired at room temperature 21±2° C. using a HEKA EPC-10USB amplifier (HEKA electronics, Lambrecht, Germany), low pass filtered at 10 kHz and digitized at 100 kHz. Pipette potential was zeroed prior to seal formation and capacitive transients were compensated using C-fast for pipette-capacity correction and subsequently C-slow for cell capacity compensation (PatchMaster, HEKA, Lambrecht, Germany). The series resistance was compensated by at least 50% and leak pulses were applied following the test pulse to subtract mean leak current digitally online corresponding to the P/4 test pulse procedure. Patchmaster/Fitmaster software (HEKA Elektronik, Lamprecht, Germany) was used for acquisition and off-line analysis. Voltage protocols were carried out after current stabilization and equilibration was established. Standard current-voltage (I-V) curves were recorded using 100 ms pulses from a holding potential of −120 mV to a range of potentials (−90 to +40 mV) in 10 mV steps with intervals of 5s at 120 mV between pulses. Conductance-voltage curves were obtained by calculating the conductance G at each voltage V using the equation G=I/V-Vrev, with Vrev being the reversal potential, determined for each cell individually and I being the peak current measured during the test pulse. Conductance-voltage curves were fitted with a Boltzmann equation GNa=GNa, max/1+exp [(Vm−V1/2)/k] where GNa is the voltage-dependent sodium conductance, GNa, max is the maximal sodium conductance, V1/2 is the potential at which activation is half-maximal, Vm is the membrane potential, and k is the slope factor. At least 7 individual cells were tested per condition.

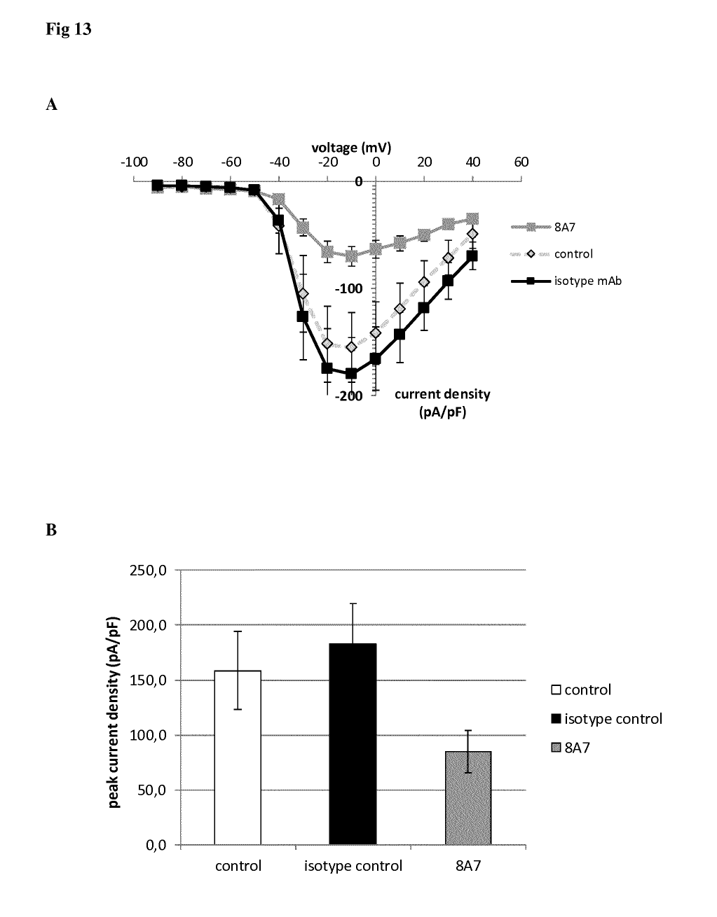

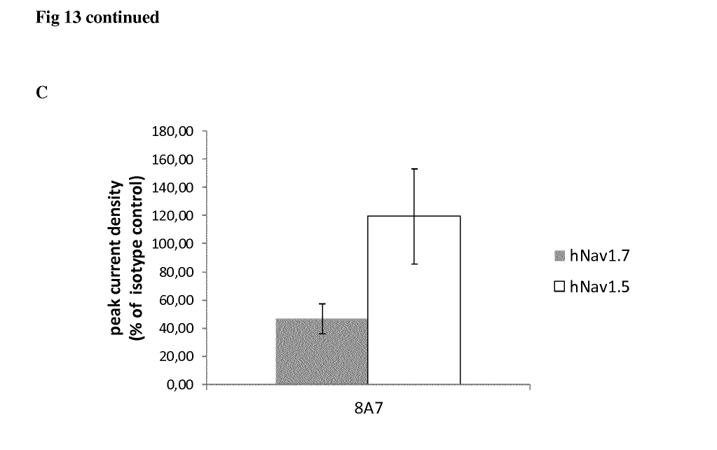

Pre-incubation of antibody 8A7 clearly alters the voltage/current density profile (FIG. 13A) and reduces peak current density (FIG. 13B) compared to isotype treated or untreated (control) cells. This antagonizing potency of 8A7 is specific for hNav1.7 since no effect is seen upon analysis of hNav1.5 expressing cells (FIG. 13C).

Antibodies Raised by DNA Immunization and Identified by Screening Using scFv Libraries

Phage selection on membrane embedded targets like GPCRs and ion channels is very difficult because the structure of these targets depends on the membrane environment. For selection using full-length GPCRs, one can use cells overexpressing the GPCR of interest, or membranes or lipoparticles derived therefrom. The density of GPCR used for selection is therefore much lower compared to using a recombinant protein. Furthermore the membrane environment always results in background problems during phage panning.

Fab display on phage is rather inefficient; the expression is approximately one Fab per 10 phage particles. ScFv display is more efficient with more than one scFv per phage resulting in avid binding of the phage to the immobilized target. The examples below demonstrate that multivalent scFv display can compensate for the low target density of full-length GPCR coatings and leads to the identification of GPCR specific VH and VL domains.

The study described below showing the advantages of scFv display over Fab display, was used to identify clones binding to the extracellular loops (ECLs) of the glucagon receptor (GCGR). The advantage of using GCGR in this study was that GCGR has a very large N-terminal domain, which can be used as a positive control during phage selections when used as a recombinant protein in parallel to full-length GCGR on lipoparticles.

### Example 15

### DNA Immunization, scFv Vector Design and Library Construction

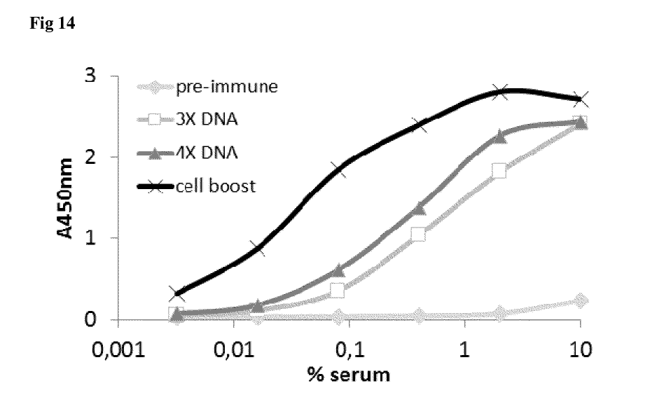

Four llamas were immunized by injection of an expression plasmid containing the GCGR-encoding cDNA. After four DNA immunizations, the animals were boosted with dromedary Caki cells overexpressing human GCGR. FIG. 14 shows an example of the llama IgG1 response of the sera of the immunized llama 73. The immune response was already high after the DNA injections and indicates that the cell boost is not essential to get a good immune response. The advantage of DNA immunization without a subsequent cell boost is that the response is specific to the target protein, without any off-target responses against membrane epitopes.

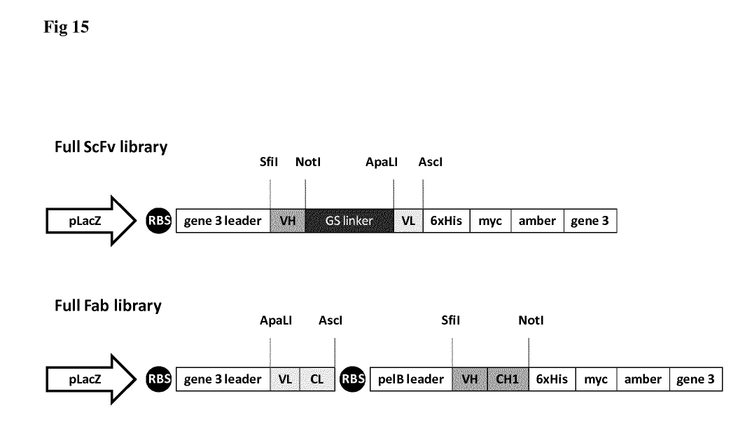

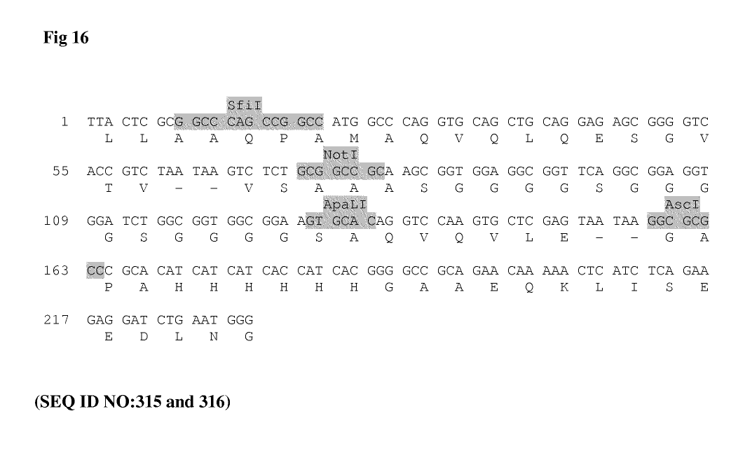

The vector for scFv display of llama IgG1 was based on the pCB3-reverse vector used for Fab display (see FIG. 15). Using the pCB3-reverse vector the light chain is fused to gene 3 instead of the heavy chain. In the scFv vector (pSc) a GS linker is introduced to couple the VH to the VL (FIG. 15). The sequence of the pSc vector is shown in FIG. 16 (SEQ ID NO: 315 and 316).

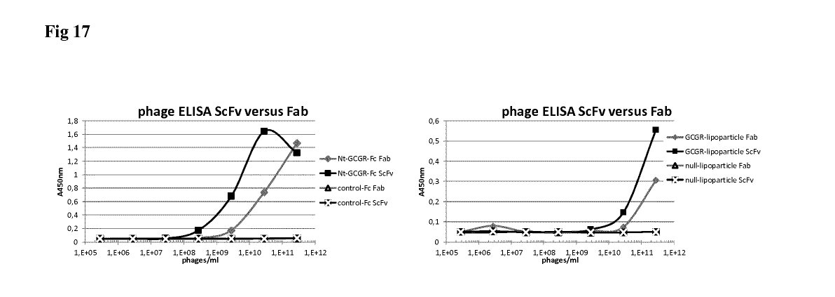

To test functional scFv display, the VH and VL sequences of a GCGR-specific clone, “1C3”, which was found by Fab display, were cloned in the scFv vector. Phage ELISA showed that scFv display of clone 1C3 may be more sensitive than Fab display (FIG. 17). Lower phage titers were needed to detect phage binding to its target in the case of scFv display as compared to Fab display.

ScFv libraries were made starting from material obtained from llama 73 immunized with GCGR DNA. As part of the immunization protocol, the llama was boosted with cells overexpressing GCGR. Fab display libraries of this llama have yielded a great diversity of GCGR-specific VH families.

In order to make the scFv libraries of conventional llama IgG1, the variable regions of the heavy and the light chains were amplified by PCR from the primary Fab libraries and cloned into the pSc vector, separately. Since heavy chain only antibodies from llama (IgG2 and IgG3) do not have a CH1 domain and the primary VH Fab libraries were built using CH specific primers, the scFv libraries should only contain the variable regions of conventional llama IgG1. To amplify the full diversity of VH sequences, 4 different anti-sense primers were designed as shown in Table 19.

TABLE 19
Primers for scFv library construction

| | | | | SEQ ID |
|---|---|---|---|---|
| Application | Primer | Sequence | | NO. |
| VH | PelB3 | ATGAAATACCTATTGCCTACGGCAGCC | | 317 |
| amplification | | | | |
| | PelB4 | CCGCTGGATTGTTATTACTC | | 318 |
| | VH anti | A TGT TGC GGC CGC GGA GGC TGA | Notl linker | 319 |
| | 1A | GGA GAC | | |
| | VH anti | A TGT TGC GGC CGC GGA GGC TGA | Notl linker | 320 |
| | 2A | GGA CAC | | |
| | VH anti | A TGT TGC GGC CGC GGA GGC GCT | Notl linker | 321 |
| | 3A | GCT AAC | | |
| | VH anti | A TGT TGC GGC CGC GGA GGC CGA | Notl linker | 322 |
| | 4A | GGA AAC | | |
| VL | M13R | GAGCGGATAACAATTTCACACAGG | | 323 |
| amplification | | | | |
| | VL anti | ACGTCAGT GGCGCGCC GGC CTT | Ascl linker | 324 |
| | | GGG CTG ACC CAG GAC | | |
| VK | M13R | GAGCGGATAACAATTTCACACAGG | | 325 |
| amplification | | | | |
| | VK anti | ACGTCAGT GGCGCGCC CTT GGC | Ascl linker | 326 |
| | | ATC AGC CCG | | |
| GS linker | GS linker | G GCC GCA AGC GGT GGA GGC GGT | Notl | 327 |
| insertion in | Sence | TCA GGC GGA GGT GGA TCT GGC | overhang | |
| pCB3-rev | | GGT GGC GGA AG | | |
| | GS linker | TGC ACT TCC GCC ACC GCC AGA | ApaLl | 328 |
| | anti | TCC ACC TCC GCC TGA ACC GCC | overhang | |
| | | TCC ACC GCT TGC G | | |

The PelB3 primer anneals upstream of the SfiI site in the Fab vector and was therefore used as a sense primer in combination with the 4 NotI-tagged antisense primers for the amplification of the VH sequences. PCR reactions yielded 500 bp fragments as expected. PCR3 did not yield any product. It is possible that the VH anti GS 3A primer does not anneal to the sequences of the repertoire of llama 73.

The products were isolated from agarose gel and digested with Sfi and NotI for restriction cloning in pSc. For the amplification of the light chains, two antisense primers were designed: one for the VL sequences and one for the VK sequences (see Table 19). These primers were based on a list of all primer sequences that have been used to clone full IgG1. The M13 Reverse primer anneals upstream of the ApaLI site in the primary Fab library and was therefore used in combination with the AscI-tagged antisense primers to amplify the VL/VK repertoire. PCR reactions yielded 500 bp fragments as expected, and after ApaLI-AscI digestion these were cloned into the pSc vector. The variable regions of the heavy and the light chain were cloned into the pSc vector, separately. Full scFv libraries were built via ApaLI-AscI restriction cloning of the lambda and kappa Fv libraries into the pSc vector containing the VH library.

### Example 16

### Phage Selections: scFv Display Versus Fab Display

To determine whether scFv display of llama IgG1 is more efficient to identify GCGR-specific clones as compared to Fab display, we performed selections on GCGR lipoparticles. As a test for enrichment of GCGR binders, selection was also carried out using the recombinant N-terminal domain of GCGR as well. The recombinant N-terminal domain is a purified protein and can therefore be coated at much higher amounts than the full-length GCGR embedded in the lipoparticle membrane. Furthermore the GCGR lipoparticles are cell-derived and still contain cell membrane epitopes that can cause background during phage selections.

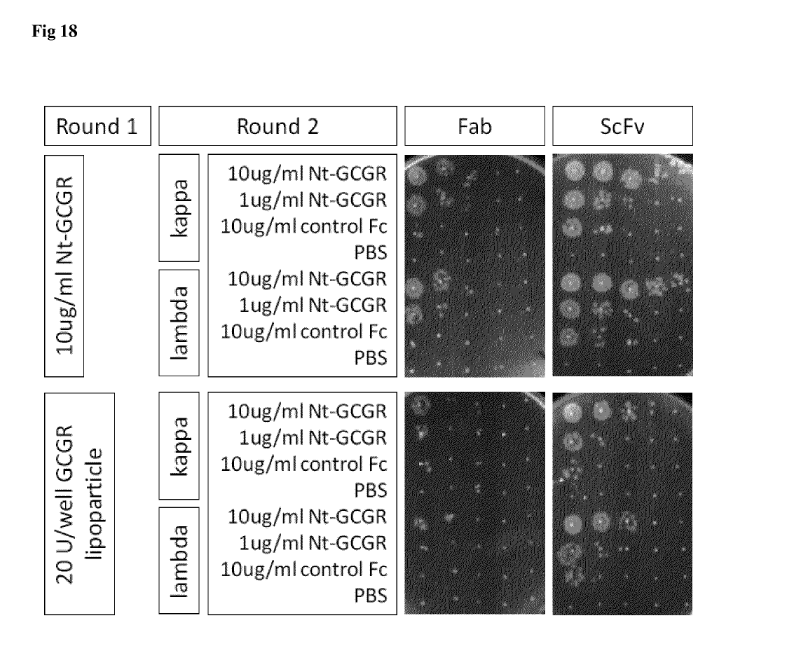

After the first round of selection, low outputs were found. In the second round on the N-terminal domain, enrichment was seen in the case of both Fab- and scFv-display (see FIG. 18).

The output numbers were however 100-fold higher for the scFv libraries over the Fab libraries. Background on the control Fc coating was also higher for the scFv libraries over the Fab libraries.

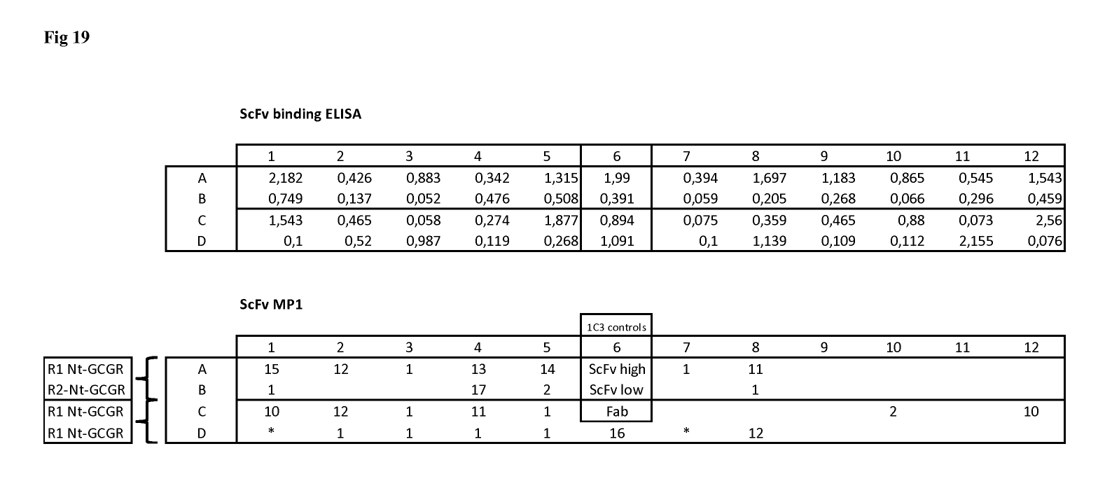

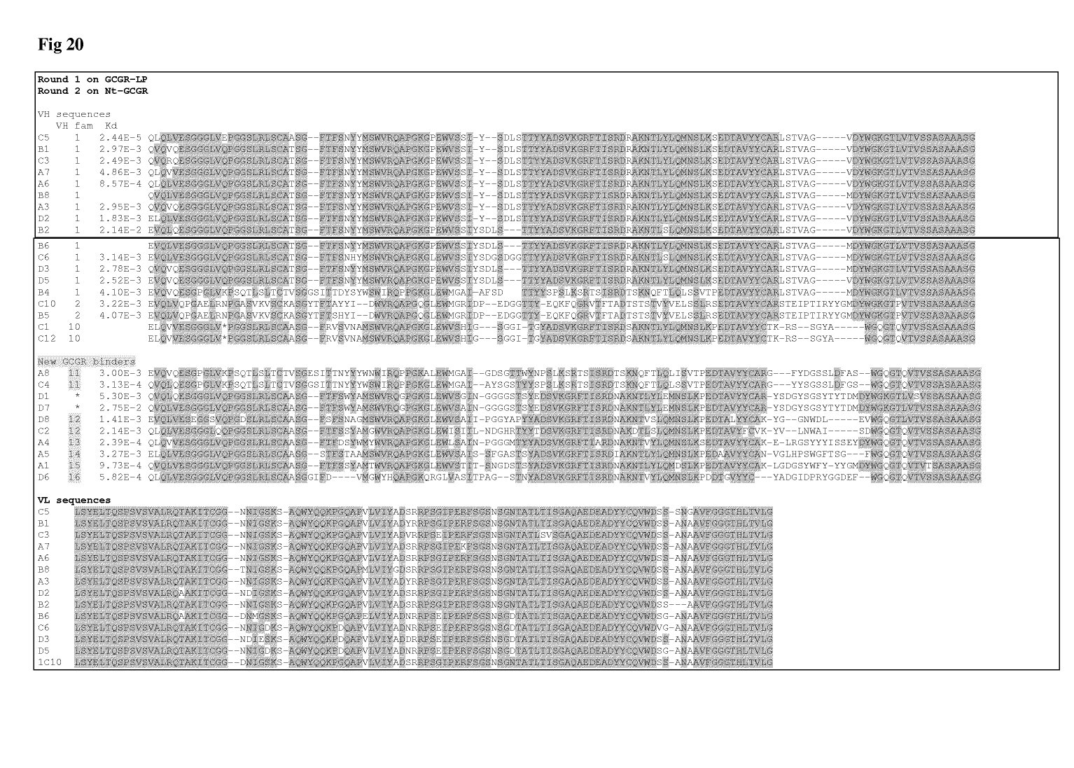

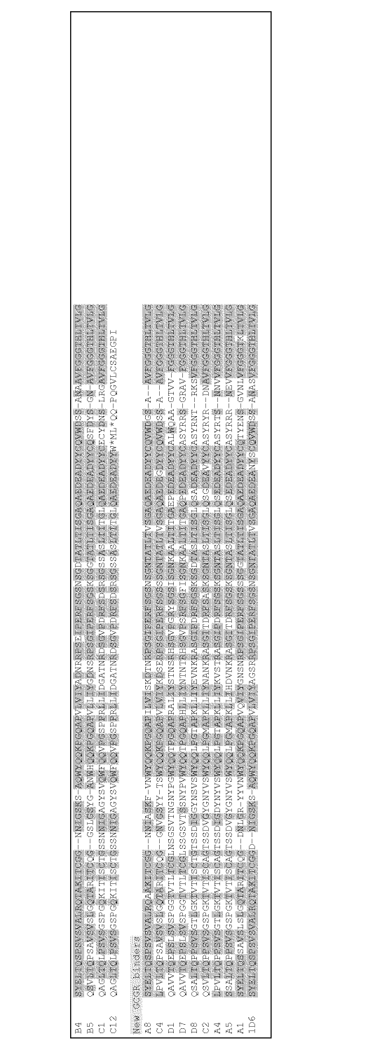

ScFv binding ELISA using periplasmic extracts showed Nt-GCGR-specific binding of 34 out of 44 clones analysed (FIG. 19). Sequence analysis showed sequences of VH family 1, 2 and 10, which were previously found after the selections with the Fab lambda library of llama 73. In addition seven new VH families were identified specifically recognizing GCGR using the lambda scFv library of llama 73. These sequences of the VH and VL domains are shown in Table 20 below and an alignment is shown in FIG. 20.

Because scFv display is multivalent, clones will be selected due to avid binding to the coated target, but might have a low affinity as a monovalent scFv. Off-rates of periplasmic scFv fragments, determined on Biacore using a chip coated with Nt-GCGR, showed off-rates similar to conventional Fab libraries from the same animal (see Table 20). One explanation for the good off-rates might be that the clones selected from the GCGR lipoparticle screening need good affinity and multivalent display, in order to compensate for the low target density on the lipoparticles.

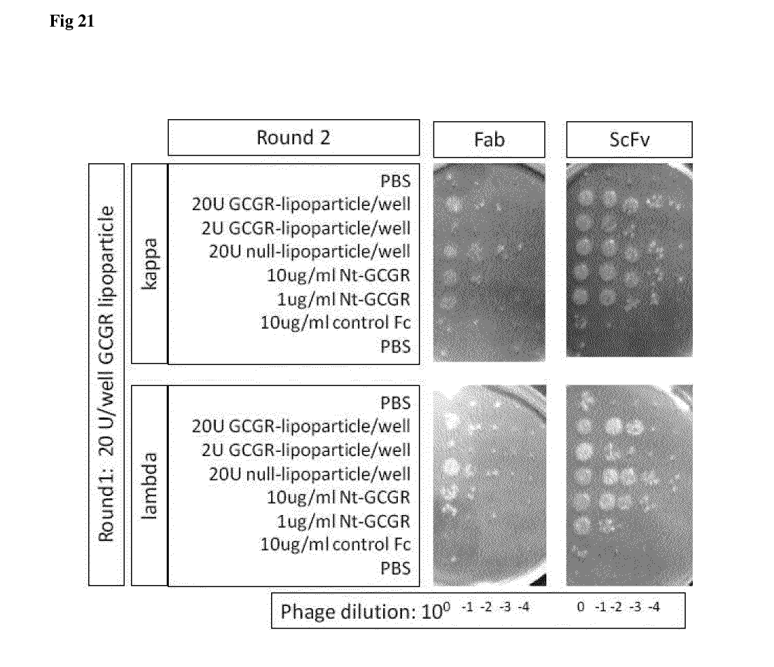

In contrast to the good enrichment observed in the second round screening on the recombinant N-terminal domain of GCGR, second round screening on GCGR lipoparticles gave very high background on the null lipoparticles (FIG. 21). ScFv binding ELISA on Nt-GCGR did not show any binders, and sequence analysis of clones picked after two rounds of selection on GCGR lipoparticles did not show any of the 10 VH families identified before.

One of the explanations for a high background of off-target binders during rounds of selection on lipoparticles is that too much input phage is used. In order to determine whether there is an optimal phage input titer, a second round selection with different amounts of input phage was performed. There was a very high background on the null lipoparticles for any of the input titers tested. As a control, the binding of a positive control, clone 1C3, was tested. ScFv 1C3 specifically and dose dependently bound to the GCGR-lipoparticles, while there was no measurable background observed on the null lipoparticles. From these data, it can be concluded that phage are not intrinsically “sticky” to lipoparticles, and that the background seen on null lipoparticles is due to clones in the scFv library. These clones can be mismatched VH-VL pairs that become sticky, or clones binding off target epitopes on the lipoparticles.

TABLE 20
ScFv sequences of Nt-GCGR binding clones

| | | | SEQ | | SEQ | |
|---|---|---|---|---|---|---|
| | | | ID | | ID | |
| scFv | Family | VH | NO: | VL | NO: | Kd¹ |
| C5 | 1 | QLQLVESGGGLVEPGGSLRLSCAASGFTFSNYYMSW | 329 | SYELTQSPSVSVALRQTAKITCGGNNIGSKSAQWYQ | 355 | 2.44E−5 |
| | | VRQAPGKGPEWVSSIYSDLSTTYYADSVKGRFTISRD | | QKPGQAPVLVIYADSRRPSGIPERFSGSNSGNTATL | | |
| | | RAKNTLYLQMNSLKSEDTAVYYCARLSTVAGVDYWG | | TISGAQAEDEADYYCQVWDSS- | | |
| | | KGTLVTVSS | | SNGAVFGGGTHLTVLG | | |
| B1 | 1 | QVQVQESGGGLVQPGGSLRLSCATSGFTFSNYYMS | 330 | SYELTQSPSVSVALRQTAKITCGGNNIGSKSAQWYQ | 356 | 2.97E−3 |
| | | WVRQAPGKGPEWVSSIYSDLSTTYYADSVKGRFTISR | | QKPGQAPVLVIYADYRRPSGIPERFSGSNSGNTATL | | |
| | | DRAKNTLYLQMNSLKSEDTAVYYCARLSTVAGVDYW | | TISGAQAEDEADYYCQVWDSSANAAVFGGGTHLTV | | |
| | | GKGTLVTVSS | | LG | | |
| C3 | 1 | QVQRQESGGGLVQPGGSLRLSCATSGFTFSNYYMS | 331 | SYELTQSPSVSVALRQTAKITCGGNNIGSKSAQWYQ | 357 | 2.49E−3 |
| | | WVRQAPGKGPEWVSSIYSDLSTTYYADSVKGRFTISR | | QKPGQAPVLVIYADVRRPSEIPERFSGSNSGNTATL | | |
| | | DRAKNTLYLQMNSLKSEDTAVYYCARLSTVAGVDYW | | SVSGAQAEDEADYYCQVWDSSANAAVFGGGTHLT | | |
| | | GKGTLVTVSS | | VLG | | |
| A7 | 1 | QLQVVESGGGLVQPGGSLRLSCATSGFTFSNYYMSW | 332 | SYELTQSPSVSVALRQTAKITCGGNNIGSKSAQWYQ | 358 | 4.86E−3 |
| | | VRQAPGKGPEWVSSIYSDLSTTYYADSVKGRFTISRD | | QKPGQAPVLVIYADSRRPSGIPEKFSGSNSGNTATL | | |
| | | RAKNTLYLQMNSLKSEDTAVYYCARLSTVAGVDYWG | | TISGAQAEDEADYYCQVWDSSANAAVFGGGTHLTV | | |
| | | KGTLVTVSS | | LG | | |
| A6 | 1 | QLQLVESGGGLVQPGGSLRLSCATSGFTFSNYYMSW | 333 | SYELTQSPSVSVALRQTAKITCGGNNIGSKSAQWYQ | 359 | 8.57E−4 |
| | | VRQAPGKGPEWVSSIYSDLSTTYYADSVKGRFTISRD | | QKPGQAPVLVIYADSRRPSGIPERFSGSNSGNTATL | | |
| | | RAKNTLYLQMNSLKSEDTAVYYCARLSTVAGVDYWG | | TISGAQAEDEADYYCQVWDSSANAAVFGGGTHLTV | | |
| | | KGTLVTVSS | | LG | | |
| B8 | 1 | QVQLVESGGGLVQPGGSLRLSCATSGFTFSNYYMSW | 334 | SYELTQSPSVSVALRQTAKITCGGTNIGSKSAQWYQ | 360 | |
| | | VRQAPGKGPEWVSSIYSDLSTTYYADSVKGRFTISRD | | QKPGQAPMLVIYGDSRRPSGIPERFSGSNSGNTATL | | |
| | | RAKNTLYLQMNSLKSEDTAVYYCARLSTVAGMDYWG | | TISGAQAEDEADYYCQVWDSSANAAVFGGGTHLTV | | |
| | | KGTLVTVSS | | LG | | |
| A3 | 1 | QVQVQESGGGLVQPGGSLRLSCATSGFTFSNYYMS | 330 | SYELTQSPSVSVALRQTAKITCGGNNIGSKSAQWYQ | 356 | 2.95E−3 |
| | | WVRQAPGKGPEWVSSIYSDLSTTYYADSVKGRFTISR | | QKPGQAPVLVIYADYRRPSGIPERFSGSNSGNTATL | | |
| | | DRAKNTLYLQMNSLKSEDTAVYYCARLSTVAGVDYW | | TISGAQAEDEADYYCQVWDSSANAAVFGGGTHLTV | | |
| | | GKGTLVTVSS | | LG | | |
| D2 | 1 | ELQLVESGGGLVQPGGSLRLSCATSGFTFSNYYMSW | 335 | SYELTQSPSVSVALRQAAKITCGGNDIGSKSAQWYQ | 361 | 1.83E−3 |
| | | VRQAPGKGPEWVSSIYSDLSTTYYADSVKGRFTISRD | | QKPGQAPVLVIYADSRRPSGIPERFSGSNSGNTATL | | |
| | | RAKNTLYLQMNSLKSEDTAVYYCARLSTVAGVDYWG | | TISGAQAEDEADYYCQVWDSSANAAVFGGGTHLTV | | |
| | | KGTLVTVSS | | LG | | |
| B2 | 1 | EVQLQESGGGLVQPGGSLRLSCATSGFTFSNYYMSW | 336 | SYELTQSPSVSVALRQTAKITCGGNNIGSKSAQWYQ | 362 | 2.14E−2 |
| | | VRQAPGKGPEWVSSIYSDLSTTYYADSVKGRFTISRD | | QKPGQAPVLVIYADSRRPSGIPERFSGSNSGNTATL | | |
| | | RAKNTLSLQMNSLKSEDTAVYYCARLSTVAGVDYWG | | TISGAQAEDEADYYCQVWDSSAAVFGGGTHLTVLG | | |
| | | KGTLVTVSS | | | | |
| B6 | 1 | EVQLVESGGGLVQPGGSLRLSCATSGFTFSNYYMSW | 337 | SYELTQSPSVSVALRQAAKITCGGDNMGSKSAQWY | 363 | |
| | | VRQAPGKGPEWVSSIYSDLSTTYYADSVKGRFTISRD | | QQKPGQAPELVIYADNRRPSEIPERFSGSNSGDTAT | | |
| | | RAKNTLYLQMNSLKSEDTAVYYCARLSTVAGMDYWG | | LTISGAQAEDEADYYCQVWDSGANAAVFGGGTHLT | | |
| | | KGTLVTVSS | | VLG | | |
| C6 | 1 | EVQLVESGGGLVQPGGSLRLSCATSGFTFSNHYMSW | 338 | SYELTQSPSVSVALRQTAKITCGGNNIGDKSAQWYQ | 364 | 3.14E−3 |
| | | VRQAPGKGLEWVSSIYSDGSDGGTTYYADSVKGRFTI | | QKPDQAPVLVIYADNRRPSEIPERFSGSNSGDTATL | | |
| | | SRDRAKNTLSLQMNSLKSEDTAVYYCARLSTVAGMDY | | TISGAQAEDEADYYCQVWDVGANAAVFGGGTHLTV | | |
| | | WGKGTLVTVSS | | LG | | |
| D3 | 1 | QVQVQESGGGLVQPGGSLRLSCATSGFTFSNYYMS | 339 | SYELTQSPSVSVALRQTAKITCGGNDIESKSAQWYQ | 365 | 2.78E−3 |
| | | WVRQAPGKGPEWVSSIYSDLSTTYYADSVKGRFTISR | | QKPDQAPVLVIYADDRRPSEIPERFSGSNSGDTATL | | |
| | | DRAKNTLYLQMNSLKSEDTAVYYCARLSTVAGMDYW | | TISGAQAEDEADYYCQVWDSSANAAVFGGGTHLTV | | |
| | | GKGTLVTVSS | | LG | | |
| D5 | 1 | EVQVQESGGGLVQPGGSLRLSCATSGFTFSNYYMSW | 340 | SYELTQSPSVSVALRQTAKITCGGNNIGDKSAQWYQ | 366 | 2.52E−3 |
| | | VRQAPGKGPEWVSSIYSDLSTTYYADSVKGRFTISRD | | QKPDQAPVLVIYADNRRPSEIPERFSGSNSGDTATL | | |
| | | RAKNTLYLQMNSLKSEDTAVYYCARLSTVAGVDYWG | | TISGAQAEDEADYYCQVWDSGANAAVFGGGTHLTV | | |
| | | KGTLVTVSS | | LG | | |
| B4 | 1 | EVQVQESGPGLVKPSQTLSLTCTVSGGSITTDYSYWS | 341 | SYELTQSPSVSVALRQTAKITCGGNNIGSKSAQWYQ | 367 | 4.10E−3 |
| | | WIRQPPGKGLEWMGAIAFSDTTYYSPSLKSRTSISRDT | | QKPGQAPVLVIYADNRRPSEIPERFSGSNSGDTATL | | |
| | | SKNQFTLQLSSVTPEDTAVYYCARLSTVAGMDYWGK | | TISGAQAEDEADYYCQVWDSSANAAVFGGGTHLTV | | |
| | | GTLVTVSSASAAASG | | LG | | |
| C10 | 2 | EVQLVQPGAELRNPGASVKVSCKASGYTFTAYYIDWV | 342 | SYELTQSPSVSVALRQTAKITCGGDNIGSKSAQWYQ | 368 | 3.22E−3 |
| | | RQAPGQGLEWMGRIDPEDGGTTYEQKFQGRVTFTAD | | QKPGQAPVLVIYADSRRPSGIPERFSGSNSGNTATL | | |
| | | TSTSTVYVELSSLRSEDTAVYYCARSTEIPTIRYYGMD | | TISGAQAEDEADYYCQVWDSSANAAVFGGGTHLTV | | |
| | | YWGKGTPVTVSSASAAASG | | LG | | |
| B5 | 2 | EVQLVQPGAELRNPGASVKVSCKASGYTFTSHYIDWV | 343 | QSVLTQPSAVSVSLGQTARITCQGGSLGSYGANWH | 369 | 4.07E−3 |
| | | RQAPGQGLEWMGRIDPEDGGTTYEQKFQGRVTFTAD | | QQKPGQAPVLLIYGDNSRPSGIPERFSGSKSGGTAT | | |
| | | TSTSTVYVELSSLRSEDTAVYYCARSTEIPTIRYYGMD | | LTISGAQAEDEADYYCQSFDYSGNAVFGGGTHLTVLG | | |
| | | YWGKGTPVTVSS | | | | |
| C1 | 10 | ELQVVESGGGLVPGGSLRLSCAASGFRVSVNAMSWV | 344 | QAGLTQLPSVSGSPGQKITISCTGSSNNIGAGYSVQ | 370 | |
| | | RQAPGKGLEWVSHIGSGGITGYADSVKGRFTISRDSA | | WFQQVPGSPPRLLIDGATNRDSGVPDRFSDSRSGS | | |
| | | KNTLYLQMNSLKPEDTAVYYCTKRSSGYAWGQGTQV | | SASLTITGLQAEDEADYYCECYDNSLRGAVFGGGTH | | |
| | | TVSS | | LTVLG | | |
| C12 | 10 | ELQVVESGGGLVPGGSLRLSCAASGFRVSVNAMSWV | 344 | QAGLTQLPSVSGSPGQKITISCTGSSNNIGAGYSVQ | 371 | |
| | | RQAPGKGLEWVSHIGSGGITGYADSVKGRFTISRDSA | | WFQQVPGSPPRLLIDGATNRDSGVPDRFSDSRSGS | | |
| | | KNTLYLQMNSLKPEDTAVYYCTKRSSGYAWGQGTQV | | SASLTITGLQAEDEADYYWMLQQPQGVLCSAEGPI | | |
| | | TVSSASAAASG | | | | |
| A8² | 11 | EVQVQESGPGLVKPSQTLSLTCTVSGESITTNYYYWN | 345 | SYELTQSPSVSVALRQAAKITCGGNNIASKFVNWYQ | 372 | 3.00E−3 |
| | | WIRQPPGKALEWMGAIGDSGTTWYNPSLKSRTSISRD | | QKPGQAPILVISKDTNRPSGIPERFSGSNSGNTATLT | | |
| | | TSKNQFTLQLISVTPEDTAVYYCARGFYDGSSLDFAS | | VSGAQAEDEADYYCQVWDGSAAVFGGGTHLTVLG | | |
| | | WGQGTQVTVSS | | | | |
| C4² | 11 | QVQLQESGPGLVKPSQTLSLTCTVSGGSITTNYYYWS | 346 | LPVLTQPSAVSVSLGQTARITCQGGNVGSYYTSWY | 373 | 3.13E−4 |
| | | WIRQPPGKGLEWMGAIAYSGSTYYSPSLKSRTSISRD | | QQKPGQAPVLVIYKDSERPSGIPERFSGSSSGNTAT | | |
| | | TSKNQFTLQLSSVTPEDTAVYYCARGYYSGSSLDFGS | | LTVSGAQAEDEGDYYCQVWDSSAAVFGGGTHLTVLG | | |
| | | WGQGTQVTVSS | | | | |
| D1² | * | QVQLQESGGGLVQPGGSLRLSCAASGFTFSWYAMS | 347 | QAVVTQEPSLSVSPGGTVTLTCGLNSGSVTNGNYP | 374 | 5.30E−3 |
| | | WVRQGPGKGLEWVSGINGGGGSTSYEDSVKGRFTIS | | GWYQQTPGQAPRALIYSTNSRHSGVPGRYSGSISG | | |
| | | RDNAKNTLYLEMNSLKPEDTAVYYCARYSDGYSGSYT | | NKAALTITGAEPEDEADYYCALWQAAGTVVFGGGT | | |
| | | YTDMDYWGKGTLVSVSS | | HLTVLG | | |
| D7² | * | QVQLVESGGGLVQPGGSLRLSCAASGFTFSWYAMS | 348 | QAVVTQEPSLSVSPGGTVTLTCGLSSGSVTSSNYPV | 375 | 2.75E−2 |
| | | WVRQGPGKGLEWVSAINGGGGSTSYEDSVKGRFTIS | | WYQQTPGQAPHLLIYNTNTRHSGVPSRFSGFISGNK | | |
| | | RDNAKNTLYLEMNSLKPEDTAVYYCARYSDGYSGSYT | | AALTITGAQPEDEADYYCASYRRSGRAVFGGGTHLT | | |
| | | YTDMDYWGKGTLVTVSS | | VLG | | |
| D8² | 12 | EVQLVESEGGSVQPGDSLRLSCAASGFSFSNAGMSW | 349 | QSALTQPPSVSGTLGKTVTISCTGTSSDIGGYNSVS | 376 | 1.41E−3 |
| | | VRQAPGKGLEWVSAIIPGGYAPYYADSVKGRFTISRD | | WYQQLPGTAPKLLIYEVNKRASGIPDRFSGSKSGDT | | |
| | | NAKNTVSLQMNSLKPEDTALYYCAKYGGNWDLEVWG | | ASLTISGLQSADEADYYCASYRNTRKSVFGGGTHLT | | |
| | | QGTLVTVSS | | VLG | | |
| C2² | 12 | QLQLVESGGGLQQPGGSLRLSCAASGFTFSSYAMGW | 350 | QSVLTQPPSVSGSPGKTVTISCAGTSSDVGYGNYVS | 377 | 2.14E−3 |
| | | VRQAPGKGLEWISIILNDGHRTYYTDSVKGRFTISRDN | | WYQQLPGMAPKLLIYNANKRASGITDRFSASKSGNT | | |
| | | AKDTLSLQMNSLKPEDTAVYFCVKYVLNWAISDWGQ | | ASLTISGLQSGDEAVYYCASYRYRDNAVFGGGTHLT | | |
| | | GTQVTVSS | | VLG | | |
| A4² | 13 | QLQVVESGGGLVQPGGSLRLSCAASGFTFDSYWMY | 351 | LPVLTQPPSVSGTLGKTVTISCAGTSSDIGDYNYVS | 378 | 2.39E−4 |
| | | WVRQAPGKGLEWLSAINPGGGMTYYADSVKGRFTIA | | WYQQLPGTAPKLLIYKVSTRASGIPDRFSGSKSGNT | | |
| | | RDNAKNTVYLQMNSLKSEDTAVYYCAKELRGSYYYIS | | ASLTISGLQSEDEADYYCASYRTSNNVVFGGGTHLT | | |
| | | SEYDYWGQGTQVTVSS | | VLG | | |
| A5² | 14 | ELQLVESGGGLVQPGGSLRLSCAASGSTFSTAAMSW | 352 | SSALTQPPSVSGSPGKTVTISCAGTSSDVGYGNYVS | 379 | 3.27E−3 |
| | | VRQAPGKGLEWVSAISSFGASTSYADSVKGRFTISRDI | | WYQQLPGMAPKLLIHDVNKRASGITDRFSGSKSGNT | | |
| | | AKNTLYLQMNSLKPEDAAVYYCANVGLHPSWGFTSG | | ASLTISGLQSEDEADYYCASYRRRNEVVFGGGTHLT | | |
| | | FWGQGTQVTVSS | | VLG | | |
| A1² | 15 | QVQLVESGGGLVQPGGSLRLSCAASGFTFSSYAMTW | 353 | SYELTQSSAVSLSLGQTARITCQGDNLGRYYVNWY | 380 | 9.73E−4 |
| | | VRQAPGKGLEWVSTITSNGDSTSYADSVKGRFTISRD | | QQKPGQAPVQVIYGNSNRPSGIPERFSGSSSGGTA | | |
| | | NAKNTLYLQMDSLKPEDTAVYYCAKLGDGSYWFYYY | | TLTISGAQAEDEADYYCQTYENSGVNLVFGGGTKLT | | |
| | | GMDYVVGQGTQVTVTSS | | VLG | | |
| D6² | 16 | QLQLVESGGGLVQPGGSLRLSCAASGGIFDVMGWYH | 354 | SYELTQSPSVSVALRQTAKITCGGDNIGSKSAQWYQ | 381 | 5.82E−4 |
| | | QAPGKQRGLVASITPAGSTNYADSVKGRFTISRDNAK | | QKPGQAPVLVIYAGSRRPSGIPERFSGSNSGNTATL | | |
| | | NTVYLQMNSLKPDDTGVYYCYADGIDPRYGGDEFWG | | TVSGAQAEDEANYSCQVWDSSANASVFGGGTHLT | | |
| | | QGTQVTVSS | | VLG | | |

¹Kd: off-rate for Nt-GCGR binding is measured on Biacore using perimlasmic extracts
* No binding to Nt-GCGR-Fc or to GCGR over expressing CHO cells
²New GCGR binders

### Example 17

### Counter Selection with Null Lipoparticles

As described above, a high background was seen on null lipoparticles following screening using lipoparticles. In order to reduce this background, input phage were pre-incubated for 30 min at room temperature with 10-fold excess of null lipoparticles in suspension, and subsequently selected on coated GCGR-lipoparticles. In parallel, selection conditions without counter-selection were performed as a control. As diagnosis for enrichment of GCGR binders, selection on the recombinant N-terminal domain was included in the selection campaign. Selections were performed with the kappa library 73 of llama 73.

After the first round of selection there was a poor output. In the second round of selection, no difference in output was seen for selection conditions with counter-selection on null lipoparticles as compared with no counter-selection. For both conditions, counter-selection and no counter-selection, enrichment was seen on the N-terminal domain, which indicates that specific binders were enriched in the first round on GCGR lipoparticles. This enrichment was a little higher in the case of counter-selection on the null lipoparticles. For the control selection on the N-terminal domain, enrichment was already seen after the second round on Nt-GCGR.

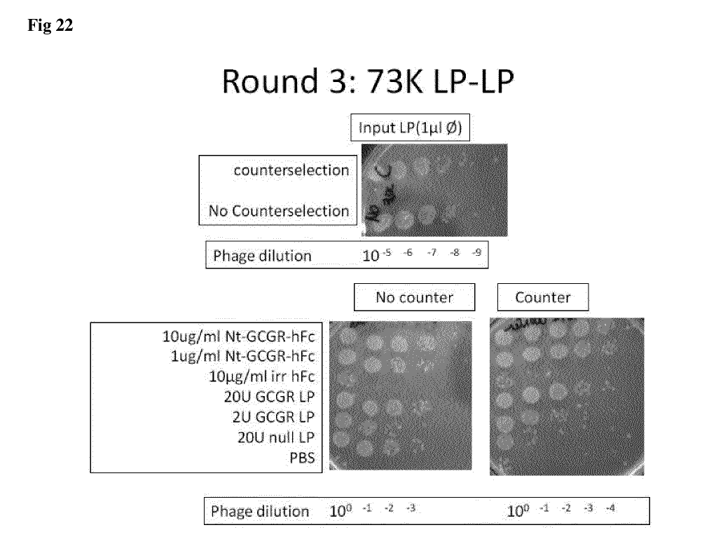

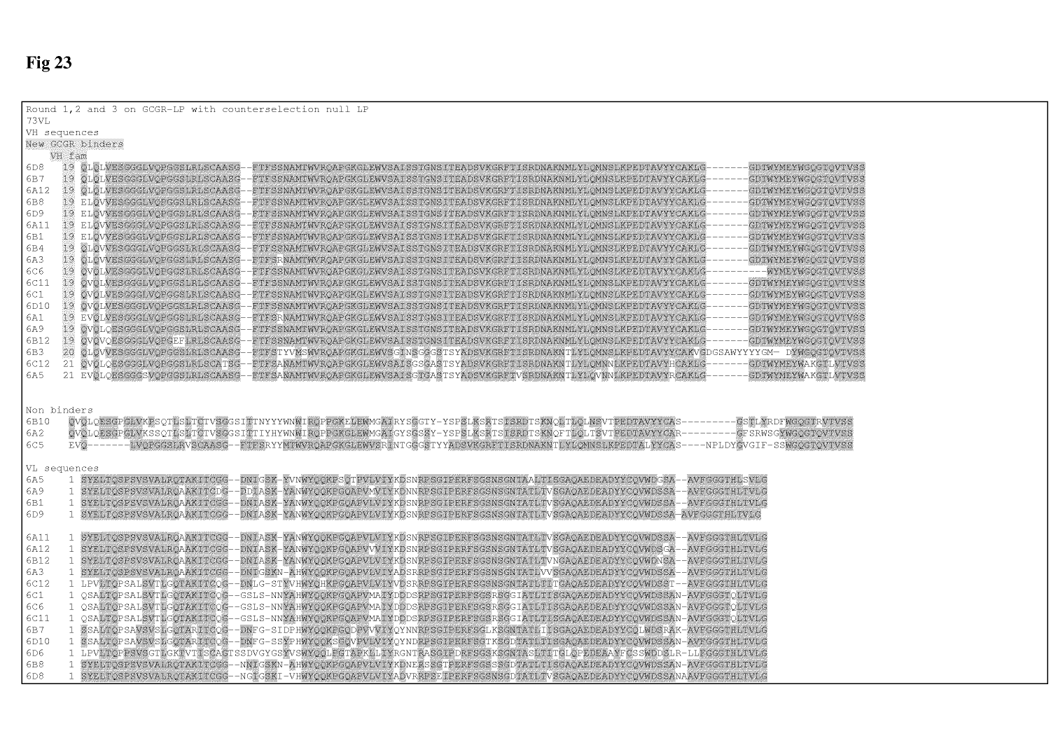

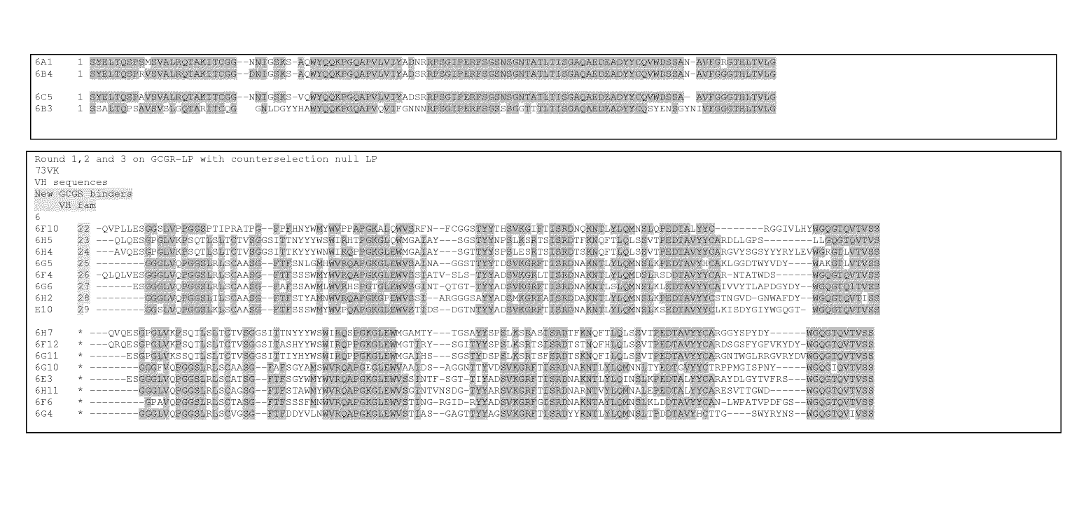

In the third round of selection, an 1000-fold enrichment was observed on the GCGR lipoparticles when counter-selections were carried out on null lipoparticles (FIG. 22). While the outputs on the GCGR lipoparticles were similar in case of counter-selection versus no counter selection, the background on the null lipoparticle was strongly reduced by counter-selection. Also the diagnosis on Nt-GCGR showed 10-fold more enrichment with counter-selection versus no counter-selection, indicating more enrichment of GCGR-specific clones during the previous rounds. In conclusion, counter-selection with null lipoparticles reduces the background on null lipoparticles dramatically, and shows good enrichment for clones binding GCGR lipoparticles.

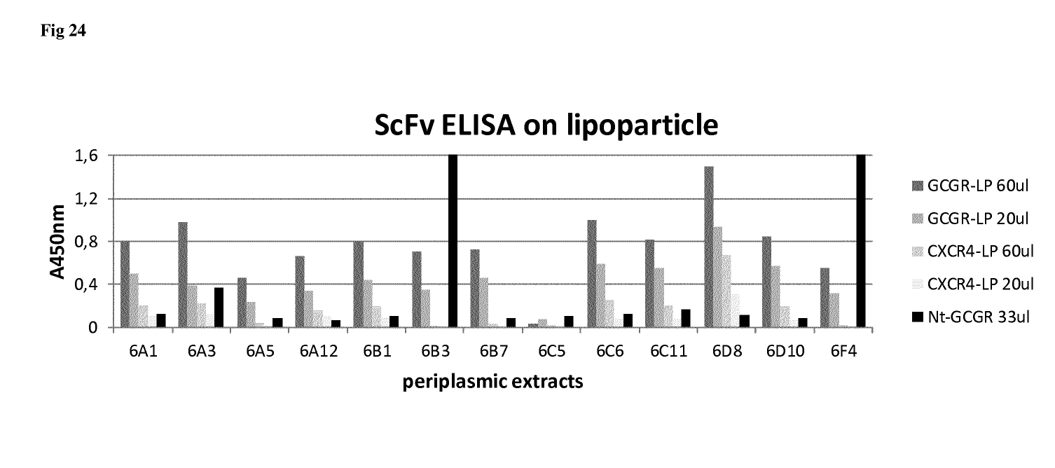

Sequences of the conventional clones identified after three rounds of selection on GCGR lipoparticles with counter selection on null lipoparticles are listed in Table 20 below and an alignment of the sequences is shown in FIG. 24.

TABLE 21
ScFv sequences of clones picked after three rounds of selection
on GCGR lipoparticle with counter selection on null lipoparticles

| | | | SEQ | | SEQ |
|---|---|---|---|---|---|
| | VH | | ID | | ID |
| scFv | Family | VH | NO: | VL | NO: |
| 6D8 | 19 | QLQLVESGGGLVQPGGSLRLSCAASGFTFSSNAMTWVR | 382 | SYELTQSPSVSVALRQTAKITCGGNGIGSKIVHWYQQKP | 394 |
| | | QAPGKGLEWVSAISSTGNSITEADSVKGRFTISRDNAKNM | | GQAPVLVIYADVRRPSEIPERFSGSNSGDTATLTVSGAQA | |
| | | LYLQMNSLKPEDTAVYYCAKLGGDTWYMEYWGQGTQVT | | EDEADYYCQVWDSSANAAVFGGGTHLTVLG | |
| | | VSS | | | |
| 6B7 | 19 | QLQLVESGGGLVQPGGSLRLSCAASGFTFSSNAMTWVR | 382 | SSALTQPSAVSVSLGQTARITCQGDNFGSIDPHWYQQKP | 395 |
| | | QAPGKGLEWVSAISSTGNSITEADSVKGRFTISRDNAKNM | | GQDPVVVIYQYNNRPSGIPERFSGLKSGNTATLIISGAQA | |
| | | LYLQMNSLKPEDTAVYYCAKLGGDTWYMEYWGQGTQVT | | EDEADYYCQLWDSRAKAVFGGGTHLTVLG | |
| | | VSS | | | |
| 6A12 | 19 | QLQLVESGGGLVQPGGSLRLSCAASGFTFSSNAMTWVR | 382 | SYELTQSPSVSVALRQAAKITCGGDNIASKYANWYQQKP | 396 |
| | | QAPGKGLEWVSAISSTGNSITEADSVKGRFTISRDNAKNM | | GQAPVVVIYKDSNRPSGIPERFSGSNSGNTATLTVSGAQ | |
| | | LYLQMNSLKPEDTAVYYCAKLGGDTWYMEYWGQGTQVT | | AEDEADYYCQVWDSGAAVFGGGTHLTVLG | |
| | | VSS | | | |
| 6B8 | 19 | ELQVVESGGGLVQPGGSLRLSCAASGFTFSSNAMTWVR | 383 | SYELTQSPSVSVALRQTAKITCGGNNIGSKNAHWYQQKP | 397 |
| | | QAPGKGLEWVSAISSTGNSITEADSVKGRFTISRDNAKNM | | GQAPVLVIYKDNERSSGTPERFSGSSSGDTATLTISGAQA | |
| | | LYLQMNSLKPEDTAVYYCAKLGGDTWYMEYWGQGTQVT | | EDEADYYCQVWDSSANAVFGGGTHLTVLG | |
| | | VSS | | | |
| 6D9 | 19 | ELQVVESGGGLVQPGGSLRLSCAASGFTFSSNAMTWVR | 383 | SYELTQSPSVSVALRQAAKITCGGDNIASKYANWYQQKP | 398 |
| | | QAPGKGLEWVSAISSTGNSITEADSVKGRFTISRDNAKNM | | GQAPVLVIYKDSNRPSGIPERFSGSNSGNTATLTVSGAQ | |
| | | LYLQMNSLKPEDTAVYYCAKLGGDTWYMEYWGQGTQVT | | AEDEADYYCQVWDSSAAVFGGGTHLTVLG | |
| | | VSS | | | |
| 6A11 | 19 | ELQVVESGGGLVQPGGSLRLSCAASGFTFSSNAMTWVR | 383 | SYELTQSPSVSVALRQAAKITCGGDNIASKYANWYQQKP | 398 |
| | | QAPGKGLEWVSAISSTGNSITEADSVKGRFTISRDNAKNM | | GQAPVLVIYKDSNRPSGIPERFSGSNSGNTATLTVSGAQ | |
| | | LYLQMNSLKPEDTAVYYCAKLGGDTWYMEYWGQGTQVT | | AEDEADYYCQVWDSSAAVFGGGTHLTVLG | |
| | | VSS | | | |
| 6B1 | 19 | ELQVVESGGGLVQPGGSLRLSCAASGFTFSSNAMTWVR | 383 | SYELTQSPSVSVALRQAAKITCGGDNIASKYANWYQQKP | 398 |
| | | QAPGKGLEWVSAISSTGNSITEADSVKGRFTISRDNAKNM | | GQAPVLVIYKDSNRPSGIPERFSGSNSGNTATLTVSGAQ | |
| | | LYLQMNSLKPEDTAVYYCAKLGGDTWYMEYWGQGTQVT | | AEDEADYYCQVWDSSAAVFGGGTHLTVLG | |
| | | VSS | | | |
| 6B4 | 19 | QLQVVESGGGLVQPGGSLRLSCAASGFTFSSNAMTWVR | 384 | SYELTQSPRVSVALRQTAKITCGGDNIGSKSAQWYQQKP | 399 |
| | | QAPGKGLEWVSAISSTGNSITEADSVKGRFTISRDNAKNM | | GQAPVLVIYADSRRPSGIPERFSGSNSGNTATLTISGAQA | |
| | | LYLQMNSLKPEDTAVYYCAKLGGDTWYMEYWGQGTQVT | | EDEADYYCQVWDSSANAVFGGGTHLTVLG | |
| | | VSS | | | |
| 6A3 | 19 | QLQVVESGGGLVQPGGSLRLSCAASGFTFSRNAMTWVR | 385 | SYELTQSPSVSVALRQAAKITCGGDNIGSKNAHWYQQKP | 400 |
| | | QAPGKGLEWVSAISSTGNSITEADSVKGRFTISRDNAKNM | | GQAPVLVIYADSRRPSGIPERFSGSNSGNTATLVVSGAQ | |
| | | LYLQMNSLKPEDTAVYYCAKLGGDTWYMEYWGQGTQVT | | AEDEADYYCQVWDSSAAVFGGGTHLTVLG | |
| | | VSS | | | |
| 6C6 | 19 | QVQLVESGGGLVQPGGSLRLSCAASGFTFSSNAMTWVR | 386 | QSALTQPSALSVTLGQTAKITCQGGSLSNNYAHWYQQKP | 401 |
| | | QAPGKGLEWVSAISSTGNSITEADSVKGRFTISRDNAKNM | | GQAPVMAIYDDDSRPSGIPERFSGSRSGGIATLTISGAQA | |
| | | LYLQMNSLKPEDTAVYYCAKLGWYMEYWGQGTQVTVSS | | EDEADYYCQVWDSSANAVFGGGTQLTVLG | |
| 6C11 | 19 | QVQLVESGGGLVQPGGSLRLSCAASGFTFSSNAMTWVR | 387 | QSALTQPSALSVTLGQTAKITCQGGSLSNNYAHWYQQKP | 401 |
| | | QAPGKGLEWVSAISSTGNSITEADSVKGRFTISRDNAKNM | | GQAPVMAIYDDDSRPSGIPERFSGSRSGGIATLTISGAQA | |
| | | LYLQMNSLKPEDTAVYYCAKLGGDTWYMEYWGQGTQVT | | EDEADYYCQVWDSSANAVFGGGTQLTVLG | |
| | | VSS | | | |
| 6C1 | 19 | QVQLVESGGGLVQPGGSLRLSCAASGFTFSSNAMTWVR | 387 | QSALTQPSALSVTLGQTAKITCQGGSLSNNYAHWYQQKP | 401 |
| | | QAPGKGLEWVSAISSTGNSITEADSVKGRFTISRDNAKNM | | GQAPVMAIYDDDSRPSGIPERFSGSRSGGIATLTISGAQA | |
| | | LYLQMNSLKPEDTAVYYCAKLGGDTWYMEYWGQGTQVT | | EDEADYYCQVWDSSANAVFGGGTQLTVLG | |
| | | VSS | | | |
| 6D10 | 19 | QVQLVESGGGLVQPGGSLRLSCAASGFTFSSNAMTWVR | 387 | SSALTQPSAVSVSLGQTARITCQGDNFGSSYPHWYQQK | 402 |
| | | QAPGKGLEWVSAISSTGNSITEADSVKGRFTISRDNAKNM | | SGQVPVLVIYQYNDRPSGIPERFSGTKSGDTATLTISGAQ | |
| | | LYLQMNSLKPEDTAVYYCAKLGGDTWYMEYWGQGTQVT | | AEDEADYYCQVWDSSANAVFGGGTHLTVLG | |
| | | VSS | | | |
| 6A1 | 19 | EVQLVESGGGLVQPGGSLRLSCAASGFTFSRNAMTWVR | 388 | SYELTQSPSMSVALRQTAKITCGGNNIGSKSAQWYQQKP | 403 |
| | | QAPGKGLEWVSAISSTGNSITEADSVKGRFTISRDNAKNM | | GQAPVLVIYADNRRPSGIPERFSGSNSGNTATLTISGAQA | |
| | | LYLQMNSLKPEDTAVYYCAKLGGDTWYMEYWGQGTQVT | | EDEADYYCQVWDSSANAVFGRGTHLTVLG | |
| | | VSS | | | |
| 6A9 | 19 | QVQLQESGGGLVQPGGSLRLSCAASGFTFSSNAMTWVR | 389 | SYELTQSPSVSVALRQAAKITCDGDDIASKYANWYQQKP | 404 |
| | | QAPGKGLEWVSAISSTGNSITEADSVKGRFTISRDNAKNM | | GQAPVMVIYKDNNRPSGIPERFSGSNSGNTATLTVSGAQ | |
| | | LYLQMNSLKPEDTAVYYCAKLGGDTWYMEYWGQGTQVT | | AEDEADYYCQVWDSSAAVFGGGTHLTVLG | |
| | | VSS | | | |
| 6B12 | 19 | QVQVQESGGGLVQPGEFLRLSCAASGFTFSSNAMTWVR | 390 | SYELTQSPSVSVALRQAAKITCGGDNIASKYANWYQQKP | 405 |
| | | QAPGKGLEWVSAISSTGNSITEADSVKGRFTISRDNAKNM | | GQAPVLVIYKDSNRPSGIPERFSGSNSGNTATLTVNGAQ | |
| | | LYLQMNSLKPEDTAVYYCAKLGGDTWYMEYWGQGTQVT | | AEDEADYYCQVWDNSAAVFGGGTHLTVLG | |
| | | VSS | | | |
| 6B3 | 20 | QLQVVESGGGLVQPGGSLRLSCAASGFTFSTYVMSWVR | 391 | SSALTQPSAVSVSLGQTARITCQGGNLDGYYHAWYQQK | 406 |
| | | QAPGKGLEWVSGINSGGGSTSYADSVKGRFTISRDNAKN | | PGQAPVQVIFGNNNRPSGIPERFSGSSSGGTTTLTISGAQ | |
| | | TLYLQMNSLKPEDTAVYYCAKVGDGSAWYYYYGMDYWG | | AEDEADYYCQSYENSGYNIVFGGGTHLTVLG | |
| | | QGTQVTVSS | | | |
| 6C12 | 21 | QVQLQESGGGLVQPGGSLRLSCATSGFTFSANAMTWVR | 392 | LPVLTQPSALSVTLGQTAKITCQGDNLGSTYVHWYQHKP | 407 |
| | | QAPGKGLEWVSAISGSGASTSYADSVKGRFTISRDNAKN | | GQAPVLVIYVDSRRPSGIPERFSGSNSGNTATLTITGAQA | |
| | | TLYLQMNNLKPEDTAVYHCAKLGGDTWYMEYWAKGTLV | | EDEADYYCQVWDSSTAVFGGGTHLTVLG | |
| | | TVSS | | | |
| 6A5 | 21 | EVQLQESGGGSVQPGGSLRLSCAASGFTFSANAMTWVR | 393 | SYELTQSPSVSVALRQTAKITCGGDNIGSKYVNWYQQKP | 408 |
| | | QAPGKGLEWVSAISGTGASTSYADSVKGRFTVSRDNAKN | | SQTPVLVIYKDSNRPSGIPERFSGSNSGNTAALTISGAQA | |
| | | TLYLQVNNLKPEDTAVYRCAKLGGDTWYMEYWAKGTLV | | EDEADYYCQVWDGSAAVFGGGTHLSVLG | |
| | | TVSS | | | |
| 6F10 | 22 | QVPLLESGGSLVPPGGSPTIPRATPGFPFHNYWMYWVPP | 409 | ND | |
| | | APGKALQWVSRFNFCGGSTYYTHSVKGIFTISRDNQKNT | | | |
| | | LYLQMNSLQPEDTALYYCRGGIVLHYWGQGTQVTVSS | | | |
| 6H5 | 23 | QLQESGPGLVKPSQTLSLTCTVSGGSITTNYYYWSWIRH | 410 | ND | |
| | | TPGKGLQWMGAIAYSGSTYYNPSLKSRTSISRDTFKNQF | | | |
| | | TLQLSSVTPEDTAVYYCARDLLGPSLLGQGTQVTVSS | | | |
| 6H4 | 24 | AVQESGPGLVKPSQTLSLTCTVSGGSITTKYYYWNWIRQ | 411 | ND | |
| | | PPGKGLEWMGAIAYSGTTYYSPSLESRTSISRDTSKNQFT | | | |
| | | LQLSSVTPEDTAVYYCARGVYSGSYYYRYLEVWGRGTLV | | | |
| | | TVSS | | | |
| 6G5 | 25 | xxxxxxGGGLVQPGGSLRLSCAASGFTFSNLGMHWVRQA | 412 | ND | |
| | | PGKGLEWVSAINAGGSTTYYTDSVKGRFTISRDNAKNTLY | | | |
| | | LQMNSLKPEDTAVYHCAKLGGDTWYVDYWAKGTLVTVSS | | | |
| 6F4 | 26 | QLQLVESGGGLVQPGGSLRLSCAASGFTFSSSWMYWVR | 413 | ND | |
| | | QAPGKGLEWVSSIATVSLSTYYADSVKGRLTISRDNAKNT | | | |
| | | LYLQMDSLRSDDTAVYYCARNTATWDSWGQGTQVTVSS | | | |
| 6G6 | 27 | xxxxxxGGGLVQPGGSLRLSCAASGFAFSSAWMLWVRHS | 414 | ND | |
| | | PGTGLEWVSGINTQTGTTYYADSVKGRFTISRDNAKNTLS | | | |
| | | LQMNSLKLEDTAVYYCAIVVYTLAPDGYDYWGQGTQITVSS | | | |
| 6H2 | 28 | xxxxxxGGGLVQPGGSLILSCAASGFTFSTYAMNWVRQAP | 415 | ND | |
| | | GKGPEWVSSIARGGGSAYYADSMKGRFAISRDDAKNTLY | | | |
| | | LQMNSLKPEDTAVYYCSTNGVDGNWAFDYWGQGTQVTI | | | |
| | | SS | | | |
| 6E10 | 29 | xxxxxxGGSLVQPGGSLKLSCAASGFTFSSSWMYWVPQA | 416 | ND | |
| | | PGKGLEWVSTIDSDGTNTYYADSVKGRFTISRDNAKNTLY | | | |
| | | LQMNSLKSEDTAVYYCLKISDYGIYWGQGTWGQGTQVTV | | | |
| | | SS | | | |
| 6H7 | * | xxxxxxSGPGLVKPSQTLSLTCTVSGGSITTNYYYWSWIRQ | 417 | ND | |
| | | SPGKGLEWMGAMTYTGSAYYSPSLKSRASISRDTFKNQF | | | |
| | | TLQLSSVTPEDTAVYYCARGGYSPYDYWGQGTQVTVSS | | | |
| 6F12 | * | xxxxxxSGPGLVKPSQTLSLTCTVSGGSITASHYYWSWIRQ | 418 | ND | |
| | | PPGKGLEWMGTIRYSGITYYSPSLKSRTSISRDTSTNQFH | | | |
| | | LQLSSVTPEDTAVYYCARDSGSFYGFVKYDYWGQGTQV | | | |
| | | TVSS | | | |
| 6G11 | * | xxxxxxSGPGLVKSSQTLSLTCTVSGGSITTIYHYWSWIRQP | 419 | ND | |
| | | PGKGLEWMGAIHSSGSTYDSPSLKSRTSFSRDTSKNQFIL | | | |
| | | QLSSVTPEDTAVYYCARGNTWGLRRGVRYDVWGQGTQ | | | |
| | | VTVSS | | | |
| 6G10 | * | xxxxxxxGVGGGFVQPGGSLRLSCAASGFAFSGYAMSWV | 420 | ND | |
| | | RQAPGEGLEWVAAIDSAGGNTTYVDSVKGRFTISRDNAK | | | |
| | | NTLYLQMNNLTYEDTGVYYCTRPPMGISPNYWGQGIQVT | | | |
| | | VSS | | | |
| 6E3 | * | xxxxxxESGGGLVQPGGSLRLSCATSGFTFSGYWMYWVR | 421 | ND | |
| | | QAPGKGLEWVSSINTFSGTTIYADSVKGRFTISRDNAKNT | | | |
| | | LYLQINSLKPEDTALYYCARAYDLGYTVFRSWGQGTQVT | | | |
| | | VSS | | | |
| 6H11 | * | xxxxxxxGVGGGLVQPGGSLRLSCAGSGFTFSTAWMYWV | 422 | ND | |
| | | RQAPGKGLEWVSGINTVNSDGTYYARSVKGRFTISRDNA | | | |
| | | RNTVYLQMNALEPEDTALYYCARESVTTGWDWGQGTQV | | | |
| | | TVSS | | | |
| 6F6 | * | xxxxxxxxxxxxxxxPGGSLRLSCTASGFTFSSSFMNWVRQA | 423 | ND | |
| | | PGKGLEWVSTINGRGIDRYYADSVKGRFGISRDNAKNTA | | | |
| | | YLQMNSLKLDDTAVYYCANLWPATVPDFGSWGQGTQVT | | | |
| | | VSS | | | |
| 6G4 | * | xxxxxxxGVGGGLVQPGGSLRLSCVGSGFTFDDYVLNWVR | 424 | ND | |
| | | QAPGKGLEWVSTIASGAGTTYYAGSVKGRFTISRDYYKN | | | |
| | | TLYLQMNSLTPDDTAVYHCTTGSWYRYNSWGQGTQVIV | | | |
| | | SS | | | |

* No binding to Nt-GCGR-Fc or to GCGR LP
xxxxxxx - sequence of region not determined

TABLE 22
Overview of new VH families and binding characteristics

| | | se- | | | Binding | | |
|---|---|---|---|---|---|---|---|
| | | quences | | | ELISA | | FACS |
| Li- | elu- | conven- | VH | Mem- | Nt- | LP- | CHO- |
| brary | tion | tional | Fam | bers | GCGR | GCGR | GCGR |
| 73 | trypsin | 14 | 19 | 11 | No | Yes | Yes |
| lambda | | | 20 | 1 | Yes | Yes | Yes |
| | 65° C. | 11 | 19 | 7 | No | Yes | Yes |
| 73 | trypsin | 7 | 21 | 1 | Yes | No | ND |
| kappa | | | 22 | 1 | Yes | No | Yes |
| | 65° C. | 12 | 23 | 1 | Yes | No | Yes |
| | | | 24 | 1 | Yes | Yes | Yes |
| | | | 25 | 1 | Yes | Yes | Yes |
| | | | 26 | 1 | Yes | Yes | Yes |
| | | | 27 | 1 | Yes | No | Yes |
| | | | 28 | 1 | Yes | No | No |

Clones originating from the output of round 3, counter-selected with excess of null lipoparticles and selected on the GCGR lipoparticles (20 units/well) were picked and grown in a 96-deep well plate (1 ml expressions). From the cultures, periplasmic fractions were prepared and tested for specific binding to the full length GCGR, and to the N-terminal GCGR domain in ELISA. As detection antibody, mouse anti-Myc 9E10 was used. As a negative control, binding to the null lipoparticles was included. Although selection was performed on full length GCGR only, binding ELISA revealed that for the kappa library, more clones were binding to the N-terminal GCGR domain than to the full length GCGR (see Table 22). For the lambda library there was one big family (family 19), all binding full length GCGR, with no binding to the Nt-domain (see Table 22). ELISA data were confirmed with periplasmic extracts from 10 ml expression culture (FIG. 24).

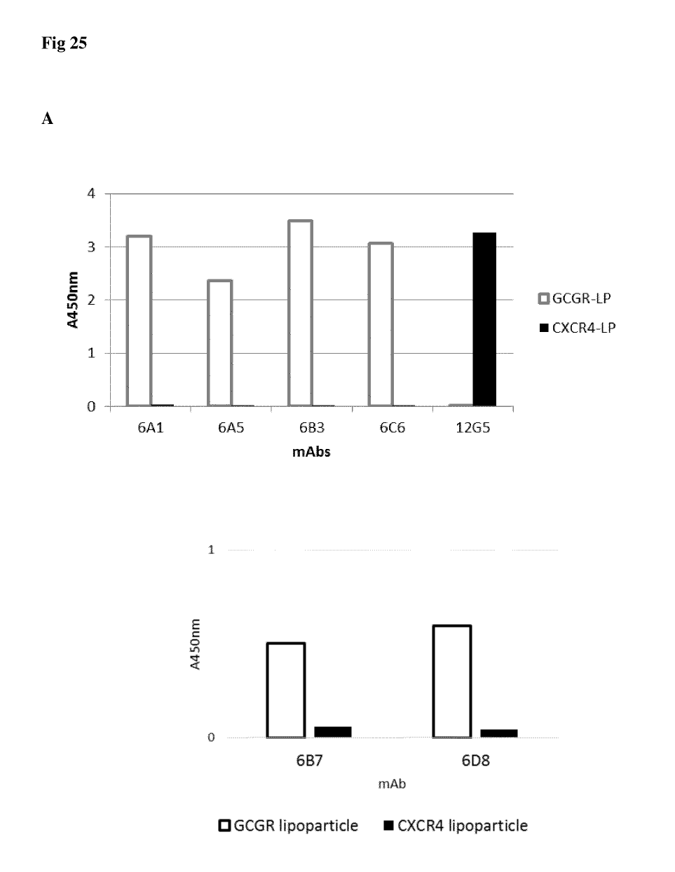

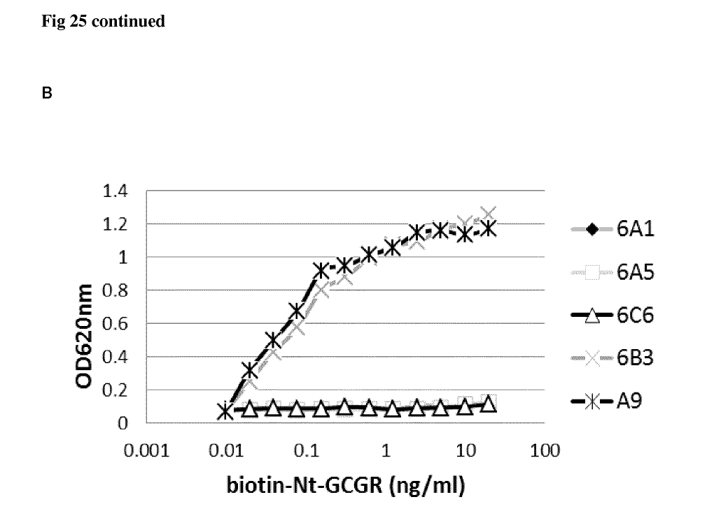

Six different scFv clones were chosen to be characterized as full IgG1. Five of these clones (6B7, 6D8, 6A5, 6C6 and 6A1) showed specific binding to GCGR-lipoparticle and not to the Nt-domain of glucagon. One clone (6B3) showed specific binding to both GCGR-lipoparticles and to Nt-GCGR (see FIG. 25).

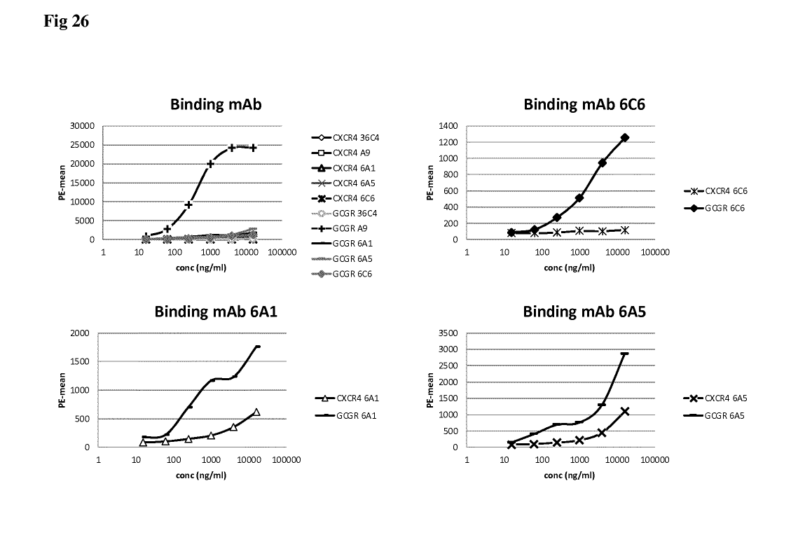

The scFv-derived mAbs were tested for binding on CHO cells overexpressing glucagon receptor. Flow cytometry data showed specific binding of mAb 6C6 and mAb 6A1 to GCGR overexpressing CHO cells (see FIG. 26). The binding of the benchmark mAb A9, which is known to bind to the N-terminal domain, was much higher. It is hypothesized that family 19 clones are binding to the loop structures. However, since the large N-terminal domain covers the loop structures, the binding of the mAbs from family 19 to the ECLs of GCGR might be difficult to measure.

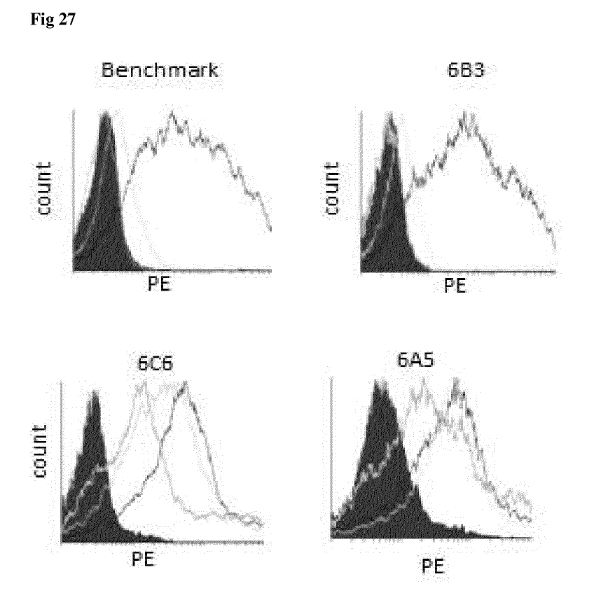

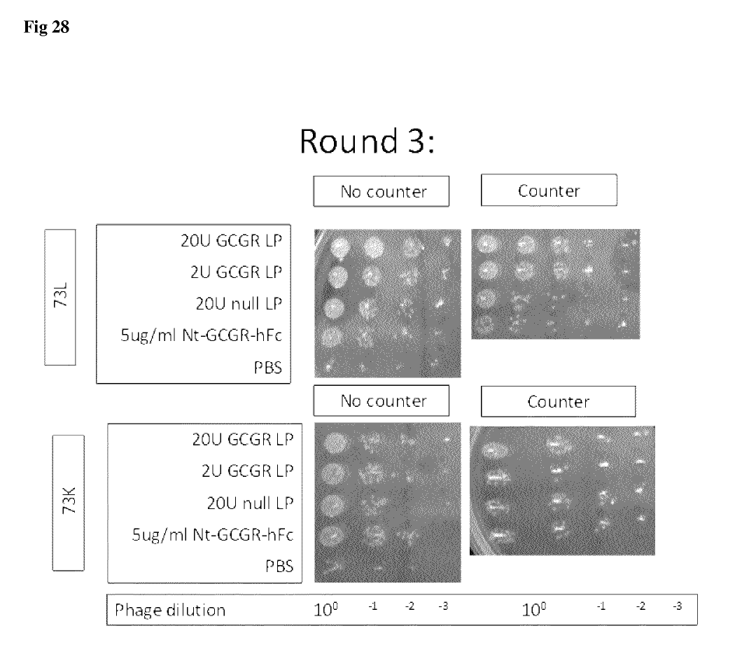

To demonstrate that mAbs 6A5 and 6C6 do not need the N-terminal domain for GCGR binding, a truncated GCGR was generated that lacks the N-terminal domain. HEK293E cells were transfected with cDNA constructs encoding GCGR with and without this Nt-domain (GCGRΔNt). Binding analysis of mAbs to the HEK293E cells overexpressing GCGR and GCGRΔNt confirmed previous results. mABs 6C6 and 6A5 bind specifically to the extracellular loops, whereas a benchmark antibody (A9) and the mAb 6B3 bind the Nt-domain. When the mAbs were pre-incubated with an excess of Nt-domain (20-fold) the binding of 6B3 and A9 to HEK293E cells overexpressing GCGR was lost, whereas mAbs 6C6 and 6A5 still showed binding to GCGR (see FIG. 27).

### Example 18

### Counter Selection with Null Lipoparticles and Nt-GCGR

To select for ECL binding clones, selections were performed on GCGR lipoparticles with counter-selection on recombinant soluble N-terminal domain. The outputs of round 1 from the previous screening campaign (see Example 17) were used i.e. counter selection with null lipoparticles and elution at 65° C. Counter-selection on the Nt-GCGR-Fc removed all the Nt-GCGR binders (5 μg/ml Nt-GCGR-Fc spotting). Enrichment on the GCGR lipoparticles, depleted of Nt-GCGR, was observed in the third round for library 73L. In the third round, when counter-selections were performed, there was an 100-fold enrichment for library 73L over the null lipoparticles and the Nt-GCGR-hFc and a 10-fold enrichment for library 73K (see FIG. 28).

Clones originating from the output of round 3 selected on the GCGR lipoparticles and counter-selected with null lipoparticles and NT-GCGR-hFc were picked and grown in a 96-deep well plate (1 ml expressions). From the cultures, periplasmic fractions were prepared and tested on specific binding to the N-terminal GCGR domain in ELISA (see Table 23). ScFv binding ELISA showed that counter-selection with the Nt-GCGR-Fc reduces the Nt-GCGR-hFc binders. No Nt-binders are seen for 73L, for 73K less than 20% Nt-binders were seen.

TABLE 23
ELISA on Nt-GCGR-hFc. 1 μg/ml Nt-GCGR-hFc was coated and 22 μl periplasmic
fraction was added. As detection antibody, mouse anti-Myc 9E10 was used.

| | 1 | 2 | 3 | 4 | 5 | 6 | 7 | 8 | 9 | 10 | 11 | 12 |
|---|---|---|---|---|---|---|---|---|---|---|---|---|
| 73L 20U GCGR LP | 0.048 | 0.044 | 0.054 | 0.076 | 0.082 | 0.083 | 0.086 | 0.096 | 0.087 | 0.08 | 0.053 | 0.038 |
| | 0.032 | 0.033 | 0.049 | 0.055 | 0.058 | 0.074 | 0.074 | 0.065 | 0.056 | 0.046 | 0.037 | 0.024 |
| | 0.034 | 0.034 | 0.052 | 0.051 | 0.059 | 0.071 | 0.066 | 0.072 | 0.066 | 0.053 | 0.042 | 0.028 |
| 73L 2U GCGR LP | 0.026 | 0.029 | 0.038 | 0.045 | 0.049 | 0.059 | 0.061 | 0.062 | 0.046 | 0.056 | 0.037 | 0.018 |
| | 0.023 | 0.027 | 0.036 | 0.047 | 0.048 | 0.055 | 0.053 | 0.055 | 0.055 | 0.041 | 0.035 | 0.019 |
| 73K 20U GCGR LP | 0.021 | 0.027 | 0.373 | 0.045 | 0.052 | 0.28 | 0.052 | 0.059 | 0.053 | 0.198 | 0.055 | 0.021 |
| | 0.018 | 0.025 | 0.317 | 0.042 | 0.044 | 0.05 | 0.044 | 0.07 | 0.052 | 0.042 | 0.034 | 0.746 |
| | 0.024 | 0.036 | 0.052 | 0.053 | 0.055 | 0.074 | 0.052 | 0.053 | 0.454 | 0.053 | 2.135 | 0.104 |

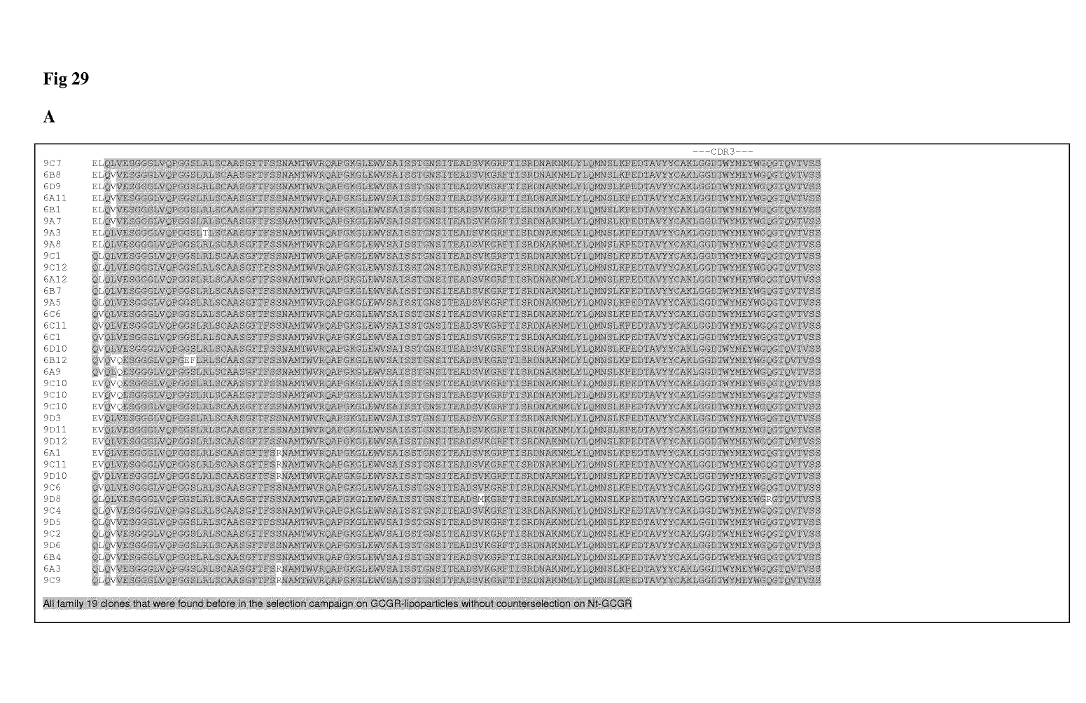

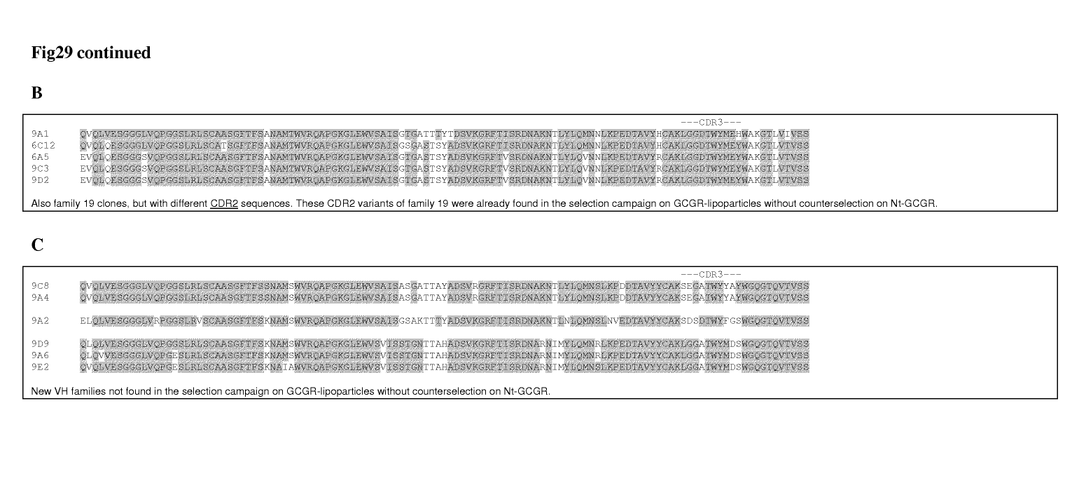

Sequence analysis showed many family 19 clones and in addition three new VH families derived from library 73L. The VH are shown in Table 24 and an alignment is shown in FIG. 29. The VL sequences (where known) are shown in Table 25.

TABLE 24
Family 19 VH sequences identified following
counter-selection on Nt-GCGR

| | | SEQ |
|---|---|---|
| | | ID |
| scFv | VH | NO: |
| 9C7 | ELQLVESGGGLVQPGGSLRLSCAASGFTFSSNAMTWVRQAPGKGLEWVSAISSTG | 425 |
| | NSITEADSVKGRFTISRDNAKNMLYLQMNSLKPEDTAVYYCAKLGGDTWYMEY | |
| | WGQGTQVTVSS | |
| 6B8 | ELQVVESGGGLVQPGGSLRLSCAASGFTFSSNAMTWVRQAPGKGLEWVSAISSTG | 383 |
| | NSITEADSVKGRFTISRDNAKNMLYLQMNSLKPEDTAVYYCAKLGGDTWYMEY | |
| | WGQGTQVTVSS | |
| 6D9 | ELQVVESGGGLVQPGGSLRLSCAASGFTFSSNAMTWVRQAPGKGLEWVSAISSTG | 383 |
| | NSITEADSVKGRFTISRDNAKNMLYLQMNSLKPEDTAVYYCAKLGGDTWYMEY | |
| | WGQGTQVTVSS | |
| 6A11 | ELQVVESGGGLVQPGGSLRLSCAASGFTFSSNAMTWVRQAPGKGLEWVSAISSTG | 383 |
| | NSITEADSVKGRFTISRDNAKNMLYLQMNSLKPEDTAVYYCAKLGGDTWYMEY | |
| | WGQGTQVTVSS | |
| 6B1 | ELQVVESGGGLVQPGGSLRLSCAASGFTFSSNAMTWVRQAPGKGLEWVSAISSTG | 383 |
| | NSITEADSVKGRFTISRDNAKNMLYLQMNSLKPEDTAVYYCAKLGGDTWYMEY | |
| | WGQGTQVTVSS | |
| 9A7 | ELQVVESGGGLVQPGGSLRLSCAASGFTFSSNAMTWVRQAPGKGLEWVSAISSTG | 383 |
| | NSITEADSVKGRFTISRDNAKNMLYLQMNSLKPEDTAVYYCAKLGGDTWYMEY | |
| | WGQGTQVTVSS | |
| 9A3 | ELQLVESGGGLVQPGGSLTLSCAASGFTFSSNAMTWVRQAPGKGLEWVSAISSTG | 426 |
| | NSITEADSVKGRFTISRDNAKNMLYLQMNSLKPEDTAVYYCAKLGGDTWYMEY | |
| | WGQGTQVTVSS | |
| 9A8 | ELQLVESGGGLVQPGGSLRLSCAASGFTFSSNAMTWVRQAPGKGLEWVSAISSTG | 427 |
| | NSITEADSVKGRFTISRDNAKNMLYLQMNSLKPEDTAVYYCAKLGGDTWYMEY | |
| | WGQGTQVTVSS | |
| 9C1 | QLQLVESGGGLVQPGGSLRLSCAASGFTFSSNAMTWVRQAPGKGLEWVSAISSTG | 382 |
| | NSITEADSVKGRFTISRDNAKNMLYLQMNSLKPEDTAVYYCAKLGGDTWYMEY | |
| | WGQGTQVTVSS | |
| 9C12 | QLQLVESGGGLVQPGGSLRLSCAASGFTFSSNAMTWVRQAPGKGLEWVSAISSTG | 382 |
| | NSITEADSVKGRFTISRDNAKNMLYLQMNSLKPEDTAVYYCAKLGGDTWYMEY | |
| | WGQGTQVTVSS | |
| 6A12 | QLQLVESGGGLVQPGGSLRLSCAASGFTFSSNAMTWVRQAPGKGLEWVSAISSTG | 382 |
| | NSITEADSVKGRFTISRDNAKNMLYLQMNSLKPEDTAVYYCAKLGGDTWYMEY | |
| | WGQGTQVTVSS | |
| 6B7 | QLQLVESGGGLVQPGGSLRLSCAASGFTFSSNAMTWVRQAPGKGLEWVSAISSTG | 382 |
| | NSITEADSVKGRFTISRDNAKNMLYLQMNSLKPEDTAVYYCAKLGGDTWYMEY | |
| | WGQGTQVTVSS | |
| 9A5 | QLQLVESGGGLVQPGGSLRLSCAASGFTFSSNAMTWVRQAPGKGLEWVSAISSTG | 382 |
| | NSITEADSVKGRFTISRDNAKNMLYLQMNSLKPEDTAVYYCAKLGGDTWYMEY | |
| | WGQGTQVTVSS | |
| 6C6 | QVQLVESGGGLVQPGGSLRLSCAASGFTFSSNAMTWVRQAPGKGLEWVSAISST | 387 |
| | GNSITEADSVKGRFTISRDNAKNMLYLQMNSLKPEDTAVYYCAKLGGDTWYME | |
| | YWGQGTQVTVSS | |
| 6C11 | QVQLVESGGGLVQPGGSLRLSCAASGFTFSSNAMTWVRQAPGKGLEWVSAISST | 387 |
| | GNSITEADSVKGRFTISRDNAKNMLYLQMNSLKPEDTAVYYCAKLGGDTWYME | |
| | YWGQGTQVTVSS | |
| 6C1 | QVQLVESGGGLVQPGGSLRLSCAASGFTFSSNAMTWVRQAPGKGLEWVSAISST | 387 |
| | GNSITEADSVKGRFTISRDNAKNMLYLQMNSLKPEDTAVYYCAKLGGDTWYME | |
| | YWGQGTQVTVSS | |
| 6D10 | QVQLVESGGGLVQPGGSLRLSCAASGFTFSSNAMTWVRQAPGKGLEWVSAISST | 387 |
| | GNSITEADSVKGRFTISRDNAKNMLYLQMNSLKPEDTAVYYCAKLGGDTWYME | |
| | YWGQGTQVTVSS | |
| 6B12 | QVQVQESGGGLVQPGEFLRLSCAASGFTFSSNAMTWVRQAPGKGLEWVSAISST | 390 |
| | GNSITEADSVKGRFTISRDNAKNMLYLQMNSLKPEDTAVYYCAKLGGDTWYME | |
| | YWGQGTQVTVSS | |
| 6A9 | QVQLQESGGGLVQPGGSLRLSCAASGFTFSSNAMTWVRQAPGKGLEWVSAISST | 389 |
| | GNSITEADSVKGRFTISRDNAKNMLYLQMNSLKPEDTAVYYCAKLGGDTWYME | |
| | YWGQGTQVTVSS | |
| 9C10 | EVQVQESGGGLVQPGGSLRLSCAASGFTFSSNAMTWVRQAPGKGLEWVSAISST | 428 |
| | GNSITEADSVKGRFTISRDNAKNMLYLQMNSLKPEDTAVYYCAKLGGDTWYME | |
| | YWGQGTQVTVSS | |
| 9D3 | EVQLVESGGGLVQPGGSLRLSCAASGFTFSSNAMTWVRQAPGKGLEWVSAISSTG | 429 |
| | NSITEADSVKGRFTISRDNAKNMLYLQMNSLKPEDTAVYYCAKLGGDTWYMEY | |
| | WGQGTQVTVSS | |
| 9D11 | EVQLVESGGGLVQPGGSLRLSCAASGFTFSSNAMTWVRQAPGKGLEWVSAISSTG | 430 |
| | NSITEADSVKGRFTISRDNAKNMLYLQMNSLKPEDTAVYYCAKLGGDTWYMEY | |
| | WGQGTQVTVSS | |
| 9D12 | EVQLVESGGGLVQPGGSLRLSCAASGFTFSSNAMTWVRQAPGKGLEWVSAISSTG | 431 |
| | NSITEADSVKGRFTISRDNAKNMLYLQMNSLKPEDTAVYYCAKLGGDTWYMEY | |
| | WGQGTQVTVSS | |
| 6A1 | EVQLVESGGGLVQPGGSLRLSCAASGFTFSRNAMTWVRQAPGKGLEWVSAISST | 388 |
| | GNSITEADSVKGRFTISRDNAKNMLYLQMNSLKPEDTAVYYCAKLGGDTWYME | |
| | YWGQGTQVTVSS | |
| 9C11 | EVQLVESGGGLVQPGGSLRLSCAASGFTFSRNAMTWVRQAPGKGLEWVSAISST | 388 |
| | GNSITEADSVKGRFTISRDNAKNMLYLQMNSLKPEDTAVYYCAKLGGDTWYME | |
| | YWGQGTQVTVSS | |
| 9D10 | QVQLVESGGGLVQPGGSLRLSCAASGFTFSRNAMTWVRQAPGKGLEWVSAISST | 432 |
| | GNSITEADSVKGRFTISRDNAKNMLYLQMNSLKPEDTAVYYCAKLGGDTWYME | |
| | YWGQGTQVTVSS | |
| 9C6 | QVQLVESGGGLVQPGGSLRLSCAASGFTFSSNAMTWVRQAPGKGLEWVSAISST | 433 |
| | GNSITEADSVKGRFTISRDNAKNMLYLQMNSLKPEDTAVYYCAKLGGDTWYME | |
| | YWGQGTQVTVSS | |
| 9D8 | QLQLVESGGGLVQPGGSLRLSCAASGFTFSSNAMTWVRQAPGKGLEWVSAISSTG | 434 |
| | NSITEADSMKGRFTISRDNAKNMLYLQMNSLKPEDTAVYYCAKLGGDTWYMEY | |
| | WGRGTQVTVSS | |
| 9C4 | QLQVVESGGGLVQPGGSLRLSCAASGFTFSSNAMTWVRQAPGKGLEWVSAISST | 384 |
| | GNSITEADSVKGRFTISRDNAKNMLYLQMNSLKPEDTAVYYCAKLGGDTWYME | |
| | YWGQGTQVTVSS | |
| 9D5 | QLQVVESGGGLVQPGGSLRLSCAASGFTFSSNAMTWVRQAPGKGLEWVSAISST | 384 |
| | GNSITEADSVKGRFTISRDNAKNMLYLQMNSLKPEDTAVYYCAKLGGDTWYME | |
| | YWGQGTQVTVSS | |
| 9C2 | QLQVVESGGGLVQPGGSLRLSCAASGFTFSSNAMTWVRQAPGKGLEWVSAISST | 384 |
| | GNSITEADSVKGRFTISRDNAKNMLYLQMNSLKPEDTAVYYCAKLGGDTWYME | |
| | YWGQGTQVTVSS | |
| 9D6 | QLQVVESGGGLVQPGGSLRLSCAASGFTFSSNAMTWVRQAPGKGLEWVSAISST | 384 |
| | GNSITEADSVKGRFTISRDNAKNMLYLQMNSLKPEDTAVYYCAKLGGDTWYME | |
| | YWGQGTQVTVSS | |
| 6B4 | QLQVVESGGGLVQPGGSLRLSCAASGFTFSSNAMTWVRQAPGKGLEWVSAISST | 384 |
| | GNSITEADSVKGRFTISRDNAKNMLYLQMNSLKPEDTAVYYCAKLGGDTWYME | |
| | YWGQGTQVTVSS | |
| 6A3 | QLQVVESGGGLVQPGGSLRLSCAASGFTFSRNAMTWVRQAPGKGLEWVSAISST | 385 |
| | GNSITEADSVKGRFTISRDNAKNMLYLQMNSLKPEDTAVYYCAKLGGDTWYME | |
| | YWGQGTQVTVSS | |
| 9C9 | QLQVVESGGGLVQPGGSLRLSCAASGFTFSRNAMTWVRQAPGKGLEWVSAISST | 385 |
| | GNSITEADSVKGRFTISRDNAKNMLYLQMNSLKPEDTAVYYCAKLGGDTWYME | |
| | YWGQGTQVTVSS | |
| 9A1 | QVQLVESGGGLVQPGGSLRLSCAASGFTFSANAMTWVRQAPGKGLEWVSAISGT | 435 |
| | GATTTYTDSVKGRFTISRDNAKNTLYLQMNNLKPEDTAVYHCAKLGGDTWYME | |
| | HWAKGTLVIVSS | |
| 6C12 | QVQLQESGGGLVQPGGSLRLSCATSGFTFSANAMTWVRQAPGKGLEWVSAISGS | 392 |
| | GASTSYADSVKGRFTISRDNAKNTLYLQMNNLKPEDTAVYHCAKLGGDTWYME | |
| | YWAKGTLVTVSS | |
| 6A5 | EVQLQESGGGSVQPGGSLRLSCAASGFTFSANAMTWVRQAPGKGLEWVSAISGT | 393 |
| | GASTSYADSVKGRFTVSRDNAKNTLYLQVNNLKPEDTAVYRCAKLGGDTWYME | |
| | YWAKGTLVTVSS | |
| 9C3 | EVQLQESGGGSVQPGGSLRLSCAASGFTFSANAMTWVRQAPGKGLEWVSAISGT | 393 |
| | GASTSYADSVKGRFTVSRDNAKNTLYLQVNNLKPEDTAVYRCAKLGGDTWYME | |
| | YWAKGTLVTVSS | |
| 9D2 | EVQLQESGGGSVQPGGSLRLSCAASGFTFSANAMTWVRQAPGKGLEWVSAISGT | 393 |
| | GASTSYADSVKGRFTVSRDNAKNTLYLQVNNLKPEDTAVYRCAKLGGDTWYME | |
| | YWAKGTLVTVSS | |
| 9C8 | QVQLVESGGGLVQPGGSLRLSCAASGFTFSSNAMSWVRQAPGKGLEWVSAISAS | 436 |
| | GATTAYADSVRGRFTISRDNAKNTLYLQMNSLKPDDTAVYYCAKSEGATWYYA | |
| | YWGQGTQVTVSS | |
| 9A4 | QVQLVESGGGLVQPGGSLRLSCAASGFTFSSNAMSWVRQAPGKGLEWVSAISAS | 437 |
| | GATTAYADSVRGRFTISRDNAKNTLYLQMNSLKPDDTAVYYCAKSEGATWYYA | |
| | YWGQGTQVTVSS | |
| 9A2 | ELQLVESGGGLVRPGGSLRVSCAASGFTFSKNAMSWVRQAPGKGLEWVSAISGS | 438 |
| | AKTTTYADSVKGRFTISRDNAKNTLNLQMNSLNVEDTAVYYCAKSDSDTWYFGS | |
| | WGQGTQVTVSS | |
| 9D9 | QLQLVESGGGLVQPGGSLRLSCAASGFTFSKNAMSWVRQAPGKGLEWVSVISST | 439 |
| | GNTTAHADSVKGRFTISRDNARNIMYLQMNRLKPEDTAVYYCAKLGGATWYMD | |
| | SWGQGTQVTVSS | |
| 9A6 | QLQVVESGGGLVQPGESLRLSCAASGFTFSKNAMSWVRQAPGKGLEWVSVISST | 440 |
| | GNTTAHADSVKGRFTISRDNARNIMYLQMNRLKPEDTAVYYCAKLGGATWYMD | |
| | SWGQGTQVTVSS | |
| 9E2 | QVQLVESGGGLVQPGESLRLSCAASGFTFSKNAIAWVRQAPGKGLEWVSVISSTG | 441 |
| | NTTAHADSVKGRFTISRDNARNIMYLQMNSLKPEDTAVYYCAKLGGATWYMDS | |
| | WGQGTQVTVSS | |

TABLE 25
VL sequences identified following
counter-selection on Nt-GCGR

| | | SEQ ID |
|---|---|---|
| scFv | VL | NO: |
| 9A4 | SYELTQSPSVSVALRQAAKITCGGDNIASKYVNWYQQKPGQAPVLVIYK | 614 |
| | DSSRPSGIPERFSGSNSGNTATLTVSGAQAEDEADYYCQVWDNSAAVFG | |
| | GGTHLTVLG | |
| 9C8 | QPVLNQPSALSVTLGQTAKITCQGGSLGSSYAHWYQQKPGQAPVLVIYA | 615 |
| | DNRRPSGTPERFSGSNSGNTATLTISGAQAEDEADYYCQVWDSSANAVF | |
| | GGGTHLTVLG | |

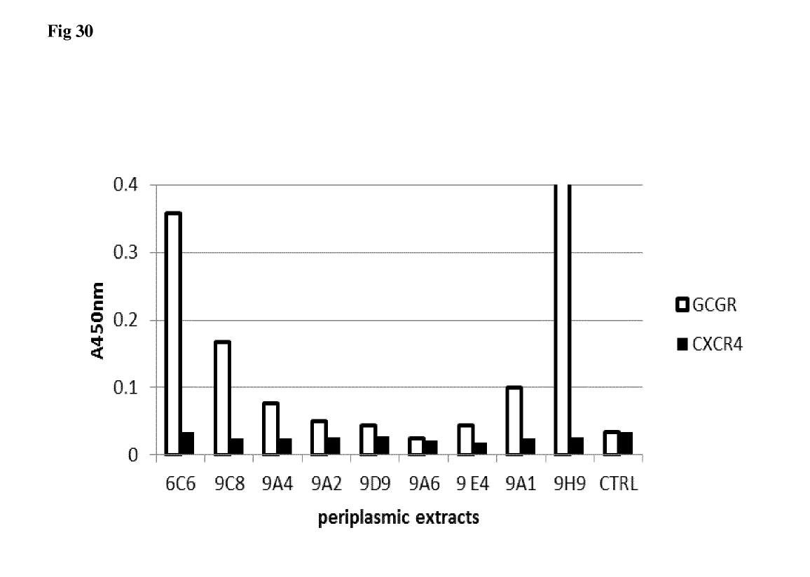

Those new families were tested on ELISA for binding to GCGR lipoparticles. There was only one family (9C8, 9A4), which bound to the GCGR lipoparticle only (see FIG. 30).

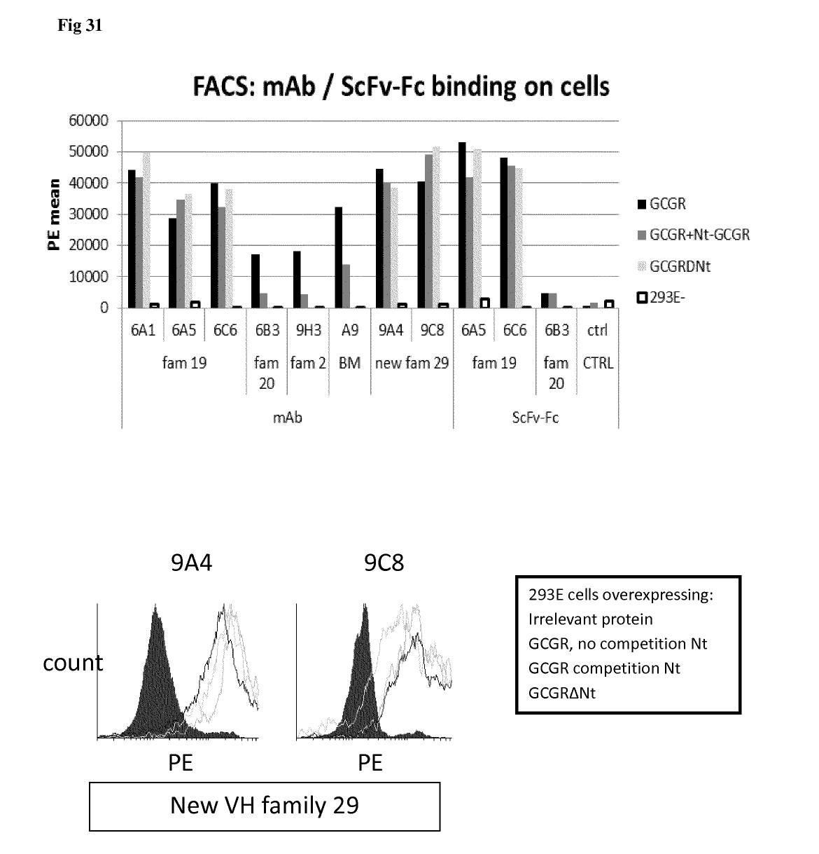

To confirm the binding of 9A4 and 9C8 to the ECD of GCGR, mAbs were produced for both clones. FIG. 31 demonstrates the binding of 9A4 and 9C8 to the ECL regions of GCGR as both mAb show binding to GCGRANT and GCGR in competition with Nt-GCGR-Fc. In conclusion, by counter-selection on the N-terminal domain of GCGR it was possible to identify a second family (family 29) of clones binding to the ECL regions of GCGR.

### Example 19

### Construction of scFv-Fc Fusions

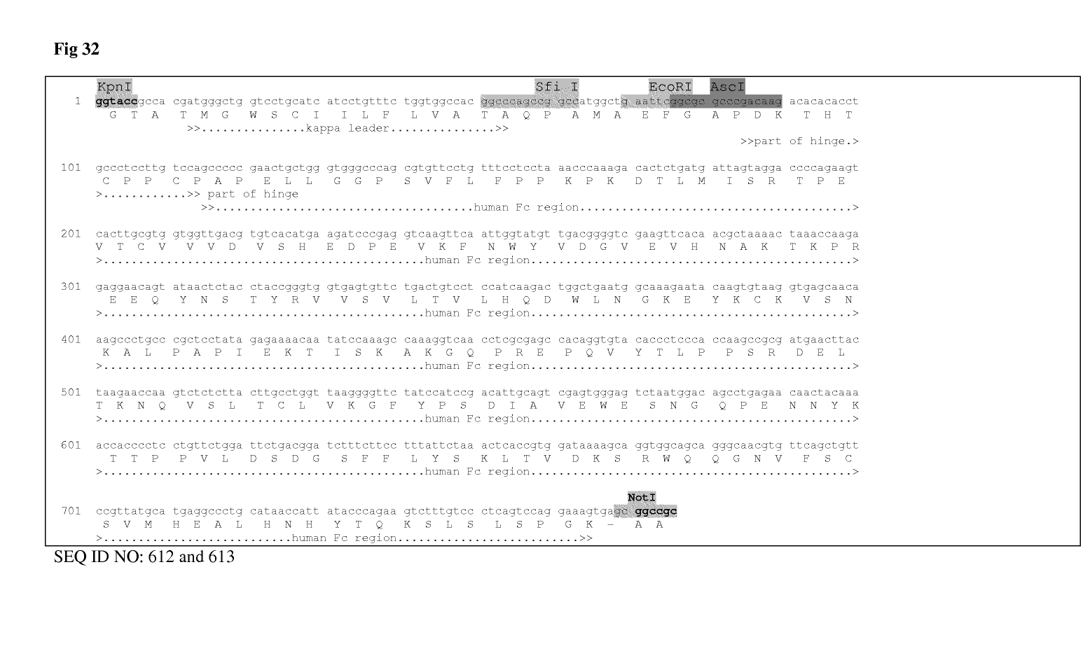

In the scFv format the VH and VL/VK are linked which means that they easily can be cloned from the phagemid vector into a mammalian expression vector for the production of a scFv-Fc fusion. The vector map of the scFv-Fc vector is depicted in FIG. 32 (SEQ ID NOs: 612 and 613). In the scFv-Fc molecule, single chain Fv (scFv) antibody fragments are fused to the IgG1 Fc (Hinge-CH2CH3) part to obtain bivalent IgG-like properties. To test this scFv-Fc approach scFv derived clones 6B3 (Nt-GCGR binder), and 6A5 and 6C6 (ECL binders) were recloned as scFv-Fc and tested for binding on GCGR-overexpressing cells by FACS (see FIG. 30). ScFv-Fc 6A5 and 6C6 both clearly bound to GCGR and GCGRΔNt on cells. ScFv-Fc 6B3 did not bind, but there were some production problems with this clone.

ScFv Fc fusion proteins could be easy tools for screening, since polyclonal phage outputs can be recloned to the scFv-Fc vector and transformed in TOP10. For up to 24 single colonies, plasmid DNA isolations can be done to generate DNA for small scale scFv-Fc in HEK293E cells.

### Example 20

### Nav1.7 Antibodies Identified by scFv Library Screening

scFv libraries were generated using cDNA isolated from libraries previously generated using material obtained from llamas immunized with DNA encoding human Nav1.7 (see Examples 5 and 6). scFv libraries were generated as described in Example 15 and these libraries were screened using recombinant Nav1.7 loop-llFc chimeras as described above. An additional loop was also used for screening, loop “B2” using the construct shown below (SEQ ID NO: 616):

GVELFLADVEGGGSIEGRDMDPHGGCTCPQCPAPELPGGPSVFVFPPKPK
DVLSISGRPEVTCVVVDVGKEDPEVNFNWYIDGVEVRTANTKPKEEQFNS
TYRVVSVLPIQHQDWLTGKEFKCKVNNKALPAPIERTISKAKGQTREPQV
YTLAPHREELAKDTVSVTCLVKGFYPADINVEWQRNGQPESEGTYANTPP
QLDNDGTYFLYSKLSVGKNTWQRGETLTCVVMHEALHNHYTQKSISQSPG
K

(Letters in italics: Nav1.7; Letters in bold: linker sequences; Underlined sequence: llama Fc)

Three specific clones were identified that bind to loop A3: clones 28A8, 28H3 and 39E1. Six specific clones were identified that bind to loop B1-C1-D1: 29A8, 40B3, 41A4, 41E4, 41F3 and 41F6. Five specific clones were identified that bind to loop B2: clones 43H2, 45B5, 45D5, 45D6 and 45G1. The sequences are shown in Tables 26-28 below.

TABLE 26
Framework regions and CDR sequences for VH domains
of Nav1.7 antibodies raised by DNA immunization and
identified by scFv screening

| | | SEQ | | SEQ | | SEQ | | SEQ |
|---|---|---|---|---|---|---|---|---|
| | | ID | | ID | | ID | | ID |
| Clone | FR1 | NO. | CDR1 | NO. | FR2 | NO. | CDR2 | NO. |
| 28A8 | evqlvesgggla | 442 | sfwmy | 452 | wvrqtpgk | 466 | tvdaagantyy | 472 |
| | qpggslrlscras | | | | glewvs | | pdtvkg | |
| | gfafg | | | | | | | |
| 28H3 | evqlvqpgaelr | 443 | ryyid | 453 | wvrqapg | 111 | ridpedggtkv | 473 |
| | npgasvkvsck | | | | qglewmg | | arkfqg | |
| | asgytft | | | | | | | |
| 29A8 | evqlvqpgaelr | 443 | ssyid | 454 | wvrqvpg | 467 | rmdpedgttk | 474 |
| | npgasvkvsck | | | | qgfewmg | | papkfqg | |
| | asgytft | | | | | | | |
| 39E1 | evqlvqpghel | 444 | nhyih | 455 | wvrqapg | 111 | ridpedggvky | 475 |
| | knpgasvkvsc | | | | qglewmg | | aqrfrg | |
| | kasgyift | | | | | | | |
| 40B3 | evqlvqpgaelr | 443 | ssyin | 456 | wlrqapgq | 468 | ridpedgrtksr | 476 |
| | npgasvkvsck | | | | gpewmg | | qnfqg | |
| | asgytft | | | | | | | |
| 41A4 | evqlvqpgaelr | 443 | kyyid | 457 | wvrqapg | 111 | lidpedgatkya | 477 |
| | npgasvkvsck | | | | qglewmg | | qkfqg | |
| | asgytft | | | | | | | |
| 41E4 | evqlvqpgtelr | 445 | nyfid | 458 | wvrqapg | 469 | sidpedggtqt | 478 |
| | npgssvkvsck | | | | | | | |
| | asgyifs | | | | qglewvg | | aqkfqg | |
| 41F3 | evqlvqpgtelr | 446 | gyyid | 459 | wvrqrpg | 470 | ridpedgdtky | 479 |
| | npgasvkvsck | | | | eglewmg | | aqkfqg | |
| | tsgytft | | | | | | | |
| 41F6 | evqlvesggglv | 99 | tyama | 460 | wvrqapg | 471 | finkygdgyads | 480 |
| | qpggslrlscaa | | | | kgpewvs | | vkg | |
| | sgftfs | | | | | | | |
| 43H2 | evqlqesggalv | 447 | rswmy | 461 | wvrqapg | 14 | amnpgggsty | 481 |
| | qpggslrlscaa | | | | kglewvs | | yadsvkg | |
| | sgftfs | | | | | | | |
| 45B5 | qvqlqesgpglv | 448 | lnggyws | 462 | wirqppgk | 15 | aiaytgstyisps | 482 |
| | kpsqtlslactvs | | | | glewmg | | lks | |
| | ggsit | | | | | | | |
| 45D5 | elqlvesggglv | 449 | dyams | 463 | wvrqapg | 14 | gttrnggrtyya | 483 |
| | qpggslrlscaa | | | | kglewvs | | esmkg | |
| | sgftfd | | | | | | | |
| 45D6 | qvqlvesggglv | 450 | sydms | 464 | wvrqapg | 471 | ainksggstyya | 484 |
| | qpggslrlscaa | | | | kgpewvs | | dsvkg | |
| | sgftfg | | | | | | | |
| 45G1 | Elqlvesggglv | 451 | nyamt | 465 | wvrqapg | 14 | ainsgggstyyg | 485 |
| | epggslrlscaa | | | | kglewvs | | davkg | |
| | sgftfs | | | | | | | |

| | | SEQ | | SEQ | | SEQ |
|---|---|---|---|---|---|---|
| | | ID | | ID | | ID |
| Clone | FR3 | NO. | CDR3 | NO. | FR4 | NO. |
| 28A8 | rftisrdnakntlylrmnslk | 486 | dgayddpair | 500 | wgqgtlvt | 513 |
| | pedtaryycvt | | gkiplas | | vss | |
| 28H3 | rvtftadtsastvylsltnlrsd | 487 | rgrgs | 501 | wgqgtrvt | 514 |
| | dtgvyycat | | | | vss | |
| 29A8 | rvtftadtsahtaymelttlr | 488 | ddglgalny | 502 | wghgtqvt | 515 |
| | vddtavyfcai | | | | vss | |
| 39E1 | rvtftvdtstttayvelhslrt | 489 | wsddddy | 503 | wgqgtqvt | 516 |
| | ddtgvyycaf | | | | vss | |
| 40B3 | rvtftadtststayvelrnlrs | 490 | gapdeyay | 504 | wgqgtqvt | 516 |
| | edtavyycva | | | | vss | |
| 41A4 | rvtfttdtststayvelsslrse | 491 | fpry | 505 | wgqgtlvt | 513 |
| | dtavyycli | | | | vss | |
| 41E4 | rviftvdtststayvevsslrp | 492 | pwny | 506 | wgqgtqvt | 516 |
| | edtgvyycvi | | | | vss | |
| 41F3 | gvtitadtstttaylelsslrse | 493 | gvdy | 147 | wgqgtqvt | 516 |
| | dtavyycal | | | | vss | |
| 41F6 | rftisrdnakntltlqmnslk | 494 | srgdhddydy | 507 | wgqgtqvt | 516 |
| | pedtavywcak | | | | vss | |
| 43H2 | rftisrdnakntlylqmnslk | 495 | agmst | 508 | wgkgtivt | 517 |
| | pedtalyycar | | | | vss | |
| 45B5 | rtsisrdasenqftlqlsavtp | 496 | hhydsnwyy | 509 | wgkgtivt | 517 |
| | edsavyycar | | ygmdy | | vss | |
| 45D5 | rftisrdndkntlylqmnsp | 497 | sdyalgstfss | 510 | wgqgtqvt | 516 |
| | ksedtgvyycak | | | | vss | |
| 45D6 | rftisrdnakntlylqmnslk | 498 | mygsswsy | 511 | wgqgtqvt | 516 |
| | pedtavyycat | | | | vss | |
| 45G1 | rftisrdnakntlylqinslkp | 499 | vgglgyvdy | 512 | wgqgtqvt | 516 |
| | edtavyycak | | | | vss | |

TABLE 27
Framework regions and CDR sequences for VL
domains of Nav1.7 antibodies raised by DNA
immunization and identified by scFv screening

| | | | | SEQ | | SEQ | | SEQ |
|---|---|---|---|---|---|---|---|---|
| | | SEQ ID | | ID | | ID | | ID |
| Clone | FR1 | NO. | CDR1 | NO. | FR2 | NO. | CDR2 | NO. |
| 28A8 | divmtqspssv | 518 | kssqsvvw | 530 | wyqqrpg | 539 | yastqqs | 547 |
| | tasvgekvtitc | | asnlktyln | | qpprlliy | | | |
| 28H3 | eivltqspssvta | 519 | kssqsvlys | 54 | wyqqrlgq | 61 | wastres | 68 |
| | sagekvtinc | | snqknyla | | sprlliy | | | |
| 29A8 | dvvltqtpgsls | 520 | kasqslvht | 531 | wllqkpgq | 177 | qvsnrgs | 187 |
| | vvpgesasisc | | dgktyly | | spqrliy | | | |
| 39E1 | sseltqpsavsv | 521 | rgdsleryg | 532 | wyqqkpg | 540 | gddirps | 548 |
| | slgqtaritc | | an | | qarvqviy | | | |
| 40B3 | eivltqtpgslsv | 522 | kasqslvhs | 167 | wllqkpgq | 177 | qvsnrgs | 187 |
| | vpgesasisc | | dgktyly | | spqrliy | | | |
| 41A4 | dlvltqipgslsv | 523 | kasqslvqt | 533 | wllqkpgq | 541 | qvsnrvs | 549 |
| | vpgesasisc | | dgktyls | | rpqlliy | | | |
| 41E4 | atmltqspgsls | 156 | kasqslvht | 165 | wllqkpgq | 541 | qvsnrgs | 187 |
| | vvpgesasisc | | dgktyls | | rpqlliy | | | |
| 41F3 | atmltqspgsls | 156 | kasqslvht | 534 | wllqkpgq | 542 | qvsnrns | 550 |
| | vvpgesasisc | | dgntyls | | rpqllit | | | |
| 41F6 | syeltqssavsv | 524 | qggslgsyg | 535 | wyqqkpg | 543 | gdnsrps | 551 |
| | slgqtaritc | | an | | qapvlliy | | | |
| 43H2 | nfmltqlpsvsg | 525 | tgsssnigg | 536 | wfqqlpgt | 544 | gnsnras | 552 |
| | spgqkvtisc | | gysvq | | ppklliy | | | |
| 45B5 | syeltqppsvse | 526 | agtssdvg | 52 | wyqqlpg | 59 | evnkras | 553 |
| | spgktvtisc | | ygnyvs | | mapkllly | | | |
| 45D5 | ssaltqppsvsg | 527 | agtssdvg | 52 | wyqqlpg | 545 | evnkras | 553 |
| | spgktvtisc | | ygnyvs | | mapslliy | | | |
| 45D6 | hsavtqppsvs | 528 | agtssdvg | 537 | wyqqlpg | 546 | gvnkras | 554 |
| | gspgktvtisc | | ygdyvs | | mapkllis | | | |
| 45G1 | nfmltqppsvs | 529 | ggedirtkn | 538 | wyqqkpg | 178 | kdserps | 555 |
| | valrqtakitc | | vh | | qapvlviy | | | |

| | | SEQ | | SEQ | | SEQ |
|---|---|---|---|---|---|---|
| | | ID | | ID | | ID |
| Clone | FR3 | NO. | CDR3 | NO. | FR4 | NO. |
| 28A8 | gipdrfsgsgsttdftltissv | 69 | qqvyrtpwt | 563 | fgqgtrleik | 577 |
| | qpedaavyyc | | | | | |
| 28H3 | gvpdrfsgsgsttdftltisnf | 556 | qqayktplt | 564 | fgqgtkveln | 578 |
| | qpedaavyyc | | | | | |
| 29A8 | gvpdrftgsgsgtdftlkisg | 199 | aqatyslys | 565 | fgsgtrleik | 85 |
| | vkaedagvyyc | | | | | |
| 39E1 | giperfsgsrlgdtatltisga | 557 | qssdssgyknd | 566 | fssrthltvlg | 579 |
| | qaedeadyyc | | | | | |
| 40B3 | gvpdrftgsgsgtdftlkisg | 199 | aqatyypyt | 567 | fgsgtrleik | 85 |
| | vkaedagvyyc | | | | | |
| 41A4 | gvpdrftgsgsgtdftlkisg | 558 | aqvtyaas | 568 | fgsgtrlemk | 580 |
| | vnaedagvyyc | | | | | |
| 41E4 | gvpdrftgsgsgtdftlkisg | 199 | aqaiyypps | 569 | fgsgtrleik | 85 |
| | vkaedagvyyc | | | | | |
| 41F3 | gvpnrftgsgsgtdftlkisg | 559 | aqvtrypvt | 570 | fgggtrleik | 581 |
| | vkpedaavyyc | | | | | |
| 41F6 | giperfsgsksggtatltisga | 560 | qsfdysgnav | 571 | fgggthltvlg | 86 |
| | qaedeadyyc | | | | | |
| 43H2 | gvpdrfsgsrsgssasltitgl | 561 | sswddslsgh | 572 | fgggtkltvlg | 87 |
| | qaedeadyyc | | pv | | | |
| 45B5 | giadrfsgsksgntasltisgl | 74 | asyrssvnvv | 573 | fgggthltvlg | 86 |
| | qsedeadyyc | | | | | |
| 45D5 | giadrfsgsksgntasltisgl | 74 | asyrsannav | 574 | fgggthltilg | 582 |
| | qsedeadyyc | | | | | |
| 45D6 | giadrfsgsksgntasltisgl | 74 | asykysnnvv | 575 | fgggthltvlg | 86 |
| | qsedeadyyc | | | | | |
| 45G1 | giperfsgsssggtatltisgv | 562 | qsvdssniv | 576 | fgggtkltvlg | 87 |
| | qaedeadyyc | | | | | |

TABLE 28
Variable Domain Sequences for Nav1.7 antibodies raised by DNA Immunization and identified by scFv screening

| | | SEQ | | SEQ |
|---|---|---|---|---|
| | | ID | | ID |
| Clone | VH | NO. | VL | NO. |
| 28A8 | evqlvesggglaqpggslrlscrasgfafgsfwmywvrqtpgkglewvstvdaagan | 583 | divmtqspssvtasvgekvtitckssqsvvwasnlktylnwyqqrpgqpprlliyyastqqsgip | 597 |
| | tyypdtvkgrftisrdnakntlylrmnslkpedtaryycvtdgayddpairgkiplasw | | drfsgsgsttdftltissvqpedaavyycqqvyrtpwtfgqgtrleik | |
| | gqgtivtvss | | | |
| 28H3 | evqlvqpgaelrnpgasvkvsckasgytftryyidwvrqapgqglewmgridpedg | 584 | eivltqspssvtasagekvtinckssqsvlyssnqknylawyqqrlgqsprlliywastresgvpdr | 598 |
| | gtkvarkfqgrvtftadtsastvylsltnlrsddtgvyycatrgrgswgqgtrvtvss | | fsgsgsttdftltisnfqpedaavyycqqayktpltfgqgtkveln | |
| 29A8 | evqlvqpgaelrnpgasvkvsckasgytftssyidwvrqvpgqgfewmgrmdpe | 585 | dvvltqtpgslsvvpgesasisckasqslvhtdgktylywllqkpgqspqrliyqvsnrgsgvpdrf | 599 |
| | dgttkpapkfqgrvtftadtsahtaymelttlrvddtavyfcaiddglgalnywghgt | | tgsgsgtdftlkisgvkaedagvyycaqatyslysfgsgtrleik | |
| | qvtvss | | | |
| 39E1 | evqlvqpghelknpgasvkvsckasgyiftnhyihwvrqapgqglewmgridped | 586 | sseltqpsavsvslgqtaritcrgdsleryganwyqqkpgqarvqviygddirpsgiperfsgsrlg | 600 |
| | ggvkyaqrfrgrvtftvdtstttayvelhslrtddtgvyycafwsddddywgqgtqvt | | dtatltisgaqaedeadyycqssdssgykndfssrthltvlg | |
| | vss | | | |
| 40B3 | evqlvqpgaelrnpgasvkvsckasgytftssyinwlrqapgqgpewmgridpedg | 587 | eivltqtpgslsvvpgesasisckasqslvhsdgktylywllqkpgqspqrliyqvsnrgsgvpdrf | 601 |
| | rtksrqnfqgrvtftadtststayvelrnlrsedtavyycvagapdeyaywgqgtqvt | | tgsgsgtdftlkisgvkaedagvyycaqatyypytfgsgtrleik | |
| | vss | | | |
| 41A4 | evqlvqpgaelrnpgasvkvsckasgytftkyyidwvrqapgqglewmglidpedg | 588 | dlvltqipgslsvvpgesasisckasqslvqtdgktylswllqkpgqrpqlliyqvsnrvsgvpdrft | 602 |
| | atkyaqkfqgrvtfttdtststayvelsslrsedtavyyclifprywgqgtlvtvss | | gsgsgtdftlkisgvnaedagvyycaqvtyaasfgsgtrlemk | |
| 41E4 | evqlvqpgtelrnpgssvkvsckasgyifsnyfidwvrqapgqglewvgsidpedgg | 589 | atmltqspgslsvvpgesasisckasqslvhtdgktylswllqkpgqrpqlliyqvsnrgsgvpdrf | 603 |
| | tqtaqkfqgrviftvdtststayvevsslrpedtgvyycvipwnywgqgtqvtvss | | tgsgsgtdftlkisgvkaedagvyycaqaiyyppsfgsgtrleik | |
| 41F3 | evqlvqpgtelrnpgasvkvscktsgytftgyyidwvrqrpgeglewmgridpedg | 590 | atmltqspgslsvvpgesasisckasqslvhtdgntylswllqkpgqrpqllitqvsnrnsgvpnrf | 604 |
| | dtkyaqkfqggvtitadtstttaylelsslrsedtavyycalgvdywgqgtqvtvss | | tgsgsgtdftlkisgvkpedaavyycaqvtrypvtfgggtrleik | |
| 41F6 | evqlvesggglvqpggslrlscaasgftfstyamawvrqapgkgpewvsfinkygdg | 591 | syeltqssavsvslgqtaritcqggslgsyganwyqqkpgqapvlliygdnsrpsgiperfsgsks | 605 |
| | yadsvkgrftisrdnakntltlqmnslkpedtavywcaksrgdhddydywgqgtqv | | ggtatltisgaqaedeadyycqsfdysgnavfgggthltvlg | |
| | tvss | | | |
| 43H2 | evqlqesggalvqpggslrlscaasgftfsrswmywvrqapgkglewvsamnpgg | 592 | nfmltqlpsvsgspgqkvtisctgsssnigggysvqwfqqlpgtppklliygnsnrasgvpdrfsg | 606 |
| | gstyyadsvkgrftisrdnakntlylqmnslkpedtalyycaragmstwgkgtlvtvss | | srsgssasltitglqaedeadyycsswddslsghpvfgggtkltvlg | |
| 45B5 | qvqlqesgpglvkpsqtlslactvsggsitlnggywswirqppgkglewmgaiaytgs | 593 | syeltqppsysespgktvtiscagtssdvgygnyvswyqqlpgmapklllyevnkrasgiadrfs | 607 |
| | tyispslksrtsisrdasenqftlqlsavtpedsavyycarhhydsnwyyygmdywg | | gsksgntasltisglqsedeadyycasyrssvnvvfgggthltvlg | |
| | kgtlvtvss | | | |
| 45D5 | elqlvesggglvqpggslrlscaasgftfddyamswvrqapgkglewvsgttrnggrt | 594 | ssaltqppsvsgspgktvtiscagtssdvgygnyvswyqqlpgmapslliyevnkrasgiadrfs | 608 |
| | yyaesmkgrftisrdndkntlylqmnspksedtgvyycaksdyalgstfsswgqgtq | | gsksgntasltisglqsedeadyycasyrsannavfgggthltilg | |
| | vtvss | | | |
| 45D6 | qvqlvesggglvqpggslrlscaasgftfgsydmswvrqapgkgpewvsainksggs | 595 | hsavtqppsvsgspgktvtiscagtssdvgygdyvswyqqlpgmapkllisgvnkrasgiadrfs | 609 |
| | tyyadsvkgrftisrdnakntlylqmnslkpedtavyycatmygsswsywgqgtqv | | gsksgntasltisglqsedeadyycasykysnnvvfgggthltvlg | |
| | tvss | | | |
| 45G1 | elqlvesggglvepggslrlscaasgftfsnyamtwvrqapgkglewvsainsgggst | 596 | nfmltqppsvsvalrqtakitcggedirtknvhwyqqkpgqapvlviykdserpsgiperfsgss | 610 |
| | yygdavkgrftisrdnakntlylqinslkpedtavyycakvgglgyvdywgqgtqvtv | | sggtatltisgvqaedeadyycqsvdssnivfgggtkltvlg | |
| | ss | | | |

1. An antibody or antigen binding fragment thereof, which binds to a target protein, comprising at least one complementarity determining region (CDR) wherein:
(a) the target protein has a length of at least 1115 amino acids and the at least one CDR is derived from an antibody raised by immunization of a host animal with a DNA molecule comprising an open reading frame of at least 3345 nucleotides encoding:
(i) the full-length target protein;
(ii) a protein having at least 70% identity to the full-length target protein; or
(iii) a fragment of the full-length target protein;
(b) the target protein has at least 8 transmembrane domains and the at least one CDR is derived from an antibody raised by immunization of a host animal with a DNA molecule comprising an open reading frame encoding:
(i) the full-length target protein;
(ii) a protein having at least 70% identity to the full-length target protein; or
(iii) a fragment of the full-length target protein having at least 8 transmembrane domains;
or
(c) the target protein is naturally encoded by a nucleotide sequence which is difficult to replicate in a common E. coli strain and the at least one CDR is derived from an antibody raised by immunization of a host animal with a DNA molecule comprising an open reading frame, which is difficult to replicate in a common E. coli strain, encoding:
(i) the full-length target protein;
(ii) a protein having at least 70% identity to the full-length target protein; or
(iii) a fragment of the full-length target protein.

2. The antibody or antigen binding fragment of claim 1 wherein the common E. coli strain has a genotype: TetrD(mcrA)183 D(mcrCB-hsdSMR-mrr)173 endA1 supE44 thi-1 recA1 gyrA96 relA1 lac Hte [F′ proAB lacIqZDM15 Tn10 (Tetr) Amy Camr] (XL-10 Gold, Stratagene).

3. The antibody or antigen binding fragment of claim 1 or claim 2 wherein the protein is an ion channel.

4. The antibody or antigen binding fragment of any of claims 1-3 comprising at least one heavy chain variable domain (VH) and wherein the VH domain CDR1 and/or the VH domain CDR2 and/or the VH domain CDR3 is derived from said antibody raised by DNA immunization of a host animal.

5. The antibody or antigen binding fragment of any of claims 1-3 comprising at least one heavy chain variable domain (VH) derived from said antibody raised by DNA immunization of a host animal.

6. The antibody or antigen binding fragment of any of claims 1-5 comprising at least one light chain variable domain (VL) and wherein the VL domain CDR1 and/or the VL domain CDR2 and/or the VL domain CDR3 is derived from said antibody raised by DNA immunization of a host animal.

7. The antibody or antigen binding fragment of any of claims 1-5 comprising at least one light chain variable domain (VL) derived from said antibody raised by DNA immunization of a host animal.

8. The antibody or antigen binding fragment of any of claims 1-7 wherein the target protein is a voltage-gated ion channel selected from the Naᵥ and Caᵥ ion channels.

9. The antibody or antigen binding fragment of claim 8 wherein the ion channel is voltage-gated sodium ion channel selected from Nav1.1 to Nav1.9.

10. The antibody or antigen binding fragment of claim 9 wherein the ion channel is human Nav1.7.

11. An antibody or antigen binding fragment thereof, which binds to the voltage-gated sodium channel human Nav1.7, said antibody or antigen binding fragment comprising at least one heavy chain variable domain (VH) and at least one light chain variable domain (VL), wherein said VH and VL domain, when tested as a mAb, exhibit an affinity of binding for an extracellular region of human Nav1.7, which is at least 10-fold higher than a reference antibody selected from the group consisting of: UCB_932; UCB_983; and UCB_1066, as described in US2011/0135662.

12. An antibody or antigen binding fragment thereof, which binds to the voltage-gated sodium channel human Nav1.7, said antibody or antigen binding fragment comprising at least one heavy chain variable domain (VH) and at least one light chain variable domain (VL), wherein said VH and VL domain, when tested as a mAb, exhibit an affinity of binding for an extracellular region of human Nav1.7, (EC₅₀ as measured by ELISA) of less than 2 nM.

13. An antibody or antigen binding fragment thereof, which binds to the voltage-gated sodium channel human Nav1.7, said antibody or antigen binding fragment comprising at least one heavy chain variable domain (VH) and at least one light chain variable domain (VL), wherein said VH and VL domain, when tested as a mAb, exhibit an off-rate (k_{off} measured by Biacore) for an extracellular region of human Nav1.7 of less than 5×10⁻³ s⁻¹.

14. The antibody or antigen binding fragment of any of claims 11-13 which binds to an extracellular loop of human Nav1.7 selected from:
A3 as represented by SEQ ID NO: 262 (corresponding to amino acid residues 269 to 319 of SEQ ID NO:260);
B1 as represented by SEQ ID NO: 263 (corresponding to amino acid residues 753 to 765 of SEQ ID NO:260);
C1 as represented by SEQ ID NO: 264 (corresponding to amino acid residues 1201 to 1214 of SEQ ID NO:260);
D1 as represented by SEQ ID NO: 265 (corresponding to amino acid residues 1523 to 1536 of SEQ ID NO:260);
C3 as represented by SEQ ID NO: 266 (corresponding to amino acid residues 1334 to 1420 of SEQ ID NO:260); and
B2 as represented by SEQ ID NO: 611 (corresponding to amino acid residues 809 to 818 of SEQ ID NO:260).

15. The antibody or antigen binding fragment of claim 14 which exhibits selective binding to one extracellular loop of human Nav1.7 selected from:
A3 as represented by SEQ ID NO: 262 (corresponding to amino acid residues 269 to 319 of SEQ ID NO:260);
B1 as represented by SEQ ID NO: 263 (corresponding to amino acid residues 753 to 765 of SEQ ID NO:260);
C1 as represented by SEQ ID NO: 264 (corresponding to amino acid residues 1201 to 1214 of SEQ ID NO:260);
D1 as represented by SEQ ID NO: 265 (corresponding to amino acid residues 1523 to 1536 of SEQ ID NO:260);
C3 as represented by SEQ ID NO: 266 (corresponding to amino acid residues 1334 to 1420 of SEQ ID NO:260); and
B2 as represented by SEQ ID NO: 611 (corresponding to amino acid residues 809 to 818 of SEQ ID NO:260).

16. The antibody or antigen binding fragment of any of claims 11-15 which does not cross-react with human Nav1.5.

17. The antibody or antigen binding fragment of any of claims 11-16 which does not cross-react with human Nav1.2.

18. The antibody or antigen binding fragment of any of claims 11-17 which does not cross-react with any other voltage-gated ion channels.

19. The antibody or antigen binding fragment of any of claims 10-18 wherein the variable domain of the heavy chain comprises at least one complementarity determining region (CDR) selected from SEQ ID NOs: 8-13, 16-21, 28-34, 100-109, 119-129, 141-149, 289, 291, 293, 452-465, 472-485, 500-512 or sequence variants thereof, wherein the sequence variant comprises one, two or three amino acid substitutions in the recited sequence.

20. The antibody or antigen binding fragment of claim 19, comprising a combination of variable heavy chain CDR3 (HCDR3), variable heavy chain CDR2 (HCDR2) and variable heavy chain CDR1 (HCDR1) wherein the combination is selected from the group consisting of:
(i) HCDR3 comprising SEQ ID NO: 28; HCDR2 comprising SEQ ID NO: 16; HCDR1 comprising SEQ ID NO: 8;
(ii) HCDR3 comprising SEQ ID NO: 29; HCDR2 comprising SEQ ID NO: 16; HCDR1 comprising SEQ ID NO: 8;
(iii) HCDR3 comprising SEQ ID NO: 30; HCDR2 comprising SEQ ID NO: 17; HCDR1 comprising SEQ ID NO: 9;
(iv) HCDR3 comprising SEQ ID NO: 31; HCDR2 comprising SEQ ID NO: 18; HCDR1 comprising SEQ ID NO: 10;
(v) HCDR3 comprising SEQ ID NO: 32; HCDR2 comprising SEQ ID NO: 19; HCDR1 comprising SEQ ID NO: 11;
(vi) HCDR3 comprising SEQ ID NO: 33; HCDR2 comprising SEQ ID NO: 20; HCDR1 comprising SEQ ID NO: 12;
(vii) HCDR3 comprising SEQ ID NO: 34; HCDR2 comprising SEQ ID NO: 21; HCDR1 comprising SEQ ID NO: 13;
(viii) HCDR3 comprising SEQ ID NO: 141; HCDR2 comprising SEQ ID NO: 119; HCDR1 comprising SEQ ID NO: 100;
(ix) HCDR3 comprising SEQ ID NO: 142; HCDR2 comprising SEQ ID NO: 120; HCDR1 comprising SEQ ID NO: 101;
(x) HCDR3 comprising SEQ ID NO: 143; HCDR2 comprising SEQ ID NO: 121; HCDR1 comprising SEQ ID NO: 102;
(xi) HCDR3 comprising SEQ ID NO: 144; HCDR2 comprising SEQ ID NO: 122; HCDR1 comprising SEQ ID NO: 103;
(xii) HCDR3 comprising SEQ ID NO: 145; HCDR2 comprising SEQ ID NO: 123; HCDR1 comprising SEQ ID NO: 104;
(xiii) HCDR3 comprising SEQ ID NO: 146; HCDR2 comprising SEQ ID NO: 124; HCDR1 comprising SEQ ID NO: 105;
(xiv) HCDR3 comprising SEQ ID NO: 145; HCDR2 comprising SEQ ID NO: 125; HCDR1 comprising SEQ ID NO: 106;
(xv) HCDR3 comprising SEQ ID NO: 147; HCDR2 comprising SEQ ID NO: 126; HCDR1 comprising SEQ ID NO: 107;
(xvi) HCDR3 comprising SEQ ID NO: 148; HCDR2 comprising SEQ ID NO: 127; HCDR1 comprising SEQ ID NO: 108;
(xvii) HCDR3 comprising SEQ ID NO: 149; HCDR2 comprising SEQ ID NO: 128; HCDR1 comprising SEQ ID NO: 109;
(xviii) HCDR3 comprising SEQ ID NO: 149; HCDR2 comprising SEQ ID NO: 129; HCDR1 comprising SEQ ID NO: 109;
(xix) HCDR3 comprising SEQ ID NO: 293; HCDR2 comprising SEQ ID NO: 291; HCDR1 comprising SEQ ID NO: 289;
(xx) HCDR3 comprising SEQ ID NO: 500; HCDR2 comprising SEQ ID NO: 472; HCDR1 comprising SEQ ID NO: 452;
(xxi) HCDR3 comprising SEQ ID NO: 501; HCDR2 comprising SEQ ID NO: 473; HCDR1 comprising SEQ ID NO: 453;
(xxii) HCDR3 comprising SEQ ID NO: 502; HCDR2 comprising SEQ ID NO: 474; HCDR1 comprising SEQ ID NO: 454;
(xxiii) HCDR3 comprising SEQ ID NO: 503; HCDR2 comprising SEQ ID NO: 475; HCDR1 comprising SEQ ID NO: 455;
(xxiv) HCDR3 comprising SEQ ID NO: 504; HCDR2 comprising SEQ ID NO: 476; HCDR1 comprising SEQ ID NO: 456;
(xxv) HCDR3 comprising SEQ ID NO: 505; HCDR2 comprising SEQ ID NO: 477; HCDR1 comprising SEQ ID NO: 457;
(xxvi) HCDR3 comprising SEQ ID NO: 506; HCDR2 comprising SEQ ID NO: 478; HCDR1 comprising SEQ ID NO: 458;
(xxvii) HCDR3 comprising SEQ ID NO: 147; HCDR2 comprising SEQ ID NO: 479; HCDR1 comprising SEQ ID NO: 459;
(xxviii) HCDR3 comprising SEQ ID NO: 507; HCDR2 comprising SEQ ID NO: 480; HCDR1 comprising SEQ ID NO: 460;
(xxix) HCDR3 comprising SEQ ID NO: 508; HCDR2 comprising SEQ ID NO: 481; HCDR1 comprising SEQ ID NO: 461;
(xxx) HCDR3 comprising SEQ ID NO: 509; HCDR2 comprising SEQ ID NO: 482; HCDR1 comprising SEQ ID NO: 462;
(xxxi) HCDR3 comprising SEQ ID NO: 510; HCDR2 comprising SEQ ID NO: 483; HCDR1 comprising SEQ ID NO: 463;
(xxxii) HCDR3 comprising SEQ ID NO: 511; HCDR2 comprising SEQ ID NO: 484; HCDR1 comprising SEQ ID NO: 464; and
(xxxiii) HCDR3 comprising SEQ ID NO: 512; HCDR2 comprising SEQ ID NO: 485; HCDR1 comprising SEQ ID NO: 465.

21. The antibody or antigen-binding fragment of any of claims 10-22 wherein the variable domain of the light chain comprises at least one complementarity determining region (CDR) selected from SEQ ID NOs: 46-54, 62-68, 77-84, 161-172, 181-192, 204-215, 296, 298, 300, 530-538, 547-555, 563-576 or sequence variants thereof, wherein the sequence variant comprises one, two or three amino acid substitutions in the recited sequence.

22. The antibody or antigen-binding fragment of claim 21, comprising a combination of variable light chain CDR3 (LCDR3), variable light chain CDR2 (LCDR2) and variable light chain CDR1 (LCDR1) wherein the combination is selected from the group consisting of:
(i) LCDR3 comprising SEQ ID NO: 77; LCDR2 comprising SEQ ID NO: 62; LCDR1 comprising SEQ ID NO: 46;
(ii) LCDR3 comprising SEQ ID NO: 78; LCDR2 comprising SEQ ID NO: 63; LCDR1 comprising SEQ ID NO: 47;
(iii) LCDR3 comprising SEQ ID NO: 78; LCDR2 comprising SEQ ID NO: 63; LCDR1 comprising SEQ ID NO: 48;
(iv) LCDR3 comprising SEQ ID NO: 79; LCDR2 comprising SEQ ID NO: 64; LCDR1 comprising SEQ ID NO: 49;
(v) LCDR3 comprising SEQ ID NO: 80; LCDR2 comprising SEQ ID NO: 64; LCDR1 comprising SEQ ID NO: 50;
(vi) LCDR3 comprising SEQ ID NO: 81; LCDR2 comprising SEQ ID NO: 65; LCDR1 comprising SEQ ID NO: 51;
(vii) LCDR3 comprising SEQ ID NO: 82; LCDR2 comprising SEQ ID NO: 66; LCDR1 comprising SEQ ID NO: 52;
(viii) LCDR3 comprising SEQ ID NO: 83; LCDR2 comprising SEQ ID NO: 67; LCDR1 comprising SEQ ID NO: 53;
(ix) LCDR3 comprising SEQ ID NO: 84; LCDR2 comprising SEQ ID NO: 68; LCDR1 comprising SEQ ID NO: 54;
(x) LCDR3 comprising SEQ ID NO: 204; LCDR2 comprising SEQ ID NO: 181; LCDR1 comprising SEQ ID NO: 161;
(xi) LCDR3 comprising SEQ ID NO: 205; LCDR2 comprising SEQ ID NO: 182; LCDR1 comprising SEQ ID NO: 162;
(xii) LCDR3 comprising SEQ ID NO: 206; LCDR2 comprising SEQ ID NO: 183; LCDR1 comprising SEQ ID NO: 163;
(xiii) LCDR3 comprising SEQ ID NO: 207; LCDR2 comprising SEQ ID NO: 184; LCDR1 comprising SEQ ID NO: 164;
(xiv) LCDR3 comprising SEQ ID NO: 208; LCDR2 comprising SEQ ID NO: 185; LCDR1 comprising SEQ ID NO: 165;
(xv) LCDR3 comprising SEQ ID NO: 209; LCDR2 comprising SEQ ID NO: 186; LCDR1 comprising SEQ ID NO: 166;
(xvi) LCDR3 comprising SEQ ID NO: 210; LCDR2 comprising SEQ ID NO: 187; LCDR1 comprising SEQ ID NO: 167;
(xvii) LCDR3 comprising SEQ ID NO: 211; LCDR2 comprising SEQ ID NO: 188; LCDR1 comprising SEQ ID NO: 168;
(xviii) LCDR3 comprising SEQ ID NO: 212; LCDR2 comprising SEQ ID NO: 189; LCDR1 comprising SEQ ID NO: 169;
(xix) LCDR3 comprising SEQ ID NO: 213; LCDR2 comprising SEQ ID NO: 190; LCDR1 comprising SEQ ID NO: 170;
(xx) LCDR3 comprising SEQ ID NO: 214; LCDR2 comprising SEQ ID NO: 191; LCDR1 comprising SEQ ID NO: 171;
(xxi) LCDR3 comprising SEQ ID NO: 215; LCDR2 comprising SEQ ID NO: 192; LCDR1 comprising SEQ ID NO: 172;
(xxii) LCDR3 comprising SEQ ID NO: 300; LCDR2 comprising SEQ ID NO: 298; LCDR1 comprising SEQ ID NO: 296;
(xxiii) LCDR3 comprising SEQ ID NO: 563; LCDR2 comprising SEQ ID NO: 547; LCDR1 comprising SEQ ID NO: 530;
(xxiv) LCDR3 comprising SEQ ID NO: 564; LCDR2 comprising SEQ ID NO: 68; LCDR1 comprising SEQ ID NO: 54;
(xxv) LCDR3 comprising SEQ ID NO: 565; LCDR2 comprising SEQ ID NO: 187; LCDR1 comprising SEQ ID NO: 531;
(xxvi) LCDR3 comprising SEQ ID NO: 566; LCDR2 comprising SEQ ID NO: 548; LCDR1 comprising SEQ ID NO: 532;
(xxvii) LCDR3 comprising SEQ ID NO: 567; LCDR2 comprising SEQ ID NO: 187; LCDR1 comprising SEQ ID NO: 167;
(xxviii) LCDR3 comprising SEQ ID NO: 568; LCDR2 comprising SEQ ID NO: 549; LCDR1 comprising SEQ ID NO: 533;
(xxix) LCDR3 comprising SEQ ID NO: 569; LCDR2 comprising SEQ ID NO: 187; LCDR1 comprising SEQ ID NO: 165;
(xxx) LCDR3 comprising SEQ ID NO: 570; LCDR2 comprising SEQ ID NO: 550; LCDR1 comprising SEQ ID NO: 534;
(xxxi) LCDR3 comprising SEQ ID NO: 571; LCDR2 comprising SEQ ID NO: 551; LCDR1 comprising SEQ ID NO: 535;
(xxxii) LCDR3 comprising SEQ ID NO: 572; LCDR2 comprising SEQ ID NO: 552; LCDR1 comprising SEQ ID NO: 536;
(xxxiii) LCDR3 comprising SEQ ID NO: 573; LCDR2 comprising SEQ ID NO: 553; LCDR1 comprising SEQ ID NO: 52;
(xxxiv) LCDR3 comprising SEQ ID NO: 574; LCDR2 comprising SEQ ID NO: 553; LCDR1 comprising SEQ ID NO: 52;
(xxxv) LCDR3 comprising SEQ ID NO: 575; LCDR2 comprising SEQ ID NO: 554; LCDR1 comprising SEQ ID NO: 537; and
(xx) LCDR3 comprising SEQ ID NO: 576; LCDR2 comprising SEQ ID NO: 555; LCDR1 comprising SEQ ID NO: 538.

23. The antibody or antigen-binding fragment of any of claims 12-19, comprising a combination of variable heavy chain CDR3 (HCDR3), variable heavy chain CDR2 (HCDR2) and variable heavy chain CDR1 (HCDR1), variable light chain CDR3 (LCDR3), variable light chain CDR2 (LCDR2) and variable light chain CDR1 (LCDR1) wherein the combination is selected from the group consisting of:
(i) HCDR3 comprising SEQ ID NO: 28; HCDR2 comprising SEQ ID NO: 16; HCDR1 comprising SEQ ID NO: 8; LCDR3 comprising SEQ ID NO: 77; LCDR2 comprising SEQ ID NO: 62; LCDR1 comprising SEQ ID NO: 46;
(ii) HCDR3 comprising SEQ ID NO: 29; HCDR2 comprising SEQ ID NO: 16; HCDR1 comprising SEQ ID NO: 8; LCDR3 comprising SEQ ID NO: 78; LCDR2 comprising SEQ ID NO: 63; LCDR1 comprising SEQ ID NO: 47;
(iii) HCDR3 comprising SEQ ID NO: 29; HCDR2 comprising SEQ ID NO: 16; HCDR1 comprising SEQ ID NO: 8; LCDR3 comprising SEQ ID NO: 78; LCDR2 comprising SEQ ID NO: 63; LCDR1 comprising SEQ ID NO: 48;
(iv) HCDR3 comprising SEQ ID NO: 30; HCDR2 comprising SEQ ID NO: 17; HCDR1 comprising SEQ ID NO: 9; LCDR3 comprising SEQ ID NO: 79; LCDR2 comprising SEQ ID NO: 64; LCDR1 comprising SEQ ID NO: 49;
(v) HCDR3 comprising SEQ ID NO: 30; HCDR2 comprising SEQ ID NO: 17; HCDR1 comprising SEQ ID NO: 9; LCDR3 comprising SEQ ID NO: 80; LCDR2 comprising SEQ ID NO: 64; LCDR1 comprising SEQ ID NO: 50;
(vi) HCDR3 comprising SEQ ID NO: 31; HCDR2 comprising SEQ ID NO: 18; HCDR1 comprising SEQ ID NO: 10; LCDR3 comprising SEQ ID NO: 81; LCDR2 comprising SEQ ID NO: 65; LCDR1 comprising SEQ ID NO: 51;
(vii) HCDR3 comprising SEQ ID NO: 32; HCDR2 comprising SEQ ID NO: 19; HCDR1 comprising SEQ ID NO: 11; LCDR3 comprising SEQ ID NO: 82; LCDR2 comprising SEQ ID NO: 66; LCDR1 comprising SEQ ID NO: 52;
(viii) HCDR3 comprising SEQ ID NO: 33; HCDR2 comprising SEQ ID NO: 20; HCDR1 comprising SEQ ID NO: 12; LCDR3 comprising SEQ ID NO: 83; LCDR2 comprising SEQ ID NO: 67; LCDR1 comprising SEQ ID NO: 53;
(ix) HCDR3 comprising SEQ ID NO: 34; HCDR2 comprising SEQ ID NO: 21; HCDR1 comprising SEQ ID NO: 13; LCDR3 comprising SEQ ID NO: 84; LCDR2 comprising SEQ ID NO: 68; LCDR1 comprising SEQ ID NO: 54;
(x) HCDR3 comprising SEQ ID NO: 141; HCDR2 comprising SEQ ID NO: 119; HCDR1 comprising SEQ ID NO: 100; LCDR3 comprising SEQ ID NO: 204; LCDR2 comprising SEQ ID NO: 181; LCDR1 comprising SEQ ID NO: 161;
(xi) HCDR3 comprising SEQ ID NO: 142; HCDR2 comprising SEQ ID NO: 120; HCDR1 comprising SEQ ID NO: 101; LCDR3 comprising SEQ ID NO: 205; LCDR2 comprising SEQ ID NO: 182; LCDR1 comprising SEQ ID NO: 162;
(xii) HCDR3 comprising SEQ ID NO: 143; HCDR2 comprising SEQ ID NO: 121; HCDR1 comprising SEQ ID NO: 102; LCDR3 comprising SEQ ID NO: 206; LCDR2 comprising SEQ ID NO: 183; LCDR1 comprising SEQ ID NO: 163;
(xiii) HCDR3 comprising SEQ ID NO: 144; HCDR2 comprising SEQ ID NO: 122; HCDR1 comprising SEQ ID NO: 103; LCDR3 comprising SEQ ID NO: 207; LCDR2 comprising SEQ ID NO: 184; LCDR1 comprising SEQ ID NO: 164;
(xiv) HCDR3 comprising SEQ ID NO: 145; HCDR2 comprising SEQ ID NO: 123; HCDR1 comprising SEQ ID NO: 104; LCDR3 comprising SEQ ID NO: 208; LCDR2 comprising SEQ ID NO: 185; LCDR1 comprising SEQ ID NO: 165;
(xv) HCDR3 comprising SEQ ID NO: 146; HCDR2 comprising SEQ ID NO: 124; HCDR1 comprising SEQ ID NO: 105; LCDR3 comprising SEQ ID NO: 209; LCDR2 comprising SEQ ID NO: 186; LCDR1 comprising SEQ ID NO: 166;
(xvi) HCDR3 comprising SEQ ID NO: 145; HCDR2 comprising SEQ ID NO: 125; HCDR1 comprising SEQ ID NO: 106; LCDR3 comprising SEQ ID NO: 210; LCDR2 comprising SEQ ID NO: 187; LCDR1 comprising SEQ ID NO: 167;
(xvii) HCDR3 comprising SEQ ID NO: 145; HCDR2 comprising SEQ ID NO: 125; HCDR1 comprising SEQ ID NO: 106; LCDR3 comprising SEQ ID NO: 211; LCDR2 comprising SEQ ID NO: 188; LCDR1 comprising SEQ ID NO: 168;
(xviii) HCDR3 comprising SEQ ID NO: 147; HCDR2 comprising SEQ ID NO: 126; HCDR1 comprising SEQ ID NO: 107; LCDR3 comprising SEQ ID NO: 212; LCDR2 comprising SEQ ID NO: 189; LCDR1 comprising SEQ ID NO: 169;
(xix) HCDR3 comprising SEQ ID NO: 148; HCDR2 comprising SEQ ID NO: 127; HCDR1 comprising SEQ ID NO: 108; LCDR3 comprising SEQ ID NO: 213; LCDR2 comprising SEQ ID NO: 190; LCDR1 comprising SEQ ID NO: 170;
(xx) HCDR3 comprising SEQ ID NO: 149; HCDR2 comprising SEQ ID NO: 128; HCDR1 comprising SEQ ID NO: 109; LCDR3 comprising SEQ ID NO: 214; LCDR2 comprising SEQ ID NO: 191; LCDR1 comprising SEQ ID NO: 171;
(xi) HCDR3 comprising SEQ ID NO: 149; HCDR2 comprising SEQ ID NO: 129; HCDR1 comprising SEQ ID NO: 109; LCDR3 comprising SEQ ID NO: 215; LCDR2 comprising SEQ ID NO: 192; LCDR1 comprising SEQ ID NO: 172;
(xii) HCDR3 comprising SEQ ID NO: 293; HCDR2 comprising SEQ ID NO: 291; HCDR1 comprising SEQ ID NO: 289; LCDR3 comprising SEQ ID NO: 300; LCDR2 comprising SEQ ID NO: 298; LCDR1 comprising SEQ ID NO: 296;
(xxiii) HCDR3 comprising SEQ ID NO: 500; HCDR2 comprising SEQ ID NO: 472; HCDR1 comprising SEQ ID NO: 452; LCDR3 comprising SEQ ID NO: 563; LCDR2 comprising SEQ ID NO: 547; LCDR1 comprising SEQ ID NO: 530;
(xxiv) HCDR3 comprising SEQ ID NO: 501; HCDR2 comprising SEQ ID NO: 473; HCDR1 comprising SEQ ID NO: 453; LCDR3 comprising SEQ ID NO: 564; LCDR2 comprising SEQ ID NO: 68; LCDR1 comprising SEQ ID NO: 54;
(xxv) HCDR3 comprising SEQ ID NO: 502; HCDR2 comprising SEQ ID NO: 474; HCDR1 comprising SEQ ID NO: 454; LCDR3 comprising SEQ ID NO: 565; LCDR2 comprising SEQ ID NO: 187; LCDR1 comprising SEQ ID NO: 531;
(xxvi) HCDR3 comprising SEQ ID NO: 503; HCDR2 comprising SEQ ID NO: 475; HCDR1 comprising SEQ ID NO: 455; LCDR3 comprising SEQ ID NO: 566; LCDR2 comprising SEQ ID NO: 548; LCDR1 comprising SEQ ID NO: 532;
(xxvii) HCDR3 comprising SEQ ID NO: 504; HCDR2 comprising SEQ ID NO: 476; HCDR1 comprising SEQ ID NO: 456; LCDR3 comprising SEQ ID NO: 567; LCDR2 comprising SEQ ID NO: 187; LCDR1 comprising SEQ ID NO: 167;
(xxviii) HCDR3 comprising SEQ ID NO: 505; HCDR2 comprising SEQ ID NO: 477; HCDR1 comprising SEQ ID NO: 457; LCDR3 comprising SEQ ID NO: 568; LCDR2 comprising SEQ ID NO: 549; LCDR1 comprising SEQ ID NO: 533;
(xxix) HCDR3 comprising SEQ ID NO: 506; HCDR2 comprising SEQ ID NO: 478; HCDR1 comprising SEQ ID NO: 458; LCDR3 comprising SEQ ID NO: 569; LCDR2 comprising SEQ ID NO: 187; LCDR1 comprising SEQ ID NO: 165;
(xxx) HCDR3 comprising SEQ ID NO: 147; HCDR2 comprising SEQ ID NO: 479; HCDR1 comprising SEQ ID NO: 459; LCDR3 comprising SEQ ID NO: 570; LCDR2 comprising SEQ ID NO: 550; LCDR1 comprising SEQ ID NO: 534;
(xxxi) HCDR3 comprising SEQ ID NO: 507; HCDR2 comprising SEQ ID NO: 480; HCDR1 comprising SEQ ID NO: 460; LCDR3 comprising SEQ ID NO: 571; LCDR2 comprising SEQ ID NO: 551; LCDR1 comprising SEQ ID NO: 535;
(xxxii) HCDR3 comprising SEQ ID NO: 508; HCDR2 comprising SEQ ID NO: 481; HCDR1 comprising SEQ ID NO: 461; LCDR3 comprising SEQ ID NO: 572; LCDR2 comprising SEQ ID NO: 552; LCDR1 comprising SEQ ID NO: 536;
(xxxiii) HCDR3 comprising SEQ ID NO: 509; HCDR2 comprising SEQ ID NO: 482; HCDR1 comprising SEQ ID NO: 462; LCDR3 comprising SEQ ID NO: 573; LCDR2 comprising SEQ ID NO: 553; LCDR1 comprising SEQ ID NO: 52;
(xxxiv) HCDR3 comprising SEQ ID NO: 510; HCDR2 comprising SEQ ID NO: 483; HCDR1 comprising SEQ ID NO: 463; LCDR3 comprising SEQ ID NO: 574; LCDR2 comprising SEQ ID NO: 553; LCDR1 comprising SEQ ID NO: 52;
(xxxv) HCDR3 comprising SEQ ID NO: 511; HCDR2 comprising SEQ ID NO: 484; HCDR1 comprising SEQ ID NO: 464; LCDR3 comprising SEQ ID NO: 575; LCDR2 comprising SEQ ID NO: 554; LCDR1 comprising SEQ ID NO: 537; and
(xxxvi) HCDR3 comprising SEQ ID NO: 512; HCDR2 comprising SEQ ID NO: 485; HCDR1 comprising SEQ ID NO: 465; LCDR3 comprising SEQ ID NO: 576; LCDR2 comprising SEQ ID NO: 555; LCDR1 comprising SEQ ID NO: 538.

24. The antibody or antigen-binding fragment of any of claims 11-18 wherein the heavy chain variable domain has an amino acid sequence selected from the group consisting of the amino acid sequences of SEQ ID NOs: 218-226, 236-247, 302, 583-596 and amino acid sequences exhibiting at least 90%, 95%, 97%, 98% or 99% sequence identity to one of the recited sequences.

25. The antibody or antigen-binding fragment of any of claims 11-18 wherein the light chain variable domain has an amino acid sequence selected from the group consisting of the amino acid sequences of SEQ ID NO: 227-235, 248-259, 303, 597-610 and amino acid sequences exhibiting at least 90%, 95%, 97%, 98% or 99% sequence identity to one of the recited sequences.

26. The antibody or antigen-binding fragment of any of claims 11-18 wherein the antibody or antigen binding fragment thereof comprises a combination of a heavy chain variable domain (VH) and a light chain variable domain (VL) selected from the following:
(i) VH comprising the amino acid sequence of SEQ ID NO: 218 and VL comprising the amino acid sequence of SEQ ID NO: 227;
(ii) VH comprising the amino acid sequence of SEQ ID NO: 219 and VL comprising the amino acid sequence of SEQ ID NO: 228;
(iii) VH comprising the amino acid sequence of SEQ ID NO: 220 and VL comprising the amino acid sequence of SEQ ID NO: 229;
(iv) VH comprising the amino acid sequence of SEQ ID NO: 221 and VL comprising the amino acid sequence of SEQ ID NO: 230;
(v) VH comprising the amino acid sequence of SEQ ID NO: 222 and VL comprising the amino acid sequence of SEQ ID NO: 231;
(vi) VH comprising the amino acid sequence of SEQ ID NO: 223 and VL comprising the amino acid sequence of SEQ ID NO: 232;
(vii) VH comprising the amino acid sequence of SEQ ID NO: 224 and VL comprising the amino acid sequence of SEQ ID NO: 233;
(viii) VH comprising the amino acid sequence of SEQ ID NO: 225 and VL comprising the amino acid sequence of SEQ ID NO: 234;
(ix) VH comprising the amino acid sequence of SEQ ID NO: 226 and VL comprising the amino acid sequence of SEQ ID NO: 235;
(x) VH comprising the amino acid sequence of SEQ ID NO: 236 and VL comprising the amino acid sequence of SEQ ID NO: 248;
(xi) VH comprising the amino acid sequence of SEQ ID NO: 237 and VL comprising the amino acid sequence of SEQ ID NO: 249;
(xii) VH comprising the amino acid sequence of SEQ ID NO: 238 and VL comprising the amino acid sequence of SEQ ID NO: 250;
(xiii) VH comprising the amino acid sequence of SEQ ID NO: 239 and VL comprising the amino acid sequence of SEQ ID NO: 251;
(xiv) VH comprising the amino acid sequence of SEQ ID NO: 240 and VL comprising the amino acid sequence of SEQ ID NO: 252;
(xv) VH comprising the amino acid sequence of SEQ ID NO: 241 and VL comprising the amino acid sequence of SEQ ID NO: 253;
(xvi) VH comprising the amino acid sequence of SEQ ID NO: 242 and VL comprising the amino acid sequence of SEQ ID NO: 254;
(xvii) VH comprising the amino acid sequence of SEQ ID NO: 243 and VL comprising the amino acid sequence of SEQ ID NO: 255;
(xviii) VH comprising the amino acid sequence of SEQ ID NO: 244 and VL comprising the amino acid sequence of SEQ ID NO: 256;
(xix) VH comprising the amino acid sequence of SEQ ID NO: 245 and VL comprising the amino acid sequence of SEQ ID NO: 257;
(xx) VH comprising the amino acid sequence of SEQ ID NO: 246 and VL comprising the amino acid sequence of SEQ ID NO: 258;
(xxi) VH comprising the amino acid sequence of SEQ ID NO: 247 and VL comprising the amino acid sequence of SEQ ID NO: 259;
(xxii) VH comprising the amino acid sequence of SEQ ID NO: 302 and VL comprising the amino acid sequence of SEQ ID NO: 303;
(xxiii) VH comprising the amino acid sequence of SEQ ID NO: 583 and VL comprising the amino acid sequence of SEQ ID NO: 597;
(xxiv) VH comprising the amino acid sequence of SEQ ID NO: 584 and VL comprising the amino acid sequence of SEQ ID NO: 598;
(xxv) VH comprising the amino acid sequence of SEQ ID NO: 585 and VL comprising the amino acid sequence of SEQ ID NO: 599;
(xxvi) VH comprising the amino acid sequence of SEQ ID NO: 586 and VL comprising the amino acid sequence of SEQ ID NO: 600;
(xxvii) VH comprising the amino acid sequence of SEQ ID NO: 587 and VL comprising the amino acid sequence of SEQ ID NO: 601;
(xxviii) VH comprising the amino acid sequence of SEQ ID NO: 588 and VL comprising the amino acid sequence of SEQ ID NO: 602;
(xxix) VH comprising the amino acid sequence of SEQ ID NO: 589 and VL comprising the amino acid sequence of SEQ ID NO: 603;
(xxx) VH comprising the amino acid sequence of SEQ ID NO: 590 and VL comprising the amino acid sequence of SEQ ID NO: 604;
(xxxi) VH comprising the amino acid sequence of SEQ ID NO: 591 and VL comprising the amino acid sequence of SEQ ID NO: 605;
(xxxii) VH comprising the amino acid sequence of SEQ ID NO: 592 and VL comprising the amino acid sequence of SEQ ID NO: 606;
(xxxiii) VH comprising the amino acid sequence of SEQ ID NO: 593 and VL comprising the amino acid sequence of SEQ ID NO: 607;
(xxxiv) VH comprising the amino acid sequence of SEQ ID NO: 594 and VL comprising the amino acid sequence of SEQ ID NO: 608;
(xxxv) VH comprising the amino acid sequence of SEQ ID NO: 595 and VL comprising the amino acid sequence of SEQ ID NO: 609; and
(xxxvi) VH comprising the amino acid sequence of SEQ ID NO: 596 and VL comprising the amino acid sequence of SEQ ID NO: 610.

27. The antibody or antigen-binding fragment of any of claims 11-18 comprising:
(i) a VH domain comprising or consisting of an amino acid sequence selected from the group consisting of: the amino acid sequence of SEQ ID NO: 237, germlined variants and affinity variants thereof and amino acid sequences at least 90%, 95%, 97%, 98% or 99% identical thereto and a VL domain comprising or consisting of an amino acid sequence selected from the group consisting of: the amino acid sequence of SEQ ID NO: 249, germlined variants and affinity variants thereof and amino acid sequences at least 90%, 95%, 97%, 98% or 99% identical thereto; or
(ii) a VH domain comprising or consisting of an amino acid sequence selected from the group consisting of: the amino acid sequence of SEQ ID NO: 236, germlined variants and affinity variants thereof and amino acid sequences at least 90%, 95%, 97%, 98% or 99% identical thereto and a VL domain comprising or consisting of an amino acid sequence selected from the group consisting of: the amino acid sequence of SEQ ID NO: 248, germlined variants and affinity variants thereof and amino acid sequences at least 90%, 95%, 97%, 98% or 99% identical thereto; or
(iii) a VH domain comprising or consisting of an amino acid sequence selected from the group consisting of: the amino acid sequence of SEQ ID NO: 240, germlined variants and affinity variants thereof and amino acid sequences at least 90%, 95%, 97%, 98% or 99% identical thereto and a VL domain comprising or consisting of an amino acid sequence selected from the group consisting of: the amino acid sequence of SEQ ID NO: 252, germlined variants and affinity variants thereof and amino acid sequences at least 90%, 95%, 97%, 98% or 99% identical thereto.

28. The antibody or antigen-binding fragment of any of claims 11-27 wherein the VH and/or VL domains or one or more of the CDRs are derived from an animal of the Camelidae family.

29. The antibody or antigen-binding fragment of claim 28 wherein the animal is a llama (lama glama).

30. The antibody or antigen-binding fragment of any of claims 11-29 wherein at least one complementarity determining region (CDR) is derived from an antibody raised by immunization of a host animal with a DNA molecule comprising an open reading frame of at least 3345 nucleotides encoding:
(i) the full-length human Nav1.7 protein;
(ii) a protein having at least 70% identity to the full-length human Nav1.7 protein; or
(iii) a fragment of the full-length human Nav1.7 protein.

31. The antibody or antigen-binding fragment of claim 30 wherein the VH domain, the VL domain or the CDRs of the VH domain and/or VL domain are derived from an antibody raised by DNA immunization of a host animal.

32. The antibody of any of claims 1-31 wherein the antibody has no effector function.

33. An isolated polynucleotide which encodes the antibody or antigen binding fragment of any of claims 1-32.

34. An expression vector comprising the polynucleotide of claim 33 operably linked to regulatory sequences which permit expression of the antibody or antigen binding fragment thereof in a host cell or cell-free expression system.

35. A host cell or cell-free expression system containing the expression vector of claim 34.

36. A method of producing a recombinant antibody or antigen binding fragment thereof which comprises culturing the host cell or cell free expression system of claim 35 under conditions which permit expression of the antibody or antigen binding fragment and recovering the expressed antibody or antigen binding fragment.

37. A pharmaceutical composition comprising an antibody or antigen binding fragment according to any of claims 12 to 32 and at least one pharmaceutically acceptable carrier or excipient.

38. An antibody or antigen binding fragment thereof according to any of claims 1 to 32 or a pharmaceutical composition according to claim 37 for use as a medicament.

39. An antibody or antigen binding fragment thereof according to any of claims 12 to 32 or a pharmaceutical composition according to claim 37 for use in the treatment or prophylaxis of a pathological disorder that is mediated by Nav1.7 or that is associated with an increased level of Nav1.7, or that is associated with an increase or decrease in Nav1.7 channel activity.

40. An antibody or antigen binding fragment thereof according to any of claims 12 to 32 or a pharmaceutical composition according to claim 37 for use in the treatment or prophylaxis of pain.

41. A method of raising an antibody which binds a target protein wherein
(a) the target protein has a length of at least 1115 amino acids, and the method comprises immunizing a host animal with a DNA molecule comprising an open reading frame of at least 3345 nucleotides encoding:
(i) the full-length target protein;
(ii) a protein having at least 70% identity to the full-length target protein; or
(iii) a fragment of the full-length target protein;
Or
(b) the target protein has at least 8 transmembrane domains and the method comprises immunizing a host animal with a DNA molecule comprising an open reading frame encoding:
(i) the full-length target protein;
(ii) a protein having at least 70% identity to the full-length target protein; or
(iii) a fragment of the full-length target protein having at least 8 transmembrane domains;
Or
(c) the target protein is naturally encoded by a nucleotide sequence which is difficult to replicate in a common E. coli strain and the method comprises immunizing a host animal with a DNA molecule comprising an open reading frame, which is difficult to replicate in a common E. coli strain, encoding:
(i) the full-length target protein;
(ii) a protein having at least 70% identity to the full-length target protein; or
(iii) a fragment of the full-length target protein.

42. The method of claim 41 wherein the common E. coli strain has a genotype: TetrD(mcrA)183 D(mcrCB-hsdSMR-mrr)173 endA1 supE44 thi-1 recA1 gyrA96 relA1 lac Hte [F′ proAB lacIqZDM15 Tn10 (Tetr) Amy Camr] (XL-10 Gold, Stratagene).

43. The method of claim 41 or claim 42 wherein the method further comprises the steps of:
separating or isolating antibodies from the immunized host animal; and
identifying antibodies having binding specificity for the target protein.

44. The method of claim 41 or claim 42 wherein the method further comprises the steps of:
isolating material from the host animal wherein the material is a source of antibody producing cells containing nucleotide sequences from which antibodies or antigen binding fragments thereof immunoreactive with the target protein can be identified; and optionally
constructing a library or collection of nucleotide sequences derived from the immunized host, wherein the nucleotide sequences encode a VH domain and/or a VL domain of a conventional antibody; and optionally
screening to identify antibodies or antigen binding fragments thereof immunoreactive with the target protein.

45. The method of claim 44 wherein the library or collection of nucleotide sequences comprises nucleotide sequences encoding single chain Fv fragments (scFv) and the screening is carried out to identify scFv fragments immunoreactive with the target protein.

46. The method of claim 44 wherein the library or collection of nucleotide sequences comprises nucleotide sequences encoding fusion polypeptides comprising single chain Fv fragments linked to the constant region of an IgG molecule.

47. The method of any of claims 44-46 wherein the method additionally comprises the preparation of a recombinant antibody or antigen binding fragment thereof by:
determining the nucleotide sequence encoding at least one hypervariable loop or CDR of the VH and/or the VL domain immunoreactive with the target protein; and
expressing an antibody or antigen binding fragment thereof which binds to said target protein, said antibody or antigen binding fragment thereof comprising a VH and a VL domain, wherein at least one hypervariable loop or CDR of the VH or VL domain has an amino acid sequence encoded by the nucleotide sequence determined in the preceding step.

48. The method of any of claims 44-46 wherein the method additionally comprises the preparation of a recombinant antibody or antigen binding fragment thereof by:
isolating nucleic acid encoding at least one hypervariable loop or CDR of the VH and/or the VL domain immunoreactive with the target antigen,
preparing a recombinant polynucleotide comprising a nucleotide sequence encoding hypervariable loop(s) or CDR(s) having amino acid sequence(s) identical or substantially identical to the hypervariable loop(s) or CDR(s) encoded by the nucleic acid isolated in the preceding step, which recombinant polynucleotide encodes an antibody or antigen binding fragment thereof comprising a VH domain and a VL domain that specifically binds to said target antigen, and
expressing said antibody or antigen binding fragment thereof from the recombinant polynucleotide of the preceding step.

49. The method of any of claims 41-48 wherein the protein is an ion channel.

50. The method of any of claims 41-48 wherein the host animal is selected from mouse, rat, rabbit, goat, hamster, chicken, monkey or an animal from the Camelidae family.

51. The method of claim 50 wherein the host animal is llama (lama glama).

52. The method of any of claims 41-51 wherein the host animal is an outbred animal.
